(19) United States
(12) Patent Application Publication
(10) Pub. No.: US 2024/0067673 A1
(43) Pub. Date: Feb. 29, 2024

(54) MODIFIED NUCLEOSIDE OR NUCLEOTIDE

(71) Applicants: BIG SHENZHEN, SHENZHEN (CN); BGI SHENZHEN GROUP HOLDINGS CO., LIMITED, SHENZHEN (CN)

(72) Inventors: Xun XU, SHENZHEN (CN); Bo TENG, SHENZHEN (CN); Wenwei ZHANG, SHENZHEN (CN); Ao CHEN, SHENZHEN (CN); Handong LI, SHENZHEN (CN); Shengyi YAN, SHENZHEN (CN); Shitian ZHUO, SHENZHEN (CN); Liang SHEN, SHENZHEN (CN); Yonghui ZHANG, SHENZHEN (CN); Nanfeng GAO, SHENZHEN (CN); Jie ZHAO, SHENZHEN (CN); Sha LIAO, SHENZHEN (CN)

(21) Appl. No.: 18/033,037

(22) PCT Filed: Oct. 21, 2021
(86) PCT No.: PCT/CN2021/125262

§ 371 Date: Apr. 20, 2023

(30) Foreign Application Priority Data
Oct. 21, 2020 (CN) 202011134947.3

(51) Int. Cl. C07H 19/20; C07H 19/10; C12Q 1/6869

(52) U.S. Cl. CPC C07H 19/20; C07H 19/10; C12Q 1/6869

(57) ABSTRACT

A modified nucleoside or nucleotide, the 3′-OH of the modified nucleoside or nucleotide being reversibly blocked; meanwhile, the present invention also relates to a kit comprising the nucleoside or nucleotide, and a sequencing method based on the nucleoside or nucleotide.

### TECHNICAL FIELD

The invention relates to the field of nucleic acid sequencing. In particular, the present invention relates to modified nucleoside or nucleotide, wherein the 3′-OH of the modified nucleoside or nucleotide is reversibly blocked.

### BACKGROUND

The emergence of NGS sequencing overcomes the disadvantages of Sanger's high cost and time requirements, and greatly promotes the application of gene sequencing technology. At present, NGS sequencing has been deeply applied in fields such as prenatal screening, tumor diagnosis, tumor treatment, animal and plant breeding, and promotes the progress of science and medicine.

Deoxy-ribonucleoside triphosphate (dNTP) analogues carrying reversible blocking groups are key raw materials in NGS sequencing. Due to the incorporation of reversible blocking group, the 3′—OH group in dNTP can be retained, which overcomes the shortcomings of Sanger sequencing and ensures the accuracy of base recognition. It can be said that deoxy-ribonucleoside triphosphate (dNTP) analogue with reversible blocking group is the most critical technology in NGS sequencing.

Currently, many dNTP compounds carrying reversible blocking groups have been reported. The reversible blocking of dNTP is mainly realized through two kinds of ideas. The first kind of idea bases on the direct introduction of reversible blocking groups into 3′-OH of dNTP, and the advantage of such modified dNTP is that the blocking of 3′-OH ensures the blocking efficiency in sequencing. The other kind of idea bases on blocking the polymerase by base modification, rather than blocking 3′-OH, and the advantage of such idea is that the blocking groups may be selected from a wider range, rather than limited to polymerase.

In general, the two kinds of reversible blocking ideas have their own advantages and disadvantages, but the method of introducing group blocking directly into 3′-OH shows more reliability and significantly higher blocking efficiency. Therefore, such scheme is mainly used in currently marketed NGS sequencing.

### SUMMARY OF THE INVENTION

Azidomethyl is a kind of highly effective 3′-OH reversible blocking group, possessing great advantages such as good stability, mild removal conditions and fast removal speed. The excision reaction of azido methyl on 3′-OH is essentially a cascade reaction, in that the azido group undergoes the Staudinger reaction under the action of phosphorous reagent to produce an intermediate with methylene amino group on 3′-OH, which then undergoes a rapid hydrolysis reaction to free the 3′-OH. During such cascade reaction, both Staudinger reaction and the hydrolysis reaction proceed very fast, so that the azidomethyl may be removed at very fast rate (as shown in FIG. 1).

Azido group is a special chemical group with three nitrogen atoms in its chemical structure, forming a conjugated structure and in the same plane. The three nitrogen atoms are not positioned in a straight line but arranged with certain angle. The conjugated structure formed by the three nitrogen atoms in an azido group may be presented in several resonance modes, which contributes to its stability.

Azido group is generally stable. However, due to the iterative structure of the three nitrogen atoms, azido group may react and quickly release nitrogen under the appropriate reaction conditions, and thus the azide group has a very active reactivity and is prone to explosive risk. It is precisely based on the characteristic of azide group that it is widely used in various click reactions.

Based on such rapid reactivity of azido group, the inventor has designed a class of 3′-OH blocking groups carrying azido group that can undergo cascade reaction. The 3′-OH blocking group undergoes the Staudinger reaction to release amine group, which attacks the ester/carbonate protection group on 3′-OH to free 3′-OH (as shown in FIG. 2). Similarly, those groups capable of releasing a nucleophilic atom under appropriate conditions are all applicable to such design, such as —S—SR, —OCOR, —OCONHR and the like.

The invention intends to develop a class of dNTP analogues carrying ester and carbonate group on 3′-OH, which are useful in NGS sequencing. Such dNTP analogues have the general structural formula shown in FIG. 3, with 3′-OH protected by reversible blocking group and a bulk structure comprising 2′-deoxyuridine triphosphate, 2′-deoxythymosine triphosphate, 2′-deoxycytidine triphosphate, 2′-deoxyadenosine triphosphate, 7-deaza-2′-deoxyadenosine triphosphate, 2′-deoxyguanosine triphosphate and 7-deaza-2′-deoxyguanosine triphosphate.

Therefore, in the first aspect of the invention, the present invention provides compound of formula (A) or a salt thereof,

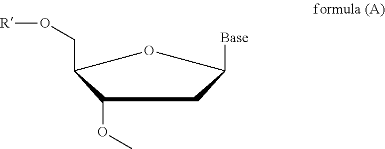

wherein:
R is a reversible blocking group, R is selected from

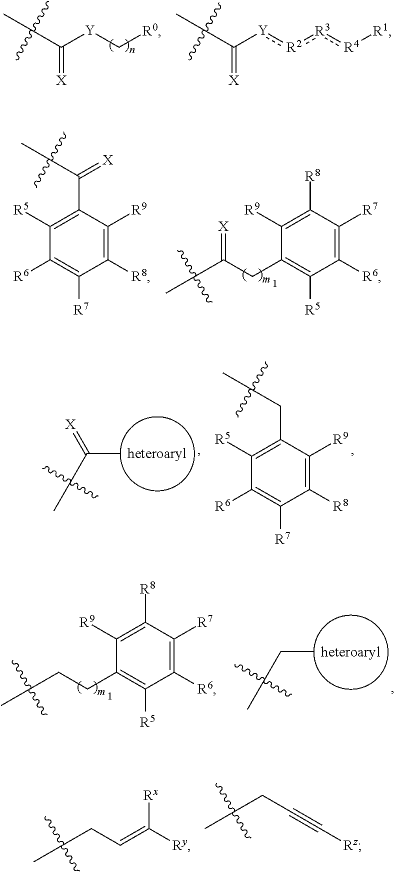

the heteroaryl is selected from the followings:

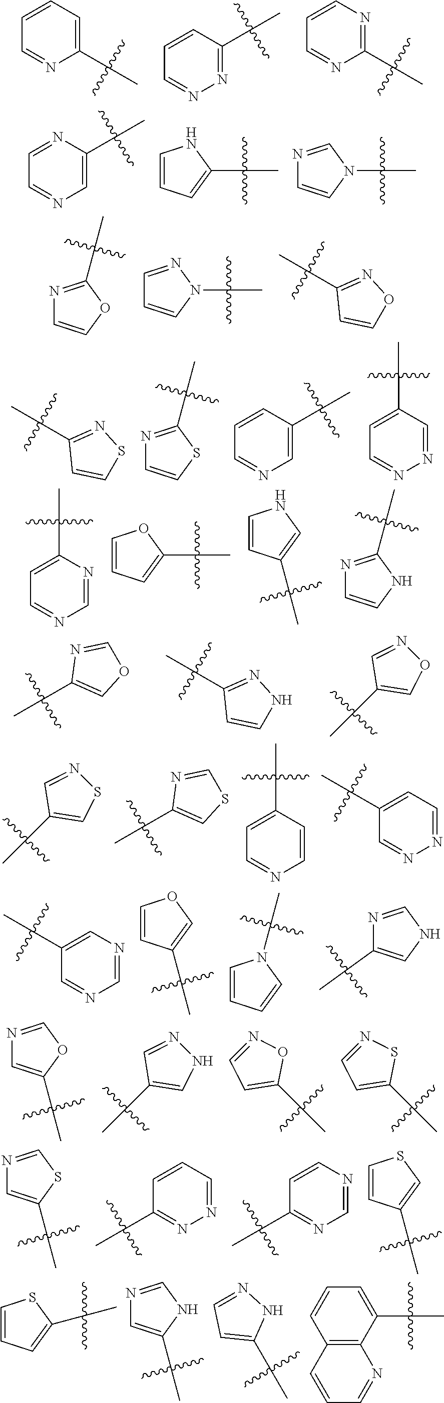

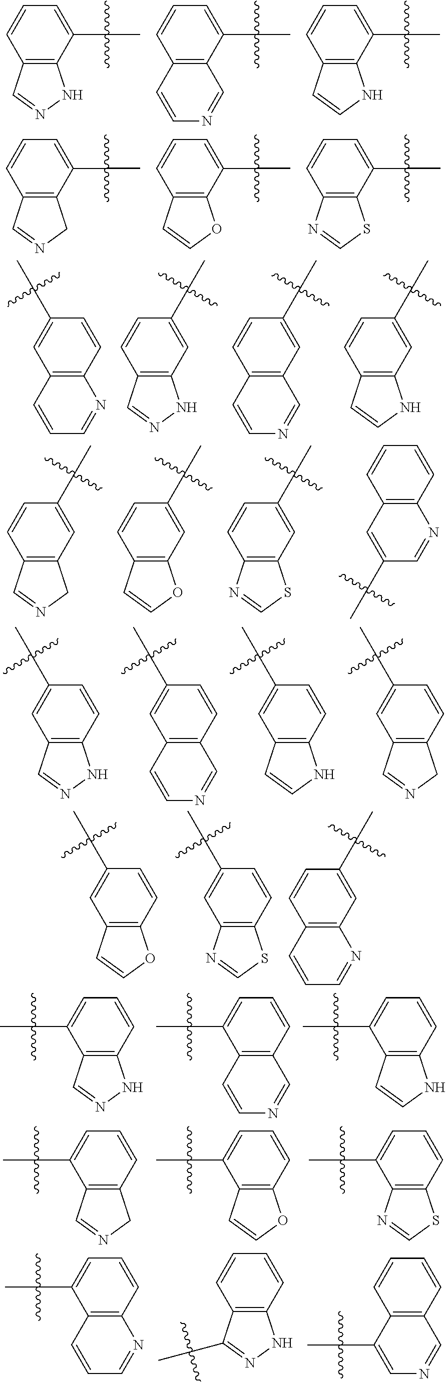

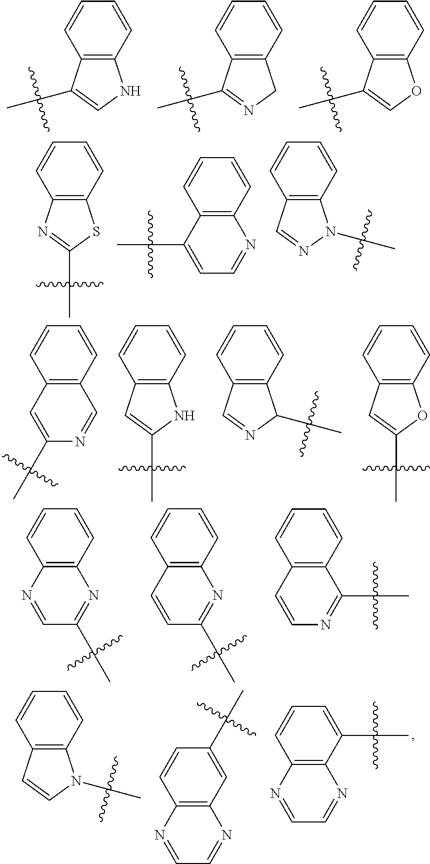

each heteroaryl is independently optionally substituted by one or more (e.g., 2, 3, 4, 5 or 6, preferably 2) R″s;
each X is independently selected from O, NH, S;
each Y is independently selected from a direct bond, O, NH, S, CH, CH₂, C(CH₃)₂;
R⁰ is selected from —N₃, —SS—C₁-C₆ alkyl (e.g., —SS-methyl, —SS-ethyl, —SS-isopropyl, —SS-tertbutyl), —ONH₂, —OCOR^{m}, —OCONHR^{m}, wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl);
R¹ is selected from —N₃, —SS—C₁-C₆ alkyl (e.g., —SS-methyl, —SS-ethyl, —SS-isopropyl, —SS-tertbutyl), —ONH₂, —OCOR^{m}, —OCONHR^{m}, wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl);
R², R³, and R⁴ are each independently selected from O, NH, S, CH, CH₂, C(CH₃)₂;
[symbol] represents a single bond [symbol] or a double bond [symbol] ;
each R″ is independently selected from H, —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy),

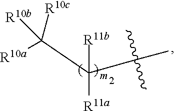

and is not H at the same time;
R⁵, R⁶, R⁷, R⁸, R⁹, R^{x}, R^{y}, and R^{z} are each independently selected from H, —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy),

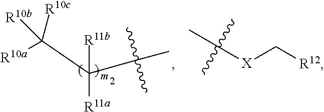

and R⁵, R⁶, R⁷, R⁸, and R⁹ are not H at the same time;
or, R⁵, R⁶, R⁷, R⁸, R⁹, R^{x}, R^{y}, and R^{z} are each independently selected from H, —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy), C₁-C₆ alkyl-C(═O)—NH₂—,

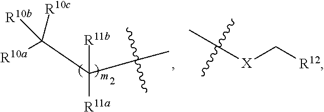

and R⁵, R⁶, R⁷, R⁸, and R⁹ are not H at the same time;

R^{10a}, R^{10b}, R^{10c}, R^{11a}, R^{11b} and R¹² are each independently selected from H, —N₃, —SS—C₁-C₆ alkyl (e.g., —SS-methyl, —SS-ethyl, —SS-isopropyl, —SS-tertbutyl or —SS-isobutyl), —ONH₂, —OCOR^{m}, —OCONHR^{m}, aliphatic alkyl (e.g., C₁-C₆ alkyl, particularly such as methyl (Me), ethyl (Et), isopropyl (iPr), tertbutyl (tBu)), aromatic alkyl (e.g., phenyl C₁-C₆ alkyl), cycloalkyl (e.g., C₃-C₆ cycloalkyl), wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl), and R^{10a}, R^{10b}, and R^{10c} are not H at the same time;
n is selected from 1, 2, 3, 4, and 5;
each m₁ is independently selected from 1, 2, 3, 4, 5, and 6;
each m₂ is independently selected from 0, 1, 2, 3, 4, 5, and 6;
R′ is selected from H, monophosphate group

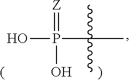

diphosphate group

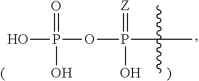

triphosphate group

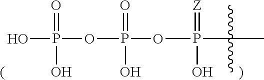

or tetraphosphate group

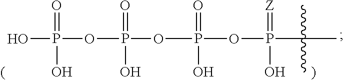

each Z is independently selected from O, S, BH;

Base is selected from a base, a deaza base or a tautomer thereof, for example, Base is selected from adenine, 7-deazaadenine, thymine, uracil, cytosine, guanine, 7-deazaguanine or a tautomer thereof.

In some embodiments, R is selected from

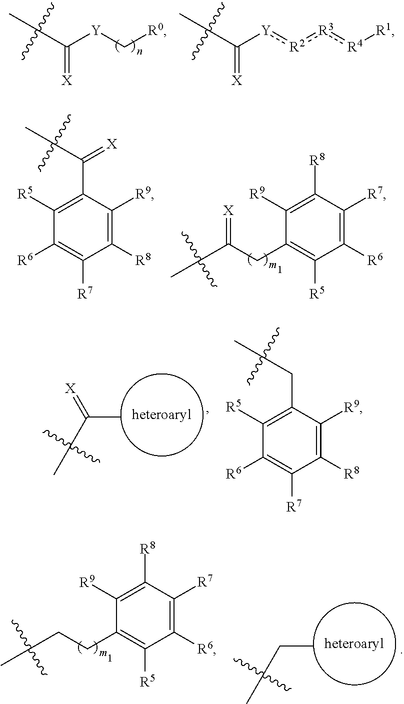

In some embodiments, R is selected from

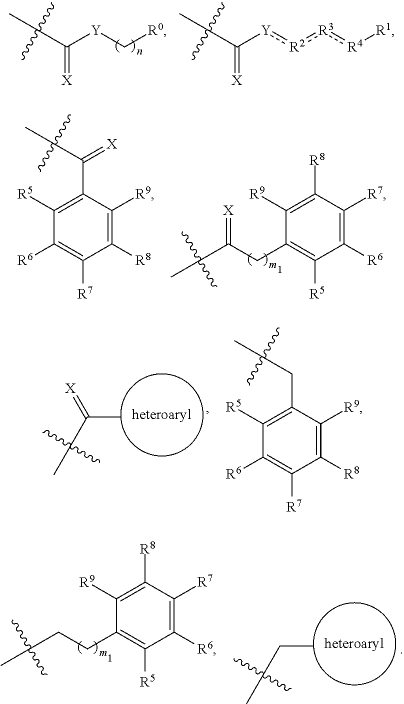

In some embodiments, R is selected from

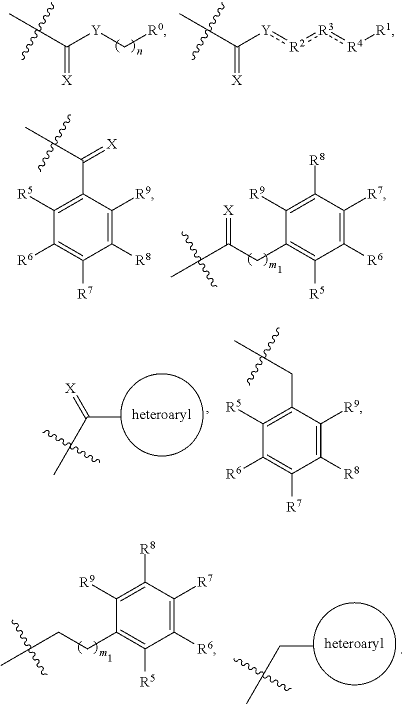

In some embodiments, R is selected from

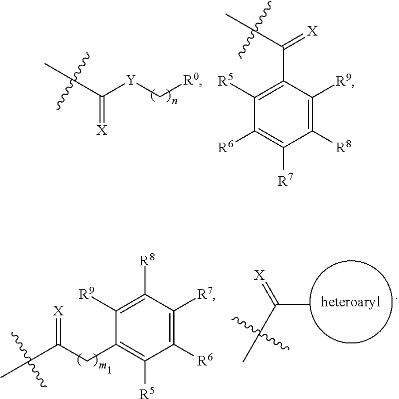

In some embodiments, the heteroaryl is selected from the followings:

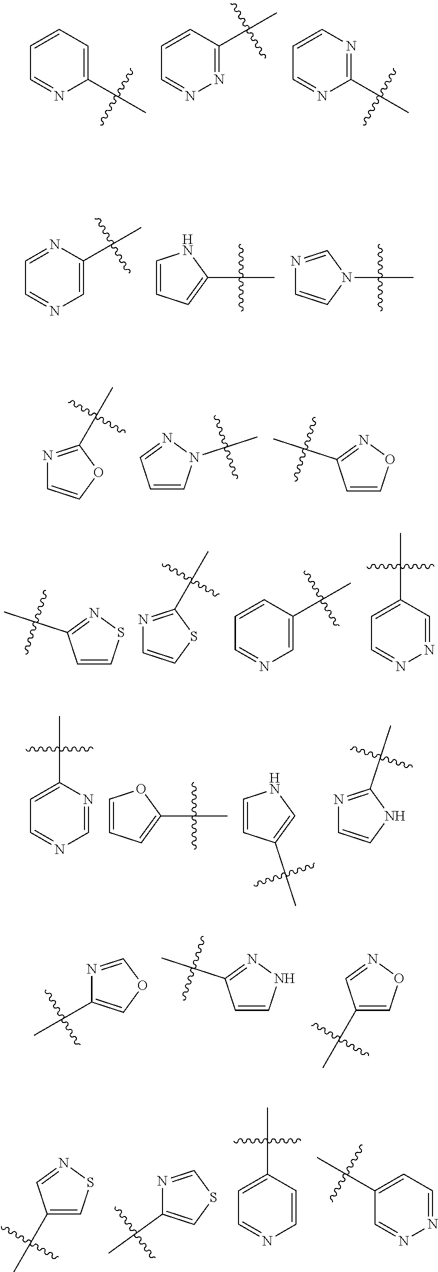

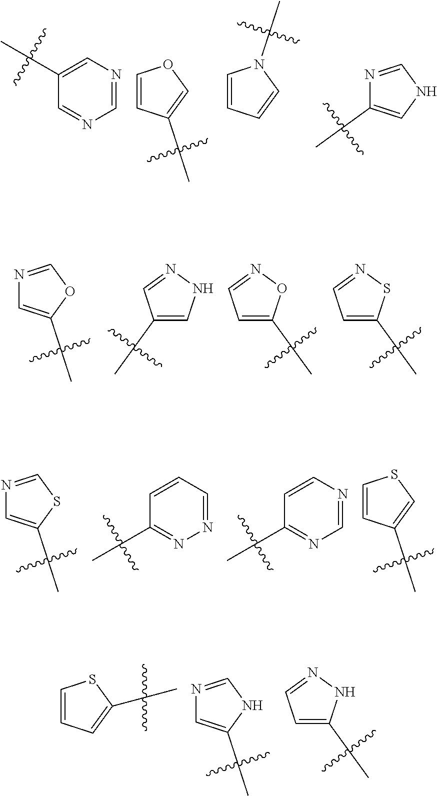

each heteroaryl is independently optionally substituted by one or more (e.g. 2, 3, 4, 5 or 6, preferably 2) R″s.

In some embodiments, the heteroaryl is selected from the followings:

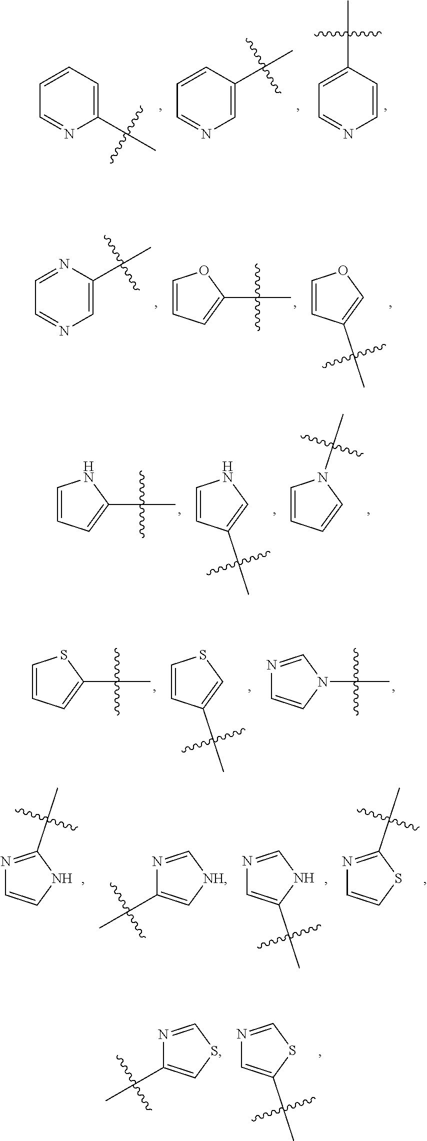

each heteroaryl is independently optionally substituted by one or more (e.g., 2, 3, 4, 5 or 6, preferably 2) R″s.

In some embodiments, the heteroaryl is selected from the followings:

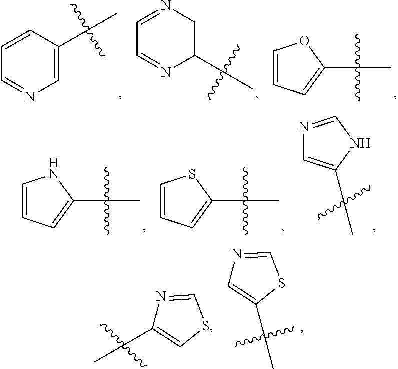

each heteroaryl is independently optionally substituted by one or more (e.g., 2, 3 4, 5 or 6, preferably 2) R″s.

In some embodiments, the heteroaryl is selected from the followings:

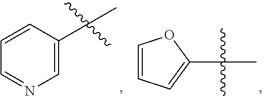 [SMILES: *c1cccnc1 ; *c1ccco1]

each heteroaryl is independently optionally substituted by one or more (e.g., 2, 3, 4, 5 or 6, preferably 2) R″s.

In some embodiments, X is O.

In some embodiments, each Y is independently selected from a direct bond or CH₂.

In some embodiments, R⁰ is —N₃ or —SS—C₁-C₆ alkyl (e.g., —SS-methyl, —SS-ethyl, —SS-isopropyl, —SS-tertbutyl).

In some embodiments, R⁰ is —N₃.

In some embodiments, R¹ is —N₃ or —SS—C₁-C₆ alkyl (e.g., —SS-methyl, —SS-ethyl, —SS-isopropyl, —SS-tertbutyl).

In some embodiments, R¹ is —N₃.

In some embodiments, R², R³, and R⁴ are each independently selected from O, NH, S, CH, CH₂, C(CH₃)₂, and meet the following conditions: when R² is selected from O or S, R³ and R⁴ are CH₂; when R³ is selected from O or S, R² and R⁴ are CH₂; when R⁴ is selected from O or S, R² and R³ are CH₂; when R² is C(CH₃)₂, R³ and R⁴ are CH₂; when R³ is C(CH₃)₂, R² and R⁴ are CH₂; when R⁴ is C(CH₃)₂, R² and R³ are CH₂; when Y is CH, R² is CH, and R³ and R⁴ are CH₂.

In some embodiments, R², R³, and R⁴ are each independently selected from CH or CH₂.

In some embodiments, R² and R³ are CH, and R⁴ is CH₂.

In some embodiments, when each heteroaryl is independently optionally substituted by one R″, each R″ is independently selected from

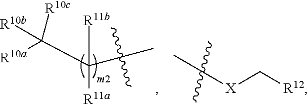

preferably, R″ is

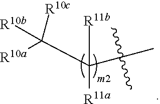

In some embodiments, when each heteroaryl is independently optionally substituted by more (e.g., 2, 3, 4, 5 or 6, preferably 2) R″s, one of the R″ is selected from

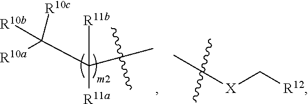

and the remaining R″(s) is(are) each independently selected from —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy).

In some embodiments, when each heteroaryl is independently optionally substituted by more (e.g., 2, 3, 4, 5 or 6, preferably 2) R″s, one of the R″ is

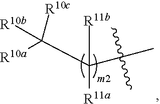

and the remaining R″(s) is(are) each independently selected from nitro, aliphatic alkyl (e.g., C₁-C₆ alkyl), F, I, Br, Cl.

In some embodiments, when each heteroaryl is independently optionally substituted by more (e.g., 2, 3, 4, 5 or 6, preferably 2) R″s, one of the R″ is

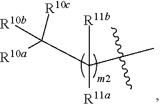

and the remaining R″(s) is(are) each independently selected from nitro, C₁-C₆ alkyl, preferably, the remaining R″(s) is(are) C₁-C₆ alkyl.

In some embodiments, any one of R⁵, R⁶, R⁷, R⁸, and R⁹ is selected from

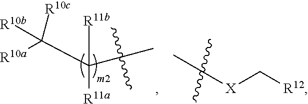

and the remaining four thereof are each independently selected from H, —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy).

In some embodiments, any one of R⁵, R⁶, R⁷, R⁸, and R⁹ is selected from

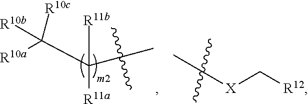

and the remaining four thereof are each independently selected from H, nitro, C₁-C₆ alkyl.

In some embodiments, of R⁵, R⁶, R⁷, R⁸, and R⁹, R⁵, R⁷ or R⁹ is selected from

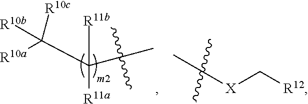

and the remaining four thereof are each independently selected from H, nitro.

In some embodiments, any one of R⁵, R⁶, R⁷, R⁸, and R⁹ is selected from

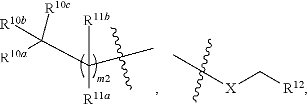

and the remaining four thereof are each independently selected from H, —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy), C₁-C₆ alkyl-C(═O)—NH₂—.

In some embodiments, any one of R⁵, R⁶, R⁷, R⁸, and R⁹ is

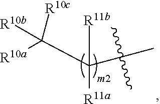

and the remaining four thereof are each independently selected from H, nitro, C₁-C₆ alkyl, C₁-C₆ alkoxy, C₁-C₆ alkyl-C(═O)—NH₂—.

In some embodiments, of R⁵, R⁶, R⁷, R⁸, and R⁹, R⁵ or R⁹ is

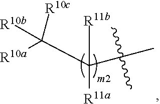

and the remaining four thereof are each independently selected from H, nitro, methoxy, acetamido.

In some embodiments, of R⁵, R⁶, R⁷, R⁸, and R⁹, R⁵ or R⁹ is

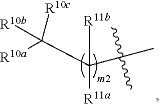

R⁷ is selected from H, nitro, methoxy, acetamido, and the remaining three thereof are H.

In some embodiments, any one of R^{x} and R^{y} is selected from

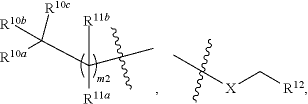

and the other is selected from H, —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy).

In some embodiments, any one of R^{x} and R^{y} is selected from

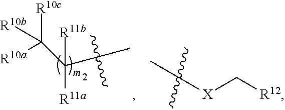

and the other is H.

In some embodiments, any one of R^{x} and R^{Y} is

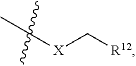

and the other is H.

In some embodiments, R^{z} is selected from

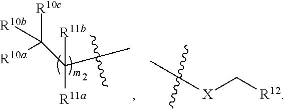

In some embodiments, R^{z} is

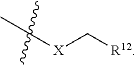

In some embodiments, any one of R^{10a}, R^{10b}, and R^{10c} is selected from —N₃, —SS—C₁-C₆ alkyl (e.g., —SS-methyl, —SS-ethyl, —SS-isopropyl, —SS-tertbutyl or —SS-isobutyl), —ONH₂, —OCOR^{m}, —OCONHR^{m}, and the other two are each independently selected from H, aliphatic alkyl (e.g., C₁-C₆ alkyl, particularly such as methyl, ethyl, isopropyl, tertbutyl), aromatic alkyl (e.g., phenyl C₁-C₆ alkyl), cycloalkyl (e.g., C₃-C₆ cycloalkyl), wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl).

In some embodiments, any one of R^{10a}, R^{10b}, and R^{10c} is —N₃ or —SS—C₁-C₆ alkyl (e.g., —SS-methyl, —SS-ethyl, —SS-isopropyl, —SS-tertbutyl or —SS-isobutyl), and the other two are each independently selected from H, aliphatic alkyl (e.g., C₁-C₆ alkyl, particularly such as methyl, ethyl, isopropyl, tertbutyl).

In some embodiments, any one of R^{10a}, R^{10b}, and R^{10c} is —N₃ or —SS-methyl, and the other two are each independently selected from H, methyl.

In some embodiments, any one of R^{10a}, R^{10b}, and R^{10c} is —N₃, —SS-methyl, —SS-ethyl, —SS-isopropyl, —SS-tertbutyl or —SS-isobutyl, and the other two are each independently selected from H, methyl.

In some embodiments, R^{11a} and R^{11b} are each independently selected from H, aliphatic alkyl (e.g., C₁-C₆ alkyl, particularly such as methyl, ethyl, isopropyl, tertbutyl).

In some embodiments, R^{11a} and R¹¹ b are H.

In some embodiments, R¹² is selected from —N₃, —SS—C₁-C₆ alkyl (e.g., —SS-methyl, —SS-ethyl, —SS-isopropyl, —SS-tertbutyl), —ONH₂, —OCOR^{m}, —OCONHR^{m}, wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl).

In some embodiments, R¹² is —N₃ or —SS—C₁-C₆ alkyl (e.g., —SS-methyl, —SS-ethyl, —SS-isopropyl, —SS-tertbutyl).

In some embodiments, R¹² is —N₃.

In some embodiments, n is selected from 1, 2, 3.

In some embodiments, n is 2.

In some embodiments, m₁ is 1.

In some embodiments, each m₂ is independently selected from 0 or 1.

In some embodiments,

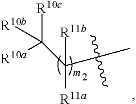

as a whole, is selected from

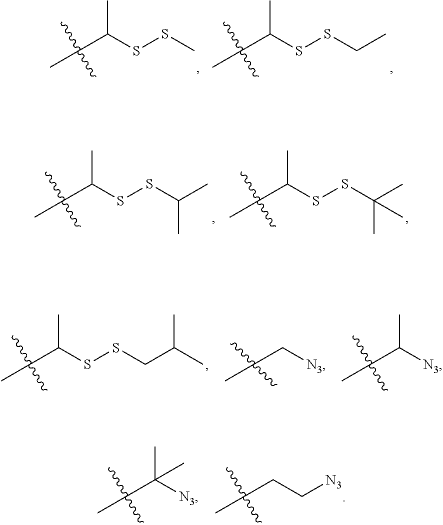

In some embodiments, R′ is a triphosphate group

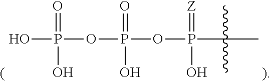

In some embodiments, Z is O.

In some embodiments, the Base is selected from

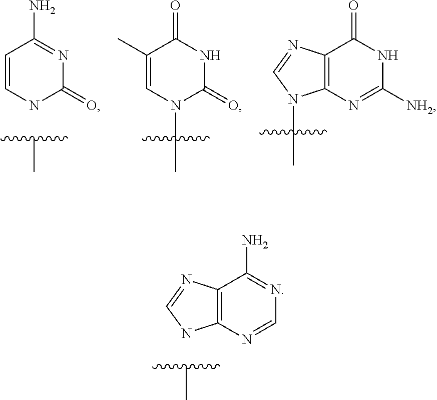

In the second aspect of the invention, the present invention provides the compound of formula (I) or a salt thereof,

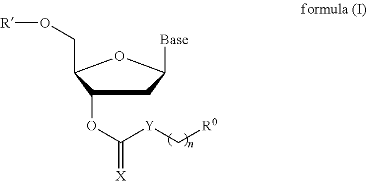

wherein:
X is selected from O, NH, S;
Y is selected from a direct bond, O, NH, S, CH, CH₂, C(CH₃)₂;
R⁰ is selected from —N₃, —SS—C₁-C₆ alkyl (e.g., —SS-methyl, —SS-ethyl, —SS-isopropyl, —SS-tertbutyl), —ONH₂, —OCOR^{m}, —OCONHR^{m}, wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl);
n is selected from 1, 2, 3, 4, and 5;

R′ is selected from H, monophosphate group

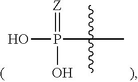

diphosphate group

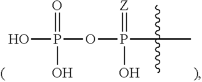

triphosphate group

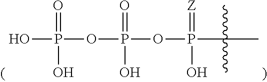

or tetraphosphate group

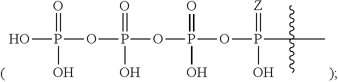

each Z is independently selected from O, S, BH;

Base is selected from a base, a deaza base or a tautomer thereof, for example, Base is selected from adenine, 7-deazaadenine, thymine, uracil, cytosine, guanine, 7-deazaguanine or a tautomer thereof.

In some embodiments, X is O.

In some embodiments, Y is CH₂.

In some embodiments, R⁰ is —N₃ or —SS—C₁-C₆ alkyl (e.g., —SS-methyl, —SS-ethyl, —SS-isopropyl, —SS-tertbutyl).

In some embodiments, R⁰ is —N₃.

In some embodiments, n is selected from 1, 2, 3.

In some embodiments, n is 2.

In some embodiments, R is a triphosphate group

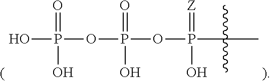

In some embodiments, Z is O.

In some embodiments, the Base is selected from

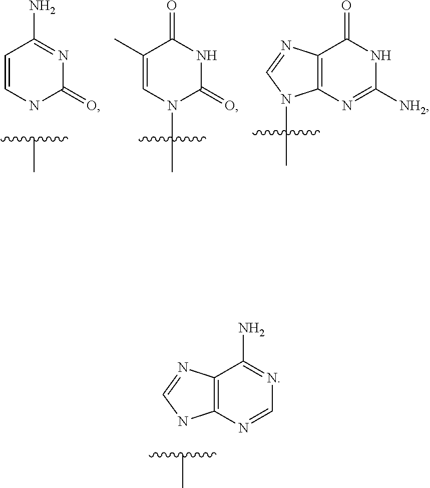

In the third aspect of the invention, the present invention provides the compound of formula (II) or a salt thereof,

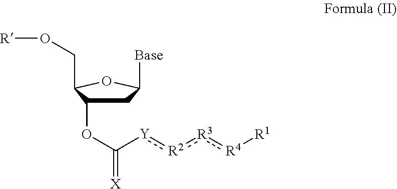

wherein:
X is selected from O, NH, S;
Y is selected from a direct bond, O, NH, S, CH, CH₂, C(CH₃)₂;
R¹ is selected from —N₃, —SS—C₁-C₆ alkyl (e.g., —SS-methyl, —SS-ethyl, —SS-isopropyl, —SS-tertbutyl), —ONH₂, —OCOR^{m}, —OCONHR^{m}, wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl);
R², R³, and R⁴ are each independently selected from O, NH, S, CH, CH₂, C(CH₃)₂;
[symbol] represents a single bond [symbol] or a double bond [symbol] ;
R′ is selected from H, monophosphate group

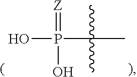

diphosphate group

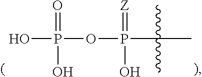

triphosphate group

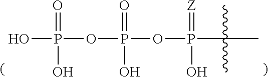

or tetraphosphate group

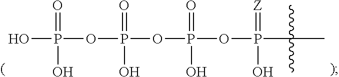

each Z is independently selected from O, S, BH;

Base is selected from a base, a deaza base or a tautomer thereof, for example, Base is selected from adenine, 7-deazaadenine, thymine, uracil, cytosine, guanine, 7-deazaguanine or a tautomer thereof.

In some embodiments, X is O.

In some embodiments, Y is a direct bond.

In some embodiments, R¹ is —N₃ or —SS—C₁-C₆ alkyl (e.g., —SS-methyl, —SS-ethyl, —SS-isopropyl, —SS-tertbutyl).

In some embodiments, R¹ is —N₃.

In some embodiments, R², R³, and R⁴ are each independently selected from O, NH, S, CH, CH₂, C(CH₃)₂, and meet the following conditions: when R² is selected from O or S, R³ and R⁴ are CH₂; when R³ is selected from O or S, R² and R⁴ are CH₂; when R⁴ is selected from O or S, R² and R³ are CH₂; when R² is C(CH₃)₂, R³ and R⁴ are CH₂; when R³ is C(CH₃)₂, R² and R⁴ are CH₂; when R⁴ is C(CH₃)₂, R² and R³ are CH₂; when Y is CH, R² is CH, and R³ and R⁴ are CH₂.

In some embodiments, R², R³, and R⁴ are each independently selected from CH or CH₂.

In some embodiments, R² and R³ are CH, and R⁴ is CH₂.

In some embodiments, R′ is a triphosphate group

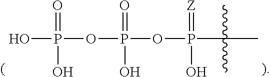

In some embodiments, Z is O.

In some embodiments, the Base is selected from

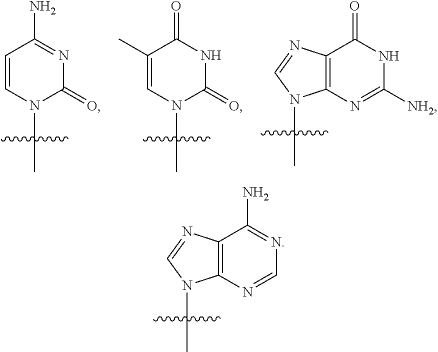

In the fourth aspect of the invention, the present invention provides the compound of formula (III) or a salt thereof,

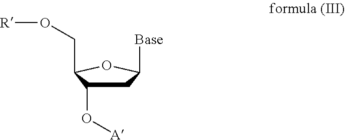

wherein:
A′ is selected from

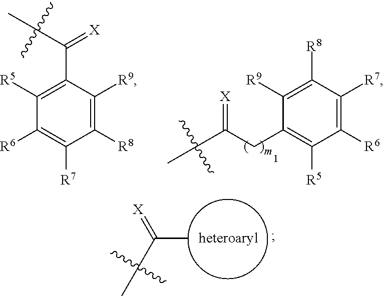

the heteroaryl is selected from the followings:

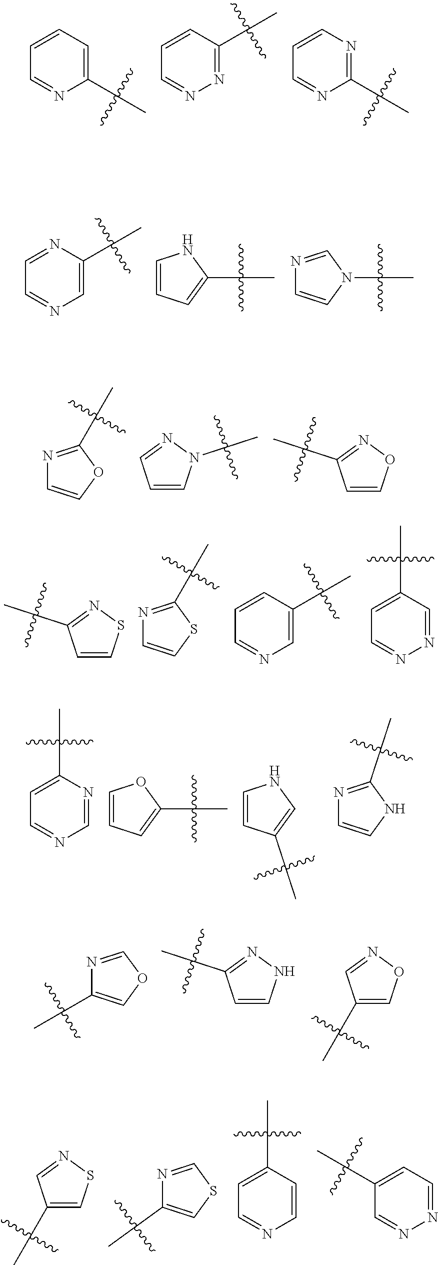

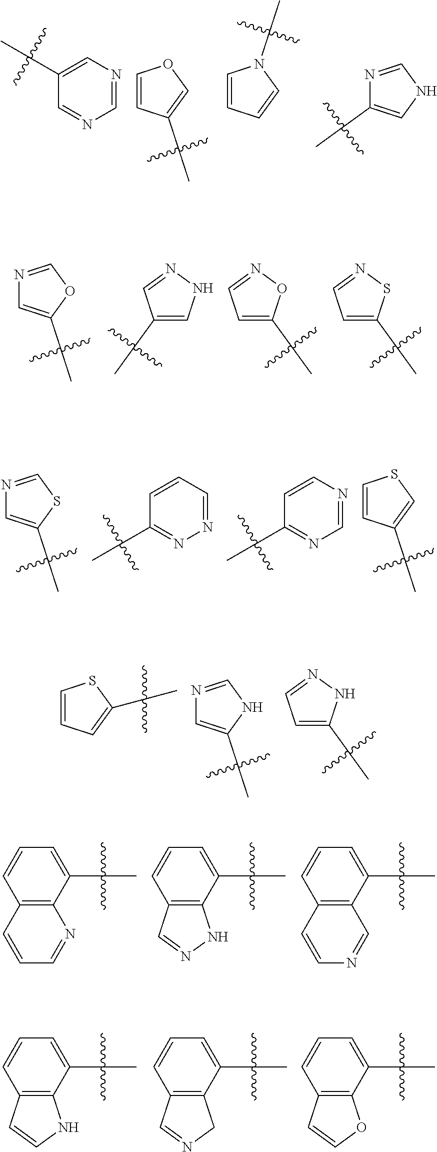

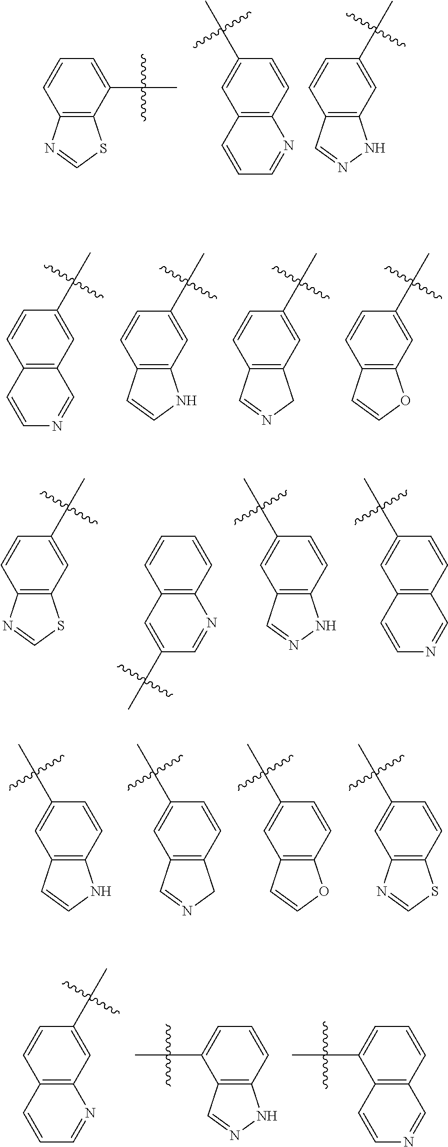

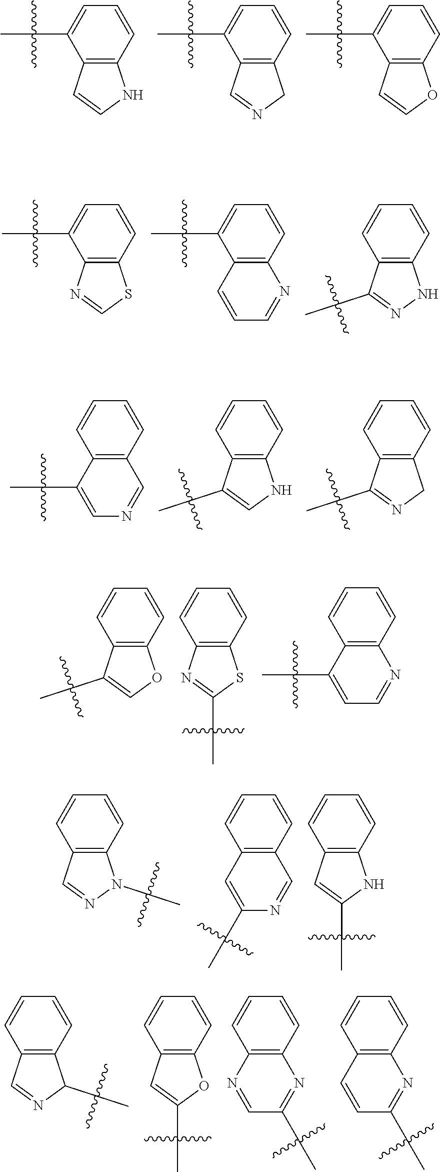

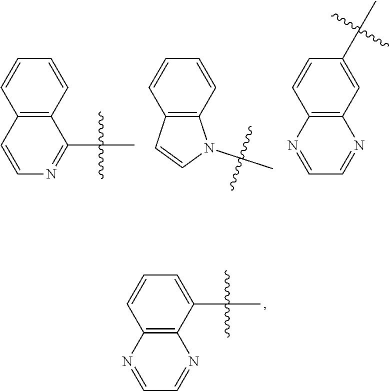 [SMILES: *c1nccc2ccccc12 ; *n1ccc2ccccc21 ; *c1ccc2nccnc2c1 ; *c1cccc2nccnc12]

each heteroaryl is independently optionally substituted by one or more (e.g., 2, 3, 4, 5 or 6, preferably 2) R″s;
each R″ is independently selected from H, —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy),

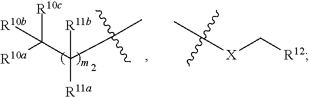

X is selected from O, NH, S;
R⁵, R⁶, R⁷, R⁸, and R⁹ are each independently selected from H, —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy),

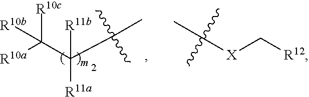

and R⁵, R⁶, R⁷, R⁸, and R⁹ are not H at the same time; or, R⁵, R⁶, R⁷, R⁸, and R⁹ are each independently selected from H, —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy), C₁-C₆ alkyl-C(═O)—NH₂—,

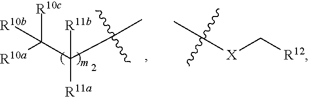

and R⁵, R⁶, R⁷, R⁸, and R⁹ are not H at the same time;
R^{10a}, R^{10b}, R^{10c}, R^{11a}, R^{11b} and R¹² are each independently selected from H, —N₃, —SS—C₁-C₆ alkyl (e.g., —SS-methyl, —SS-ethyl, —SS-isopropyl, —SS-tertbutyl or —SS-isobutyl), —ONH₂, —OCOR^{m}, —OCONHR^{m}, aliphatic alkyl (e.g., C₁-C₆ alkyl, particularly such as methyl, ethyl, isopropyl, tertbutyl), aromatic alkyl (e.g., phenyl C₁-C₆ alkyl), cycloalkyl (e.g., C₃-C₆ cycloalkyl), wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl), and R^{10a}, R^{10b}, and R^{10c} are not H at the same time;
m₁ is selected from 1, 2, 3, 4, 5, and 6;
m₂ is selected from 0, 1, 2, 3, 4, 5, and 6;
R′ is selected from H, monophosphate group

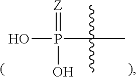

diphosphate group

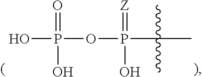

triphosphate group

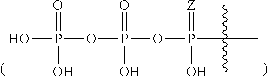

or tetraphosphate group

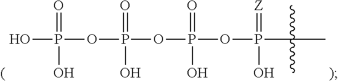

each Z is independently selected from O, S, BH;

Base is selected from a base, a deaza base or a tautomer thereof, for example, Base is selected from adenine, 7-deazaadenine, thymine, uracil, cytosine, guanine, 7-deazaguanine or a tautomer thereof.

In some embodiments, the heteroaryl is selected from the followings:

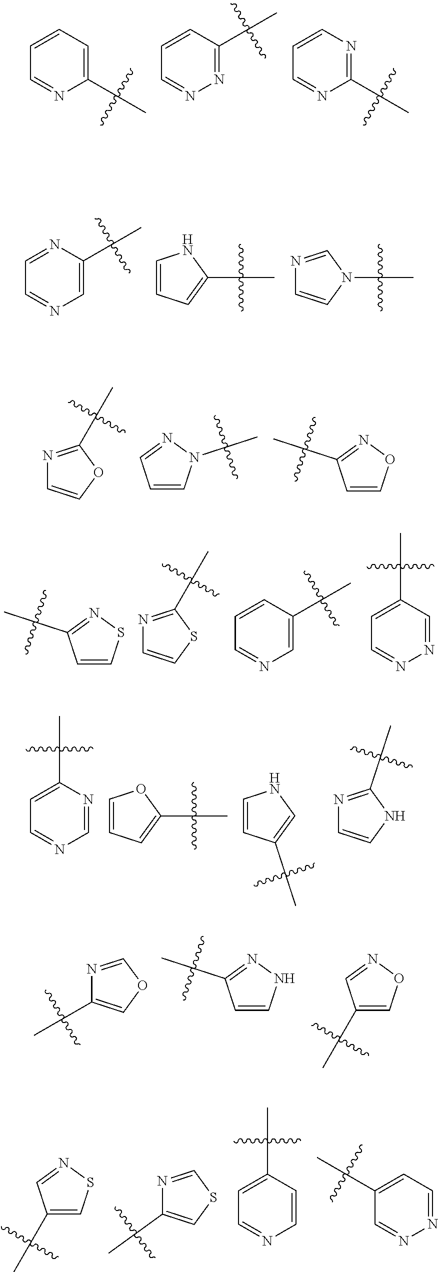

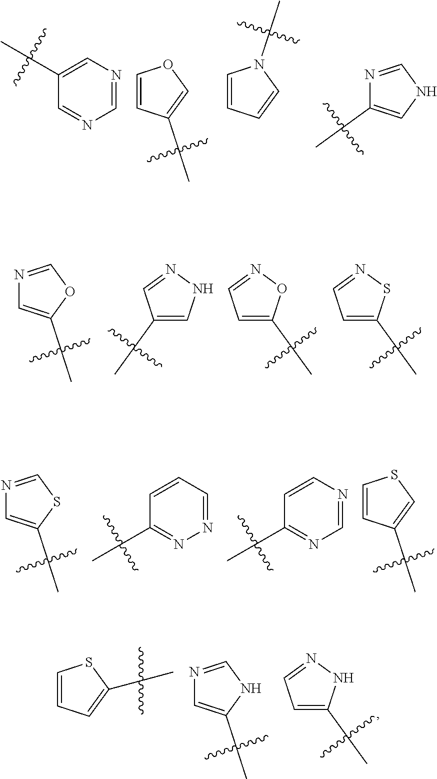

each heteroaryl is independently optionally substituted by one or more (e.g., 2, 3, 4, 5 or 6, preferably 2) R″s.

In some embodiments, the heteroaryl is selected from the followings:

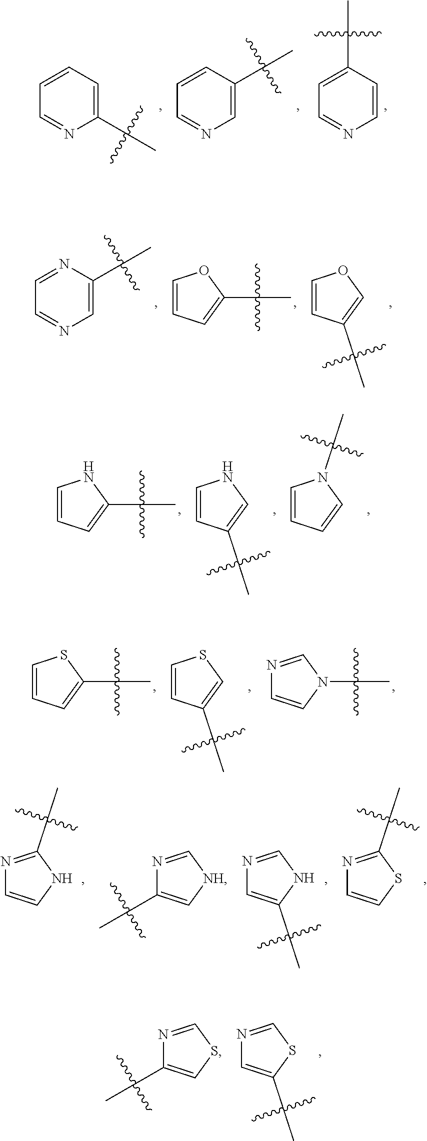

each heteroaryl is independently optionally substituted by one or more (e.g., 2, 3, 4, 5 or 6, preferably 2) R″s.

In some embodiments, the heteroaryl is selected from the followings:

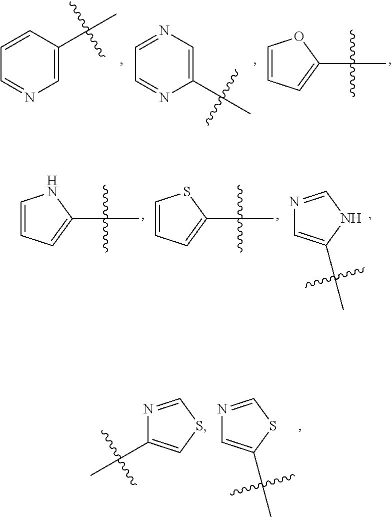

each heteroaryl is independently optionally substituted by one or more (e.g., 2, 3, 4, 5 or 6, preferably 2) R″s.

In some embodiments, the heteroaryl is selected from the followings:

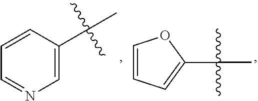 [SMILES: *c1cccnc1 ; *c1ccco1]

each heteroaryl is independently optionally substituted by one or more (e.g., 2, 3, 4, 5 or 6, preferably 2) R″s.

In some embodiments, the heteroaryl is selected from the followings:

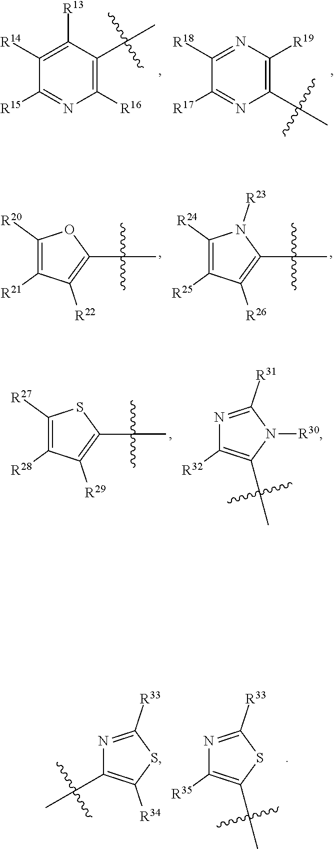

In some embodiments, the heteroaryl is selected from the followings:

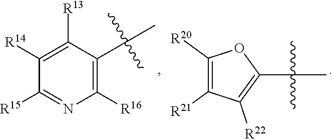

In some embodiments, when each heteroaryl is independently optionally substituted by one R″, each R″ is independently selected from

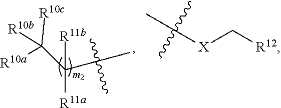

preferably, R″ is

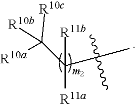

In some embodiments, when each heteroaryl is independently optionally substituted by more (e.g., 2, 3, 4, 5 or 6, preferably 2) R″s, one of the R″ is selected from

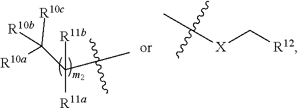

and the remaining R″(s) is(are) each independently selected from —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy).

In some embodiments, when each heteroaryl is independently optionally substituted by more (e.g., 2, 3, 4, 5 or 6, preferably 2) R″s, one of the R″ is

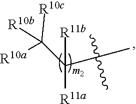

and the remaining R″(s) is(are) each independently selected from nitro, aliphatic alkyl (e.g., C₁-C₆ alkyl), F, I, Br, Cl.

In some embodiments, when each heteroaryl is independently optionally substituted by more (e.g., 2, 3, 4, 5 or 6, preferably 2) R″s, one of the R″ is

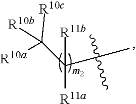

and the remaining R″(s) is(are) each independently selected from nitro, C₁-C₆ alkyl, preferably, the remaining R″(s) is(are) C₁-C₆ alkyl, more preferably, the remaining R″(s) is(are) methyl.

In some embodiments, of R¹³, R¹⁴, R¹⁵, and R¹⁶, R¹⁶ is

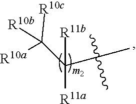

and R¹³, R¹⁴, and R¹⁵ are each independently selected from H, nitro, C₁-C₆ alkyl (e.g., methyl).

In some embodiments, of R¹³, R¹⁴, R¹⁵, and R¹⁶, R¹⁶ is

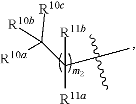

and R¹³, R¹⁴, and R¹⁵ are H.

In some embodiments, of R¹⁷, R¹⁸, and R¹⁹, R¹⁹ is

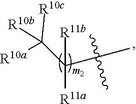

and R¹⁷ and R¹⁸ are each independently selected from H, nitro, C₁-C₆ alkyl (e.g., methyl).

In some embodiments, of R¹⁷, R¹⁸, and R¹⁹, R¹⁹ is

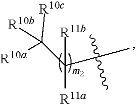

and R¹⁷ and R¹⁸ are H.

In some embodiments, of R²⁰, R²¹, and R²², R²² is

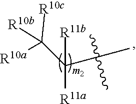

and R²⁰ and R²¹ are each independently selected from H, nitro, C₁-C₆ alkyl (e.g., methyl).

In some embodiments, of R²⁰, R²¹, and R²², R²² is

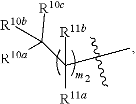

and R²⁰ and R²¹ are H.

In some embodiments, of R²³, R²⁴, R²⁵, and R²⁶, R²⁶ is

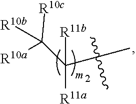

and R²³, R²⁴, and R²⁵ are each independently selected from H, nitro, C₁-C₆ alkyl (e.g., methyl).

In some embodiments, of R²³, R²⁴, R²⁵, and R²⁶, R²⁶ is

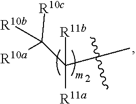

and R²³, R²⁴, and R²⁵ are H.

In some embodiments, of R²⁷, R²⁸, and R²⁹, R²⁹ is

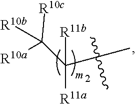

and R²⁷ and R²⁸ are each independently selected from H, nitro, C₁-C₆ alkyl (e.g., methyl).

In some embodiments, of R²⁷, R²⁸, and R²⁹, R²⁹ is

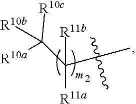

and R²⁷ and R²⁸ are H.

In some embodiments, of R³⁰, R³¹, and R³², R³² is

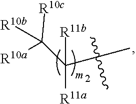

and R³⁰ and R³¹ are each independently selected from H, nitro, C₁-C₆ alkyl (e.g., methyl).

In some embodiments, of R³⁰, R³¹, and R³², R³² is

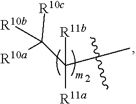

and R³⁰ and R³¹ are H.

In some embodiments, of R³³, R³⁴, and R³⁵, R³⁴ or R³⁵ is

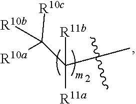

and R³³ is selected from H, nitro, C₁-C₆ alkyl (e.g., methyl).

In some embodiments, of R³³, R³⁴, and R³⁵, R³⁴ or R³⁵ is

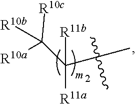

and R³³ is H or methyl.

In some embodiments, X is O.

In some embodiments, any one of R⁵, R⁶, R⁷, R⁸, and R⁹ is selected from

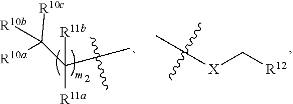

and the remaining four thereof are each independently selected from H, —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy).

In some embodiments, any one of R⁵, R⁶, R⁷, R⁸, and R⁹ is

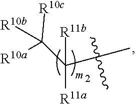

and the remaining four thereof are each independently selected from H, nitro, C₁-C₆ alkyl.

In some embodiments, of R⁵, R⁶, R⁷, R⁸, and R⁹, R⁵ or R⁹ is

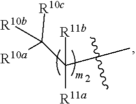

and the remaining four thereof are each independently selected from H, nitro.

In some embodiments, any one of R⁵, R⁶, R⁷, R⁸, and R⁹ is selected from

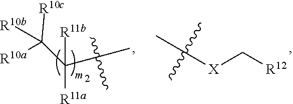

and the remaining four thereof are each independently selected from H, —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy), C₁-C₆ alkyl-C(═O)—NH₂—.

In some embodiments, any one of R⁵, R⁶, R⁷, R⁸, and R⁹ is

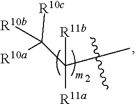

and the remaining four thereof are each independently selected from H, nitro, C₁-C₆ alkyl, C₁-C₆ alkoxy, C₁-C₆ alkyl-C(═O)—NH₂—.

In some embodiments, of R⁵, R⁶, R⁷, R⁸, and R⁹, R⁵ or R⁹ is

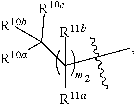

and the remaining four thereof are each independently selected from H, nitro, methoxy, acetamido.

In some embodiments, of R⁵, R⁶, R⁷, R⁸, and R⁹, R⁵ or R⁹ is

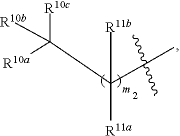

R⁷ is selected from H, nitro, methoxy, acetamido, and the remaining three thereof are H.

In some embodiments, any one of R^{10a}, R^{10b}, and R^{10c} is selected from —N₃, —SS—C₁-C₆ alkyl (e.g., —SS-methyl, —SS-ethyl, —SS-isopropyl, —SS-tertbutyl or —SS-isobutyl), —ONH₂, —OCOR^{m}, —OCONHR^{m}, and the other two are each independently selected from H, aliphatic alkyl (e.g., C₁-C₆ alkyl, particularly such as methyl, ethyl, isopropyl, tertbutyl), aromatic alkyl (e.g., phenyl C₁-C₆ alkyl), cycloalkyl (e.g., C₃-C₆ cycloalkyl), wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl).

In some embodiments, any one of R^{10a}, R^{10b}, and R^{10c} is —N₃ or —SS—C₁-C₆ alkyl (e.g., —SS-methyl, —SS-ethyl, —SS-isopropyl, —SS-tertbutyl or —SS-isobutyl), and the other two are each independently selected from H, C₁-C₆ alkyl (e.g., methyl, ethyl, isopropyl, tertbutyl).

In some embodiments, any one of R^{10a}, R^{10b}, and R^{10c} is —N₃ or —SS-methyl, and the other two are each independently selected from H, methyl.

In some embodiments, any one of R^{10a}, R^{10b}, and R^{10c} is —N₃, —SS-methyl, —SS-ethyl, —SS-isopropyl, —SS-tertbutyl, —SS-isobutyl, and the other two are each independently selected from H, methyl.

In some embodiments, R^{11a} and R^{11b} are each independently selected from H, aliphatic alkyl (e.g., C₁-C₆ alkyl, particularly such as methyl, ethyl, isopropyl, tertbutyl).

In some embodiments, R^{11a} and R^{11b} are H.

In some embodiments, R¹² is selected from —N₃, —SS—C₁-C₆ alkyl (e.g., —SS-methyl, —SS-ethyl, —SS-isopropyl, —SS-tertbutyl), —ONH₂, —OCOR^{m}, —OCONHR^{m}, wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl).

In some embodiments, R¹² is —N₃ or —SS—C₁-C₆ alkyl (e.g., —SS-methyl, —SS-ethyl, —SS-isopropyl, —SS-tertbutyl).

In some embodiments, R¹² is —N₃.

In some embodiments, m₁ is 1.

In some embodiments, m₂ is selected from 0 or 1.

In some embodiments,

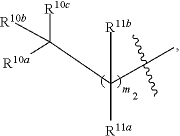

as a whole, is selected from

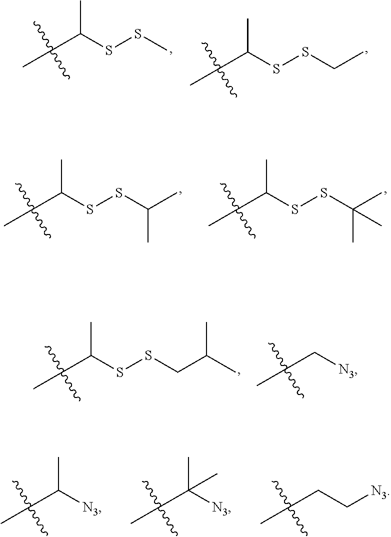

In some embodiments, R′ is a triphosphate group

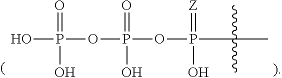

In some embodiments, Z is O.

In some embodiments, the Base is selected from

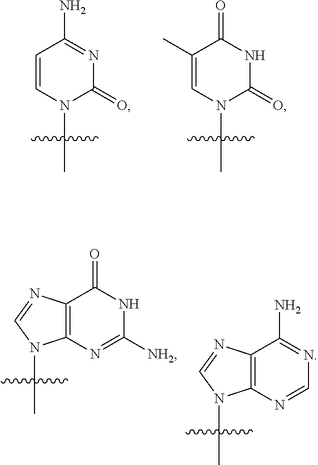

In the fifth aspect of the invention, the present invention provides the compound of formula (IV) or a salt thereof,

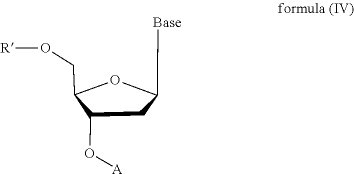

wherein:
A is selected from

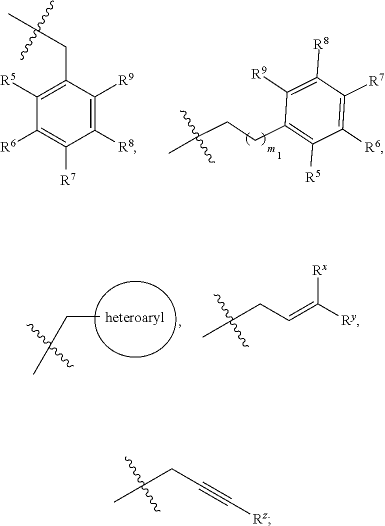

the heteroaryl is selected from the followings:

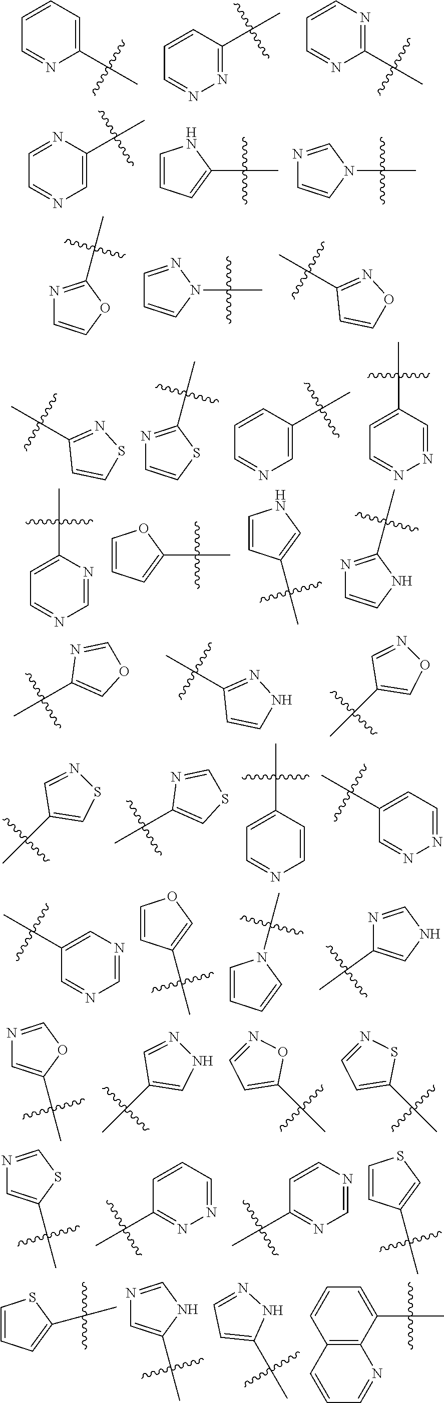

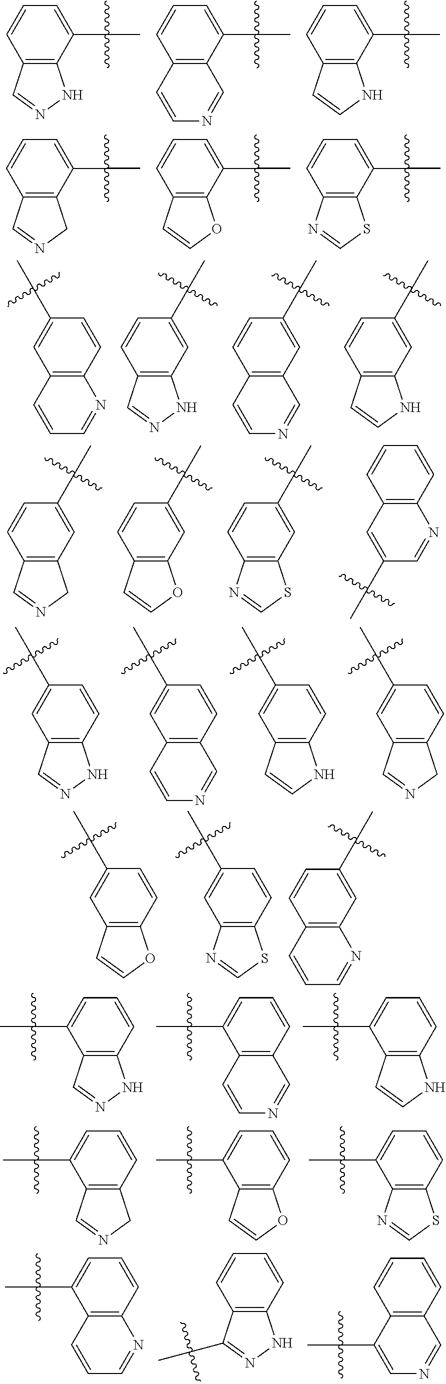

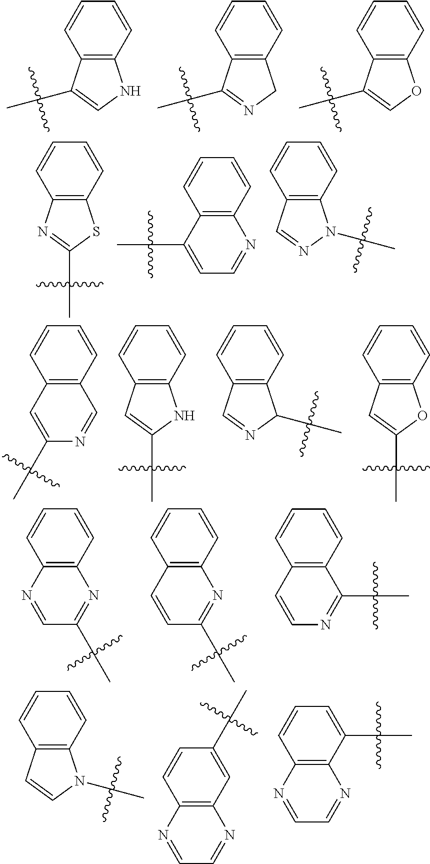

each heteroaryl is independently optionally substituted by one or more (e.g., 2, 3, 4, 5 or 6, preferably 2) R″s;
each R″ is independently selected from H, —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy),

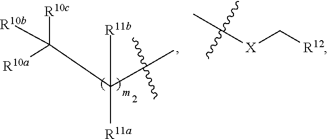

R⁵, R⁶, R⁷, R⁸, R⁹, R^{x}, R^{y}, and R^{z} are each independently selected from H, —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy),

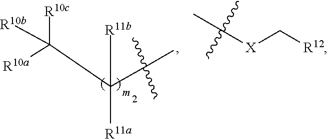

and R⁵, R⁶, R⁷, R⁸, and R⁹ are not H at the same time;
R^{10a}, R^{10b}, R^{10c}, R^{11a}, R^{11b} and R¹² are each independently selected from H, —N₃, —SS—C₁-C₆ alkyl (e.g., —SS-methyl, —SS-ethyl, —SS-isopropyl, —SS-tertbutyl), —ONH₂, —OCOR^{m}, —OCONHR^{m}, aliphatic alkyl (e.g., C₁-C₆ alkyl, particularly such as methyl, ethyl, isopropyl, tertbutyl), aromatic alkyl (e.g., phenyl C₁-C₆ alkyl), cycloalkyl (e.g., C₃-C₆ cycloalkyl), wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl), and R^{10a}, R^{10b}, and R^{10c} are not H at the same time;
X is selected from O, NH, S;
m₁ is selected from 1, 2, 3, 4, 5, and 6;
m₂ is selected from 0, 1, 2, 3, 4, 5, and 6;

R′ is selected from H, monophosphate group

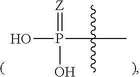

diphosphate group

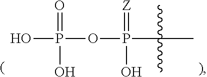

triphosphate group

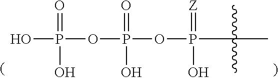

or tetraphosphate group

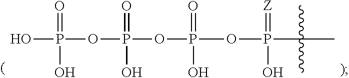

each Z is independently selected from O, S, BH;

Base is selected from a base, a deaza base or a tautomer thereof, for example, Base is selected from adenine, 7-deazaadenine, thymine, uracil, cytosine, guanine, 7-deazaguanine or a tautomer thereof.

In some embodiments, A is selected from

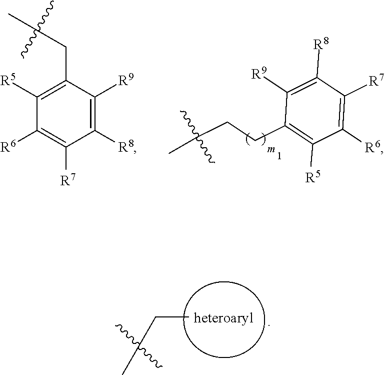

In some embodiments, A is selected from

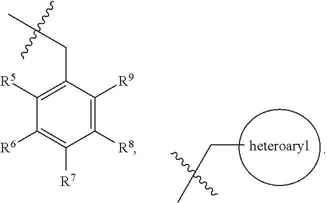

In some embodiments, A is

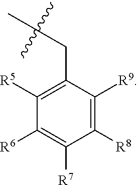

In some embodiments, the heteroaryl is selected from the followings:

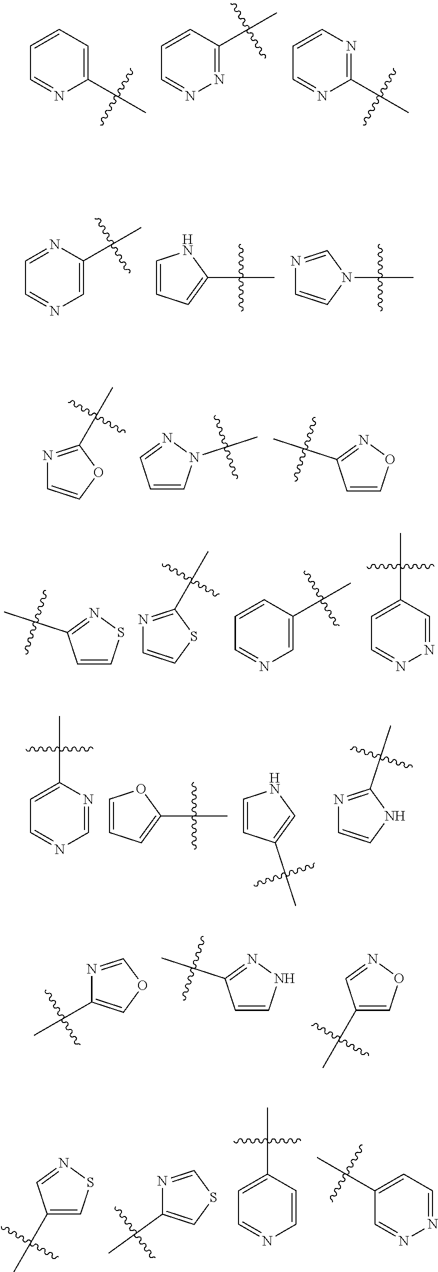

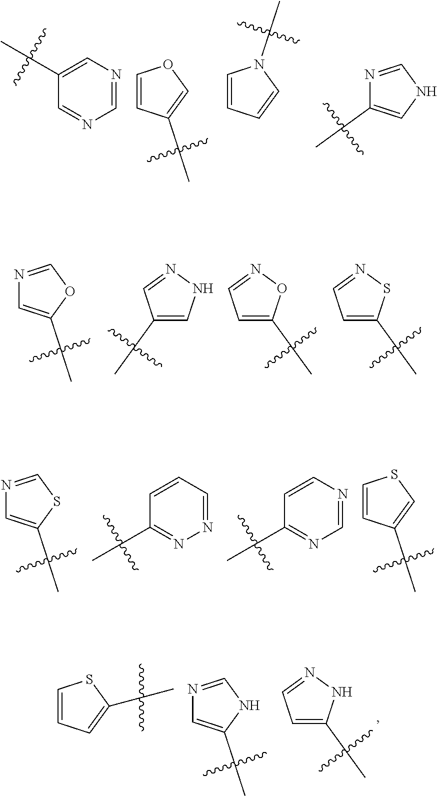

each heteroaryl is independently optionally substituted by one or more (e.g., 2, 3 4 5 or 6, preferably 2) R″s.

In some embodiments, the heteroaryl is selected from the followings:

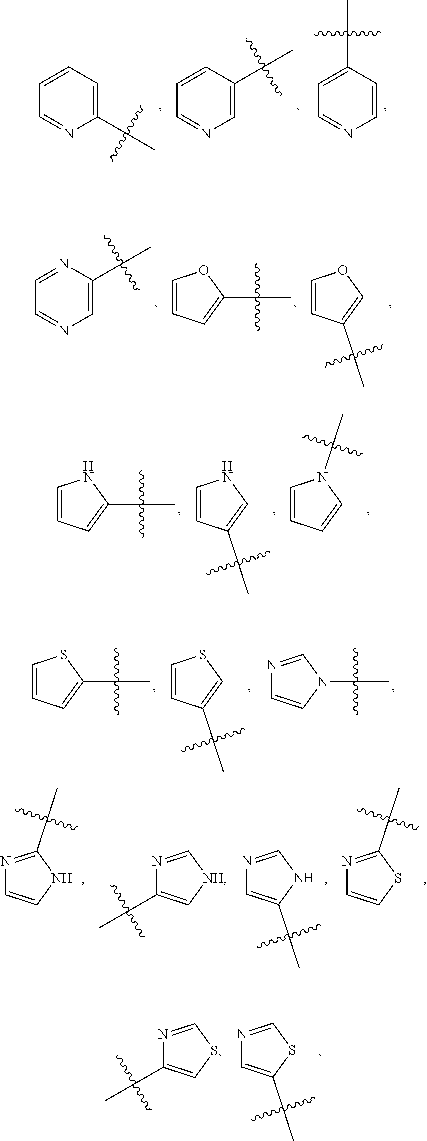

each heteroaryl is independently optionally substituted by one or more (e.g., 2, 3, 4, 5 or 6, preferably 2) R″s.

In some embodiments, the heteroaryl is selected from the followings:

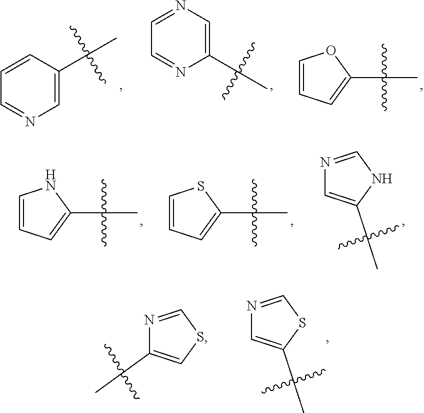

each heteroaryl is independently optionally substituted by one or more (e.g., 2, 3, 4, 5 or 6, preferably 2) R″s.

In some embodiments, when each heteroaryl is independently optionally substituted by one R″, each R″ is independently selected from

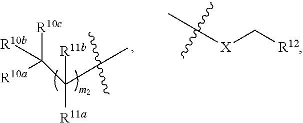

preferably, R″ is

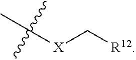

In some embodiments, when each heteroaryl is independently optionally substituted by more (e.g., 2, 3, 4, 5 or 6, preferably 2) R″s, one of the R″ is selected from

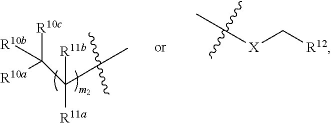

and the remaining R″(s) is(are) each independently selected from —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy).

In some embodiments, when each heteroaryl is independently optionally substituted by more (e.g., 2, 3, 4, 5 or 6, preferably 2) R″s, one of the R″ is

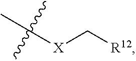

and the remaining R″(s) is(are) each independently selected from nitro, aliphatic alkyl (e.g., C₁-C₆ alkyl), F, I, Br, Cl.

In some embodiments, when each heteroaryl is independently optionally substituted by more (e.g., 2, 3, 4, 5 or 6, preferably 2) R″s, one of the R″ is

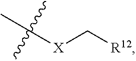

and the remaining R″(s) is(are) each independently selected from nitro, C₁-C₆ alkyl, preferably, the remaining R″(s) is(are) C₁-C₆ alkyl.

In some embodiments, any one of R⁵, R⁶, R⁷, R⁸, and R⁹ is selected from

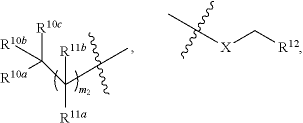

and the remaining four thereof are each independently selected from H, —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy).

In some embodiments, any one of R⁵, R⁶, R⁷, R⁸, and R⁹ is

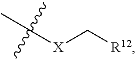

and the remaining four thereof are each independently selected from H, nitro, C₁-C₆ alkyl.

In some embodiments, of R⁵, R⁶, R⁷, R⁸, and R⁹, R⁷ is

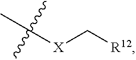

and the remaining four thereof are H.

In some embodiments, any one of R^{x} and R^{y} is selected from

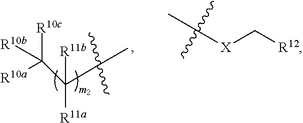

and the other is selected from H, —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy).

In some embodiments, any one of R^{x} and R^{y} is

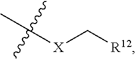

and the other is H.

In some embodiments, R^{z} is selected from

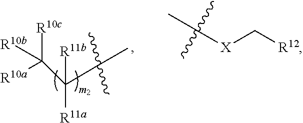

In some embodiments, R^{z} is

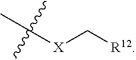

In some embodiments, any one of R^{10a}, R^{10b}, and R^{10c} is selected from —N₃, —SS—C₁-C₆ alkyl (e.g., —SS-methyl, —SS-ethyl, —SS-isopropyl, —SS-tertbutyl), —ONH₂, —OCOR^{m}, —OCONHR^{m}, and the other two are each independently selected from H, aliphatic alkyl (e.g., C₁-C₆ alkyl, particularly such as methyl, ethyl, isopropyl, tertbutyl), aromatic alkyl (e.g., phenyl C₁-C₆ alkyl), cycloalkyl (e.g., C₃-C₆ cycloalkyl), wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl).

In some embodiments, any one of R^{10a}, R^{10b}, and R^{10c} is —N₃ or —SS—C₁-C₆ alkyl (e.g., —SS-methyl, —SS-ethyl, —SS-isopropyl, —SS-tertbutyl), and the other two are each independently selected from H, aliphatic alkyl (e.g., C₁-C₆ alkyl, particularly such as methyl, ethyl, isopropyl, tertbutyl).

In some embodiments, R^{11a} and R^{11b} are each independently selected from H, aliphatic alkyl (e.g., C₁-C₆ alkyl, particularly such as methyl, ethyl, isopropyl, tertbutyl).

In some embodiments, R^{11a} and R^{11b} are H.

In some embodiments, R¹² is selected from —N₃, —SS—C₁-C₆ alkyl (e.g., —SS-methyl, —SS-ethyl, —SS-isopropyl, —SS-tertbutyl), —ONH₂, —OCOR^{m}, —OCONHR^{m}, wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl).

In some embodiments, R¹² is —N₃ or —SS—C₁-C₆ alkyl (e.g., —SS-methyl, —SS-ethyl, —SS-isopropyl, —SS-tertbutyl).

In some embodiments, R¹² is —N₃.

In some embodiments, X is O.

In some embodiments, m₁ is 1.

In some embodiments, m₂ is selected from 0 or 1.

In some embodiments, R is a triphosphate group

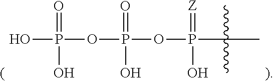

In some embodiments, Z is O.

In some embodiments the Base is selected from

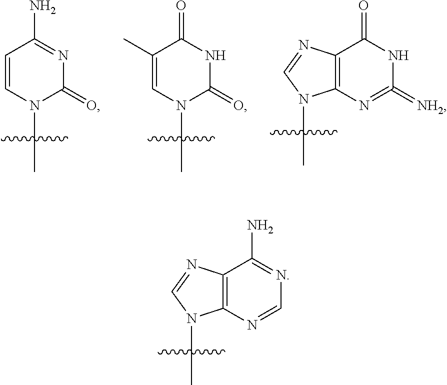

In the sixth aspect of the invention, the present invention provides the following compounds or salts thereof,

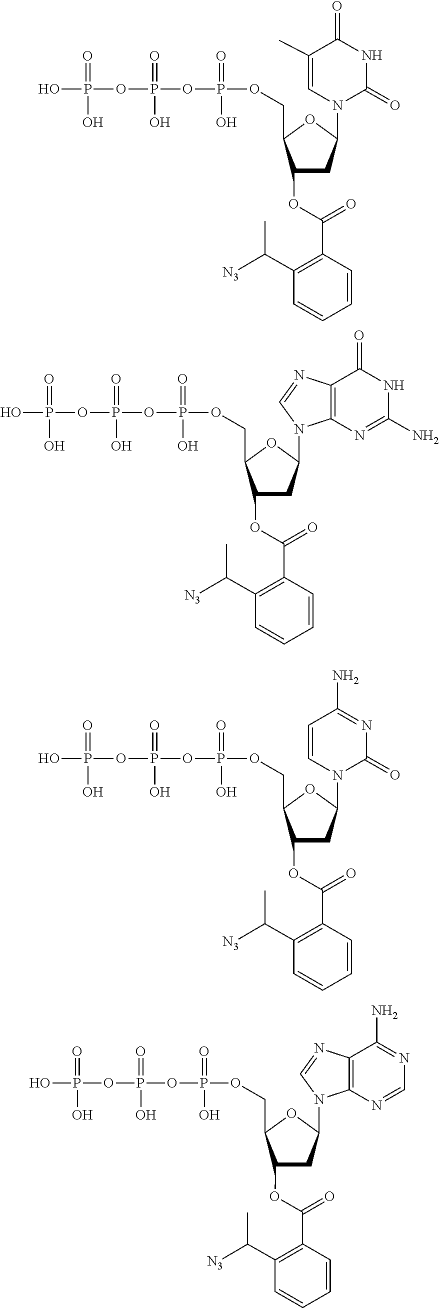

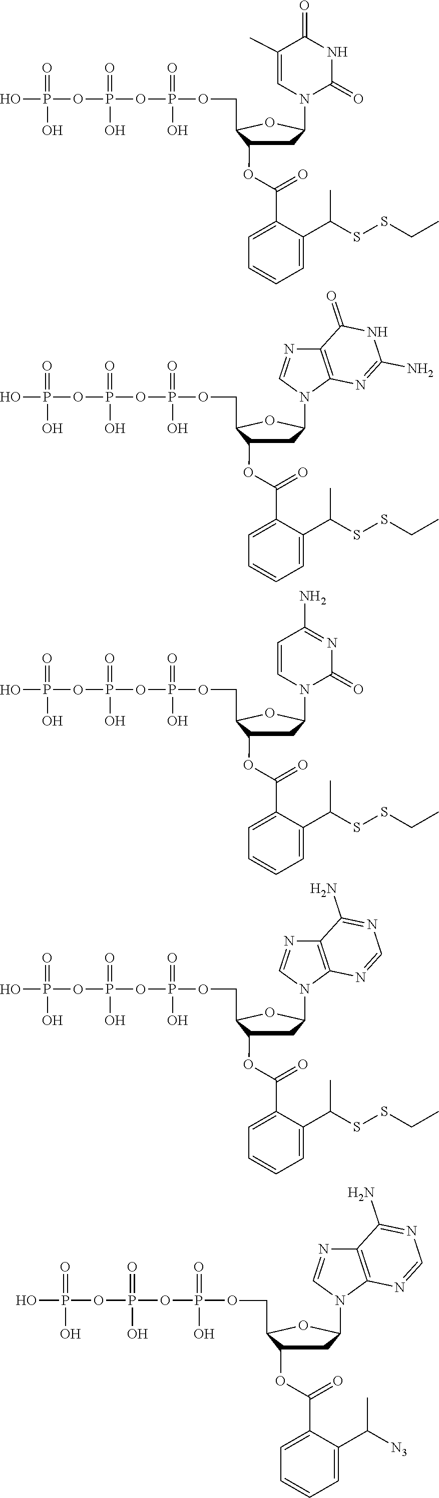

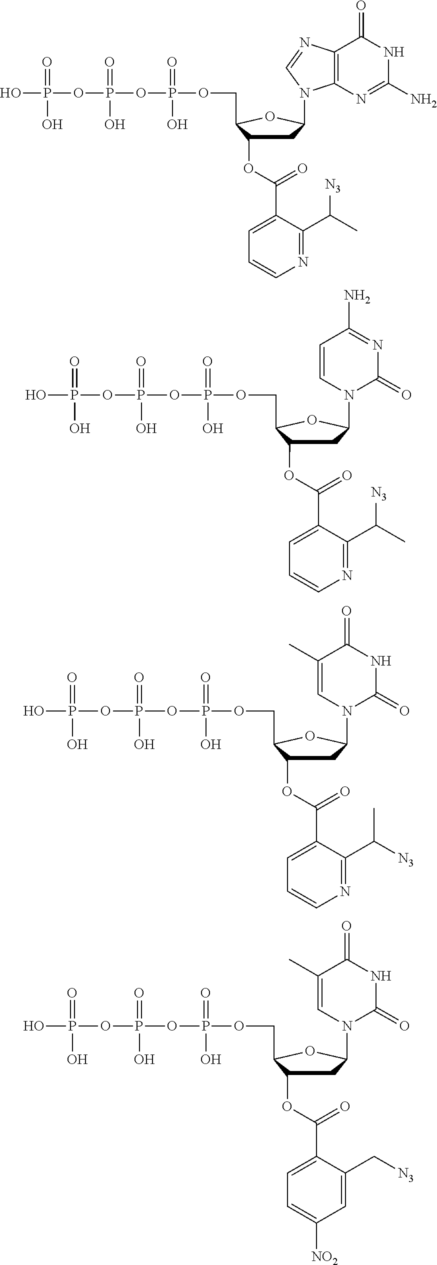

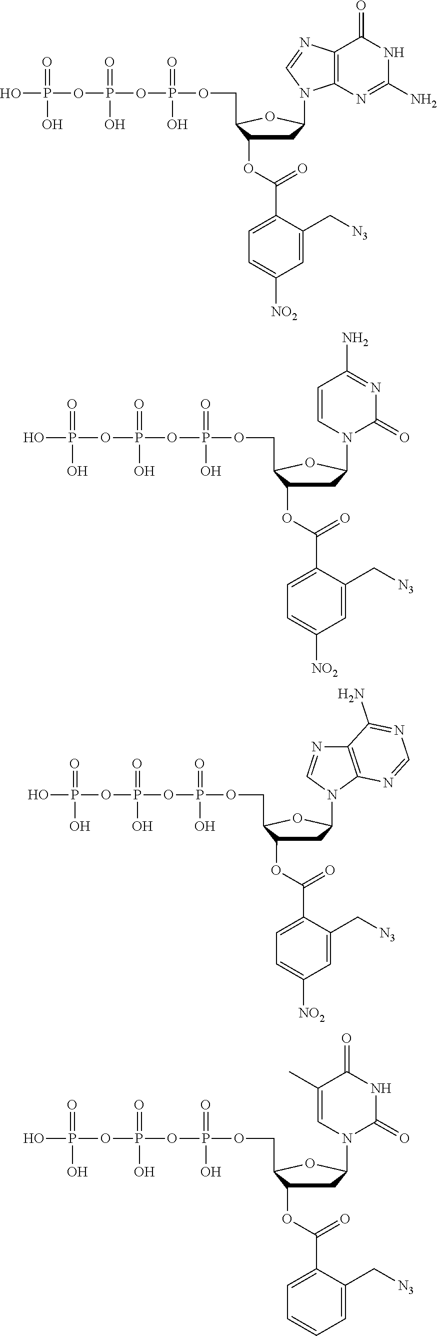

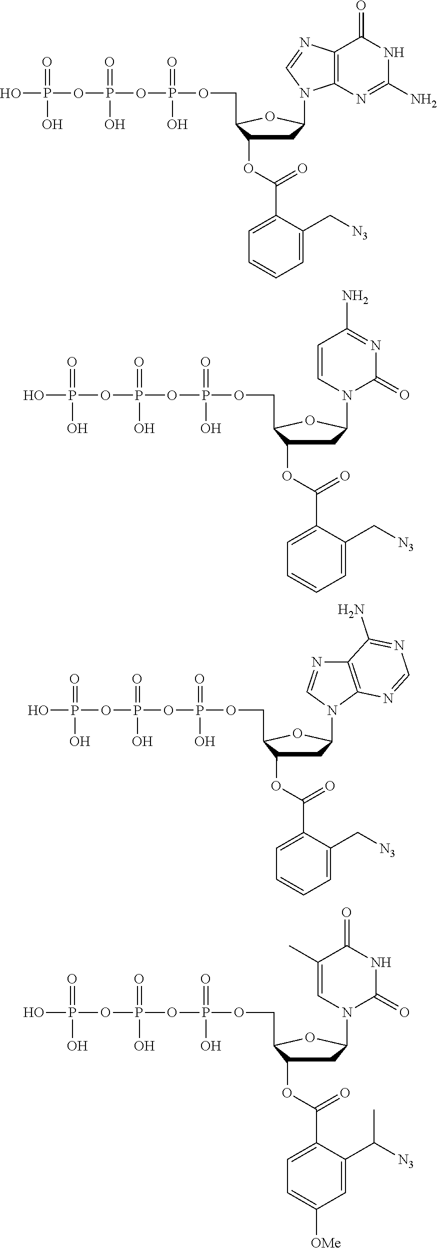

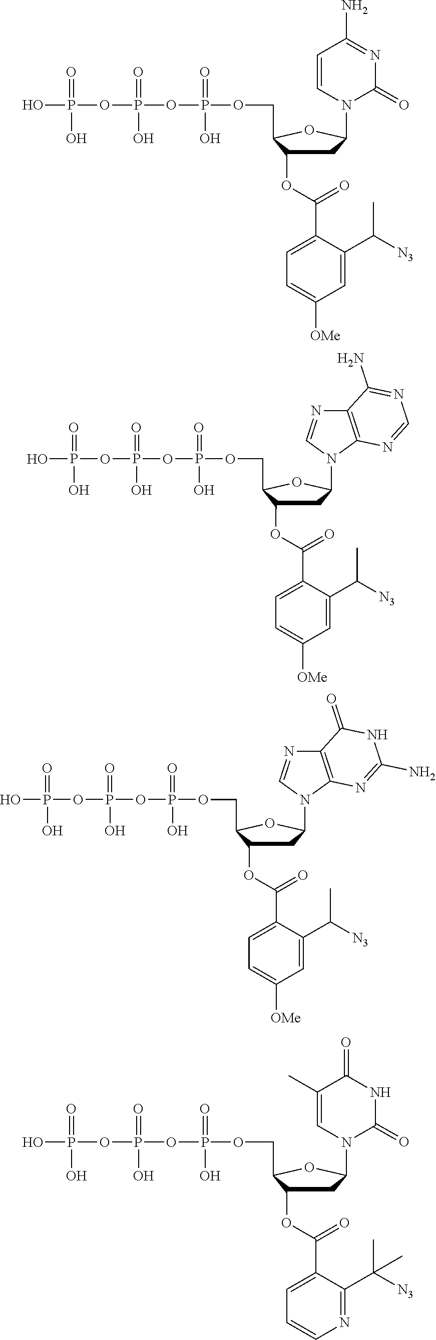

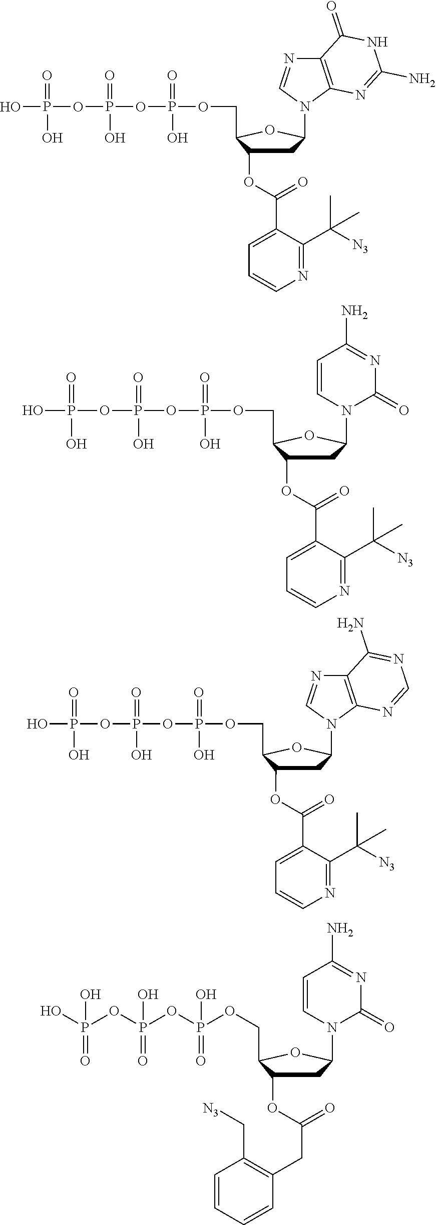

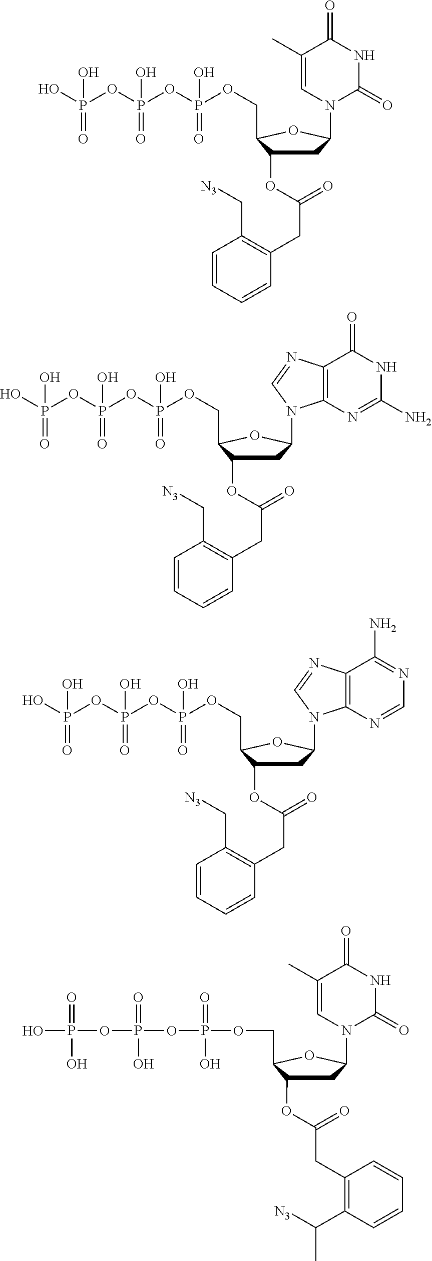

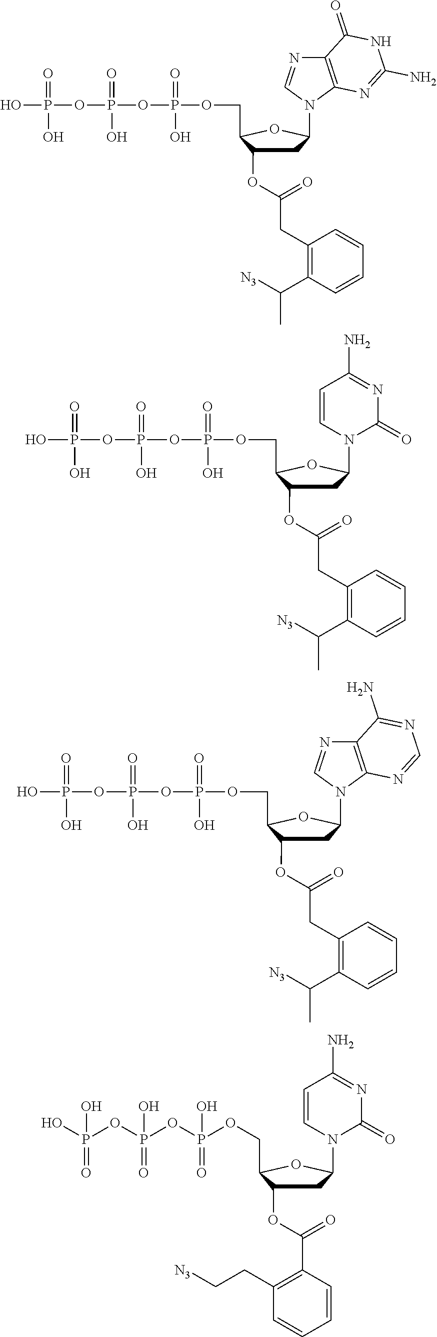

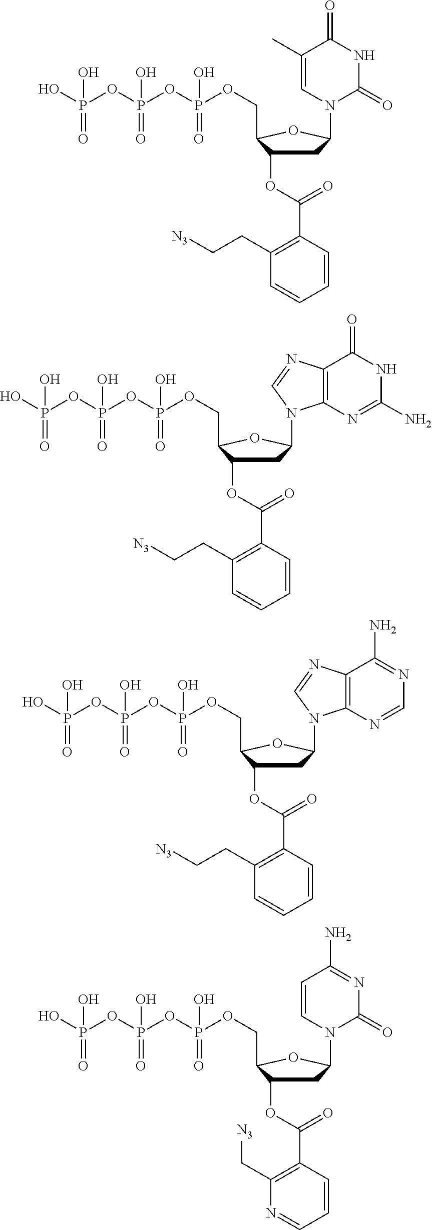

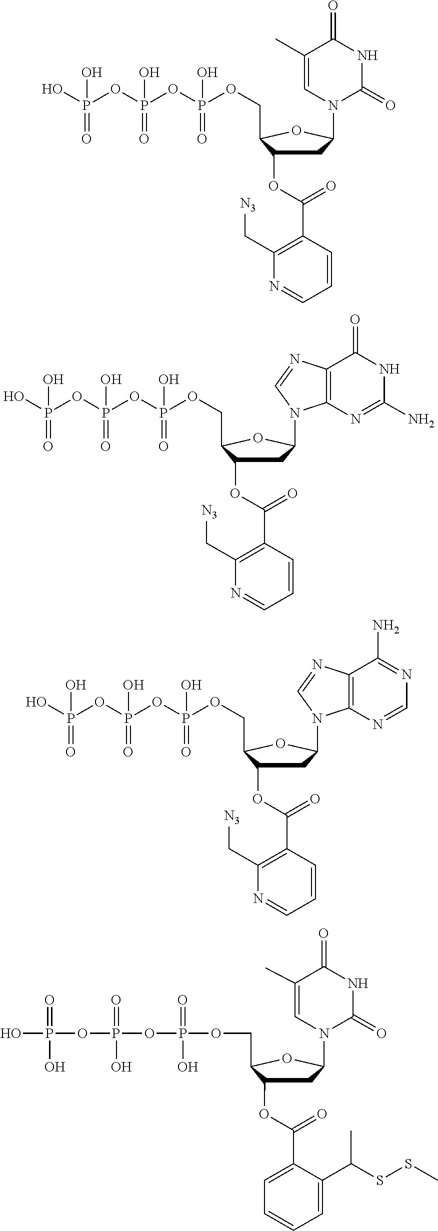

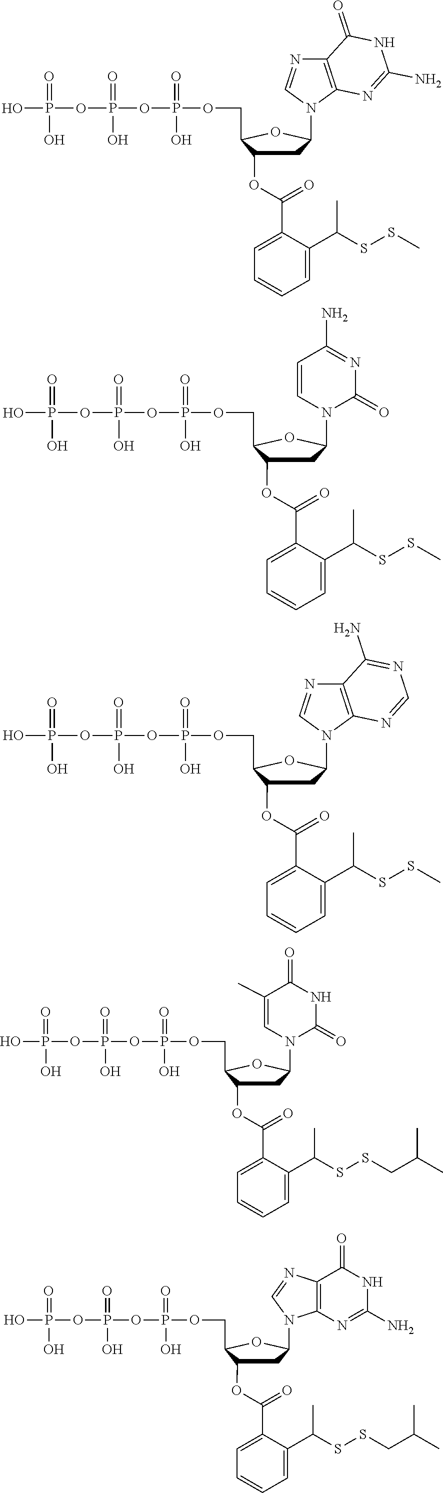

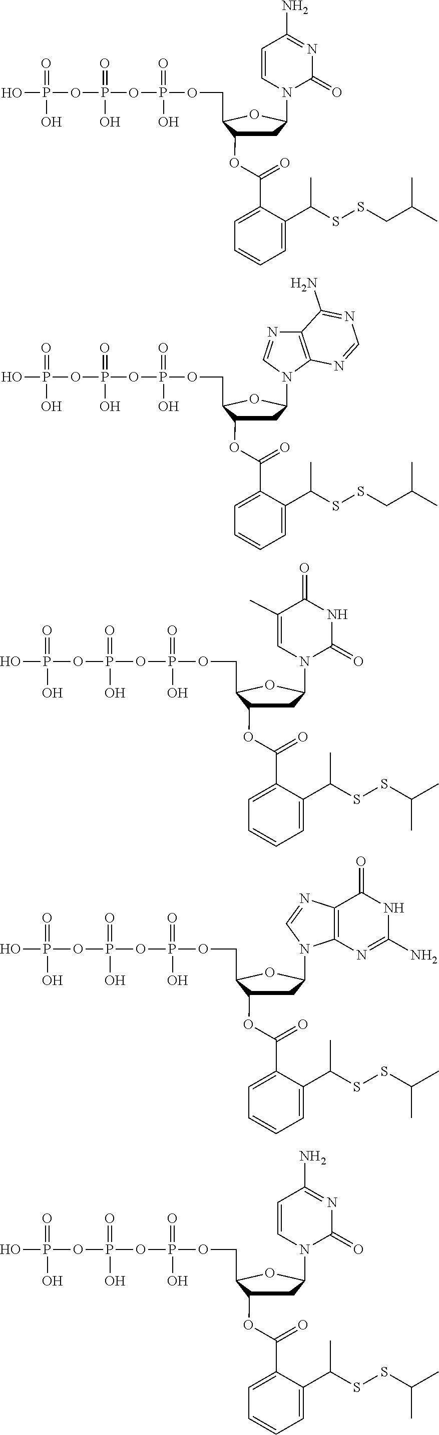

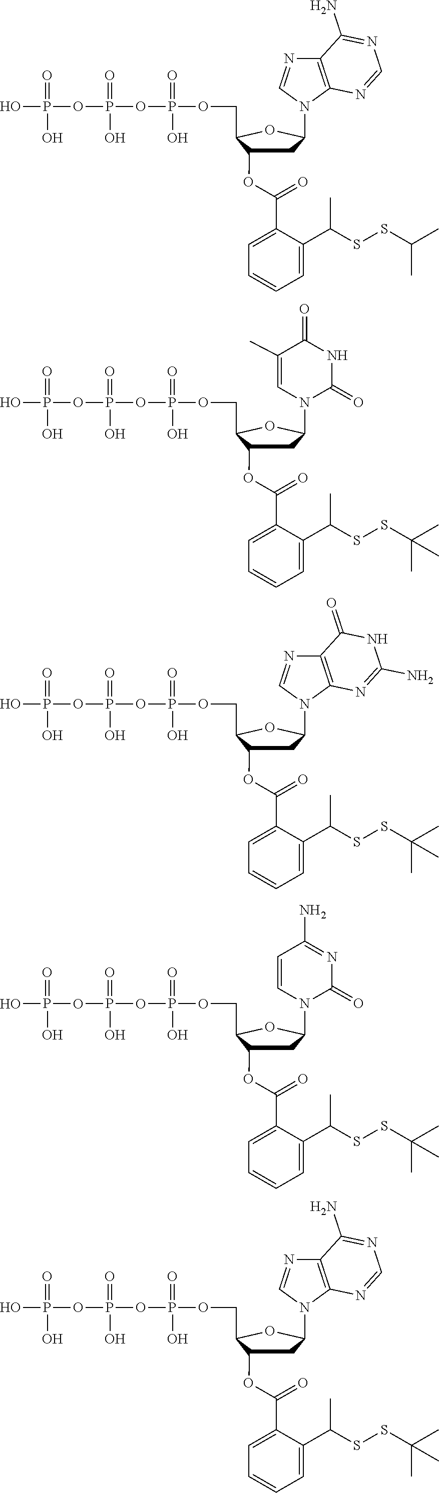

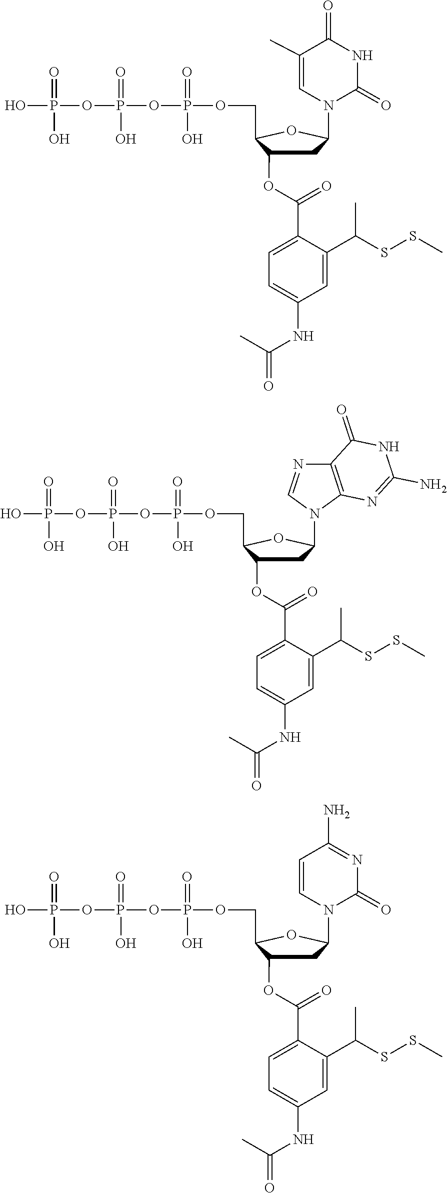

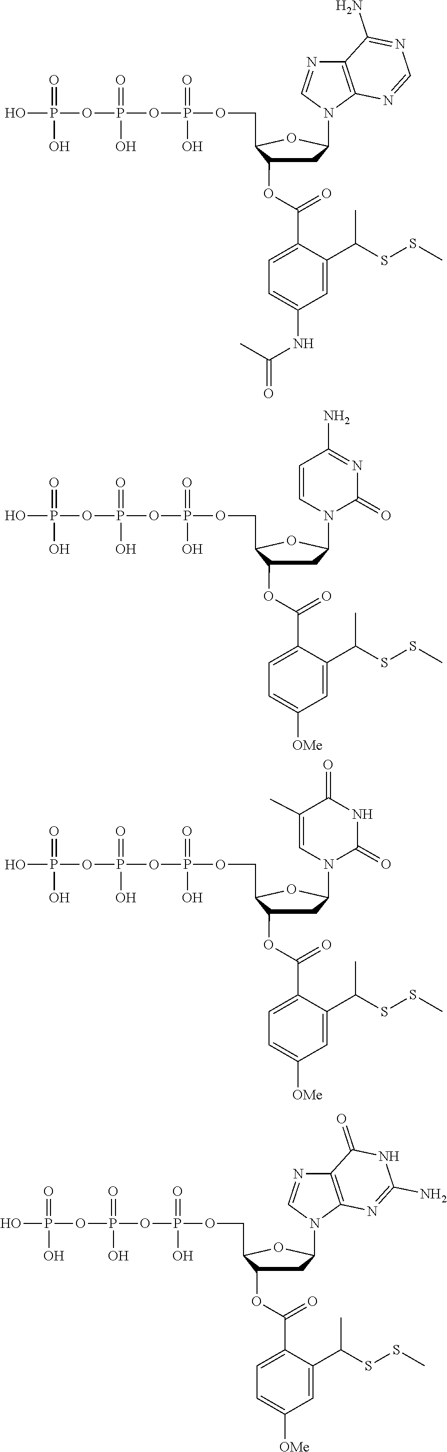

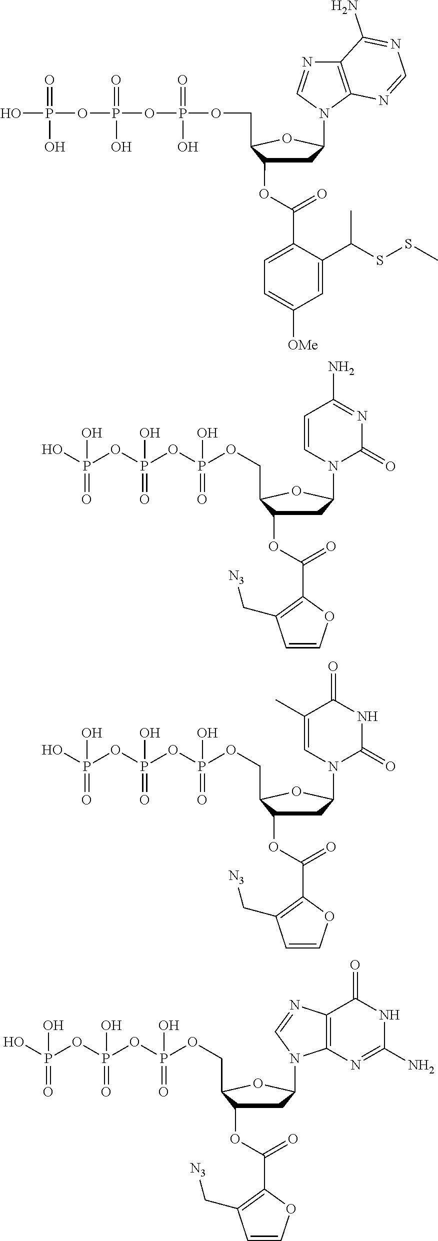

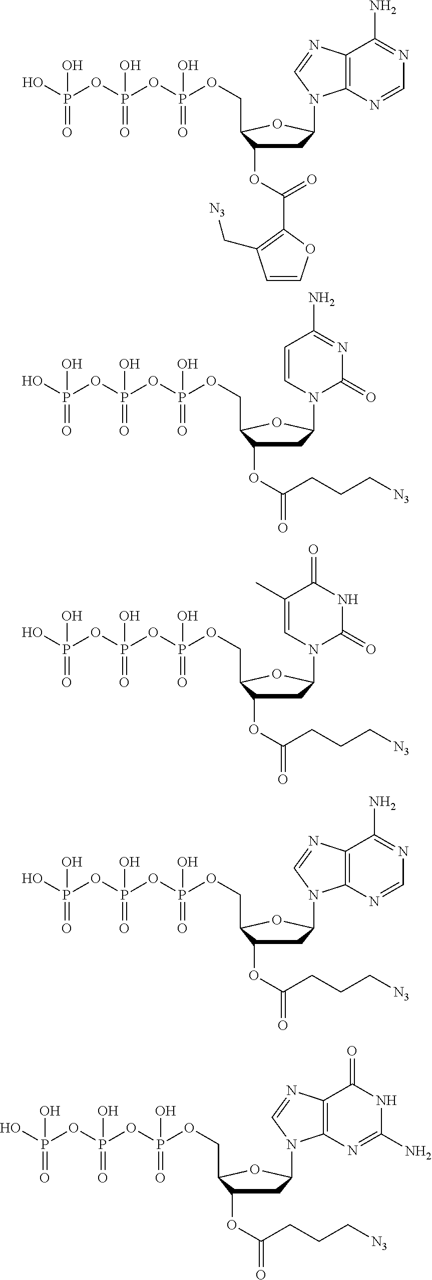

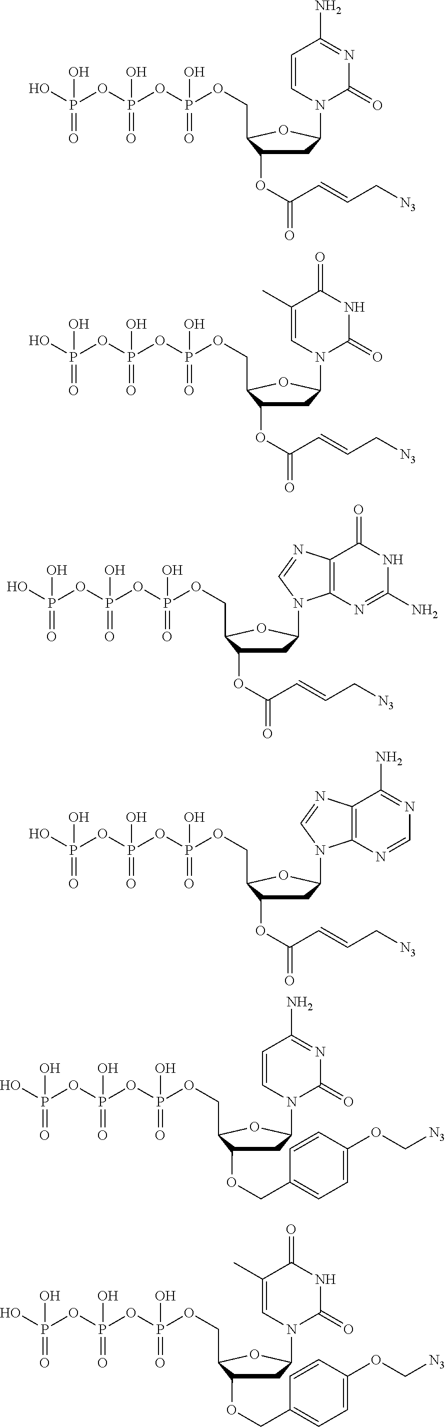

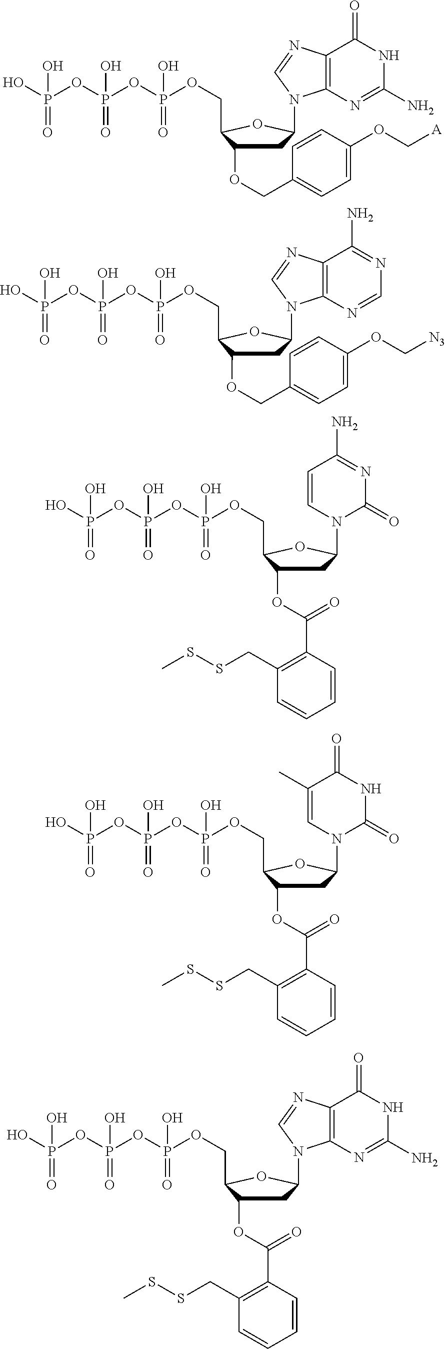

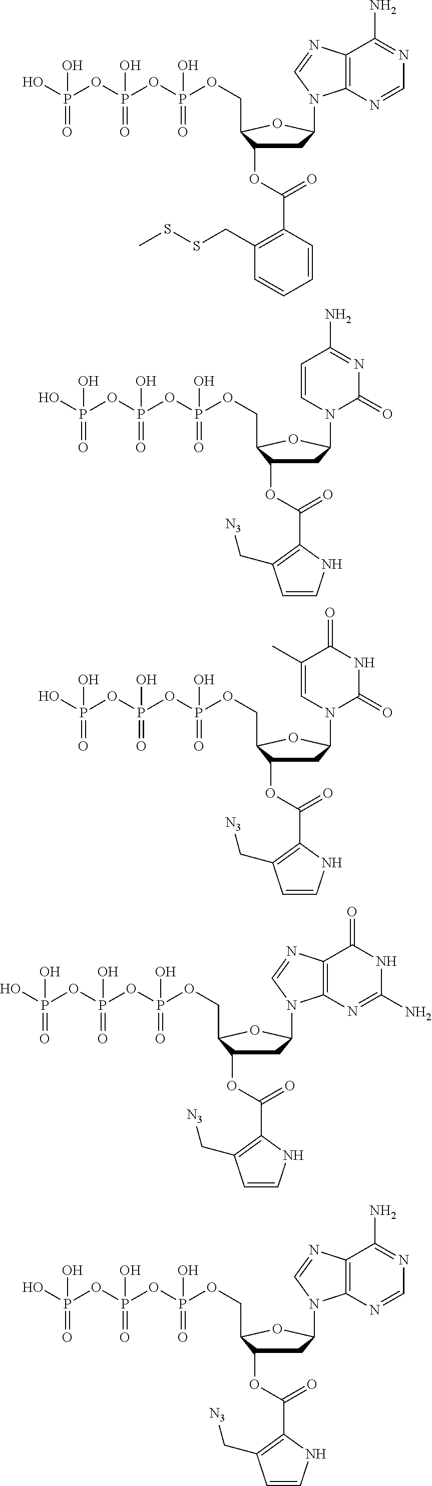

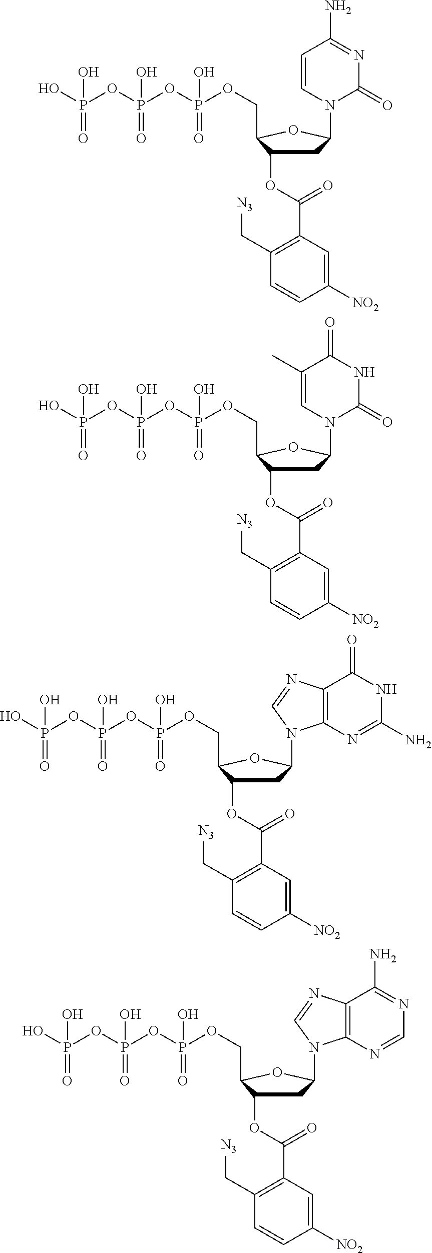

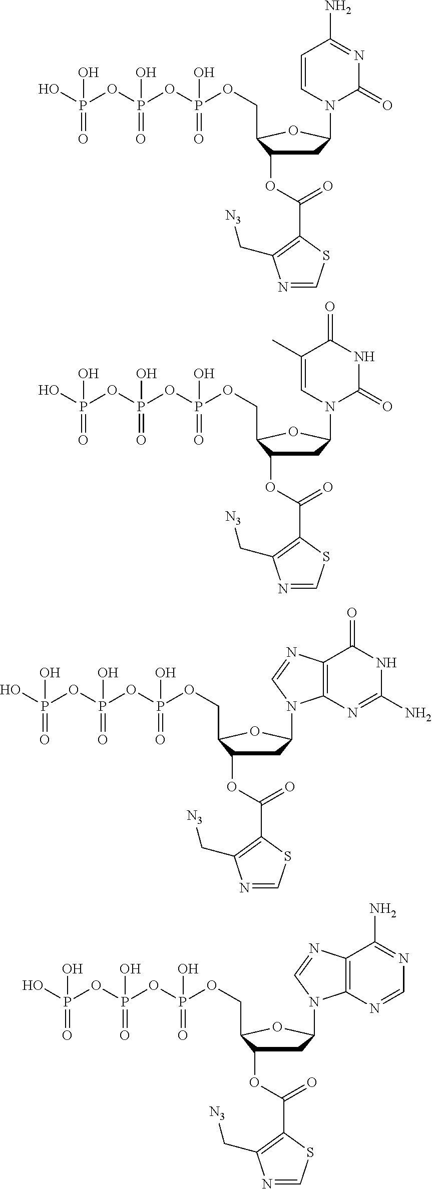

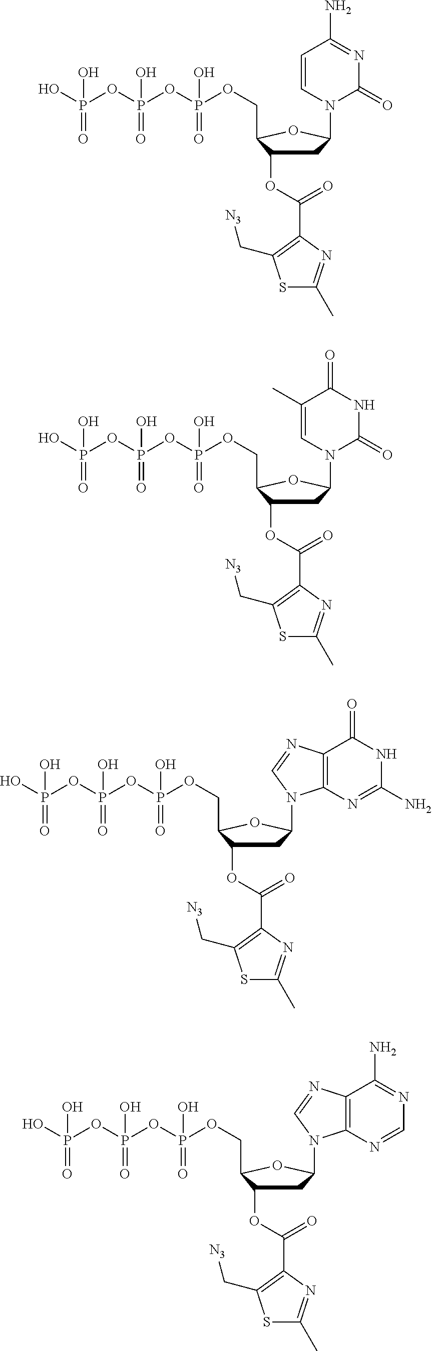

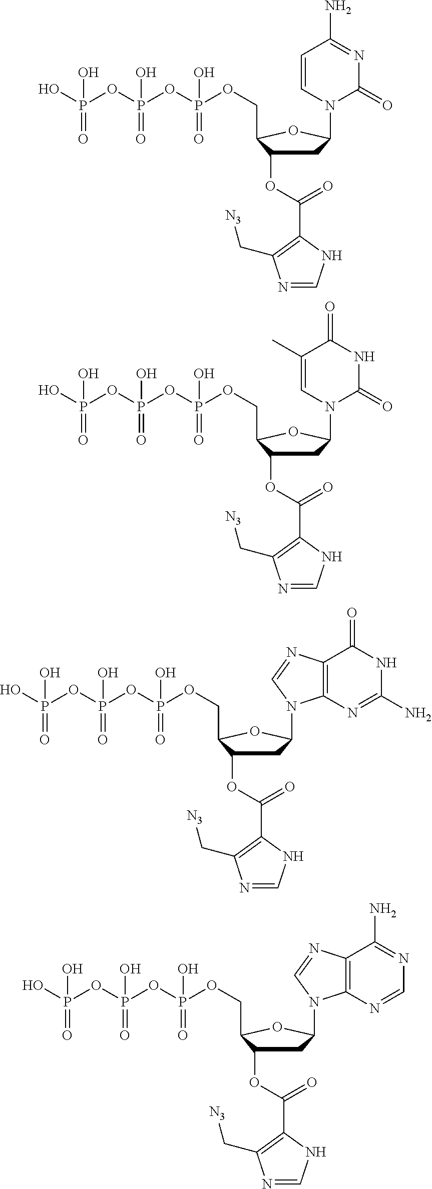

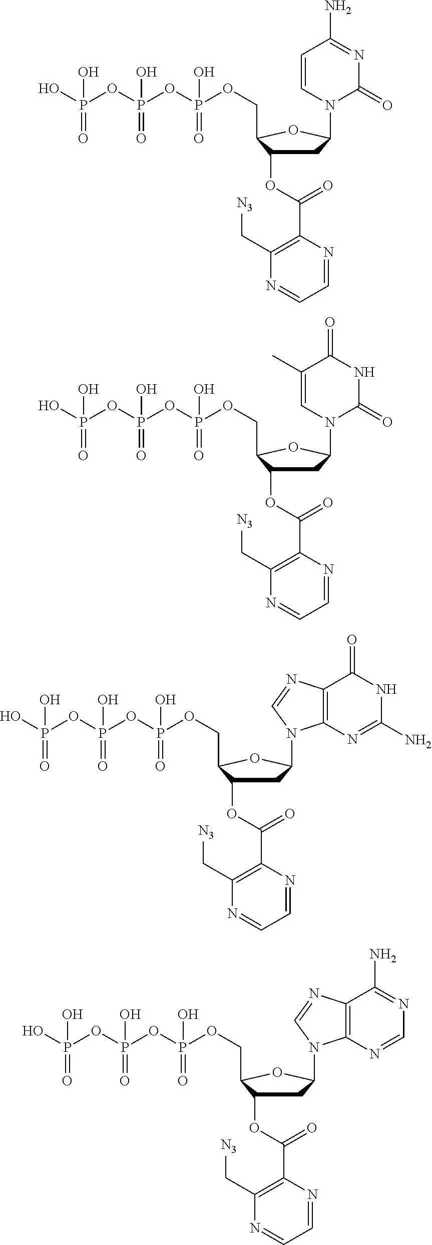

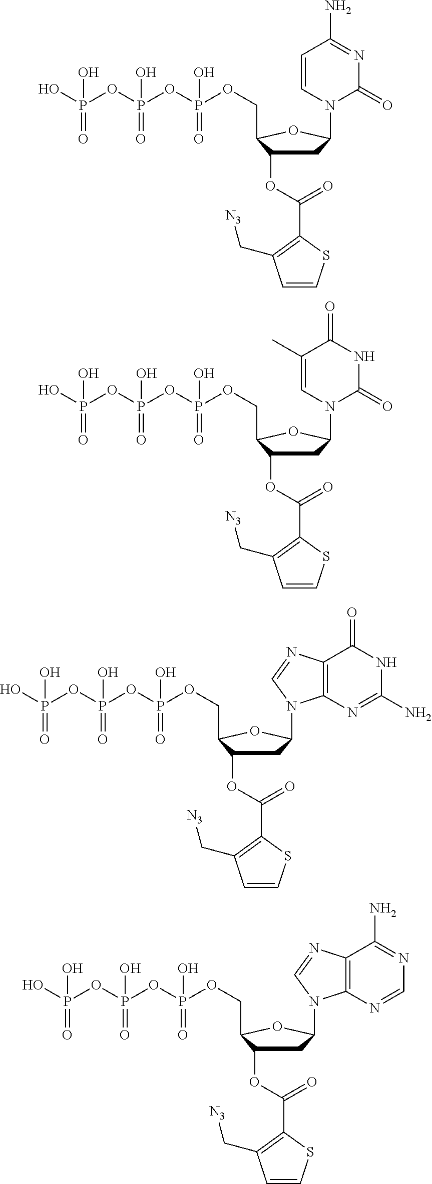

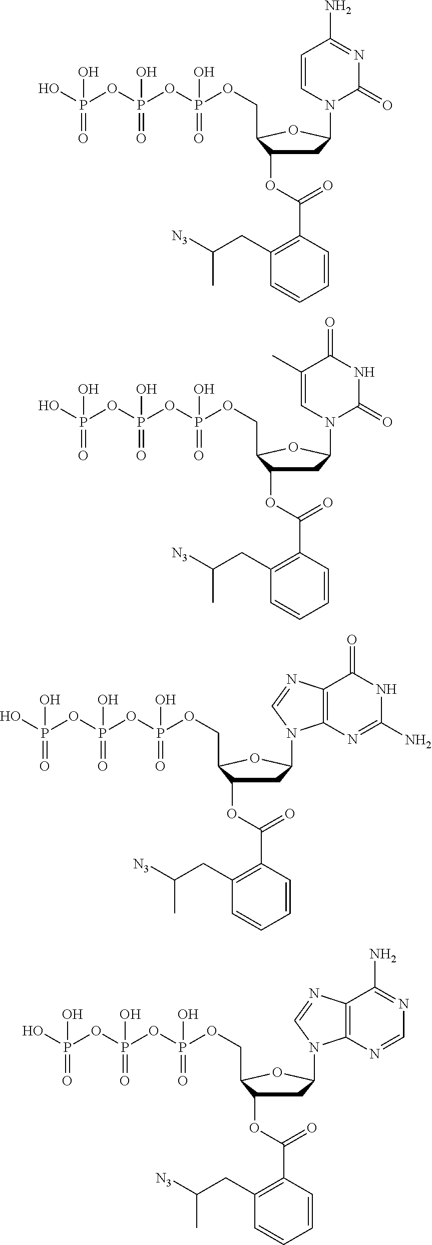

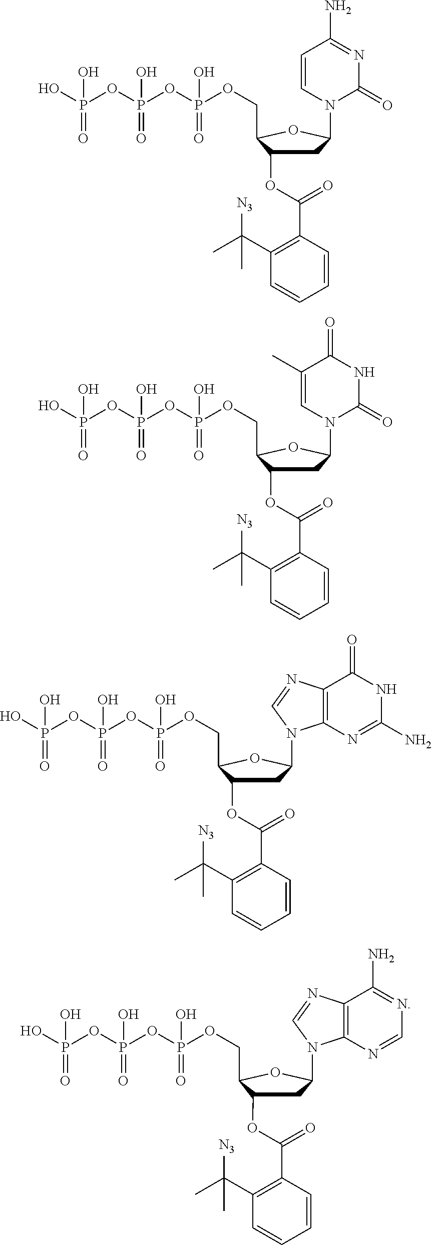

In some embodiments, the compound or a salt thereof as described above carries an additional detectable label (e.g., a fluorescent label).

In some embodiments, the additional detectable label carried by the compound or a salt thereof is introduced by an affinity reagent (e.g., antibody, aptamer, Affimer, Knottin), wherein the affinity reagent carries the detectable label, and the affinity reagent can specifically recognize and bind to an epitope of the compound or a salt thereof.

In some embodiments, the additional detectable label (e.g., a fluorescent label) is linked to the compound or a salt thereof optionally via a linker.

In some embodiments, the additional detectable label (e.g., a fluorescent label) is linked to the Base of the compound or a salt thereof optionally via a linker.

In some embodiments, when the additional detectable label (e.g., a fluorescent label) is linked to the Base of the compound or a salt thereof optionally via a linker, the structure of Base is selected from the followings:

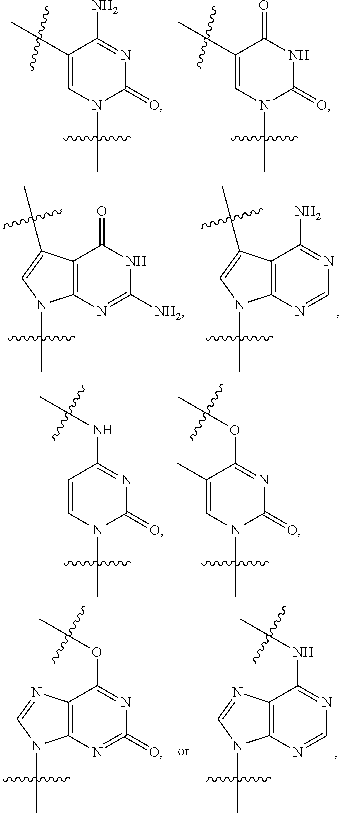

preferably selected from

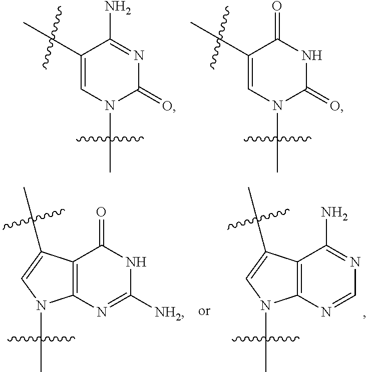 [SMILES: *c1cn(*)c(=O)nc1N ; *c1cn(*)c(=O)[nH]c1=O ; *c1cn(*)c2nc(N)[nH]c(=O)c12 ; "or" ; *c1cn(*)c2ncnc(N)c12]

In some embodiments, the linker is a cleavable linker or a non-cleavable linker.

In some embodiments, the cleavable linker is selected from a linker capable of being cleaved by electrophilic reaction, a linker capable of being cleaved by nucleophilic reaction, a linker capable of being cleaved by photolysis, a linker capable of being cleaved under reductive conditions, a linker capable of being cleaved under oxidative conditions, a safety-catch linker, a linker capable of being cleaved by elimination mechanisms, or any combination thereof.

In some embodiments, the linker has a structure of formula (B):

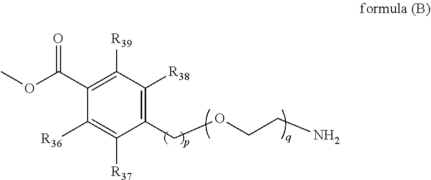

wherein:
R³⁶, R³⁷, R³⁸, R³⁹ are each independently selected from H, —N₃, N₃—C₁-C₆ alkyl, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy), the C₁-C₆ alkyl is optionally substituted by C₁-C₆ alkyl;
p is selected from 1, 2, 3, 4, 5, or 6;
q is selected from any integer between 1 and 12.

In some particular embodiments, any one of R³⁶, R³⁷, R³⁸, and R³⁹ is N₃—C₁-C₆ alkyl, and wherein the C₁-C₆ alkyl is optionally substituted by C₁-C₆ alkyl, the others are each independently selected from H, —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy).

In some particular embodiments, R³⁶ is N₃-C₁-C₆ alkyl, and wherein the C₁-C₆ alkyl is optionally substituted by C₁-C₆ alkyl, R³⁷, R³⁸, and R³⁹ are each independently selected from H, C₁-C₆ alkyl.

In some particular embodiments, R³⁶ is N₃-methyl optionally substituted by methyl, and R³⁷, R³⁸, and R³⁹ are H.

In some particular embodiments, p is selected from 1, 2, or 3.

In some particular embodiments, p is 1.

In some particular embodiments, q is selected from 2, 3, 4, 5, or 6.

In some particular embodiments, q is 4.

In some embodiments, methyl end of the linker of formula (B) is connected to the Base, and the amino end thereof is connected to the additional detectable label (e.g., a fluorescent label). For example,

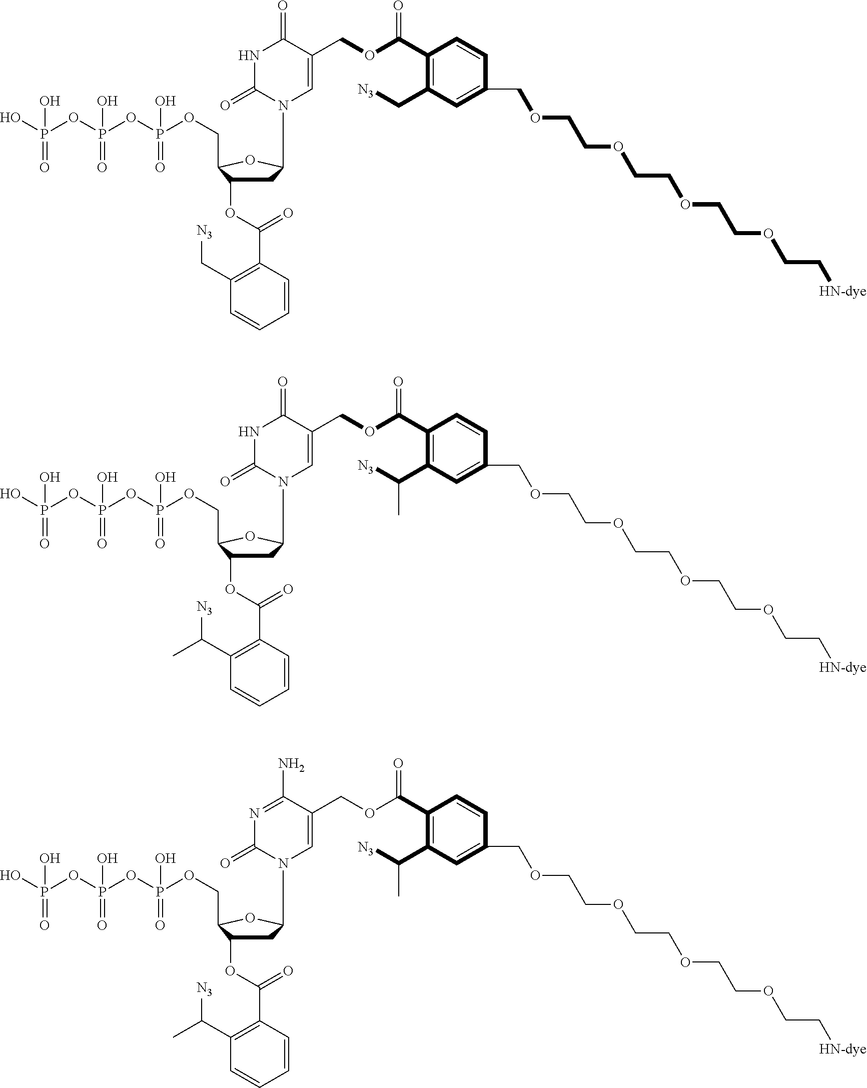

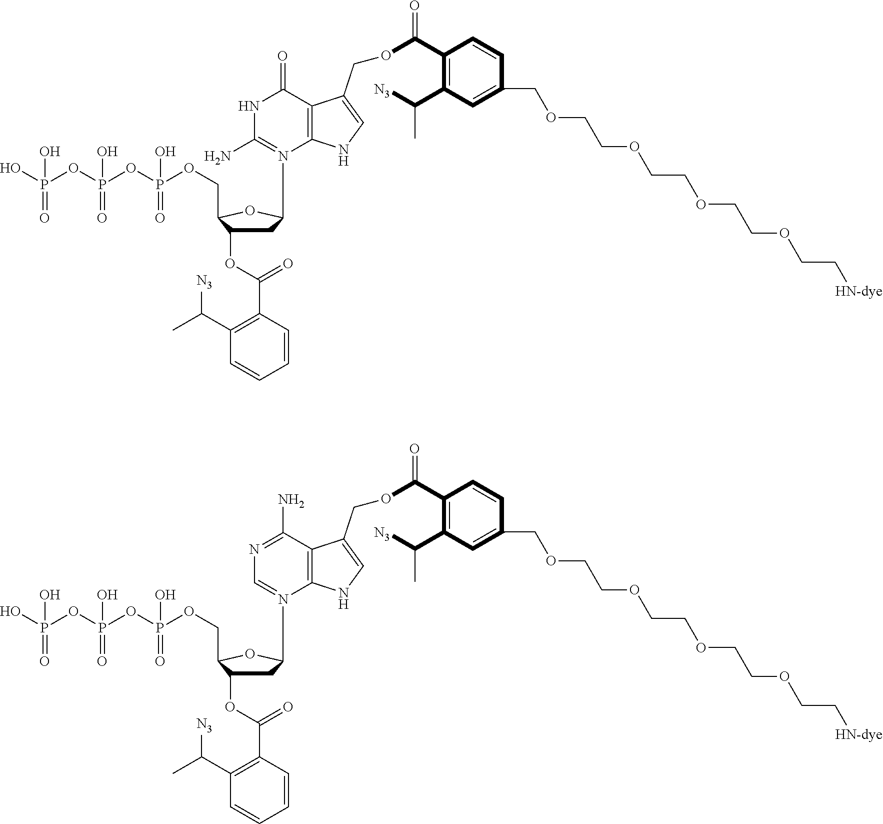

In some embodiments, the linker of formula (B) has the following structure

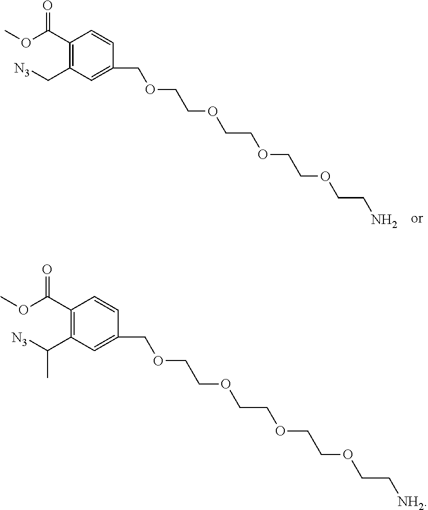 [SMILES: COC(=O)c1ccc(COCCOCCOCCOCCN)cc1CN=[N+]=[N-] ; "or" ; COC(=O)c1ccc(COCCOCCOCCOCCN)cc1C(C)N=[N+]=[N-]]

In some embodiments, the linker has the structure of formula (C):

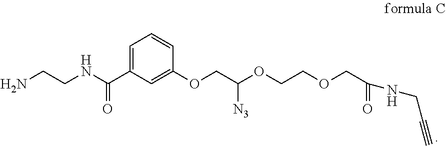 [SMILES: C#CCNC(=O)COCCOC(COc1cccc(C(=O)NCCN)c1)N=[N+]=[N-]]

In some embodiments, the alkynyl end of the linker of formula (C) is connected to the Base, and the amino end thereof is connected to the additional detectable label (e.g., a fluorescent label). For example,

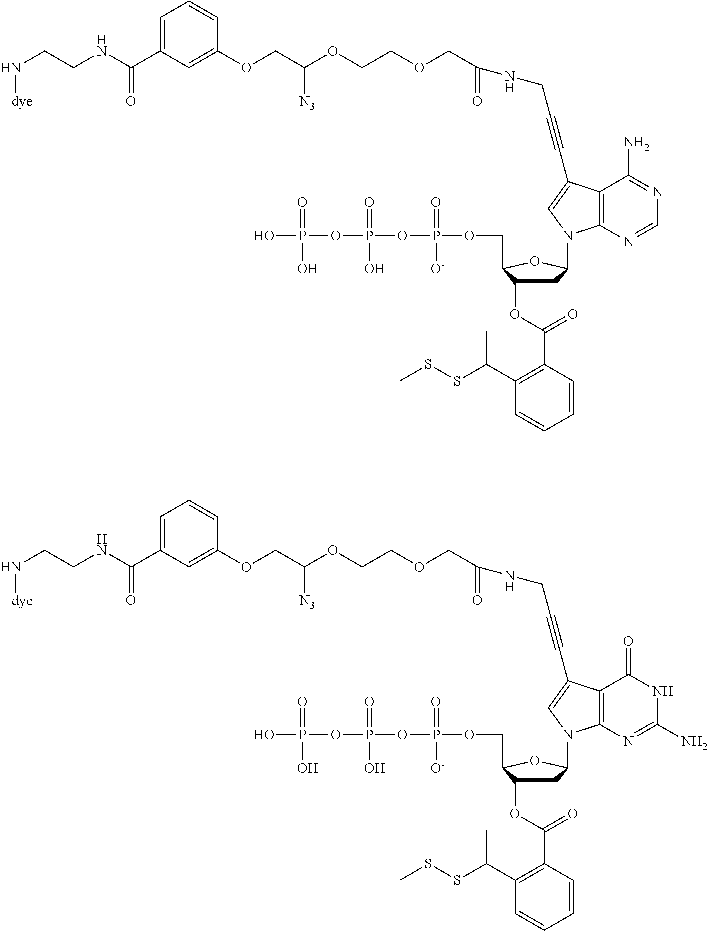

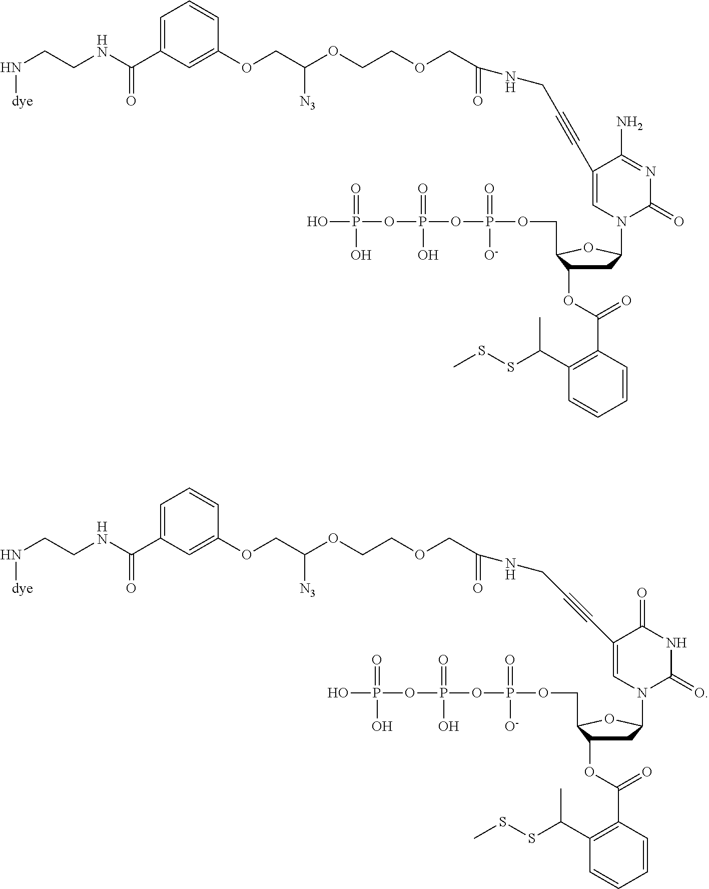

In some embodiments, the linker has the structure of formula (D):

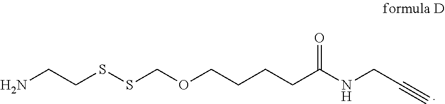 [SMILES: C#CCNC(=O)CCCCOCSSCCN]

In some embodiments, the alkynyl end of the linker of formula (D) is connected to the Base, and the amino end thereof is connected to the additional detectable label (e.g., a fluorescent label). For example,

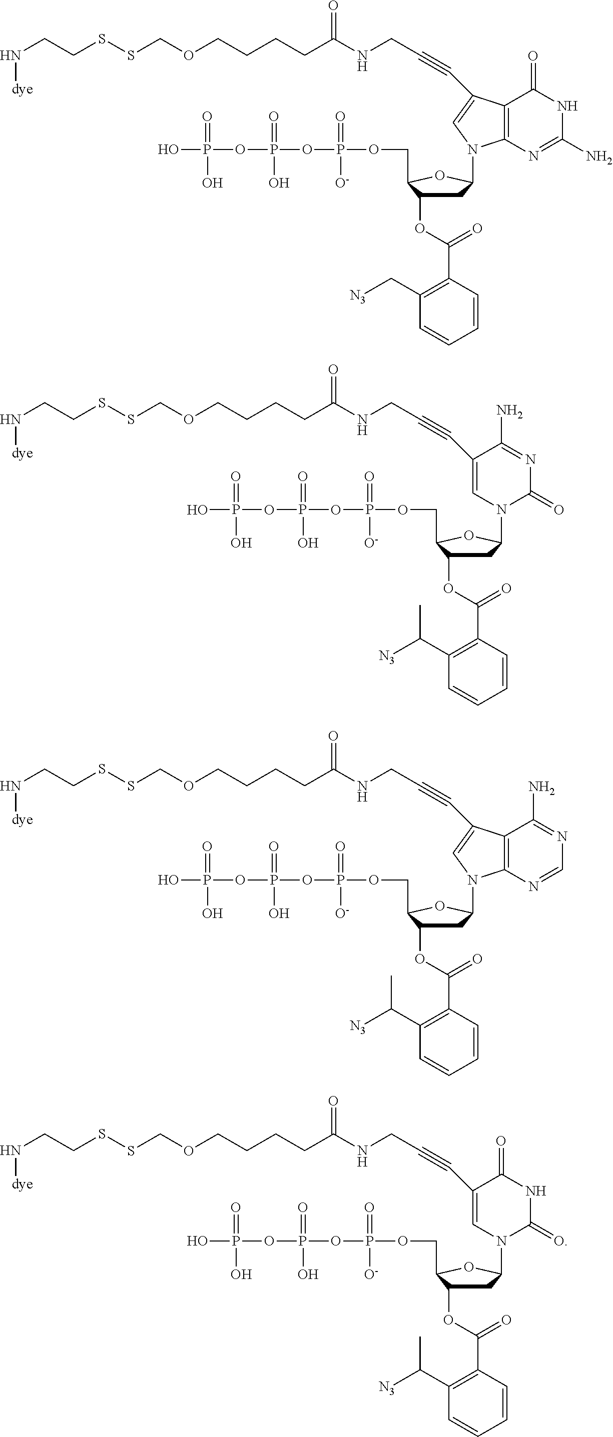

In some embodiments, the detectable label is selected from the followings:

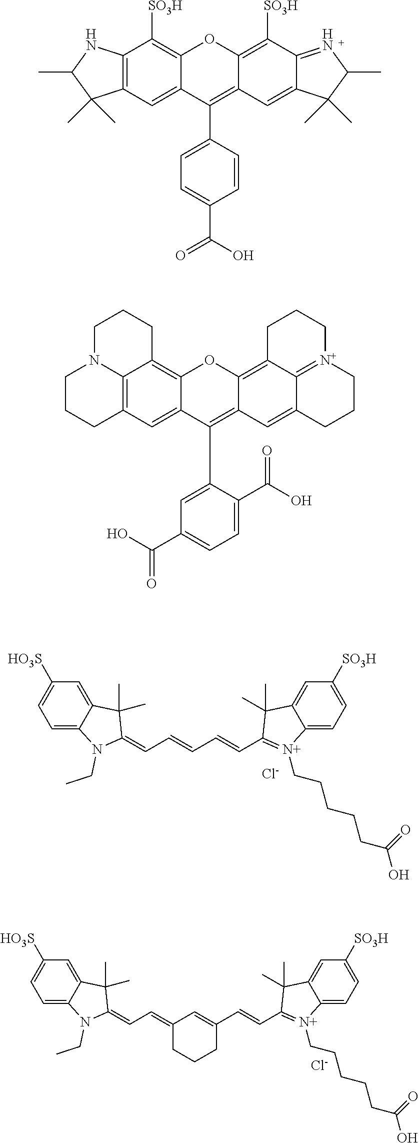

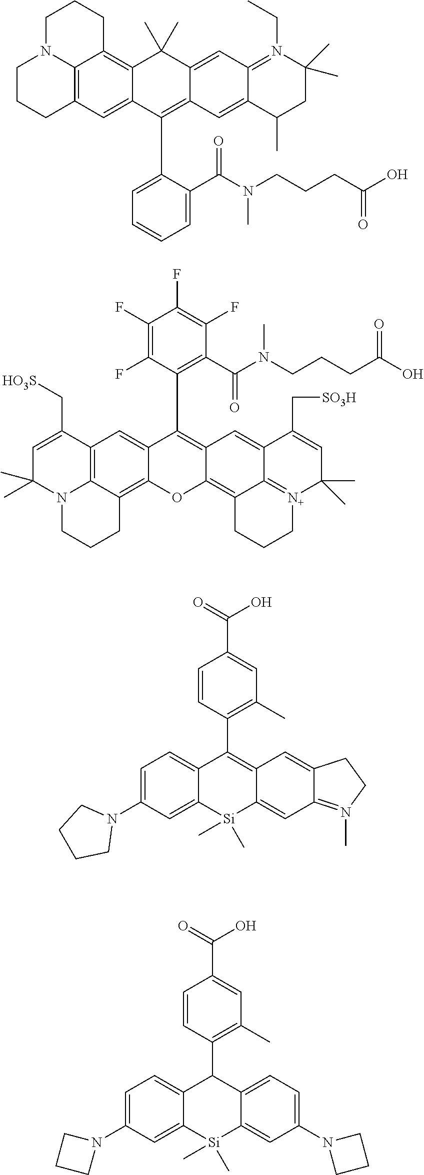

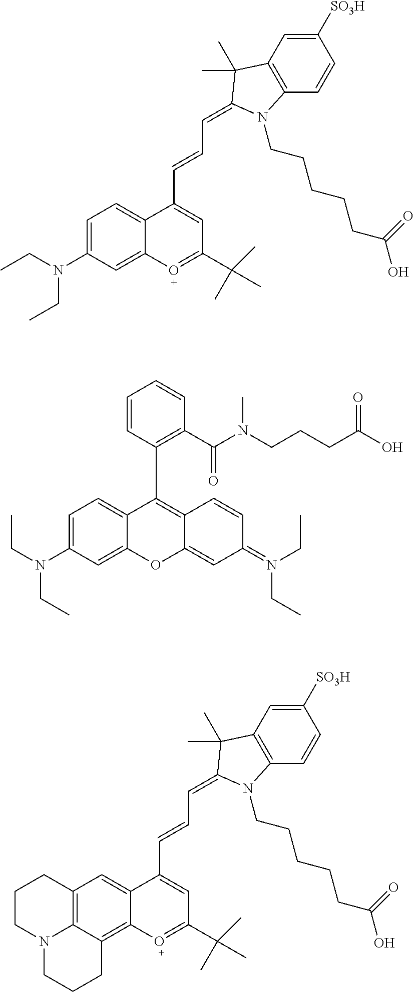

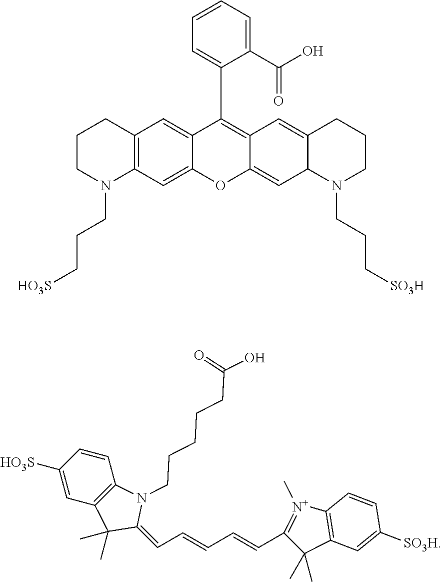 [SMILES: O=C(O)c1ccccc1C1=C2C=C3CCCN(CCCS(=O)(=O)O)C3C=C2Oc2cc3c(cc21)CCCN3CCCS(=O)(=O)O ; C[N+]1=C(/C=C/C=C/C=C2\N(CCCCCC(=O)O)c3ccc(S(=O)(=O)O)cc3C2(C)C)C(C)(C)c2cc(S(=O)(=O)O)ccc21]

In some embodiments, if the Base is different, the detectable label (e.g., a fluorescent label) varies.

In some embodiments, the compound or a salt thereof carrying the additional detectable label has the following structure:

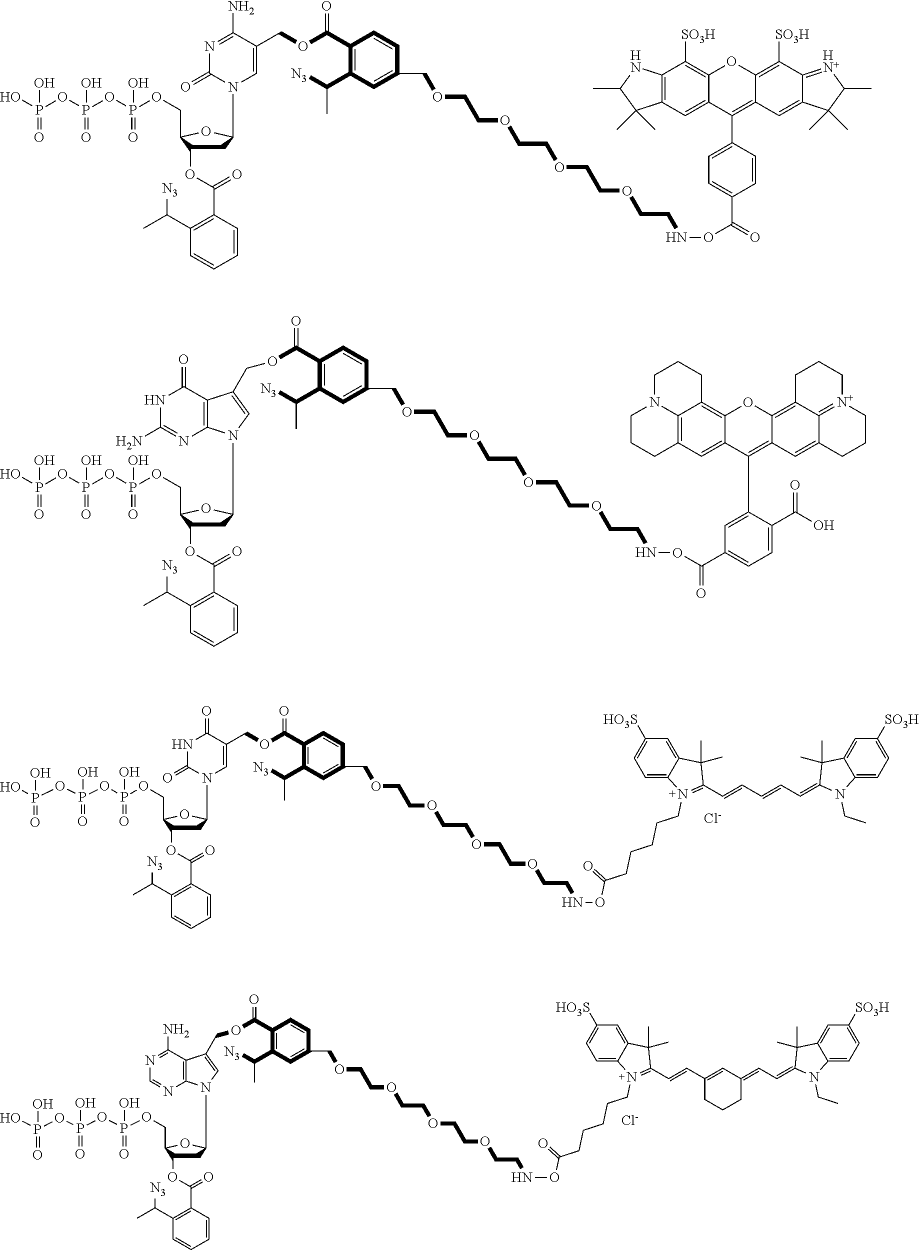

In some embodiments, the compound or a salt thereof carrying additional detectable label has the following structure:

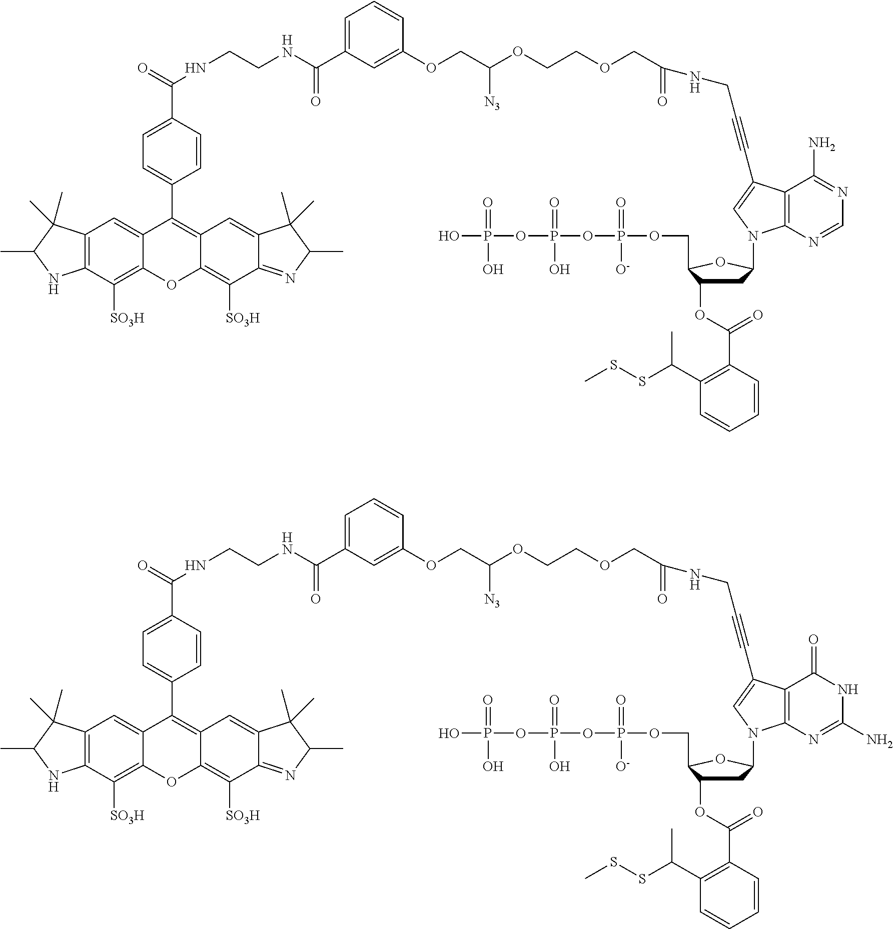

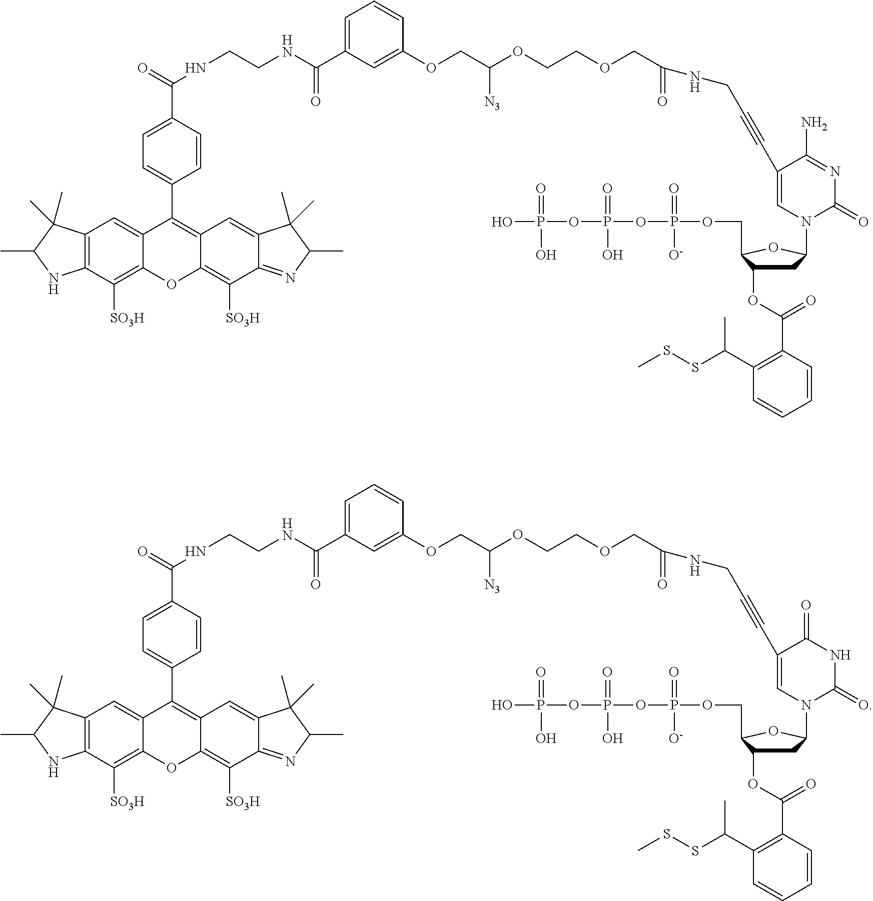

In some embodiments, the compound or a salt thereof carrying additional detectable label has the following structure:

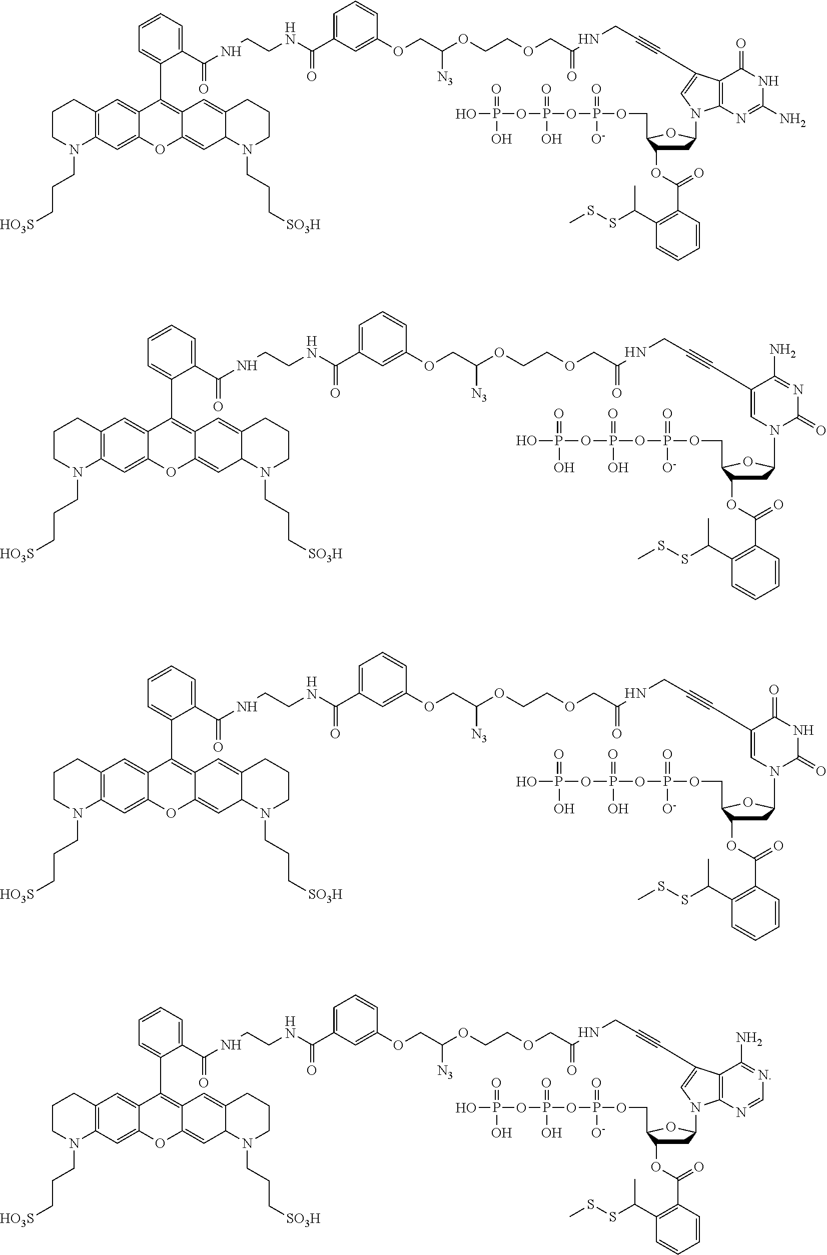

In some embodiments, the compound or a salt thereof carrying additional detectable label has the following structure

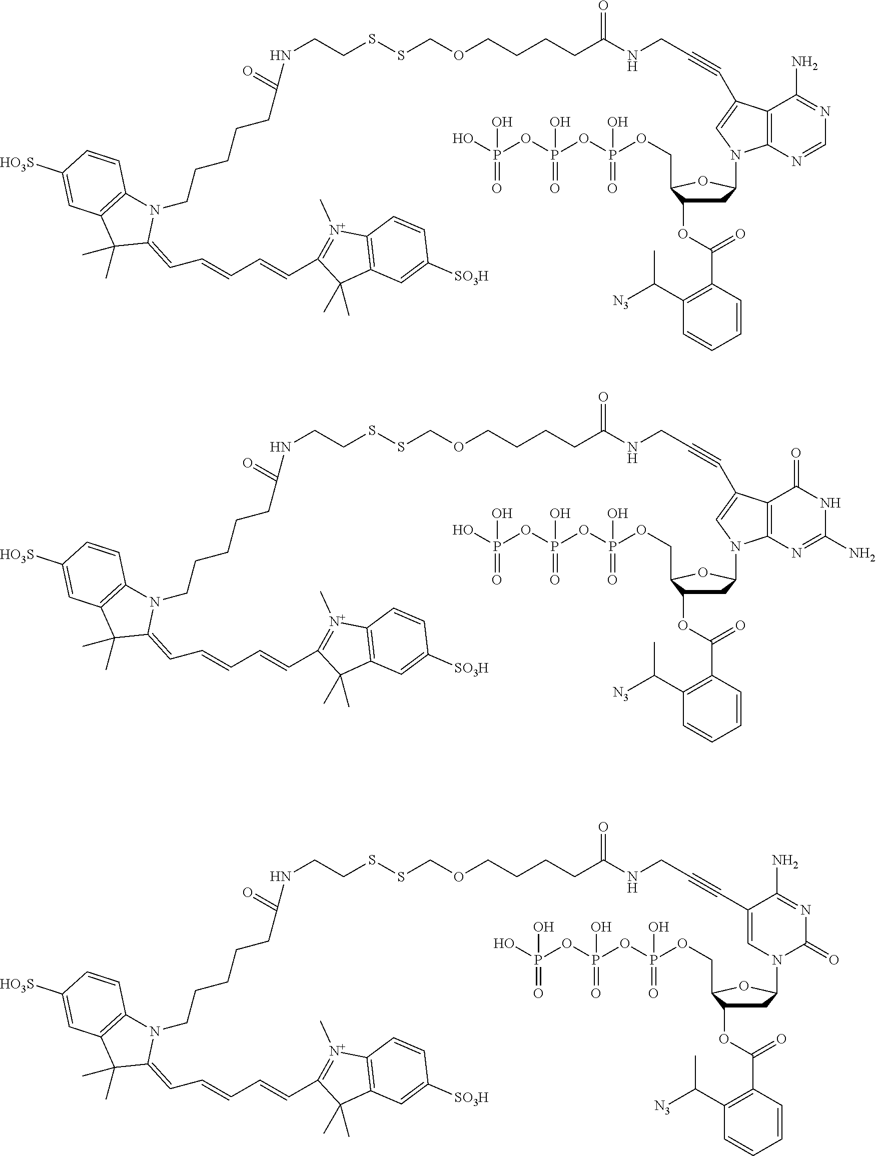

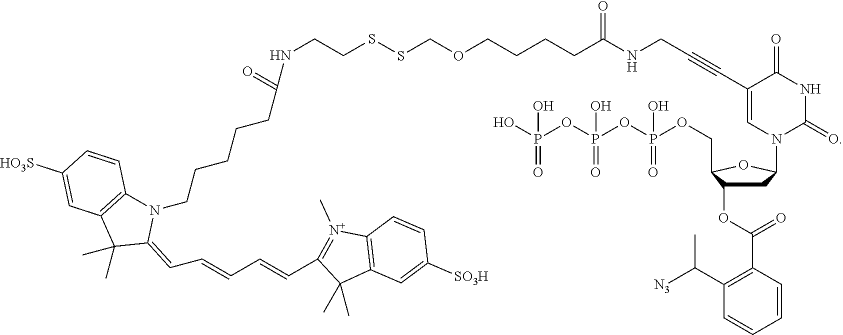

In the seventh aspect of the invention, the present invention provides a method for terminating a nucleic acid synthesis, which comprises incorporating the compound or a salt thereof as described above into nucleic acid molecule to be terminated.

In some embodiments, the incorporation of the compound or a salt thereof is achieved by a terminal transferase, a terminal polymerase or a reverse transcriptase.

In some embodiments, the method comprises incorporating the compound or a salt thereof into the nucleic acid molecule to be terminated by using a polymerase.

In some embodiments, the method comprises performing the nucleotide polymerization reaction using a polymerase under conditions where the polymerase is allowed to carry out the nucleotide polymerization reaction, thereby incorporating the compound or a salt thereof into the 3′ end of the nucleic acid molecule to be terminated.

In the eighth aspect of the invention, the present invention provides a method for preparing a growing polynucleotide complementary to a target single-stranded polynucleotide in a sequencing reaction, which comprises incorporating the compound or a salt thereof as described above into the growing complementary polynucleotide, wherein the incorporation of the compound or a salt thereof prevents any subsequent nucleotides from being introduced into the growing complementary polynucleotide.

In some embodiments, the incorporation of the compound or a salt thereof is achieved by a terminal transferase, a terminal polymerase or a reverse transcriptase.

In some embodiments, the method comprises incorporating compound or a salt thereof into the growing complementary polynucleotide by using a polymerase.

In some embodiments, the method comprises performing the nucleotide polymerization reaction using a polymerase under conditions where the polymerase is allowed to carry out the nucleotide polymerization reaction, thereby incorporating the compound or a salt thereof into the 3′ end of the growing complementary polynucleotide.

In the ninth aspect of the invention, the present invention provides a method for determining the sequence of a target single-stranded polynucleotide, which comprises: 1) monitoring the sequential incorporation of nucleotides complementary to the target single-stranded polynucleotide in a growing nucleic acid strand, wherein at least one complementary nucleotide incorporated is the compound or a salt thereof as described above, and the compound or a salt thereof carries an additional detectable label (e.g., a fluorescent label), and,

2) detecting the detectable label.

In some embodiments, the additional detectable label (e.g., a fluorescent label) is linked to the compound or a salt thereof optionally via a linker.

In some embodiments, the linker is as described above.

In some embodiments, the additional detectable label is as described above.

In some embodiments, If the Base is different, the detectable label (e.g., a fluorescent label) carried by the compound or a salt thereof varies.

In some embodiments, prior to introducing a next complementary nucleotide, the reversible blocking group (R) and the detectable label in the compound or a salt thereof are removed.

In some embodiments, the reversible blocking group (R) and the detectable label are removed simultaneously.

In some embodiments, the reversible blocking group (R) and the detectable label are removed sequentially; for example, after the detectable label is removed, the reversible blocking group is removed, or, after the reversible blocking group is removed, the detectable label is removed.

In some embodiments, the method comprises the following steps:
(a) providing a plurality of different nucleotides, wherein the plurality of different nucleotides are the compound or a salt thereof as described above, wherein each nucleotide carries an additional detectable label that can be distinguished from an additional detectable label carried by another nucleotide during detection;
(b) incorporating the plurality of different nucleotides into sequence complementary to a target single-stranded polynucleotide;
(c) detecting the additional detectable labels carried by the nucleotides in step (b) to determine the types of nucleotides incorporated;
(d) removing the reversible blocking groups and the detectable labels carried by the nucleotides in step (b); and
(e) optionally repeating steps (b)-(d) one or more times;
whereby the sequence of the target single-stranded polynucleotide is determined.

In some embodiments, the method comprises the following steps:
(1) providing a first nucleotide, a second nucleotide, a third nucleotide and a fourth nucleotide, wherein at least one of the four nucleotides is the compound or a salt thereof as described above, the Bases contained in the four nucleotides are different from each other, and the four nucleotides carry additional detectable label (e.g., a fluorescent label), preferably, the additional detectable label carried by the four nucleotides is introduced by an affinity reagent (e.g., antibody, aptamer, Affimer, Knottin), wherein the affinity reagent carries the detectable label, and the affinity reagent can specifically recognize and bind to an epitope of each nucleotide, or preferably, the four nucleotides are linked to the additional detectable label optionally via a linker, more preferably, the Base of the four nucleotides is linked to the additional detectable label optionally via a linker, most preferably, the additional detectable labels carried by the four nucleotides are different from each other;
(2) contacting the four nucleotides with a target single-stranded polynucleotide; removing the nucleotides that are not incorporated into the growing nucleic acid strand; detecting the detectable labels carried by the nucleotides that are incorporated into the growing nucleic acid strand; removing the reversible blocking groups and the detectable labels carried by the nucleotides that are incorporated into the growing nucleic acid strand;
optionally, it also includes (3): repeat the step (2) one or more times.

In some embodiments, the method comprises the following steps:
(a) providing a mixture comprising a duplex, at least one compound or a salt thereof as described above, polymerase and an excision reagent; wherein the duplex comprises growing nucleic acid strand and a nucleic acid strand to be sequenced; wherein the compound or a salt thereof carries an additional detectable label (e.g., a fluorescent label), preferably, the additional detectable label carried by the compound or a salt thereof is introduced by an affinity reagent (e.g., antibody, aptamer, Affimer, Knottin), wherein the affinity reagent carries the detectable label, and the affinity reagent can specifically recognize and bind to an epitope of the compound or a salt thereof, or preferably, the compound or a salt thereof is linked to the additional detectable label optionally via a linker, or more preferably, the Base of the compound or a salt thereof is linked to the additional detectable label optionally via a linker;
(b) carrying out a reaction comprising the following steps (i), (ii) and (iii), optionally, repeating the steps for one or more times:
step (i): incorporating the compound or a salt thereof into the growing nucleic acid strand using a polymerase to form a nucleic acid intermediate containing the reversible blocking group and the detectable label;
step (ii): detecting the detectable label contained in the nucleic acid intermediate;
step (iii): removing the reversible blocking group and/or the detectable label contained in the nucleic acid intermediate by using the excision reagent.

In some embodiments, the removal of the reversible blocking group and the removal of the detectable label are performed simultaneously, or, the removal of the reversible blocking group and the removal of the detectable label are performed sequentially (for example, the reversible blocking group is removed first, or the detectable label is removed first).

In some embodiments, the excision reagent used for the removal of the reversible blocking group is the same as that used for the removal of the detectable label.

In some embodiments, the excision reagent used for the removal of the reversible blocking group is different from that used for the removal of the detectable label.

In some embodiments, the duplex is linked to a support.

In some embodiments, the growing nucleic acid strand is a primer.

In some embodiments, the primer is annealed to the nucleic acid strand to be sequenced to form the duplex.

In some embodiments, the duplex, the compound or a salt thereof, and the polymerase together form a reaction system containing a solution phase and a solid phase.

In some embodiments, the Bases contained in the compounds or salts thereof are different from each other.

In some embodiments, the additional detectable label carried by the compound or a salt thereof are different from each other.

In some embodiments, the compound or a salt thereof is incorporated into the growing nucleic acid strand using a polymerase under conditions where the polymerase is allowed to carry out a nucleotide polymerization reaction, thereby forming a nucleic acid intermediate containing reversible blocking group and the detectable label.

In some embodiments, the polymerase is selected from KOD polymerase or its mutants thereof (e.g., KOD POL151, KOD POL157, KOD POL171, KOD POL174, KOD POL376, KOD POL391).

In some embodiments, before any step of detecting the detectable label contained in the nucleic acid intermediate, the solution phase of the reaction system in the previous step is removed, and the duplex linked to the support is retained.

In some embodiments, the excision reagent is in contact with the duplex or the growing nucleic acid strand in the reaction system containing a solution phase and a solid phase.

In some embodiments, the excision reagent can remove the reversible blocking group and the additional detectable label carried by the compound that is incorporated into the growing nucleic acid strand, without affecting the phosphodiester bond on the backbone of the duplex.

In some embodiments, after any step of removing the reversible blocking group and/or additional detectable label contained in the nucleic acid intermediate, the solution phase of the reaction system in this step is removed.

In some embodiments, a washing operation is performed after any step comprising a removal operation.

In some embodiments, after step (ii), the method further comprises: determining the type of compound incorporated into the growing nucleic acid strand in step (i) according to the signal detected in step (ii), and determining the type of nucleotide at a corresponding position in the nucleic acid strand to be sequenced based on the principle of base complementary pairing.

In the tenth aspect of the invention, the present invention provides a kit comprising at least one compound or a salt thereof as described above.

In some embodiments, the kit comprises a first compound, a second compound, a third compound and a fourth compound, wherein the first, second, third and fourth compounds are each independently the compound or a salt thereof as described above.

In some embodiments, in the first compound, the Base is selected from adenine, 7-deazaadenine or a tautomer thereof (e.g.,

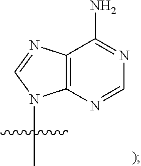 [SMILES: *n1cnc2c(N)ncnc21]

in the second compound, the Base is selected from thymine, uracil or a tautomer thereof (e.g.

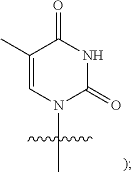 [SMILES: *n1cc(C)c(=O)[nH]c1=O]

in the third compound, the Base is selected from cytosine or a tautomer thereof (e.g.,

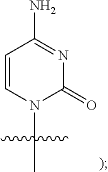 [SMILES: *n1ccc(N)nc1=O]

in the fourth compound, the Base is selected from guanine, 7-deazaguanine or a tautomer thereof (e.g.,

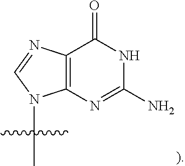 [SMILES: *n1cnc2c(=O)[nH]c(N)nc21]

In some embodiments, the first, second, third and fourth compounds carry an additional detectable label.

In some embodiments, the additional detectable labels carried by the first, second, third and fourth compounds are introduced by an affinity reagent (e.g., antibody, aptamer, Affimer, Knottin), wherein the affinity reagent carries the detectable label, and the affinity reagent can specifically recognize and bind to the epitope of the first, second, third or fourth compound.

In some embodiments, the first, second, third and fourth compounds are linked to the additional detectable label optionally via a linker.

In some embodiments, the Base of the first, second, third or fourth compound is linked to the additional detectable label optionally via a linker.

In some embodiments, the Bases contained in the first, second, third and fourth compounds are different from each other.

In some embodiments, the additional detectable labels carried by the first, second, third and fourth compounds are different from each other.

In some embodiments, the linker is as described above.

In some embodiments, the detectable label is as described above.

In some embodiments, the kit further comprises an reagent for pretreating the nucleic acid molecule; a support for linking nucleic acid molecule to be sequenced; a reagent for linking the nucleic acid molecule to be sequenced to the support (for example, covalently or non-covalently linking); a primer for initiating a nucleotide polymerization reaction; a polymerase for carrying out the nucleotide polymerization reaction; one or more buffer solutions; one or more washing solutions; or any combination thereof.

In the eleventh aspect of the invention, the present invention provides the use of the compound or a salt thereof as described above or the kit as described above for determining the sequence of a target single-stranded polynucleotide.

### BRIEF DESCRIPTION OF DRAWINGS

FIG. 1 illustrates the reaction for removing azidomethyl, as an example of the present invention;

FIG. 2 illustrates the nucleophilic cyclization cascade reaction for the reversible cleavage, as an example of the present invention;

FIG. 3 illustrates reversible blocking nucleotide analogues with 3′-OH labels, as an example of the present invention.

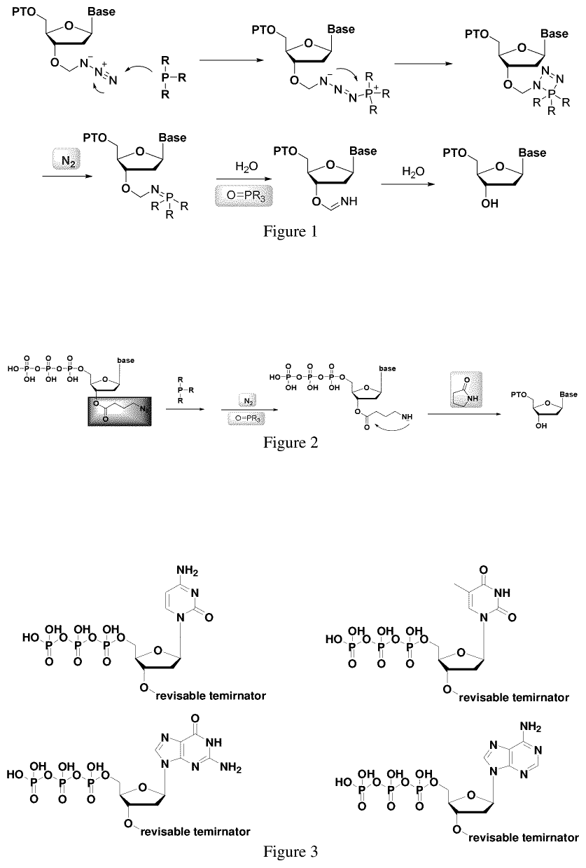

### DETAILED DESCRIPTION OF THE INVENTION

The embodiments of the invention are described in detail below through specific embodiments, but in no case should they be interpreted as restrictions on the invention.

Unless otherwise specified, the above groups and substituents have common meanings in the field of pharmaceutical chemistry.

In various parts of the specification, the substituents of the compounds disclosed in the invention are disclosed according to the type or range of the group. It is particularly noted that the present invention includes each independent sub-combination of each and every member of these group types and ranges. For example, the term “C₁-C₆ alkyl” particularly refers to independently disclosed methyl, ethyl, C₃ alkyl, C₄ alkyl, C₅ alkyl and C₆ alkyl.

In addition, it should be noted that, unless otherwise clearly pointed out, the expressions “each . . . independently is/are selected from“and” . . . are each independently is/are selected from”, as used throughout the description, are interchangeable and both should be interpreted in a broad sense, i.e. it can mean that, in different groups, the specific options expressed by the same or different symbols are independent from each other, or it can mean that, in the same group, the specific options expressed by the same or different symbols are independent from each other.

The term “aliphatic alkyl” means any straight or branched saturated group containing from 1 to 20 carbon atoms, for example, C₁-C₁₂ alkyl, preferably C₁-C₆ alkyl.

The term “C₁-C₆ alkyl” means any straight or branched saturated group containing from 1 to 6 carbon atoms, such as, methyl, ethyl, n-propyl, isopropyl, n-butyl, isobutyl, tertbutyl, sec-butyl, n-amyl, tert-amyl, n-hexyl, etc.

The term “alkoxy” means any above-described alkyl (e.g., C₁-C₆ alkyl, etc.), which is connected to the rest of the molecule through the oxygen atom (—O—).

The term “cycloalkyl” means an saturated cyclic hydrocarbyl of 3-10 membered monocyclic ring system, for example, C₃-C₈ cycloalkyl, preferably C₃-C₆ cycloalkyl.

The term “C₃-C₆ cycloalkyl” means an saturated cyclic hydrocarbyl of 3-6 membered monocyclic ring system. C₃-C₆ cycloalkyl may be cyclopropyl, cyclobutyl, cyclopentyl, cyclohexyl, etc.

The term “aromatic alkyl” means an arylalkyl or heteroarylalkyl, wherein the alkyl is as defined above.

The term “heteroaryl” means an aromatic heterocycle, generally a 5-, 6-, 7-, or 8-membered heterocycle containing from one to three heteroatoms selected from N, O or S; and the heteroaryl ring may optionally be further fused to an aromatic or non-aromatic carbon ring or heterocycle. The non-limiting examples of the heteroaryl are such as pyridyl, pyrazinyl, pyrimidinyl, pyridazinyl, indolyl, imidazolyl, thiazolyl, isothiazolyl, thioxazolyl, pyrrolyl, phenyl-pyrrolyl, furanyl, phenyl-furanyl, oxazolyl, isoxazolyl, pyrazolyl, thiophenyl, benzofuranyl, benzothiophenyl, benzo-1,3-dioxolane (benzodioxane), isodihydroindolyl, benzimidazolyl, indazolyl, quinolinyl, isoquinolinyl, 1,2,3-triazolyl, 1-phenyl-1,2,3-triazolyl, 2,3-dihydroindolyl, 2,3-dihydrobenzofuranyl, 2,3-dihydrobenzothiophenyl, benzopyranyl, 2,3-dihydrobenzoxazinyl, 2,3-dihydroquinoxalinyl, etc.

In the present invention,

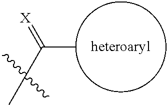

refers to group

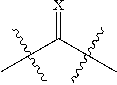

connected to a heteroaryl. Other similar structures can be correspondingly understood with reference to the above contents.

In the present invention, R may be selected from

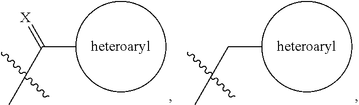

wherein each heteroaryl may be same or different. Further, when each heteroaryl is independently optionally substituted by one or more (e.g., 2, 3, 4, 5 or 6, preferably 2) R″s, it means that, the substituent R″s on different heteroaryl may be same or different, and, the substituent R″s on the same heteroaryl may be same or different.

In the present invention, “each R″ is independently selected from H, —N₃, etc., and is not H at the same time”, it means that, if the heteroaryl is substituted by one or more (e.g., 2, 3, 4, 5 or 6, preferably 2) R″s, at least one substituent R″ is not H.

The term “aryl” means carbon-ring aromatic group having 6-14 carbon atoms, such as, C₆-C₁₀ aryl, preferably phenyl.

It is obvious to those skilled in the art from all the above descriptions that, any group having a combined name, such as “phenyl C₁-C₆ alkyl”, should be understood as being conventionally constructed from the parts from which it is derived, such as constructed from C₁-C₆ alkyl substituted by phenyl, wherein the C₁-C₆ alkyl group is as defined above.

As used herein, the term “salt of the compound of formula (A), formula (I), formula (II), formula (III) or formula (IV)” may be exemplified by those organic addition salts of the organic acids forming the anion, including but not limiting to formate, acetate, propionate, benzoate, maleate, fumarate, succinate, tartrate, citrate, ascorbate, α-ketoglutarate, α-glycerophosphate, alkyl sulfonate or aryl sulfonate; preferably, the alkyl sulfonate is methanesulfonate or ethanesulfonate; the aryl sulfonate is benzenesulfonate or p-toluenesulfonate. Or alternatively, the term also means those inorganic salts, including but not limiting to hydrochloride, hydrobromide, hydroiodide, nitrate, bicarbonate and carbonate, sulfate or phosphate, etc.

In the present invention, R may be selected from

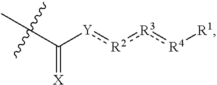

wherein [symbol] represents a single bond [symbol] or a double bond [symbol] , and R², R³ and R⁴ are each independently selected from O, NH, S, CH, CH₂, C(CH₃)₂. It should be understood that, the bond between R² and R³ is a double bond only when both R² and R³ are CH; when R² and R³ are each independently selected from atoms or groups other than CH (i.e., O, NH, S, CH₂, C(CH₃)₂), the bond between R² and R³ is a single bond. For example, the expression “R², R³, and R⁴ are each independently selected from CH or CH₂” means: both R² and R³ are CH, and the bond between R² and R³ is a double bond, and R⁴ is CH₂, and the bond between R³ and R⁴ is a single bond; or, both R³ and R⁴ are CH, and the bond between R³ and R⁴ is a double bond, and R² is CH₂, and the bond between R² and R³ is a single bond. Similarly, the single bond and/or double bond between Y and R², or between R³ and R⁴, may be correspondingly understood with reference to the above contents.

The term “a direct bond” means that the group on both sides are directly connected. For example, in

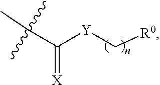

when Y is a direct bond,

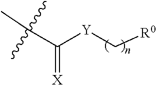

will be

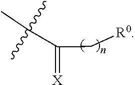

For the compound of formula (B)

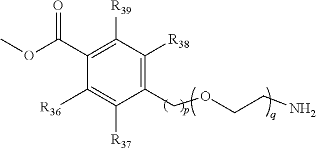

of the invention, the expression “R³⁶ is N₃—C₁-C₆ alkyl, and wherein the C₁-C₆ alkyl is optionally substituted by C₁-C₆ alkyl”, it means that, R³⁶ is N₃—C₁-C₆ alkyl, and the hydrogen atoms on C₁-C₆ alkyl may further be replaced by other C₁-C₆ alkyl. For example, when R³⁶ is N₃-methyl, and the methyl is optionally substituted by methyl, the compound of formula (B) may have the following structure

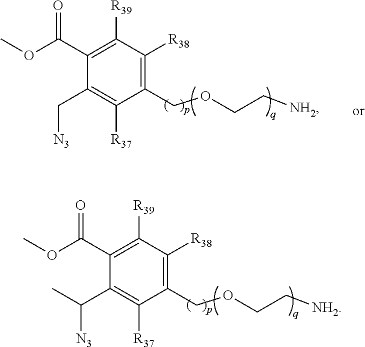

Other similar expressions may be correspondingly understood with reference to the above contents.

In the method of the invention, any substance composed of two chains, namely, the growing nucleic acid chain and the nucleic acid chain to be sequenced, may be called “a duplex”, regardless of the length of the growing nucleic acid chain or the nucleic acid chain to be sequenced, and wherein the nucleic acid chain to be sequenced may be longer than the length of the growing nucleic acid chain.

In the method of the invention, the nucleic acid molecule to be sequenced may be any target nucleic acid molecule. In some preferred embodiments, the nucleic acid molecules to be sequenced include deoxyribonucleotides, ribonucleotides, modified deoxyribonucleotides, modified ribonucleotides, or any combination thereof. In the method of the invention, the nucleic acid molecule to be sequenced is not limited to any type. In some preferred embodiments, the nucleic acid molecule to be sequenced is DNA or RNA. In some preferred embodiments, the nucleic acid molecules to be sequenced may be genomic DNA, mitochondrial DNA, chloroplast DNA, mRNA, cDNA, miRNA, or siRNA. In some preferred embodiments, the nucleic acid molecule to be sequenced is linear or circular. In some preferred embodiments, the nucleic acid molecule to be sequenced is double-stranded or single-stranded. For example, the nucleic acid molecule to be sequenced may be single-stranded DNA (ssDNA), double-stranded DNA (dsDNA), single-stranded RNA (ssRNA), double-stranded RNA (dsRNA), or a hybrid of DNA and RNA. In some preferred embodiments, the nucleic acid molecule to be sequenced is single-stranded DNA. In some preferred embodiments, the nucleic acid molecule to be sequenced is double-stranded DNA.

In the method of the invention, the nucleic acid molecule to be sequenced is not limited by its source. In some preferred embodiments, the nucleic acid molecules to be sequenced may be obtained from any source, such as any cell, tissue or organism (e.g., viruses, bacteria, fungi, plants and animals). In some preferred embodiments, the nucleic acid molecules to be sequenced originate from mammals (e.g., humans, non-human primates, rodents or canines), plants, birds, reptiles, fish, fungi, bacteria or viruses.

The methods for extracting or obtaining nucleic acid molecules from cells, tissues or organisms are well known to those skilled in the art. Suitable methods include but are not limited to such as ethanol precipitation and chloroform extraction.

For a detailed description of such methods, see, for example, J. Sambrook et al., Molecular Cloning: A Laboratory Manual, 2nd Edition, Cold Spring Harbor Laboratory Press, 1989; and F. M. Ausubel et al., Short Protocols in Molecular Biology, 3rd Edition, John Wiley&Sons, Inc., 1995. In addition, various commercial kits may be used for extracting nucleic acid molecules from various sources (e.g., cells, tissues or organisms).

In the method of the invention, the nucleic acid molecule to be sequenced is not limited by its length. In some preferred embodiments, the length of the nucleic acid molecule to be sequenced may be at least 10 bp, at least 20 bp, at least 30 bp, at least 40 bp, at least 50 bp, at least 100 bp, at least 200 bp, at least 300 bp, at least 400 bp, at least 500 bp, at least 1000 bp, or at least 2000 bp. In some preferred embodiments, the length of nucleic acid molecules to be sequenced may be 10-20 bp, 20-30 bp, 30-40 bp, 40-50 bp, 50-100 bp, 100-200 bp, 200-300 bp, 300-400 bp, 400-500 bp, 500-1000 bp, 1000-2000 bp, or more than 2000 bp. In some preferred embodiments, the nucleic acid molecule to be sequenced can have a length of 10-1000 bp, to facilitate high-throughput sequencing.

In the method for polynucleotides preparation or sequencing of the invention, suitable polymerase may be used for performing nucleotide polymerization reaction. In some exemplary embodiments, the polymerase can synthesize new DNA strands using DNA as a template (such as, a DNA polymerase). In some exemplary embodiments, the polymerase can synthesize new DNA strands using RNA as a template (e.g., a reverse transcriptase). In some exemplary embodiments, the polymerase can synthesize new RNA chains using DNA or RNA as templates (e.g., a RNA polymerase). Therefore, in some preferred embodiments, the polymerase is selected from DNA polymerase, RNA polymerase, and reverse transcriptase. Appropriate polymerase can be selected according to actual needs to carry out nucleotide polymerization reaction. In some preferred embodiments, the polymerization reaction is a polymerase chain reaction (PCR). In some preferred embodiments, the polymerization reaction is a reverse transcription reaction.

In the method of the present invention, KOD polymerase or its mutants may be used for the nucleotide polymerization reaction. KOD polymerase or its mutants (e.g., KOD POL151, KOD POL157, KOD POL171, KOD POL174, KOD POL376, KOD POL391) may lead to acceptable Incorporation efficiency for the modified nucleoside or nucleotide of the invention. KOD POL391 and KOD POL171 lead to acceptable Incorporation efficiency for the modified nucleotides of the invention. In some embodiments, the Incorporation efficiency of KOD POL391 or KOD POL171 for the modified nucleotides of the invention is more than 70%, such as 70%-80%, 80%-90% or 90%-100%.

In the method for polynucleotide preparation or sequencing of the invention, the polymerization reaction of nucleotides is carried out under suitable conditions. The suitable polymerization conditions include the composition of the solution phase, the concentration of each component, the pH of the solution phase, and the polymerization temperature. Polymerization is performed under suitable conditions to obtain acceptable and even higher Incorporation efficiency.

In the present invention, the hydroxyl (—OH) at 3′-position of the deoxyribose in the compound of formula (A) is protected (by R), thereby can terminate the polymerization caused by polymerase (such as DNA polymerase). For example, when the compound of formula (A) is introduced into the 3′-end of the growing nucleic acid chain, because there is no free hydroxyl (—OH) at the 3′-position of the deoxyribose of the compound, the polymerase cannot proceed to the next round of polymerization, and the polymerization is terminated. In such case, only one base will be incorporated into the growing nucleic acid chain in each round of polymerization.

In addition, the protective group (R) of the hydroxyl (—OH) at the 3′-position of the deoxyribose in the compound of formula (A) can be removed to restore the free hydroxyl (—OH). Then the growing nucleic acid chain may undergo next round of polymerization reaction using polymerase and the compound of formula (A) to incorporate therein a base again.

That is, the hydroxyl (—OH) at the 3′-position of the deoxyribose in the compound of formula (A) is reversibly blocked: when the compound of formula (A) is incorporated into the 3′ end of the growing nucleic acid chain, it will terminate the polymerization caused by the polymerase and stop any further extension of the growing nucleic acid chain; and, after the blocking group contained in the compound of formula (A) is removed, the polymerase can continue the polymerization of the growing nucleic acid chain to extend the nucleic acid chain.

Some embodiments described herein relate to the use of conventional detectable labels. Detection may be realized by any suitable method, including fluorescence spectroscopy or other optical means. Preferred label is a fluorescent label, namely a fluorophore, which emits radiation of certain wavelength after absorbing energy. Many suitable fluorescent labels are known in the art. For example, Welch et al., (Chem. Eur. J. 5(3):951-960, 1999) discloses dansyl-functionalised fluorescent moieties that can be used in the present invention. Zhu et al. (Cytometry 28:206-211, 1997) describes the use of the fluorescent labels Cy3 and Cy5, which can also be used in the present invention. Labels suitable for use are also disclosed in Prober et al. (Science 238:336-341, 1987); Connell et al. (BioTechniques 5(4):342-384, 1987), Ansorge et al. (Nucl. Acids Res. 15(11):4593-4602, 1987) and Smith et al. (Nature 321:674, 1986). Other commercially available fluorescent labels include, but are not limited to, fluorescein, rhodamine (including TMR, texas red and Rox), alexa, BODIPY, acridine, coumarin, pyrene, benzanthracene and the cyanins.

Multiple labels can also be used in the present application, for example, bi-fluorophore FRET cassettes (Tet. Let. 46:8867-8871, 2000). Multi-fluor dendrimeric systems (J. Am. Chem. Soc. 123:8101-8108, 2001) can also be used. Although fluorescent labels are preferred, other forms of detectable labels will be apparent as useful to those of ordinary skill in the art. For example, microparticles, including quantum dots (Empodocles et al., Nature 399:126-130, 1999), gold nanoparticles (Reichert et al., Anal. Chem. 72:6025-6029, 2000) and microbeads (Lacoste et al., Proc. Natl. Acad. Sci USA 97(17):9461-9466, 2000) can all be used.

Multi-component labels can also be used in the present application. A multi-component label is one which is dependent on the interaction with a further compound for detection. The most common multi-component label used in biology is the biotin-streptavidin system. Biotin is used as the label attached to the nucleotide or modified nucleotide. Streptavidin is then added separately to enable detection to occur. Other multi-component systems can be used. For example, dinitrophenol has a commercially available fluorescent antibody that can be used for detection.

In some embodiments described herein, carrying the detectable labels described above on the modified nucleotides or nucleoside molecules can be achieved through the incorporation of affinity reagents (e.g., antibodies, aptamers, Affimer, and Knottin), wherein the affinity reagent can specifically recognize and bind to the epitopes of the modified nucleotides or nucleoside molecules. The specific principle is shown in WO2018129214A1, which is herein incorporated by reference in its entirety.

In other embodiments described herein, the modified nucleotide or nucleoside molecule may be linked to a detectable label described above. In some such embodiments, the linker used may be cleaved. The use of cleavable linker ensures that the labels can be removed as needed after the detection, and thus avoiding any interference signals from any subsequently added labeled nucleotides or nucleosides.

In other embodiments, the linker used is not cleavable. Because in each circumstance where the labeled nucleotide of the present invention is incorporated, it is not necessary to subsequently incorporate any nucleotide, so it is not necessary to remove the label from the nucleotide.

Those skilled in the art will be aware of the utility of dideoxynucleoside triphosphates in Sanger sequencing methods, and related protocols (Sanger-type), which rely upon randomized chain-termination at particular type of nucleotide. The nucleotides of the present application, it will be recognized, may be of utility in Sanger methods and related protocols since the same effect achieved by using ddNTPs may be achieved by using the 3′-OH protecting groups described herein: both prevent incorporation of subsequent nucleotides. Similarly, the modified nucleosides and nucleotides of the present application may be of utility in high-throughput sequencing, especially in the high-throughput sequencing platform based on sequencing by synthesis. By using the reversible blocking nucleotides and analogues of the present application, the types of incorporated nucleotides may be determined one by one during the sequencing process.

Cleavable linkers are known in the art, and conventional chemistry can be applied to attach a linker to a nucleotide or modified nucleotide and a label. The linker can be cleaved by any suitable method, including exposure to acids, bases, nucleophiles, electrophiles, radicals, metals, reducing or oxidizing agents, light, temperature, enzymes etc. The linker as discussed herein may also be cleaved with the same catalyst used to cleave the 3′-O-protecting group bond. Suitable linkers can be adapted from standard chemical protecting groups, as disclosed in Greene & Wuts, Protective Groups in Organic Synthesis, John Wiley & Sons. Further suitable cleavable linkers used in solid-phase synthesis are disclosed in Guillier et al. (Chem. Rev. 100:2092-2157, 2000).

The use of the term “cleavable linker” is not meant to imply that the whole linker is required to be removed from, e.g., the nucleotide or modified nucleotide. Where the detectable label is attached to the nucleotide or modified nucleotide, the nucleoside cleavage site can be located at a position on the linker that ensures that part of the linker remains attached to the nucleotide or modified nucleotide after cleavage.

Where the detectable label is attached to the nucleotide or modified nucleotide, the linker can be attached at any position on the nucleotide or modified nucleotide provided that Watson-Crick base pairing can still be carried out.

A. Linkers Capable of being Cleaved by Electrophilic Reaction

Linkers capable of being cleaved by electrophilic reaction are typically cleaved by protons and include cleavages sensitive to acids. Suitable linkers include the modified benzylic systems such as trityl, p-alkoxybenzyl esters and p-alkoxybenzyl amides. Other suitable linkers include tert-butyloxycarbonyl (Boc) groups and the acetal system.

The use of thiophilic metals, such as nickel, silver or mercury, in the cleavage of thioacetal or other sulfur-containing protecting groups can also be considered for the preparation of suitable linker molecules.

B. Linkers Capable of being Cleaved by Nucleophilic Reaction

Nucleophilic cleavage is also a well recognised method in the preparation of linker molecules. Groups such as esters that are labile in water (i.e., can be cleaved simply at basic pH) and groups that are labile to non-aqueous nucleophiles, can be used. Fluoride ions can be used to cleave silicon-oxygen bonds in groups such as triisopropyl silane (TIPS) or t-butyldimethyl silane (TBDMS).

C. Linker Capable Of Being Cleaved By Photolysis Linkers capable of being cleaved by photolysis have been used widely in carbohydrate chemistry. It is preferable that the light required to activate cleavage does not affect the other components of the modified nucleotides. For example, if a fluorophore is used as the label, it is preferable if this absorbs light of a different wavelength to that required to cleave the linker molecule. Suitable linkers include those based on O-nitrobenzyl compounds and nitroveratryl compounds. Linkers based on benzoin chemistry can also be used (Lee et al., J. Org. Chem. 64:3454-3460, 1999).

D. Linkers Capable Of Being Cleaved Under Reductive Conditions

There are many linkers known that are susceptible to reductive cleavage.

Catalytic hydrogenation using palladium-based catalysts has been used to cleave benzyl and benzyloxycarbonyl groups. Disulfide bond reduction is also known in the art.

E. Linkers Capable Of Being Cleaved Under Oxidative Conditions

Oxidation-based approaches are well known in the art. These include oxidation of p-alkoxybenzyl groups and the oxidation of sulfur and selenium linkers. The use of aqueous iodine to cleave disulfides and other sulfur or selenium-based linkers is also within the scope of the invention.

F. Safety-Catch Linkers

Safety-catch linkers are those that cleave in two steps. In a preferred system the first step is the generation of a reactive nucleophilic center followed by a second step involving an intra-molecular cyclization that results in cleavage. For example, levulinic ester linkages can be treated with hydrazine or photochemistry to release an active amine, which can then be cyclised to cleave an ester elsewhere in the molecule (Burgess et al., J. Org. Chem. 62:5165-5168, 1997).

G. Linker Capable of being Cleaved by Elimination Mechanisms

Elimination reactions can also be used. For example, the base-catalysed elimination of groups such as Fmoc and cyanoethyl, and palladium-catalysed reductive elimination of allylic systems, can be used.

In some embodiments, the linker can comprise a spacer unit. The length of the linker is unimportant provided that the label is held a sufficient distance from the nucleotide so as not to interfere with any interaction between the nucleotide and an enzyme.

In some embodiments, the linker may consist of the similar functionality as the 3′-OH protecting group. This will make the deprotection and deprotecting process more efficient, as only a single treatment will be required to remove both the label and the protecting group. Particularly preferred linkers are phosphine-cleavable azide containing linkers.

The present invention will be further explained below in conjunction with specific examples. Unless otherwise specified, the reagents used in the following examples can be commercially available. In addition, for each modification of 3′-OH, if one or more of the nucleotide analogues, dTTP, dATP, dCTP and dGTP, are prepared, those skilled in the art can undoubtedly prepare and obtain the remaining one or several nucleotide analogues.

### I. Preparation Examples

### Example 1

The structure of the target compound is:

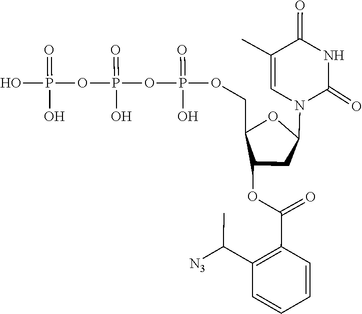 [SMILES: Cc1cn([C@H]2C[C@@H](OC(=O)c3ccccc3C(C)N=[N+]=[N-])[C@@H](COP(=O)(O)OP(=O)(O)OP(=O)(O)O)O2)c(=O)[nH]c1=O]

(1) Step 1

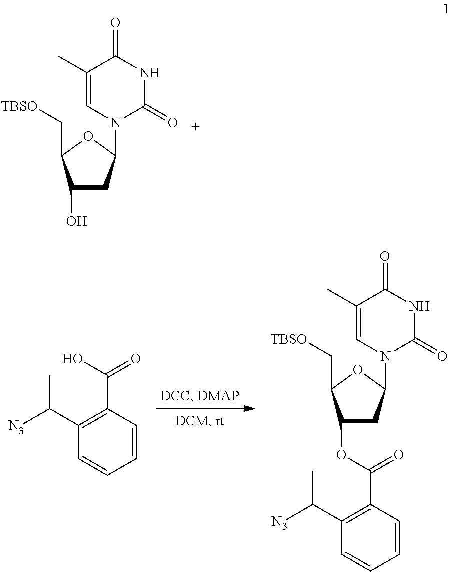 [SMILES: Cc1cn([C@H]2C[C@@H](O)[C@@H](CO[Si](C)(C)C(C)(C)C)O2)c(=O)[nH]c1=O ; CC(N=[N+]=[N-])c1ccccc1C(=O)O ; Cc1cn([C@H]2C[C@@H](OC(=O)c3ccccc3C(C)N=[N+]=[N-])[C@@H](CO[Si](C)(C)C(C)(C)C)O2)c(=O)[nH]c1=O]

To a dried 100 mL round bottom flask equipped with a magnetic stirrer and a rubber stopper with argon balloon, were added T-nucleoside (500 mg, 1.4 mmol, 1 eq) (available from Beijing OKeanos Tech. Co., Ltd., cat. no. OK-N-18102) and azido protection group (268 mg, 1.4 mmol, 1 eq) (available from Beijing OKeanos Tech. Co., Ltd., cat. no. OK-H-20001) separately, followed by adding with syringe 20 mL of anhydrous methylene chloride as solvent. The reaction mixture was stirred at room temperature to facilitate dissolution, followed by adding DCC (435 mg, 2.1 mmol, 1.5 eq) and DMAP (20 mg, 0.14 mmol, 0.1 eq) separately, and allowed to react under nitrogen balloon for providing protection atmosphere at room temperature for further 4 h. The reaction was monitored with TLC at half an hour interval, until the starting materials were substantially depleted. The reaction mixture was concentrated with a rotary evaporator, followed by product separation by silica gel column chromatography (Hexane/EA=10/1˜1/1, silica gel column diameter 3.5 cm, filler height 10 cm). The yield of this step was 95%.

¹H NMR (400 MHz, CDCl₃) δ 8.41 (1H, s); 7.97 (1H, m); 7.61 (3H, m); 7.40 (1H, m); 7.27 (1H, s); 6.45 (1H, m); 5.67 (1H, m); 5.50 (1H, m); 4.28 (1H, m); 4.02 (1H, m); 2.59 (1H, m); 2.24 (1H, m); 1.96 (3H, s); 1.55 (3H, d, J=6.8 Hz); 0.97 (9H, s); 0.10 (6H, s).

(2) Step 2

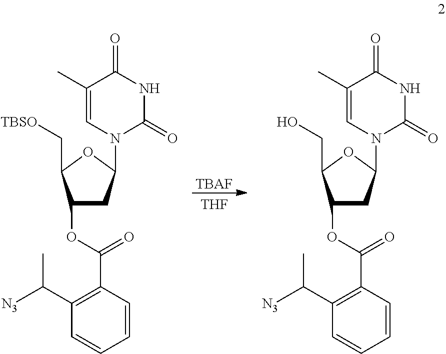

To a dried 100 mL round bottom flask equipped with a magnetic stirrer and a rubber stopper with argon balloon was added the product from Step 1 (750 mg, 1.4 mmol, 1 eq), followed by adding with syringe 20 mL of anhydrous THF as solvent. The reaction mixture was stirred to facilitate dissolution, followed by slowly adding with syringe 1 mol/L solution of TBAF (1.8 mL, 1.8 mmol, 1.3 eq) dropwise while in ice bath. The flask was brough to room temperature and stirred continuously for about 2 h. The reaction was monitored with TLC at half an hour interval, until the dot of starting material on TLC plate disappeared. The reaction mixture was concentrated with a rotary evaporator, followed by product separation by silica gel column chromatography (Hexane/EA=10/1˜1/1 to methylene chloride/anhydrous methanol=100/1˜10/1, silica gel column diameter 3.5 cm, filler height 10 cm). The yield of this step was 70%.

¹H NMR (400 MHz, DMSO-d₆) δ 11.34 (1H, s); 7.89 (1H, m); 7.78 (1H, s); 7.70-7.62 (2H, m); 7.50-7.46 (1H, m); 6.27-6.24 (1H, m); 5.58-5.55 (1H, m), 5.26 (1H, t); 4.19 (1H, br.); 3.71 (2H, br.); 1.79 (3H, d); 1.49 (3H, d, J=6.8 Hz).

(3) Step 3

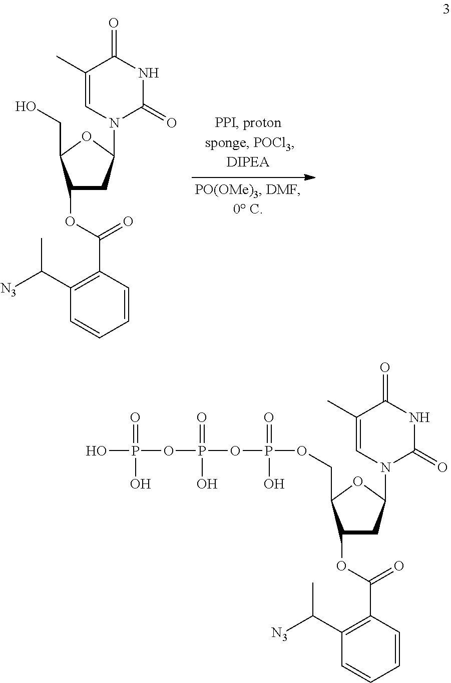 [SMILES: Cc1cn([C@H]2C[C@@H](OC(=O)c3ccccc3C(C)N=[N+]=[N-])[C@@H](CO)O2)c(=O)[nH]c1=O ; Cc1cn([C@H]2C[C@@H](OC(=O)c3ccccc3C(C)N=[N+]=[N-])[C@@H](COP(=O)(O)OP(=O)(O)OP(=O)(O)O)O2)c(=O)[nH]c1=O]

To a dried 100 mL two-necked 1 #flask equipped with a magnetic stirrer and a three-way valve with argon balloon, were added product from Step 2 (350 mg, 0.85 mmol, 1 eq) and proton sponge (363.5 mg, 1.7 mmol, 2 eq). To a dried 100 mL two-necked 2 #flask equipped with a magnetic stirrer and a three-way valve with argon balloon was added tributyl ammonium pyrophosphate (932.5 mg, 1.7 mmol, 2 eq). The two-necked 1 #flask was filled with argon, followed by adding 15 mL of trimethyl phosphate with stirring to facilitate dissolution. The reaction was brought to 0° C., followed by slowly adding with syringe phosphorus oxychloride (120 μL, 1.3 mmol, 1.5 eq) and stirring continuously at 0° C. for 1.5 h. The two-necked 2 #flask was filled with argon, followed by adding with syringe 2 mL of anhydrous DMF and DIPEA (740 μL, 4.25 mmol, 5 eq) with stirring at 0° C. to facilitate dissolution. The liquid in 1 #flask was transferred with syringe into the 2 #flask. The mixture was maintained at 0° C. and continuously stirred for 3 h, followed by adding 20 mL of solution of TEAB (0.1 mol/L) to quench the reaction. The reaction mixture was diluted with 50 mL of deionized water, and then separated by DEAE resin column prepared-in-advance and eluted with gradient elution (H₂O:TEAB(1 mol/L)=10/1˜0/1, DEAE column diameter 4.5 cm, filler height 8 cm). Fractions were identified with HPLC and MS, and those fractions containing the triphosphorylated product were collected and concentrated under vacuum to 10 mL, followed by preparative liquid chromatography separation with gradient elution (CH₃CN:TEAB(0.1 mol/L)=2/98˜98/2). The high-purity product solution recovered from the separation was concentrated with a rotary evaporator under vacuum, transferred to a plastic centrifuge tube, and then freeze-dried with a lyophilizer into a powder solid. MW=655. The yield of this step was 50%.

¹H NMR (400 MHz, DMSO-d₆) δ 11.32 (s, 1H), 7.97 (s, 1H), 7.93-7.88 (m, 1H), 7.72-7.60 (m, 2H), 7.52-7.46 (m, 1H), 6.40-6.29 (m, 1H), 5.64-5.54 (m, 2H), 4.33 (s, 1H), 4.17-4.01 (m, 2H), 2.54-2.50 (m, 1H), 2.45-2.36 (m, 1H), 1.85 (s, 3H), 1.49 (dd, J=6.7, 1.0 Hz, 3H).

### Example 2

The structure of the target compound is:

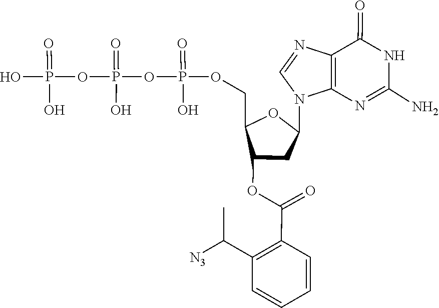 [SMILES: CC(N=[N+]=[N-])c1ccccc1C(=O)O[C@@H]1C[C@H](n2cnc3c(=O)[nH]c(N)nc32)O[C@@H]1COP(=O)(O)OP(=O)(O)OP(=O)(O)O]

(1) Step 1

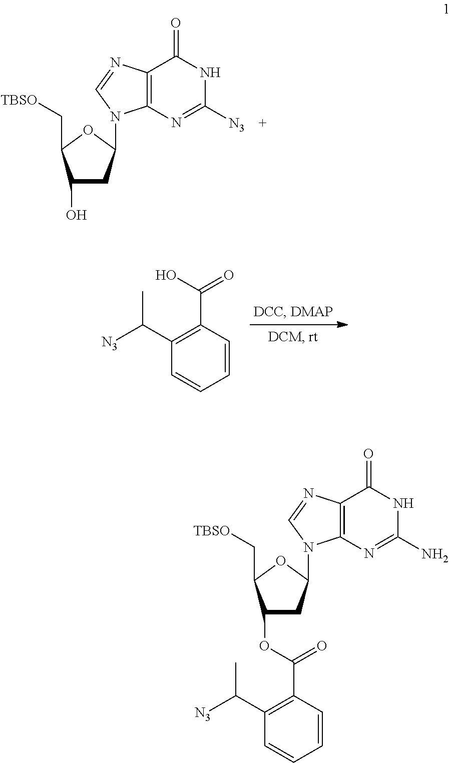 [SMILES: CC(C)(C)[Si](C)(C)OC[C@H]1O[C@@H](n2cnc3c(=O)[nH]c(N=[N+]=[N-])nc32)C[C@H]1O ; CC(N=[N+]=[N-])c1ccccc1C(=O)O ; CC(N=[N+]=[N-])c1ccccc1C(=O)O[C@@H]1C[C@H](n2cnc3c(=O)[nH]c(N)nc32)O[C@@H]1CO[Si](C)(C)C(C)(C)C]

To a dried 100 mL round bottom flask equipped with a magnetic stirrer and a rubber stopper with argon balloon were added G-nucleoside (600 mg, 1.58 mmol, 1 eq) (available from Beijing Okeanos Tech. Co., Ltd., cat. No. OK-N-18103) and azido protection group (302 mg, 1.58 mmol, 1 eq) (available from Beijing Okeanos Tech. Co., Ltd., cat. No. OK-H-20001) separately, followed by adding with syringe 20 mL of anhydrous methylene chloride as solvent. The reaction mixture was stirred at room temperature to facilitate dissolution, followed by adding DCC (488 mg, 2.37 mmol, 1.5 eq) and DMAP (20 mg, 0.16 mmol, 0.1 eq) separately under nitrogen balloon for providing protection atmosphere, and allowed to react at room temperature for further 4 h. The reaction was monitored with TLC at half an hour interval, until the starting materials were substantially depleted. The reaction mixture was concentrated with a rotary evaporator, followed by product separation by silica gel column chromatography (Hexane/EA=10/1˜1/1, silica gel column diameter 3.5 cm, filler height 10 cm). The yield of this step was 95%.

¹H NMR (500 MHz, DMSO-d₆) δ 10.65 (s, 1H), 7.99-7.84 (m, 2H), 7.79-7.61 (m, 2H), 7.57-7.45 (m, 1H), 6.47 (s, 2H), 6.20 (ddd, J=9.1, 5.8, 3.6 Hz, 1H), 5.65-5.49 (m, 2H), 4.27 (qd, J=4.8, 1.9 Hz, 1H), 3.86 (dd, J=4.6, 2.2 Hz, 2H), 2.99-2.80 (m, 1H), 2.78-2.61 (m, 1H), 1.50 (d, J=6.7 Hz, 3H), 0.88 (s, 9H), 0.07 (dd, J=4.5, 1.3 Hz, 6H).

(2) Step 2

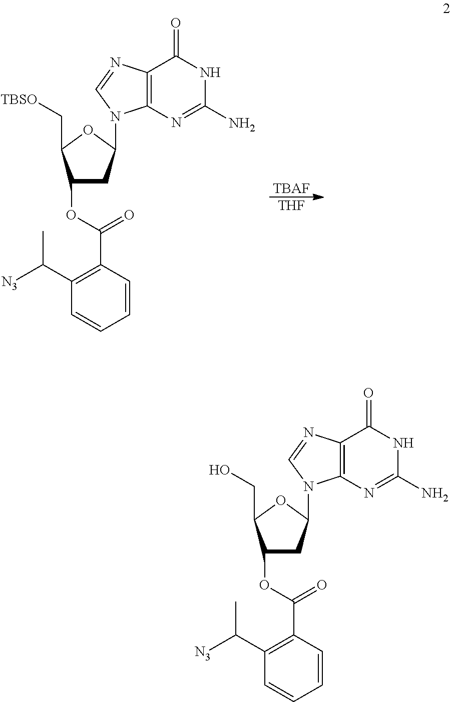

To a dried 100 mL round bottom flask equipped with a magnetic stirrer and a

rubber stopper with argon balloon was added the product from Step 1 (850 mg, 1.54 mmol, 1 eq) followed by adding with syringe 20 mL of anhydrous THF as solvent and stirring at room temperature to facilitate dissolution. The reaction mixture was cooled in ice bath and slowly added with syringe 1 mol/L solution of TBAF (2.0 mL, 2.0 mmol, 1.3 eq) dropwise. The flask was brough to room temperature and stirred continuously for about 2 h. The reaction was monitored with TLC at half an hour interval, until the dot of starting material on TLC plate disappeared. The reaction mixture was concentrated with a rotary evaporator, followed by product separation by silica gel column chromatography (Hexane/EA=10/1˜1/1 to methylene chloride/anhydrous methanol=100/1˜10/1, silica gel column diameter 3.5 cm, filler height 10 cm). The yield of this step was 70%.

¹H NMR (500 MHz, DMSO-d₆) δ 10.68 (H, s), 7.99 (1H, s), 7.91 (1H, m), 7.67 (2H, m), 7.46 (1H, m), 6.52 (2H, s), 6.17 (1H, m), 5.58 (2H, m), 5.25 (1H, m), 4.23 (1H, t), 3.66 (2H, t), 2.89 (1H, m), 2.61 (1H, m), 1.49 (3H, d, J=6.8 Hz).

(3) Step 3

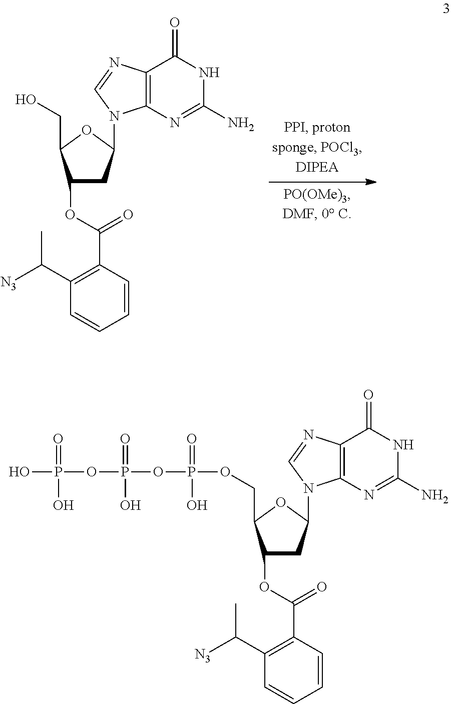 [SMILES: CC(N=[N+]=[N-])c1ccccc1C(=O)O[C@@H]1C[C@H](n2cnc3c(=O)[nH]c(N)nc32)O[C@@H]1CO ; CC(N=[N+]=[N-])c1ccccc1C(=O)O[C@@H]1C[C@H](n2cnc3c(=O)[nH]c(N)nc32)O[C@@H]1COP(=O)(O)OP(=O)(O)OP(=O)(O)O]

To a dried 100 mL two-necked 1 #flask equipped with a magnetic stirrer and a three-way valve with argon balloon were added product from Step 2 (375 mg, 0.85 mmol, 1 eq) and proton sponge (363.5 mg, 1.7 mmol, 2 eq). To a dried 100 mL two-necked 2 #flask equipped with a magnetic stirrer and a three-way valve with argon balloon was added tributyl ammonium pyrophosphate (932.5 mg, 1.7 mmol, 2 eq). The two-necked 1 #flask was filled with argon, added with 15 mL of trimethyl phosphate with stirring to facilitate dissolution. The reaction was brought to 0° C., followed by slowly adding with syringe phosphorus oxychloride (120 μL, 1.3 mmol, 1.5 eq) and stirring continuously at 0° C. for 1.5 h. The two-necked 2 #flask was filled with argon, followed by adding with syringe 5 mL of anhydrous DMF and DIPEA (740 μL, 4.25 mmol, 5 eq) with stirring at 0° C. to facilitate dissolution. The liquid in 1 #flask was transferred with syringe into the 2 #flask. The mixture was maintained at 0° C. and continuously stirred for 3 h, followed by adding 20 mL of solution of TEAB (0.1 mol/L) to quench the reaction. The reaction mixture was diluted with 50 mL of deionized water, and then separated by DEAE resin column prepared-in-advance and eluted with gradient elution (H₂O/TEAB(1 mol/L)=10/1˜0/1, DEAE column diameter 4.5 cm, filler height 8 cm).

The fractions containing the triphosphated product were collected and concentrated with a rotary evaporator under vacuum, followed by preparative liquid chromatography separation with gradient elution (CH₃CN:TEAB(0.1 mol/L)=2/98˜98/2). The high-purity product solution recovered from the separation was concentrated, transferred to a plastic centrifuge tube, and then freeze-dried with a lyophilizer into a powder solid. MW=680. The yield of this step was 45%. ¹H NMR (400 MHz, DMSO-d₆) δ 11.12 (s, 1H), 8.05 (d, J=3.1 Hz, 1H), 7.93 (d, J=7.6 Hz, 1H), 7.73-7.62 (m, 2H), 7.50 (t, J=7.6 Hz, 1H), 6.87 (s, 2H), 6.25-6.19 (m, 1H), 5.72 (d, J=4.9 Hz, 1H), 5.61 (q, J=6.6 Hz, 1H), 4.36-4.33 (m, 1H), 4.25-4.18 (m, 1H), 4.06-4.01 (m, 1H), 3.27-3.12 (m, 1H), 2.60-2.52 (m, 1H), 1.50 (dd, J=6.7, 2.0 Hz, 3H).

### Example 3

The structure of the target compound is:

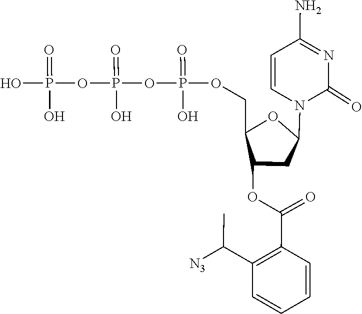 [SMILES: CC(N=[N+]=[N-])c1ccccc1C(=O)O[C@@H]1C[C@H](n2ccc(N)nc2=O)O[C@@H]1COP(=O)(O)OP(=O)(O)OP(=O)(O)O]

(1) Step 1

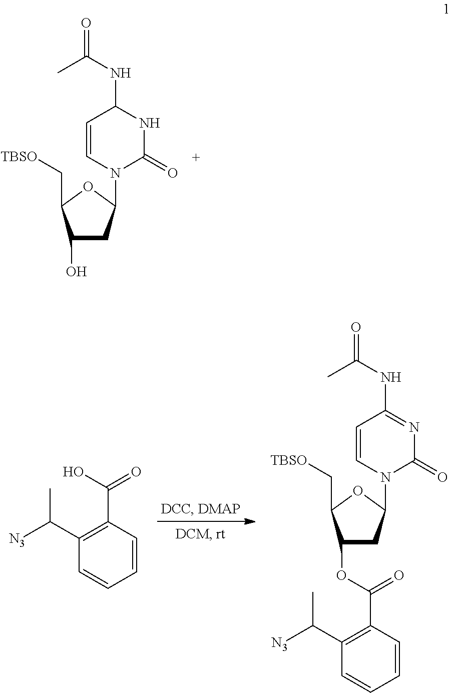 [SMILES: CC(=O)NC1C=CN([C@H]2C[C@@H](O)[C@@H](CO[Si](C)(C)C(C)(C)C)O2)C(=O)N1 ; CC(N=[N+]=[N-])c1ccccc1C(=O)O ; CC(=O)Nc1ccn([C@H]2C[C@@H](OC(=O)c3ccccc3C(C)N=[N+]=[N-])[C@@H](CO[Si](C)(C)C(C)(C)C)O2)c(=O)n1]

To a dried 100 mL round bottom flask equipped with a magnetic stirrer and a rubber stopper with argon balloon were added C-nucleoside (600 mg, 1.56 mmol, 1 eq) (available from Beijing Okeanos Tech. Co., Ltd., cat. No. OK-N-18104) and azido protection group (297 mg, 1.56 mmol, 1 eq) (available from Beijing Okeanos Tech. Co., Ltd., cat. No.: OK-H-20001) separately, followed by adding with syringe 20 mL of anhydrous methylene chloride as solvent. The reaction mixture was stirred at room temperature to facilitate dissolution, followed by adding DCC (480 mg, 2.34 mmol, 1.5 eq) and DMAP (20 mg, 0.16 mmol, 0.1 eq) separately under nitrogen balloon for providing protection atmosphere, and allowed to react at room temperature for further 4 h. The reaction was monitored with TLC at half an hour interval, until the starting materials were substantially depleted. The reaction mixture was concentrated with a rotary evaporator, followed by product separation by silica gel column chromatography (Hexane/EA=10/1˜1/1, silica gel column diameter 3.5 cm, filler height 10 cm). The yield of this step was 95%.

¹H NMR (400 MHz, DMSO-d₆) δ 10.91 (1H, s); 8.23 (1H, d, J=7.52 Hz); 7.89 (1H, d, J=6.80 Hz); 7.66 (2H, m); 7.48 (1H, m); 7.23 (1H, d, J=7.48); 6.19 (1H, t), 5.55 (1H, m); 5.44 (1H, d, J=6.04 Hz), 4.42 (1H, t); 3.92 (2H, m); 2.74 (1H, m); 2.32 (1H, m); 1.47 (3H, d, J=6.7 Hz); 0.86 (9H, s); 0.09 (6H, s).

(2) Step 2

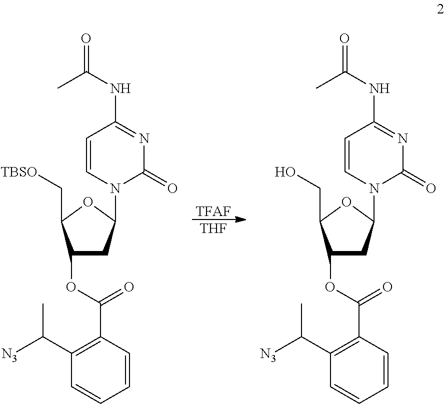

To a dried 100 mL round bottom flask equipped with a magnetic stirrer and a rubber stopper with argon balloon was added the product from Step 1 (860 mg, 1.54 mmol, 1 eq), followed by adding with syringe 20 mL of anhydrous THF as solvent. The reaction mixture was stirred to facilitate dissolution, cooled in ice bath, followed by slowly adding with syringe 1 mol/L solution of TBAF (2.0 mL, 2.0 mmol, 1.3 eq), dropwise. The flask was brough to room temperature and stirred continuously for about 2 h. The reaction was monitored with TLC at half an hour interval until the dot of starting material on TLC plate disappeared. The reaction mixture was concentrated with a rotary evaporator, followed by product separation by silica gel column chromatography (Hexane/EA=10/1˜1/1 to methylene chloride/anhydrous methanol=100/1˜10/1, silica gel column diameter 3.5 cm, filler height 10 cm). The yield of this step was 70%.

¹H NMR (400 MHz, DMSO-d₆) δ 10.90 (1H, s); 8.34 (1H, d, J=7.48 Hz); 7.89 (1H, d, J=7.84 Hz); 7.66 (2H, m); 7.48 (1H, m); 7.23 (1H, d, J=7.48 Hz); 6.22 (1H, t); 5.57 (1H, m); 5.47 (1H, d, J=6.00 Hz); 5.25 (1H, s); 4.32 (1H, m); 3.72 (1H, s); 2.67 (1H, m); 2.31 (1H, m); 1.45 (3H, d, J=6.7 Hz).

(3) Step 3

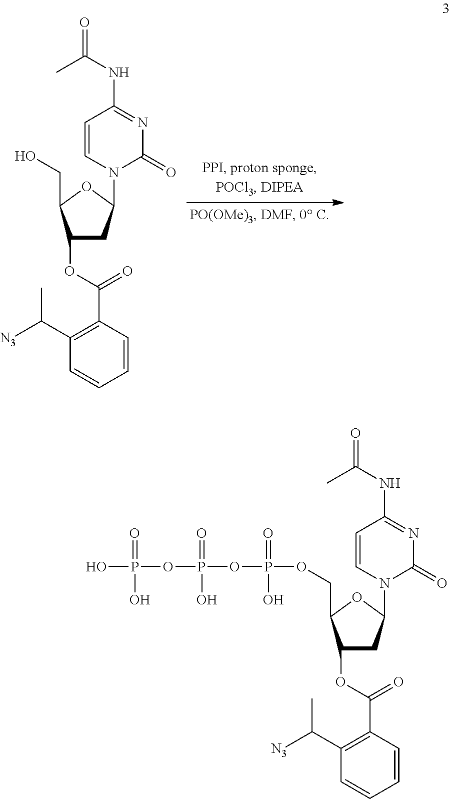 [SMILES: CC(=O)Nc1ccn([C@H]2C[C@@H](OC(=O)c3ccccc3C(C)N=[N+]=[N-])[C@@H](CO)O2)c(=O)n1 ; CC(=O)Nc1ccn([C@H]2C[C@@H](OC(=O)c3ccccc3C(C)N=[N+]=[N-])[C@@H](COP(=O)(O)OP(=O)(O)OP(=O)(O)O)O2)c(=O)n1]

To a dried 100 mL two-necked 1 #flask equipped with a magnetic stirrer and a three-way valve with argon balloon were added product from Step 2 (377 mg, 0.85 mmol, 1 eq) and proton sponge (363.5 mg, 1.7 mmol, 2 eq). To a dried 100 mL two-necked 2 #flask equipped with a magnetic stirrer and a three-way valve with argon balloon was added tributyl ammonium pyrophosphate (932.5 mg, 1.7 mmol, 2 eq). The two-necked 1 #flask was filled with argon, followed by adding 15 mL of trimethyl phosphate with stirring to facilitate dissolution. The reaction was brought to 0° C., followed by slowly adding with syringe phosphorus oxychloride (120 μL, 1.3 mmol, 1.5 eq) and stirring continuously at 0° C. for 1.5 h. The two-necked 2 #flask was filled with argon, followed by adding with syringe 2 mL of anhydrous DMF and DIPEA (740 μL, 4.25 mmol, 5 eq) with stirring at 0° C. to facilitate dissolution. The mixture was maintained at 0° C. and continuously stirred for 3 h, followed by adding 20 mL of solution of TEAB (0.1 mol/L) to quench the reaction. The reaction mixture was diluted with 50 mL of deionized water, and then separated by DEAE resin column prepared-in-advance and eluted with gradient elution (H₂O:TEAB(1 mol/L)=10/1˜0/1, DEAE column diameter 4.5 cm, filler height 8 cm). The fractions containing the triphosphated product were collected and concentrated with a rotary evaporator, followed by preparative liquid chromatography separation with gradient elution (CH₃CN:TEAB(0.1 mol/L)=2/98˜98/2). The high-purity product solution recovered from the separation was concentrated with a rotary evaporator under vacuum, transferred to a plastic centrifuge tube, and then freeze-dried with a lyophilizer into a powder solid. MW=684. The yield of this step was 40%.

(4) Step 4

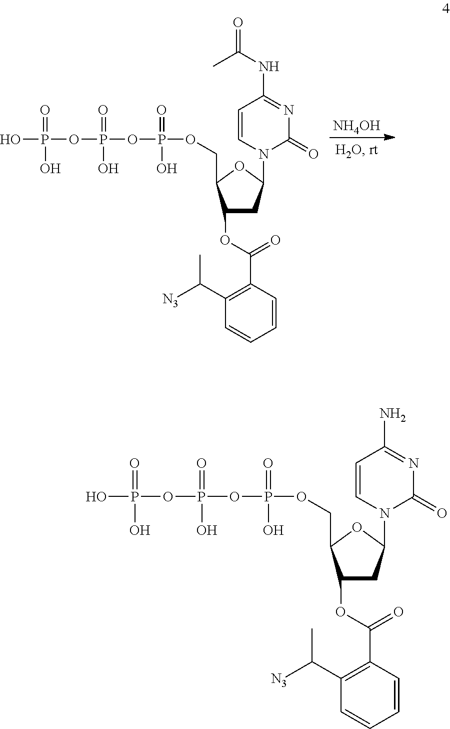

To a dried 100 mL round bottom flask equipped with a magnetic stirrer were added the triphosphated product from Step 3 (500 mg, 0.73 mmol, 1 eq) and 5 ml of deionized water. The mixture was stirred at room temperature to complete dissolution and slowly added with 25% aqueous ammonia (2.5 g, 36.5 mmol, 50 eq), followed by stirring at room temperature for about 6 h. Ammonia in the solution was removed by a rotary evaporator under vacuum and the residue was diluted with deionized water to about 10 mL. After separation with preparative liquid chromatography with gradient elution (CH₃CN:TEAB(0.1 mol/L)=2/98˜98/2), the resulted high-purity product solution was concentrated with a rotary evaporator under vacuum, transferred to a plastic centrifuge tube, and freeze-dried with a lyophilizer into a powder solid. MW=642. The yield of this step was 90%.

¹H NMR (400 MHz, DMSO-d₆) δ 7.99 (dd, J=7.5, 2.2 Hz, 1H), 7.90 (d, J=7.8 Hz, 1H), 7.71-7.63 (m, 2H), 7.54-7.42 (m, 1H), 7.30 (s, 1H), 7.10 (s, 1H), 6.41-6.28 (m, 1H), 5.81 (d, J=7.5 Hz, 1H), 5.65-5.50 (m, 2H), 4.33 (s, 1H), 4.18-3.96 (m, 2H), 2.46-2.26 (m, 2H), 1.49 (dd, J=6.7, 1.4 Hz, 3H).

### Example 4

The structure of the target compound is:

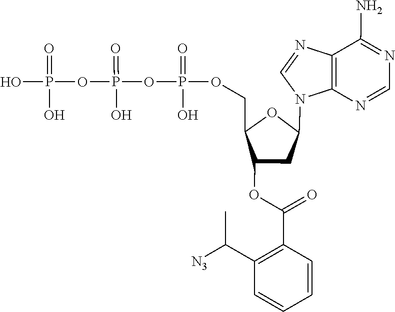 [SMILES: CC(N=[N+]=[N-])c1ccccc1C(=O)O[C@@H]1C[C@H](n2cnc3c(N)ncnc32)O[C@@H]1COP(=O)(O)OP(=O)(O)OP(=O)(O)O]

(1) Step 1

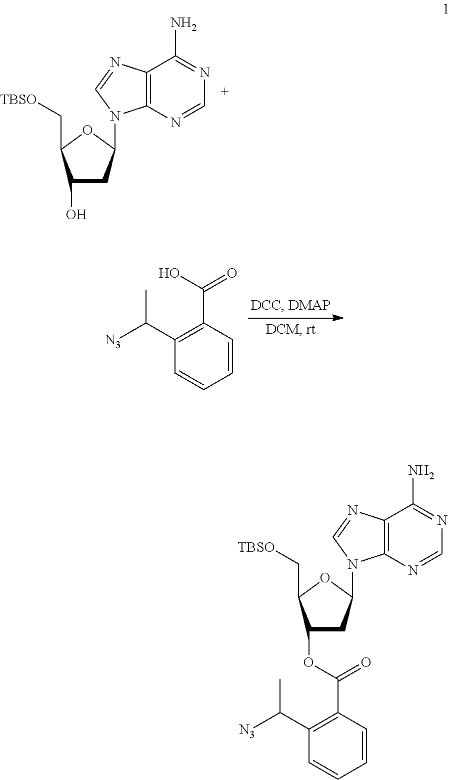

To a dried 100 mL round bottom flask equipped with a magnetic stirrer and a rubber stopper with argon balloon were added A-nucleoside (600 mg, 1.65 mmol, 1 eq) (available from Beijing Okeanos Tech. Co., Ltd., cat. No. OK-N-18101) and azido protection group (314 mg, 1.65 mmol, 1 eq) (available from Beijing Okeanos Tech. Co., Ltd., cat. No. OK-H-20001) separately, followed by adding with syringe 20 mL of anhydrous methylene chloride as solvent. The reaction mixture was stirred at room temperature to facilitate dissolution, followed by adding DCC (508 mg, 2.5 mmol, 1.5 eq) and DMAP (24 mg, 0.17 mmol, 0.1 eq) separately under nitrogen balloon for providing protection atmosphere, and allowed to react at room temperature for further 4 h. The reaction was monitored with TLC at half an hour interval, until the starting materials were substantially depleted. The reaction mixture was concentrated with a rotary evaporator, followed by product separation by silica gel column chromatography (Hexane/EA=10/1˜1/1, silica gel column diameter 3.5 cm, filler height 10 cm). The yield of this step was 95%.

¹H NMR (500 MHz, CDCl₃) δ 8.36 (1H, s); 8.28 (1H, d, J=4.4 Hz); 7.98 (1H, m); 7.65 (2H, m); 7.41 (1H, m); 6.60 (1H, t); 6.0 (2H, br.), 5.68 (2H, m); 4.40 (1H, br.); 4.01 (2H, br.); 1.93 (1H, m); 1.68 (1H, m); 1.56 (3H, d, J=6.7 Hz); 0.94 (9H, s); 0.15 (6H, s).

(2) Step 2

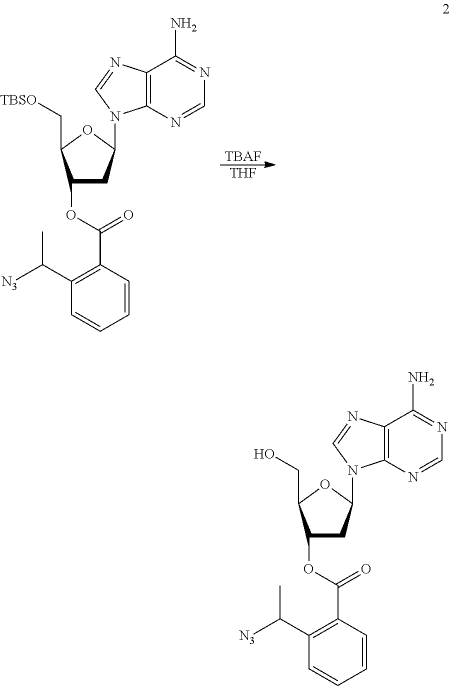

To a dried 100 mL round bottom flask equipped with a magnetic stirrer and a rubber stopper with argon balloon was added the product from Step 1 (828 mg, 1.54 mmol, 1 eq), followed by adding with syringe 20 mL of anhydrous THF as solvent. The reaction mixture was stirred to facilitate dissolution, cooled in ice bath, followed by slowly adding with syringe 1 mol/L solution of TBAF (2.0 mL, 2.0 mmol, 1.3 eq) dropwise. The flask was brough to room temperature and stirred continuously for about 2 h. The reaction was monitored with TLC at half an hour interval, until the dot of starting material on TLC plate disappeared. The reaction mixture was concentrated with a rotary evaporator, followed by product separation by silica gel column chromatography (Hexane/EA=10/1˜1/1 to methylene chloride/anhydrous methanol=100/1˜10/1, silica gel column diameter 3.5 cm, filler height 10 cm). The yield of this step was 70%.

¹H NMR (500 MHz, DMSO-d₆) δ 10.66 (s, 1H), 8.00 (d, J=1.5 Hz, 1H), 7.92 (ddd, J=8.0, 3.0, 1.4 Hz, 1H), 7.82-7.59 (m, 2H), 7.51 (t, J=7.5 Hz, 1H), 6.46 (s, 2H), 6.20 (dt, J=9.5, 5.4 Hz, 1H), 5.58 (ddd, J=15.6, 6.2, 2.5 Hz, 2H), 5.21 (td, J=5.6, 1.8 Hz, 1H), 4.24 (dd, J=4.3, 1.7 Hz, 1H), 3.77-3.55 (m, 2H), 2.91 (qd, J=9.1, 5.8 Hz, 1H), 2.62 (dt, J=13.7, 6.6 Hz, 1H), 1.51 (dd, J=6.7, 1.7 Hz, 3H).

(3) Step 3

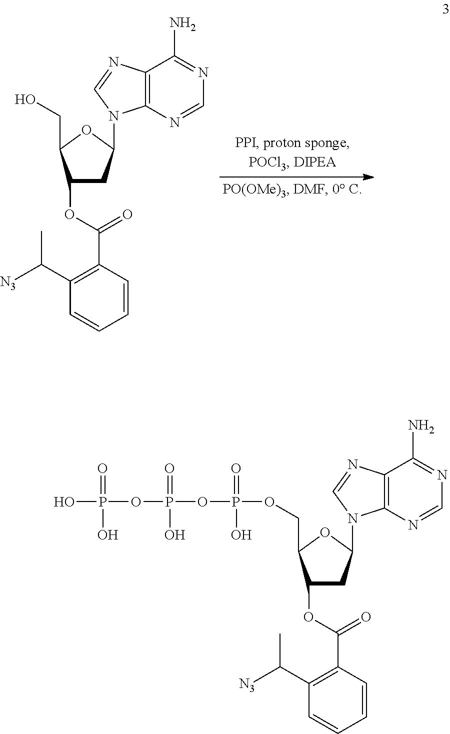 [SMILES: CC(N=[N+]=[N-])c1ccccc1C(=O)O[C@@H]1C[C@H](n2cnc3c(N)ncnc32)O[C@@H]1CO ; CC(N=[N+]=[N-])c1ccccc1C(=O)O[C@@H]1C[C@H](n2cnc3c(N)ncnc32)O[C@@H]1COP(=O)(O)OP(=O)(O)OP(=O)(O)O]

To a dried 100 mL two-necked 1 #flask equipped with a magnetic stirrer and a three-way valve with argon balloon were added product from Step 2 (360 mg, 0.85 mmol, 1 eq) and proton sponge (363.5 mg, 1.7 mmol, 2 eq). To a dried 100 mL two-necked 2 #flask equipped with a magnetic stirrer and a three-way valve with argon balloon was added tributyl ammonium pyrophosphate (932.5 mg, 1.7 mmol, 2 eq). The two-necked 1 #flask was filled with argon, followed by adding 15 mL of trimethyl phosphate with stirring to facilitate dissolution. The reaction was brought to 0° C., followed by slowly adding with syringe phosphorus oxychloride (120 μL, 1.3 mmol, 1.5 eq) and stirring continuously at 0° C. for 1.5 h. The two-necked 2 #flask was filled with argon, followed by adding with syringe 5 mL of anhydrous DMF and DIPEA (740 μL, 4.25 mmol, 5 eq) with stirring at 0° C. to facilitate dissolution. The mixture was maintained at 0° C. and continuously stirred for 3 h, followed by adding 20 mL of solution of TEAB (0.1 mol/L) to quench the reaction. The reaction mixture was diluted with 50 mL of deionized water, and then separated by DEAE resin column prepared-in-advance and eluted with gradient elution (H₂O:TEAB(1 mol/L)=10/1˜0/1, DEAE column diameter 4.5 cm, filler height 8 cm). The fractions containing the triphosphated product were collected and concentrated with a rotary evaporator under vacuum, followed by preparative liquid chromatography separation with gradient elution (CH₃CN:TEAB(0.1 mol/L)=2/98˜98/2). The high-purity product solution recovered from the separation was concentrated with a rotary evaporator under vacuum, transferred to a plastic centrifuge tube, and then freeze-dried with a lyophilizer into a powder solid. MW=664. The yield of this step was 40%.

¹H NMR (400 MHz, DMSO-d₆) δ 8.62 (d, J=3.6 Hz, 1H), 8.15 (d, J=1.5 Hz, 1H), 7.97 (d, J=7.8 Hz, 1H), 7.72-7.65 (m, 2H), 7.55-7.47 (m, 1H), 7.29 (s, 2H), 6.51-6.46 (m, 1H), 5.74 (d, J=5.3 Hz, 1H), 5.68-5.58 (m, 1H), 4.44-4.41 (m, 1H), 4.14-4.01 (m, 2H), 3.19-3.11 (m, 1H), 2.75-2.65 (m, 1H), 1.51 (dd, J=6.7, 1.5 Hz, 3H).

### Example 5

The structure of the target compound is:

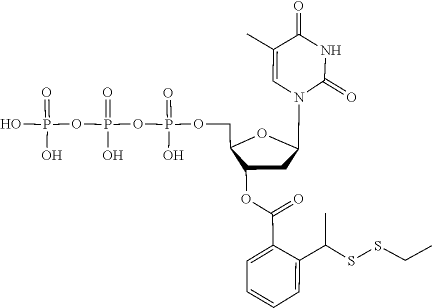 [SMILES: CCSSC(C)c1ccccc1C(=O)O[C@@H]1C[C@H](n2cc(C)c(=O)[nH]c2=O)O[C@@H]1COP(=O)(O)OP(=O)(O)OP(=O)(O)O]

(1) Step 1

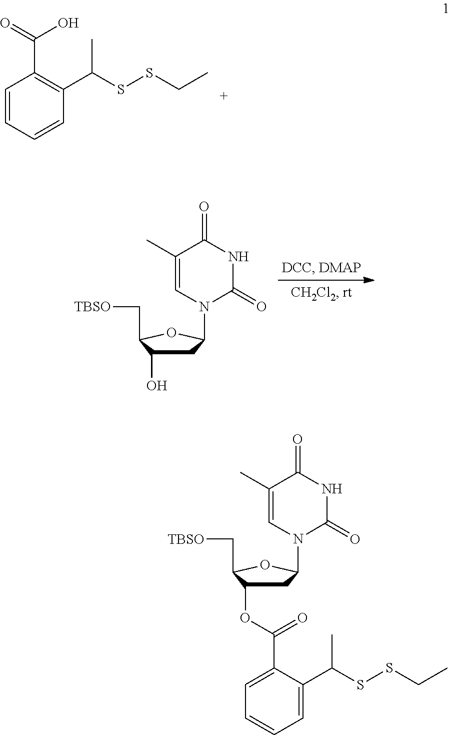

To a dried 100 mL round bottom flask equipped with a magnetic stirrer and a rubber stopper with argon balloon were added T-nucleoside (500 mg, 1.40 mmol, 1 eq) (available from Beijing Okeanos Tech. Co., Ltd., cat. No. OK-N-18102) and disulfaneyl protection group (340 mg, 1.40 mmol, 1 eq) (available from Beijing Okeanos Tech. Co., Ltd., cat. No.: OK-H-21003) separately, followed by adding with syringe 20 mL of anhydrous methylene chloride as solvent. The reaction mixture was stirred at room temperature to facilitate dissolution, followed by adding DCC (433 mg, 2.10 mmol, 1.5 eq) and DMAP (17 mg, 0.14 mmol, 0.1 eq) separately under nitrogen balloon for providing protection atmosphere, and allowed to react at room temperature for further 6 h. The reaction was monitored with TLC at half an hour interval, until the starting materials were substantially depleted. The reaction mixture was concentrated with a rotary evaporator, followed by product separation by silica gel column chromatography (Hexane/EA=10/1˜1/1, silica gel column diameter 3.5 cm, filler height 10 cm), yield 85%. ¹H NMR (400 MHz, CDCl₃) δ 8.61 (d, J=4.7 Hz, 1H), 7.84 (t, J=8.3 Hz, 1H), 7.63-7.57 (m, 2H), 7.52 (t, J=7.6 Hz, 1H), 7.33 (dd, J=11.0, 4.1 Hz, 1H), 6.47-6.40 (m, 1H), 5.52-5.45 (m, 1H), 5.31-5.20 (m, 1H), 4.30 (d, J=21.8 Hz, 1H), 4.07-3.94 (m, 2H), 3.51-3.44 (m, 1H), 2.63-2.53 (m, 1H), 2.29-2.20 (m, 2H), 1.94 (s, 3H), 1.69 (d, J=7.0 Hz, 3H), 1.12-1.07 (m, 3H), 0.96 (s, 9H), 0.17 (s, 6H).

(2) Step 2

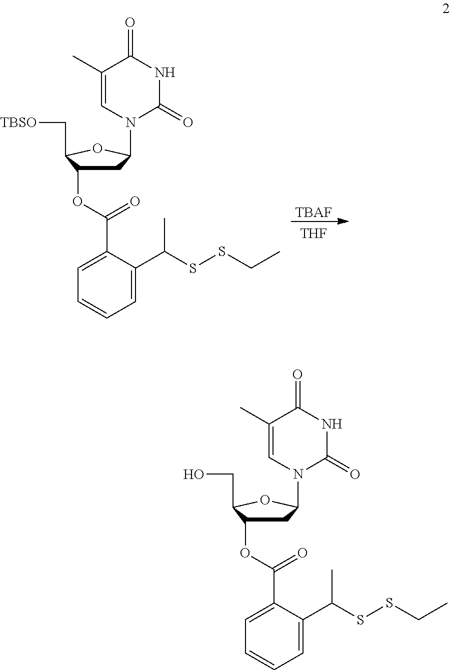

To a dried 100 mL round bottom flask equipped with a magnetic stirrer and a rubber stopper with argon balloon was added the product from Step 1 (690 mg, 1.19 mmol, 1 eq), followed by adding with syringe 20 mL of anhydrous THF as solvent. The reaction mixture was stirred at room temperature to facilitate dissolution, cooled in ice bath, followed by slowly adding with syringe 1 mol/L solution of TBAF (1.6 mL, 1.6 mmol, 1.3 eq) dropwise. The flask was brough to room temperature and stirred continuously for about 2 h. The reaction was monitored with TLC at half an hour interval, until the dot of starting material on TLC plate disappeared. The reaction mixture was concentrated with a rotary evaporator, followed by product separation by silica gel column chromatography (Hexane/EA=5/1˜1/1 to methylene chloride/anhydrous methanol=100/1˜10/1, silica gel column diameter 3.5 cm, filler height 10 cm). The yield of this step was 80%.

¹H NMR (500 MHz, CDCl₃) δ 8.57 (s, 1H), 7.87-7.80 (m, 1H), 7.60 (dd, J=7.8, 1.9 Hz, 1H), 7.57-7.50 (m, 2H), 7.39-7.29 (m, 1H), 6.35-6.25 (m, 1H), 5.64-5.56 (m, 1H), 5.23 (p, J=7.1 Hz, 1H), 4.29 (dd, J=14.3, 2.4 Hz, 1H), 4.02 (dd, J=4.9, 2.5 Hz, 2H), 2.59-2.55 (m, 2H), 2.29-2.19 (m, 2H), 1.94 (d, J=0.6 Hz, 3H), 1.70 (d, J=7.0 Hz, 3H), 1.11 (td, J=7.4, 2.6 Hz, 3H).

(3) Step 3

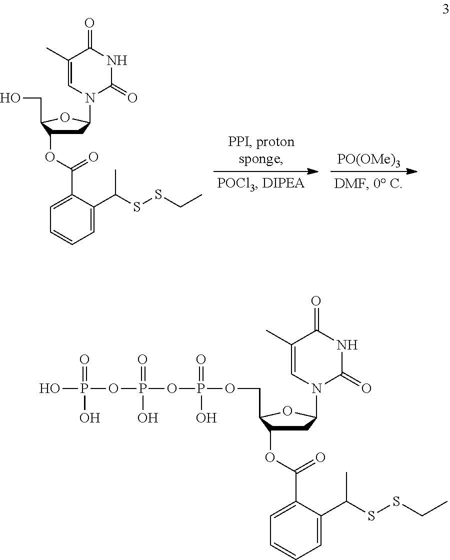

To a dried 100 mL two-necked 1 #flask equipped with a magnetic stirrer and a three-way valve with argon balloon were added product from Step 2 (396 mg, 0.85 mmol, 1 eq) and proton sponge (363.5 mg, 1.7 mmol, 2 eq). To a dried 100 mL two-necked 2 #flask equipped with a magnetic stirrer and a three-way valve with argon balloon was added tributyl ammonium pyrophosphate (932.5 mg, 1.7 mmol, 2 eq). The two-necked 1 #flask was filled with argon, followed by adding 15 mL of trimethyl phosphate with stirring to facilitate dissolution. The reaction was brought to 0° C., followed by slowly adding with syringe phosphorus oxychloride (120 μL, 1.3 mmol, 1.5 eq) and stirring continuously at 0° C. for 1.5 h. The two-necked 2 #flask was filled with argon, followed by adding with syringe 5 mL of anhydrous DMF and DIPEA (740 μL, 4.25 mmol, 5 eq) at 0° C. with stirring to facilitate dissolution. The mixture was maintained at 0° C. and continuously stirred for 3 h, followed by adding 20 mL of solution of TEAB (0.1 mol/L) to quench the reaction. The reaction mixture was diluted with 50 mL of deionized water, and then separated by DEAE resin column prepared-in-advance and eluted with gradient elution (H₂O:TEAB(1 mol/L)=10/1˜0/1, DEAE column diameter 4.5 cm, filler height 8 cm). The fractions containing the triphosphated product were collected and concentrated with a rotary evaporator under vacuum, followed by preparative liquid chromatography separation with gradient elution (CH₃CN:TEAB(0.1 mol/L)=2/98˜98/2). The high-purity product solution recovered from the separation was concentrated with a rotary evaporator under vacuum, transferred to a plastic centrifuge tube, and then freeze-dried with a lyophilizer into a powder solid. MW=706. The yield of this step was 40%.

¹H NMR (400 MHz, DMSO-d₆) δ 11.30 (s, 1H), 7.96 (dd, J=6.0, 1.0 Hz, 1H), 7.86 (d, J=7.7 Hz, 1H), 7.68-7.56 (m, 2H), 7.46-7.41 (m, 1H), 6.38-6.27 (m, 1H), 5.60 (d, J=5.3 Hz, 1H), 5.16-5.11 (m, 1H), 4.31 (d, J=20.8 Hz, 1H), 4.18-4.00 (m, 2H), 2.58-2.51 (m, 1H), 2.44-2.22 (m, 3H), 1.85 (s, 3H), 1.65 (d, J=7.0 Hz, 3H), 1.09-1.03 (m, 3H).

### Example 6

The structure of the target compound is:

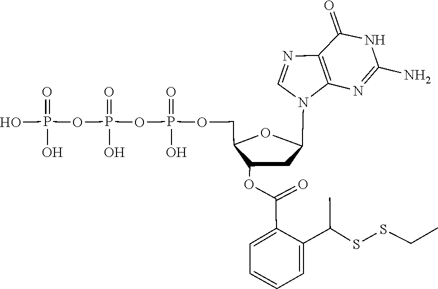 [SMILES: CCSSC(C)c1ccccc1C(=O)O[C@@H]1C[C@H](n2cnc3c(=O)[nH]c(N)nc32)O[C@@H]1COP(=O)(O)OP(=O)(O)OP(=O)(O)O]

(1) Step 1

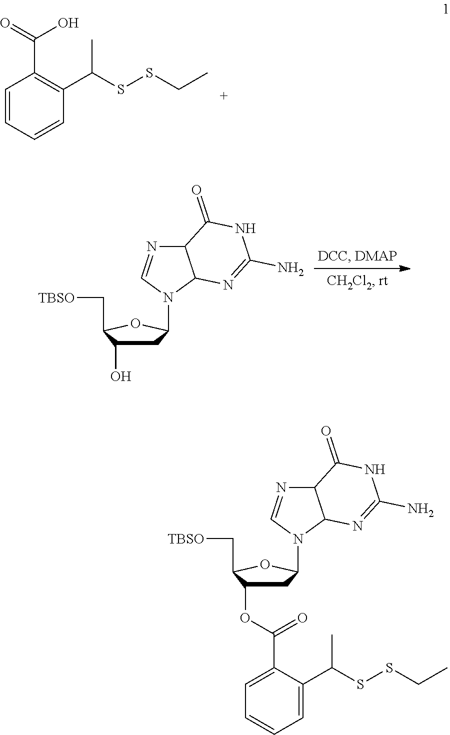

To a dried 100 mL round bottom flask equipped with a magnetic stirrer and a rubber stopper with argon balloon were added G-nucleoside (600 mg, 1.58 mmol, 1 eq) (available from Beijing Okeanos Tech. Co., Ltd., cat. No. OK-N-18103) and disulfaneyl protection group (605 mg, 1.58 mmol, 1 eq) (available from Beijing Okeanos Tech. Co., Ltd., cat. No. OK-H-21003) separately, followed by adding with syringe 20 mL of anhydrous methylene chloride as solvent. The reaction mixture was stirred at room temperature to facilitate dissolution, followed by adding DCC (488 mg, 2.37 mmol, 1.5 eq) and DMAP (20 mg, 0.16 mmol, 0.1 eq) separately under nitrogen balloon for providing protection atmosphere, and allowed to react at room temperature for further 4 h. The reaction was monitored with TLC at half an hour interval, until the starting materials were substantially depleted. The reaction mixture was concentrated with a rotary evaporator, followed by product separation by silica gel column chromatography (Hexane/EA=10/1-1/2, silica gel column diameter 3.5 cm, filler height 10 cm). The yield of this step was 80%.

¹H NMR (500 MHz, CDCl₃) δ 12.00 (s, 1H), 7.93 (t, J=9.2 Hz, 1H), 7.88-7.83 (m, 1H), 7.59 (dd, J=13.6, 8.0 Hz, 1H), 7.53 (t, J=7.6 Hz, 1H), 7.33 (t, J=7.5 Hz, 1H), 6.37-6.33 (m, 2H), 5.62 (s, 1H), 5.30-5.22 (m, 1H), 4.36 (dd, J=10.9, 1.5 Hz, 1H), 4.01-3.89 (m, 2H), 3.51-3.40 (m, 1H), 2.78-2.66 (m, 2H), 2.33-2.22 (m, 2H), 1.70 (d, J=6.9 Hz, 3H), 1.13 (d, J=2.5 Hz, 3H), 0.92 (s, 9H), 0.12 (s, 6H).

(2) Step 2

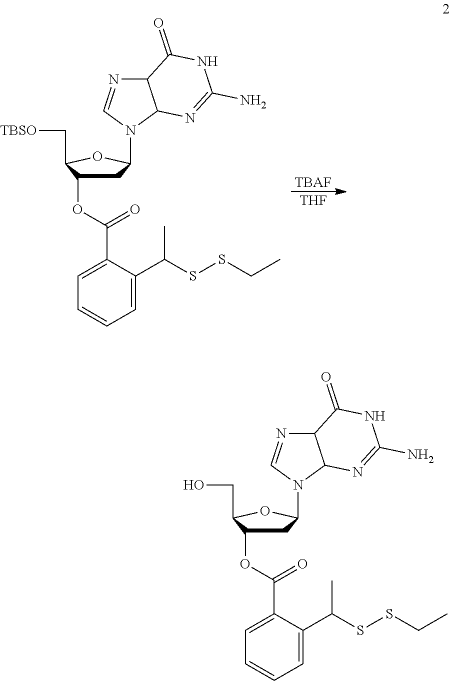

To a dried 100 mL round bottom flask equipped with a magnetic stirrer and a rubber stopper with argon balloon was added the product from Step 1 (932 mg, 1.54 mmol, 1 eq), followed by adding with syringe 20 mL of anhydrous THF as solvent. The reaction mixture was stirred to facilitate dissolution, cooled in ice bath, followed by slowly adding with syringe 1 mol/L solution of TBAF (2.0 mL, 2.0 mmol, 1.3 eq) dropwise. The flask was brough to room temperature and stirred continuously for about 2 h. The reaction was monitored with TLC at half an hour interval, until the dot of starting material on TLC plate disappeared. The reaction mixture was concentrated with a rotary evaporator, followed by product separation by silica gel column chromatography (Hexane/EA=10/1˜1/1 to methylene chloride/anhydrous methanol=100/1˜10/1, silica gel column diameter 3.5 cm, filler height 10 cm). The yield of this step was 70%.

¹H NMR (400 MHz, CDCl₃) δ 11.71 (s, 1H), 7.97 (d, J=7.8 Hz, 1H), 7.62 (d, J=7.9 Hz, 1H), 7.52 (t, J=7.5 Hz, 2H), 7.32 (t, J=7.2 Hz, 1H), 6.36-6.02 (m, 1H), 5.41-5.22 (m, 2H), 3.03-2.97 (m, 3H), 2.34 (dd, J=14.6, 7.2 Hz, 2H), 2.26-2.20 (m, 1H), 2.06-1.97 (m, 1H), 1.69 (d, J=6.9 Hz, 3H), 1.14 (t, J=7.3 Hz, 3H).

(3) Step 3

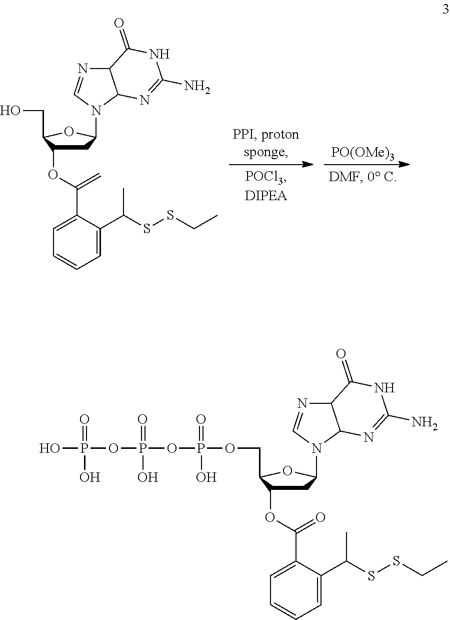

To a dried 100 mL two-necked 1 #flask equipped with a magnetic stirrer and a three-way valve with argon balloon were added product from Step 2 (417 mg, 0.85 mmol, 1 eq) and proton sponge (363.5 mg, 1.7 mmol, 2 eq). To a dried 100 mL two-necked 2 #flask equipped with a magnetic stirrer and a three-way valve with argon balloon was added tributyl ammonium pyrophosphate (932.5 mg, 1.7 mmol, 2 eq). The two-necked 1 #flask was filled with argon, followed by adding 15 mL of trimethyl phosphate with stirring to facilitate dissolution. The reaction was brought to 0° C., followed by slowly adding with syringe phosphorus oxychloride (120 μL, 1.3 mmol, 1.5 eq) and stirring continuously at 0° C. for 1.5 h. The two-necked 2 #flask was filled with argon, followed by adding with syringe 5 mL of anhydrous DMF and DIPEA (740 μL, 4.25 mmol, 5 eq) with stirring at 0° C. to facilitate dissolution. The mixture was maintained at 0° C. and continuously stirred for 3 h, followed by adding 20 mL of solution of TEAB (0.1 mol/L) to quench the reaction. The reaction mixture was continuously stirred for 0.5 h, diluted with 50 mL of deionized water, and then separated by DEAE resin column prepared-in-advance and eluted with gradient elution (H₂O:TEAB(1 mol/L)=10/1˜0/1, DEAE column diameter 4.5 cm, filler height 8 cm).

The fractions containing the triphosphated product were collected and concentrated under vacuum to 10 mL, followed by preparative liquid chromatography separation with gradient elution (CH₃CN:TEAB(0.1 mol/L)=2/98˜98/2). The high-purity product solution recovered from the separation was concentrated with a rotary evaporator under vacuum, transferred to a plastic centrifuge tube, and then freeze-dried with a lyophilizer into a powder solid. MW=706. The yield of this step was 40%.

¹H NMR (400 MHz, DMSO-d₆) δ 10.82 (s, 1H), 8.03 (d, J=5.5 Hz, 1H), 7.88 (d, J=7.8 Hz, 1H), 7.69-7.58 (m, 2H), 7.46-7.42 (m, 1H), 6.72 (s, 2H), 6.25-6.18 (m, 1H), 5.70 (t, J=5.0 Hz, 1H), 5.22-5.10 (m, 1H), 4.40-4.27 (m, 1H), 4.21 (dd, J=11.0, 5.0 Hz, 1H), 4.01 (dd, J=7.0, 3.7 Hz, 1H), 2.59-2.52 (m, 1H), 2.48-2.30 (m, 3H), 1.65 (d, J=7.0 Hz, 3H), 1.07 (t, J=5.1 Hz, 3H).

### Example 7

The structure of the target compound is:

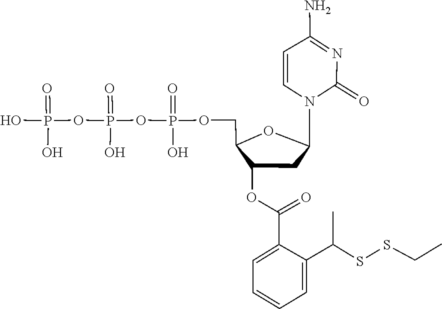 [SMILES: CCSSC(C)c1ccccc1C(=O)O[C@@H]1C[C@H](n2ccc(N)nc2=O)O[C@@H]1COP(=O)(O)OP(=O)(O)OP(=O)(O)O]

(1) Step 1

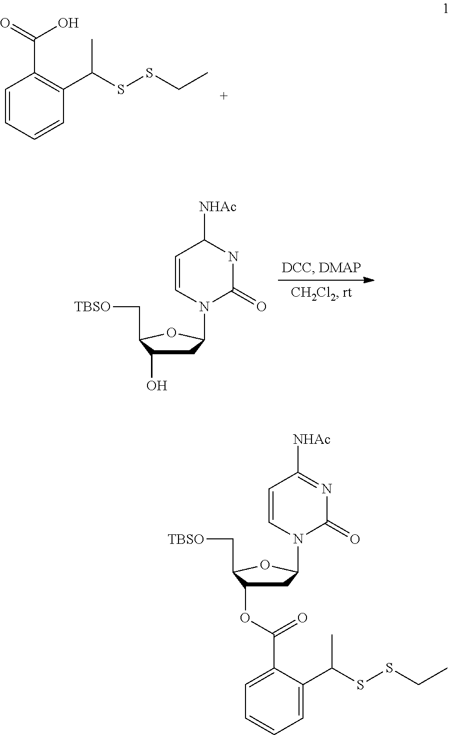

To a dried 100 mL round bottom flask equipped with a magnetic stirrer and a rubber stopper with argon balloon were added C-nucleoside (608 mg, 1.58 mmol, 1 eq) (available from Beijing Okeanos Tech. Co., Ltd., cat. No. OK-N-18104) and disulfaneyl protection group (605 mg, 1.58 mmol, 1 eq) (available from Beijing Okeanos Tech. Co., Ltd., cat. No.: OK-H-21003) separately, followed by adding with syringe 20 mL of anhydrous methylene chloride as solvent. The reaction mixture was stirred at room temperature to facilitate dissolution, followed by adding DCC (488 mg, 2.37 mmol, 1.5 eq) and DMAP (20 mg, 0.16 mmol, 0.1 eq) separately under nitrogen balloon for providing protection atmosphere, and allowed to react at room temperature for further 4 h. The reaction was monitored with TLC at half an hour interval, until the starting materials were substantially depleted. The reaction mixture was concentrated with a rotary evaporator, followed by product separation by silica gel column chromatography (Hexane/EA=10/1-1/2, silica gel column diameter 3.5 cm, filler height 10 cm). The yield of this step was 90%.

¹H NMR (500 MHz, CDCl₃) δ 9.53 (s, 1H), 8.35 (d, J=6.0 Hz, 1H), 7.87 (ddd, J=7.8, 4.4, 1.3 Hz, 1H), 7.59 (t, J=7.9 Hz, 1H), 7.54-7.50 (m, 1H), 7.41 (d, J=6.0 Hz, 1H), 7.32 (td, J=7.7, 1.2 Hz, 1H), 6.44-6.38 (m, 1H), 5.50-5.48 (m, 1H), 5.29-5.21 (m, 1H), 4.45-4.36 (m, 1H), 4.01 (s, 2H), 3.50-3.44 (m, 1H), 2.93-2.86 (m, 1H), 2.24-2.12 (m, 2H), 1.69 (dd, J=7.0, 0.6 Hz, 3H), 1.11 (td, J=7.4, 3.3 Hz, 3H), 0.93 (s, 9H), 0.13 (s, 6H).

(2) Step 2

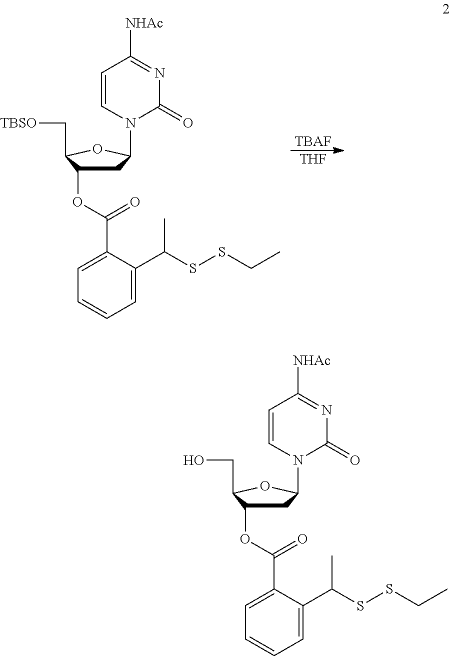

To a dried 100 mL round bottom flask equipped with a magnetic stirrer and a rubber stopper with argon balloon was added the product from Step 1 (938 mg, 1.54 mmol, 1 eq), followed by adding with syringe 20 mL of anhydrous THF as solvent. The reaction mixture was stirred to facilitate dissolution, cooled in ice bath, followed by slowly adding with syringe 1 mol/L solution of TBAF (2.0 mL, 2.0 mmol, 1.3 eq) dropwise. The flask was brough to room temperature and stirred continuously for about 2 h. The reaction was monitored with TLC at half an hour interval until the dot of starting material on TLC plate disappeared. The reaction mixture was concentrated with a rotary evaporator, followed by product separation by silica gel column chromatography (Hexane/EA=10/1˜1/1 to methylene chloride/anhydrous methanol=100/1˜10/1, silica gel column diameter 3.5 cm, filler height 10 cm). The yield of this step was 80%.

¹H NMR (400 MHz, CDCl₃) δ 9.50 (s, 1H), 8.39 (s, 1H), 7.85 (t, J=7.2 Hz, 1H), 7.61-7.51 (m, 3H), 7.34 (t, J=7.3 Hz, 1H), 6.30 (s, 1H), 5.63 (d, J=2.8 Hz, 1H), 5.27-5.19 (m, 1H), 4.40 (d, J=15.5 Hz, 1H), 4.15-3.94 (m, 2H), 3.40 (s, 1H), 2.83 (td, J=14.3, 4.8 Hz, 1H), 2.60-2.45 (m, 1H), 2.27-2.14 (m, 2H), 1.69 (d, J=6.8 Hz, 3H), 1.10 (q, J=6.8 Hz, 3H).

(3) Step 3

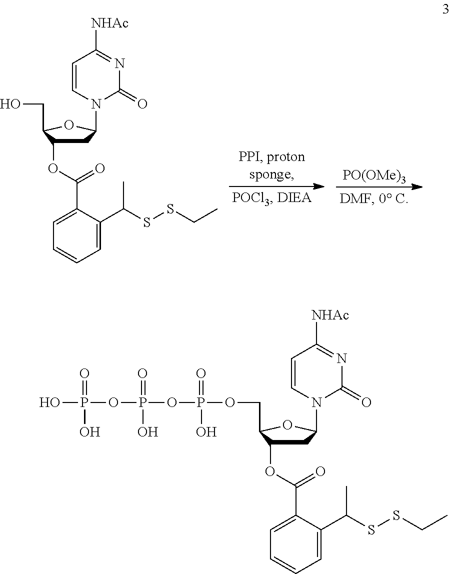

To a dried 100 mL two-necked 1 #flask equipped with a magnetic stirrer and a three-way valve with argon balloon were added product from Step 2 (421 mg, 0.85 mmol, 1 eq) and proton sponge (363.5 mg, 1.7 mmol, 2 eq). To a dried 100 mL two-necked 2 #flask equipped with a magnetic stirrer and a three-way valve with argon balloon was added tributyl ammonium pyrophosphate (932.5 mg, 1.7 mmol, 2 eq). The two-necked 1 #flask was filled with argon, followed by adding 15 mL of trimethyl phosphate with stirring to facilitate dissolution. The reaction was brought to 0° C., followed by slowly adding with syringe phosphorus oxychloride (120 μL, 1.3 mmol, 1.5 eq) and stirring continuously at 0° C. for 1.5 h. The two-necked 2 #flask was filled with argon, followed by adding with syringe 5 mL of anhydrous DMF and DIPEA (740 μL, 4.25 mmol, 5 eq) with stirring at 0° C. to facilitate dissolution. The mixture was maintained at 0° C. and continuously stirred for 3 h, followed by adding 20 mL of solution of TEAB (0.1 mol/L) to quench the reaction. The reaction mixture was continuously stirred for 0.5 h. The reaction mixture was diluted with 50 mL of deionized water, and then separated by DEAE resin column prepared-in-advance and eluted with gradient elution (H₂O:TEAB(1 mol/L)=10/1˜0/1, DEAE column diameter 4.5 cm, filler height 8 cm). The fractions containing the triphosphated product were collected and concentrated with a rotary evaporator under vacuum, followed by preparative liquid chromatography separation with gradient elution (CH₃CN:TEAB(0.1 mol/L)=2/98˜98/2). The high-purity product solution recovered from the separation was concentrated with a rotary evaporator under vacuum, transferred to a plastic centrifuge tube, and then freeze-dried with a lyophilizer into a powder solid. MW=733. The yield of this step was 45%.

(4) Step 4

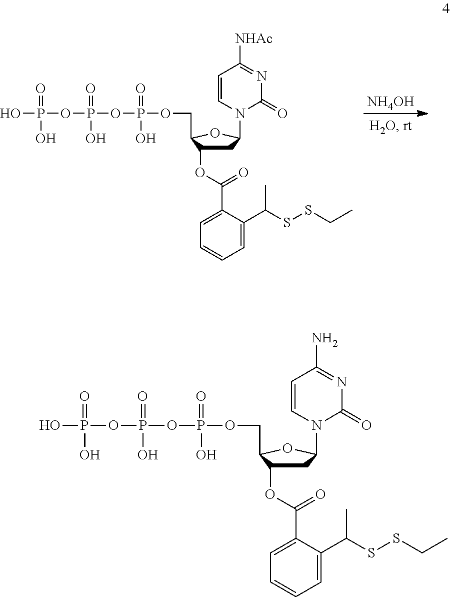

To a dried 100 mL round bottom flask equipped with a magnetic stirrer were added the triphosphated product from Step 3 (537 mg, 0.73 mmol, 1 eq) and 5 ml of deionized water. The mixture was stirred at room temperature to complete dissolution, followed by adding 25% aqueous ammonia (2.5 g, 36.5 mmol, 50 eq) and stirring at room temperature for about 2 h. Ammonia in the solution was removed by a rotary evaporator under vacuum and the residue was diluted with deionized water to about 10 mL. After separation with preparative liquid chromatography with gradient elution (CH₃CN:TEAB(0.1 mol/L)=2/98˜98/2). The high-purity product solution recovered from the separation was concentrated with a rotary evaporator under vacuum, transferred to a plastic centrifuge tube, and then freeze-dried with a lyophilizer into a powder solid. MW=691. The yield of this step was 90%.

¹H NMR (400 MHz, DMSO-d₆) δ 7.97 (dd, J=7.4, 6.2 Hz, 1H), 7.85 (d, J=8.7 Hz, 1H), 7.66-7.58 (m, 2H), 7.45-7.41 (m, 1H), 7.31 (s, 1H), 7.10 (s, 1H), 6.39-6.29 (m, 1H), 5.81 (d, J=7.4 Hz, 1H), 5.53 (d, J=5.0 Hz, 1H), 5.17-5.10 (m, 1H), 4.31 (d, J=19.8 Hz, 1H), 4.13-4.01 (m, 2H), 2.46-2.23 (m, 4H), 1.65 (d, J=7.0 Hz, 3H), 1.09-1.03 (m, 3H).

### Example 8

The structure of the target compound is:

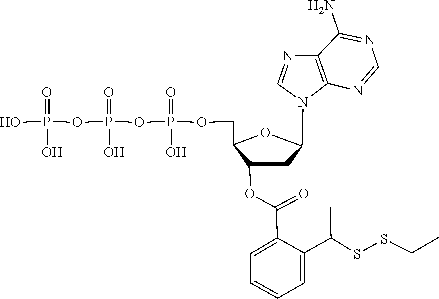 [SMILES: CCSSC(C)c1ccccc1C(=O)O[C@@H]1C[C@H](n2cnc3c(N)ncnc32)O[C@@H]1COP(=O)(O)OP(=O)(O)OP(=O)(O)O]

(1) Step 1

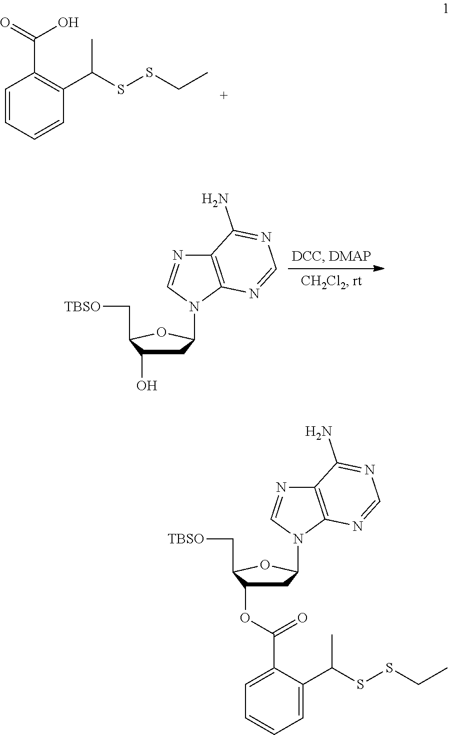

To a dried 100 mL round bottom flask equipped with a magnetic stirrer and a rubber stopper with argon balloon were added A-nucleoside (577 mg, 1.58 mmol, 1 eq) (available from Beijing Okeanos Tech. Co., Ltd., cat. No. OK-N-18101) and disulfaneyl protection group (605 mg, 1.58 mmol, 1 eq) (available from Beijing Okeanos Tech. Co., Ltd., cat. No. OK-H-21003) separately, followed by adding with syringe 20 mL of anhydrous methylene chloride as solvent. The reaction mixture was stirred at room temperature to facilitate dissolution, followed by adding DCC (488 mg, 2.37 mmol, 1.5 eq) and DMAP (20 mg, 0.16 mmol, 0.1 eq) separately under nitrogen balloon for providing protection atmosphere, and allowed to react at room temperature for further 4 h. The reaction was monitored with TLC at half an hour interval until the starting materials were substantially depleted. The reaction mixture was concentrated with a rotary evaporator, followed by product separation by silica gel column chromatography (Hexane/EA=10/1-1/2, silica gel column diameter 3.5 cm, filler height 10 cm). The yield of this step was 85%.

¹H NMR (500 MHz, CDCl₃) δ 8.34 (s, 1H), 8.24 (d, J=11.2 Hz, 1H), 7.87 (ddd, J=7.8, 4.4, 1.3 Hz, 1H), 7.60 (d, J=7.9 Hz, 1H), 7.52 (t, J=7.6 Hz, 1H), 7.32 (td, J=7.7, 1.2 Hz, 1H), 6.58 (t, J=7.1 Hz, 1H), 6.21 (s, 2H), 5.68-5.61 (m, 1H), 5.27 (p, J=7.0 Hz, 1H), 4.45-4.36 (m, 1H), 4.04-3.95 (m, 2H), 2.83-2.73 (m, 2H), 2.31-2.18 (m, 2H), 1.69 (dd, J=7.0, 0.6 Hz, 3H), 1.11 (td, J=7.4, 3.3 Hz, 3H), 0.93 (s, 9H), 0.13 (t, J=1.7 Hz, 6H).

(2) Step 2

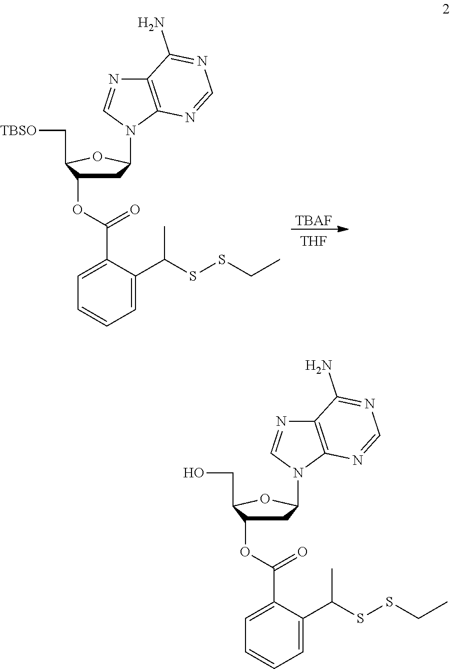

To a dried 100 mL round bottom flask equipped with a magnetic stirrer and a rubber stopper with argon balloon was added the product from Step 1 (907 mg, 1.54 mmol, 1 eq), followed by adding with syringe 20 mL of anhydrous THF as solvent. The reaction mixture was stirred to facilitate dissolution, cooled in ice bath, followed by slowly adding with syringe 1 mol/L solution of TBAF (2.0 mL, 2.0 mmol, 1.3 eq) dropwise. The flask was brough to room temperature and stirred continuously for about 2 h. The reaction was monitored with TLC at half an hour interval until the dot of starting material on TLC plate disappeared. The reaction mixture was concentrated with a rotary evaporator, followed by product separation by silica gel column chromatography (Hexane/EA=10/1˜1/1 to methylene chloride/anhydrous methanol=100/1˜10/1, silica gel column diameter 3.5 cm, filler height 10 cm). The yield of this step was 80%.

¹H NMR (500 MHz, CDCl₃) δ 8.37 (s, 1H), 7.93 (s, 1H), 7.89-7.85 (m, 1H), 7.65-7.61 (m, 1H), 7.56 (td, J=7.7, 1.3 Hz, 1H), 7.39-7.33 (m, 1H), 6.37 (dd, J=9.3, 5.3 Hz, 1H), 6.02 (s, 2H), 5.83 (t, J=5.5 Hz, 1H), 5.31-5.23 (m, 1H), 4.48 (d, J=14.2 Hz, 1H), 4.09-3.97 (m, 2H), 3.33-3.28 (m, 1H), 2.63 (dd, J=14.0, 4.9 Hz, 1H), 2.41-2.31 (m, 2H), 1.73 (d, J=7.0 Hz, 3H), 1.17 (td, J=7.4, 2.3 Hz, 3H).

(3) Step 3

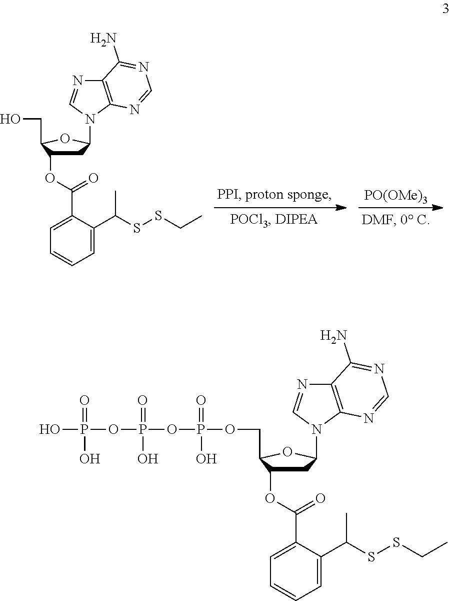

To a dried 100 mL two-necked 1 #flask equipped with a magnetic stirrer and a three-way valve with argon balloon were added product from Step 2 (404 mg, 0.85 mmol, 1 eq) and proton sponge (363.5 mg, 1.7 mmol, 2 eq). To a dried 100 mL two-necked 2 #flask equipped with a magnetic stirrer and a three-way valve with argon balloon was added tributyl ammonium pyrophosphate (932.5 mg, 1.7 mmol, 2 eq). The two-necked 1 #flask was filled with argon, followed by adding 15 mL of trimethyl phosphate with stirring to facilitate dissolution. The reaction was brought to 0° C., followed by slowly adding with syringe phosphorus oxychloride (120 μL, 1.3 mmol, 1.5 eq) and stirring continuously at 0° C. for 1.5 h. The two-necked 2 #flask was filled with argon, followed by adding with syringe 5 mL of anhydrous DMF and DIPEA (740 μL, 4.25 mmol, 5 eq) with stirring at 0° C. to facilitate dissolution. The mixture was maintained at 0° C. and continuously stirred for 3 h, followed by adding 20 mL of solution of TEAB (0.1 mol/L) to quench the reaction. The reaction mixture was continuously stirred for 0.5 h, diluted with 50 mL of deionized water, and then separated by DEAE resin column prepared-in-advance and eluted with gradient elution (H₂O:TEAB(1 mol/L)=10/1˜0/1, DEAE column diameter 4.5 cm, filler height 8 cm).

The fractions containing the triphosphated product were collected and concentrated with a rotary evaporator under vacuum, followed by preparative liquid chromatography separation with gradient elution (CH₃CN:TEAB(0.1 mol/L)=2/98˜98/2). The high-purity product solution recovered from the separation was concentrated with a rotary evaporator under vacuum, transferred to a plastic centrifuge tube, and then freeze-dried with a lyophilizer into a powder solid. MW=715. The yield of this step was 40%.

¹H NMR (400 MHz, DMSO-d₆) δ 8.60 (s, 1H), 8.15 (s, 1H), 8.05 (d, J=6.5 Hz, 1H), 7.69 (t, J=8.2 Hz, 1H), 7.60-7.54 (m, 2H), 7.27 (s, 2H), 6.25-6.18 (m, 1H), 5.70 (t, J=5.0 Hz, 1H), 5.22-5.10 (m, 1H), 4.40-4.27 (m, 1H), 4.21 (dd, J=11.0, 5.0 Hz, 1H), 4.01 (dd, J=7.0, 3.7 Hz, 1H), 2.59-2.52 (m, 1H), 2.48-2.30 (m, 3H), 1.65 (d, J=7.0 Hz, 3H), 1.07 (t, J=5.1 Hz, 3H).

### Example 9

(1) Step 1

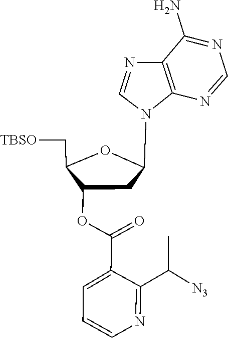 [SMILES: CC(N=[N+]=[N-])c1ncccc1C(=O)O[C@@H]1C[C@H](n2cnc3c(N)ncnc32)O[C@@H]1CO[Si](C)(C)C(C)(C)C]

The product was synthesized similarly to Example 4 step 1, wherein 2-(1-azidoethyl)benzoic acid was replaced by 2-(1-azidoethyl) nicotinic acid (available from Beijing Okeanos Tech. Co., Ltd., cat. No.: OK-H-20002), 85% yield, MW=539. ¹H NMR (400 MHz, DMSO-d₆) δ 8.86 (dd, J=4.7, 1.7 Hz, 1H), 8.38 (dt, J=7.9, 1.7 Hz, 1H), 8.33 (s, 1H), 8.16 (d, J=0.9 Hz, 1H), 7.58 (dd, J=7.9, 4.7 Hz, 1H), 7.32 (s, 2H), 6.47 (ddd, J=8.5, 6.1, 2.9 Hz, 1H), 5.66 (dq, J=6.2, 2.0 Hz, 1H), 5.41-5.24 (m, 1H), 4.44-4.23 (m, 1H), 4.05-3.77 (m, 2H), 3.13 (ddd, J=14.3, 8.3, 6.3 Hz, 1H), 2.87-2.68 (m, 1H), 1.61 (d, J=6.7 Hz, 3H), 0.86 (s, 9H), 0.04 (d, J=3.9 Hz, 6H).

(2) Step 2

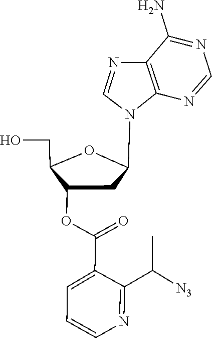 [SMILES: CC(N=[N+]=[N-])c1ncccc1C(=O)O[C@@H]1C[C@H](n2cnc3c(N)ncnc32)O[C@@H]1CO]

The product is synthesized similarly to Example 4 Step 2, wherein 2-(1-azidoethyl)benzoic acid was replaced by 2-(1-azidoethyl) nicotinic acid (available from Beijing Okeanos Tech. Co., Ltd., cat. No.: OK-H-20002), 75% yield, MW=425. ¹H NMR (500 MHz, DMSO-d₆) δ 8.86 (dd, J=4.8, 1.8 Hz, 1H), 8.45-8.31 (m, 2H), 8.16 (d, J=1.1 Hz, 1H), 7.59 (dd, J=7.9, 4.7 Hz, 1H), 7.38 (s, 2H), 6.47 (ddd, J=9.4, 5.8, 4.2 Hz, 1H), 5.71-5.62 (m, 1H), 5.57 (ddd, J=7.5, 4.8, 2.8 Hz, 1H), 5.33 (p, J=6.7 Hz, 1H), 4.34 (ddt, J=7.6, 4.1, 2.2 Hz, 1H), 3.83-3.60 (m, 2H), 3.09 (ddd, J=14.5, 9.0, 5.9 Hz, 1H), 2.85-2.65 (m, 1H), 1.61 (dd, J=6.7, 2.7 Hz, 3H).

(3) Step 3

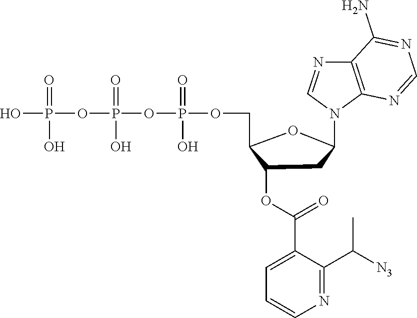 [SMILES: CC(N=[N+]=[N-])c1ncccc1C(=O)O[C@@H]1C[C@H](n2cnc3c(N)ncnc32)O[C@@H]1COP(=O)(O)OP(=O)(O)OP(=O)(O)O]

The product was synthesized similarly to Example 4 step 3, wherein 2-(1-azidoethyl)benzoic acid was replaced by 2-(1-azidoethyl) nicotinic acid (available from Beijing Okeanos Tech. Co., Ltd., cat. No.: OK-H-20002), 40% yield, MW=665. ¹H NMR (400 MHz, DMSO-d₆) δ 8.85 (d, J=3.6 Hz, 1H), 8.66 (d, J=3.4 Hz, 1H), 8.40 (d, J=7.3 Hz, 1H), 8.15 (s, 1H), 7.58 (dd, J=7.9, 4.8 Hz, 1H), 7.31 (s, 2H), 6.54-6.45 (m, 1H), 5.77 (d, J=5.1 Hz, 1H), 5.37-5.28 (m, 1H), 4.44 (d, J=4.5 Hz, 1H), 4.14-4.02 (m, 2H), 3.23-3.12 (m, 1H), 2.76-2.66 (m, 1H), 1.61 (dd, J=6.7, 2.1 Hz, 3H).

### Example 10

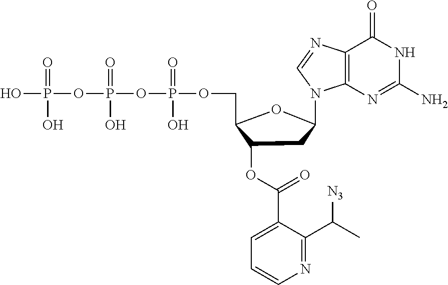 [SMILES: CC(N=[N+]=[N-])c1ncccc1C(=O)O[C@@H]1C[C@H](n2cnc3c(=O)[nH]c(N)nc32)O[C@@H]1COP(=O)(O)OP(=O)(O)OP(=O)(O)O]

The product was synthesized similarly to Example 2 Steps 1-3, wherein 2-(1-azidoethyl)benzoic acid was replaced by 2-(1-azidoethyl) nicotinic acid (available from Beijing Okeanos Tech. Co., Ltd., cat. No.: OK-H-20002)), yield for the last step was 40%, MW=681.

¹H NMR (400 MHz, DMSO-d₆) δ 10.77 (s, 1H), 8.84 (d, J=4.6 Hz, 1H), 8.39-8.29 (m, 1H), 8.02 (d, J=5.5 Hz, 1H), 7.57 (dd, J=7.8, 4.8 Hz, 1H), 6.71 (s, 2H), 6.28-6.16 (m, 1H), 5.73 (d, J=4.9 Hz, 1H), 5.35-5.28 (m, 1H), 4.36 (dd, J=10.4, 6.0 Hz, 1H), 4.24-4.15 (m, 1H), 4.01-3.97 (m, 1H), 3.25-3.13 (m, 1H), 2.61-2.55 (m, 1H), 1.60 (dd, J=6.6, 2.6 Hz, 3H).

### Example 11

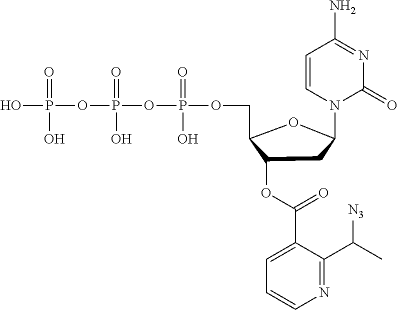 [SMILES: CC(N=[N+]=[N-])c1ncccc1C(=O)O[C@@H]1C[C@H](n2ccc(N)nc2=O)O[C@@H]1COP(=O)(O)OP(=O)(O)OP(=O)(O)O]

The product was synthesized similarly to Example 3 Steps 1-4, wherein 2-(1-azidoethyl)benzoic acid was replaced by 2-(1-azidoethyl) nicotinic acid (available from Beijing Okeanos Tech. Co., Ltd., cat. No.: OK-H-20002), yield for the last two steps was 30%, MW=641.

¹H NMR (400 MHz, DMSO-d₆) δ 8.83 (d, J=3.5 Hz, 1H), 8.33 (d, J=7.8 Hz, 1H), 7.95 (dd, J=7.4, 2.9 Hz, 1H), 7.55 (dd, J=7.9, 4.7 Hz, 1H), 7.30 (s, 1H), 7.08 (s, 1H), 6.37-6.34 (m, 1H), 5.82 (d, J=7.4 Hz, 1H), 5.54 (d, J=4.9 Hz, 1H), 5.39-5.20 (m, 1H), 4.39-4.32 (m, 1H), 4.14-3.98 (m, 2H), 2.47-2.41 (m, 1H), 2.36-2.24 (m, 1H), 1.59 (dd, J=6.7, 1.7 Hz, 3H).

### Example 12

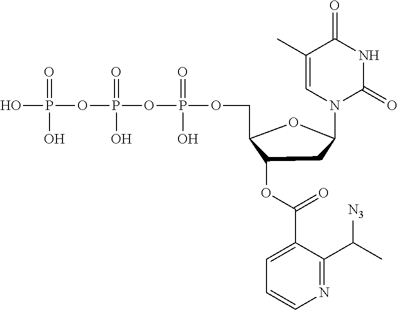 [SMILES: Cc1cn([C@H]2C[C@@H](OC(=O)c3cccnc3C(C)N=[N+]=[N-])[C@@H](COP(=O)(O)OP(=O)(O)OP(=O)(O)O)O2)c(=O)[nH]c1=O]

The product was synthesized similarly to Example 1 Steps 1-3, wherein 2-(1-azidoethyl)benzoic acid was replaced by 2-(1-azidoethyl) nicotinic acid (available from Beijing Okeanos Tech. Co., Ltd., cat. No.: OK-H-20002), yield for the last step was 45%, MW=656.

¹H NMR (400 MHz, DMSO-d₆) δ 11.31 (s, 1H), 8.84 (dd, J=4.6, 1.1 Hz, 1H), 8.34 (d, J=7.9 Hz, 1H), 7.98 (s, 1H), 7.56 (dd, J=7.9, 4.8 Hz, 1H), 6.39-6.32 (m, 1H), 5.63 (d, J=5.2 Hz, 1H), 5.33-5.26 (m, 1H), 4.35 (s, 1H), 4.15-4.04 (m, 2H), 2.55-2.52 (m, 1H), 2.46-2.38 (m, 1H), 1.84 (s, 3H), 1.58 (dd, J=6.6, 1.3 Hz, 3H).

### Example 13

(1) Step 1

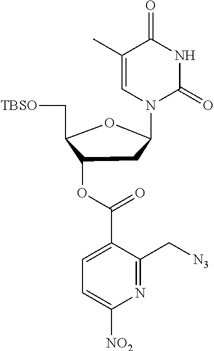 [SMILES: Cc1cn([C@H]2C[C@@H](OC(=O)c3ccc([N+](=O)[O-])nc3CN=[N+]=[N-])[C@@H](CO[Si](C)(C)C(C)(C)C)O2)c(=O)[nH]c1=O]

The product was synthesized similarly to Example 1 step 1, wherein 2-(1-azidoethyl)benzoic acid was replaced by 2-(azidomethyl)-4-nitrobenzoic acid (available from Beijing Okeanos Tech. Co., Ltd., cat. No.: OK-H-21004), the yield of this step was 85%, MW=560.

¹H NMR (500 MHz, CDCl₃) δ 8.43 (d, J=2.3 Hz, 1H), 8.34-8.23 (m, 2H), 8.20 (d, J=8.6 Hz, 1H), 7.57 (d, J=1.2 Hz, 1H), 6.45 (dd, J=9.3, 5.2 Hz, 1H), 5.54 (d, J=5.9 Hz, 1H), 5.04-4.84 (m, 2H), 4.28 (q, J=1.8 Hz, 1H), 4.12-3.92 (m, 2H), 3.48 (s, 1H), 2.60 (dd, J=14.0, 5.3 Hz, 1H), 2.28 (ddd, J=14.0, 9.3, 6.1 Hz, 2H), 1.95 (d, J=1.2 Hz, 3H), 0.97 (s, 9H), 0.18 (d, J=1.1 Hz, 6H).

(2) Steps 2-3

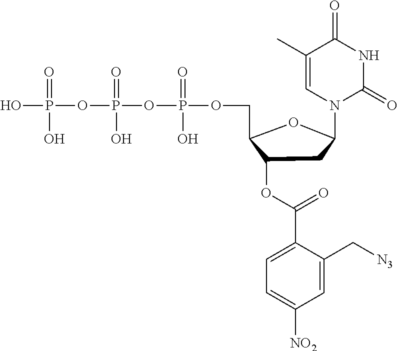 [SMILES: Cc1cn([C@H]2C[C@@H](OC(=O)c3ccc([N+](=O)[O-])cc3CN=[N+]=[N-])[C@@H](COP(=O)(O)OP(=O)(O)OP(=O)(O)O)O2)c(=O)[nH]c1=O]

The product was synthesized similarly to Example 1 Steps 2-3, wherein 2-(1-azidoethyl)benzoic acid was replaced by 2-(azidomethyl)-4-nitrobenzoic acid (available from Beijing Okeanos Tech. Co., Ltd., cat. No.: OK-H-21004), yield for the last step was 40%, MW=686.

¹H NMR (400 MHz, DMSO-d₆) δ 11.30 (s, 1H), 8.41 (d, J=2.0 Hz, 1H), 8.33 (dd, J=8.6, 2.2 Hz, 1H), 8.23 (d, J=8.6 Hz, 1H), 7.95 (s, 1H), 6.36 (dd, J=9.0, 6.0 Hz, 1H), 5.65 (d, J=5.2 Hz, 1H), 4.97 (s, 2H), 4.37 (s, 1H), 4.15-4.04 (m, 2H), 2.56-2.52 (m, 1H), 2.44 (dd, J=14.0, 5.9 Hz, 1H), 1.84 (s, 3H).

### Example 14

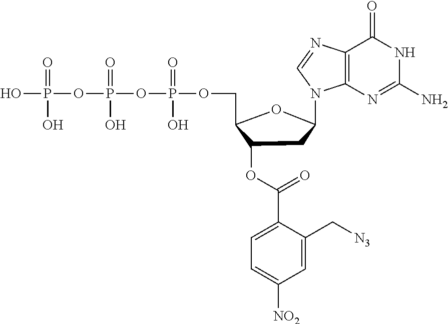 [SMILES: [N-]=[N+]=NCc1cc([N+](=O)[O-])ccc1C(=O)O[C@@H]1C[C@H](n2cnc3c(=O)[nH]c(N)nc32)O[C@@H]1COP(=O)(O)OP(=O)(O)OP(=O)(O)O]

The product was synthesized similarly to Example 2 Steps 1-3, wherein 2-(1-azidoethyl)benzoic acid was replaced by 2-(azidomethyl)-4-nitrobenzoic acid (available from Beijing Okeanos Tech. Co., Ltd., cat. No.: OK-H-21004), yield for the last step was 40%, MW=711.

¹H NMR (400 MHz, DMSO-d₆) δ 11.00 (s, 1H), 8.42 (d, J=2.3 Hz, 1H), 8.34 (dd, J=8.6, 2.3 Hz, 1H), 8.25 (d, J=8.6 Hz, 1H), 8.03 (s, 1H), 6.82 (s, 2H), 6.23 (dd, J=9.2, 5.9 Hz, 1H), 5.76 (d, J=4.2 Hz, 1H), 4.99 (s, 2H), 4.39 (t, J=4.8 Hz, 1H), 4.25-4.00 (m, 2H), 3.31-3.14 (m, 1H), 2.59 (dd, J=14.0, 6.4 Hz, 1H).

### Example 15

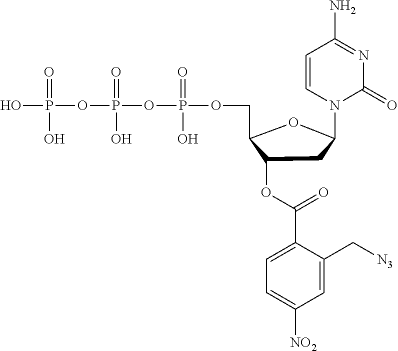 [SMILES: [N-]=[N+]=NCc1cc([N+](=O)[O-])ccc1C(=O)O[C@@H]1C[C@H](n2ccc(N)nc2=O)O[C@@H]1COP(=O)(O)OP(=O)(O)OP(=O)(O)O]

The product was synthesized similarly to Example 3 Steps 1-4, wherein 2-(1-azidoethyl)benzoic acid was replaced by 2-(azidomethyl)-4-nitrobenzoic acid (available from Beijing Okeanos Tech. Co., Ltd., cat. No.: OK-H-21004), yield for the last two steps was 30%, MW=671.

¹H NMR (400 MHz, DMSO-d₆) δ 8.41 (d, J=2.1 Hz, 1H), 8.33 (dd, J=8.5, 2.3 Hz, 1H), 8.23 (d, J=8.6 Hz, 1H), 7.95 (d, J=7.5 Hz, 1H), 7.28 (s, 1H), 7.07 (s, 1H), 6.37 (dd, J=9.1, 5.6 Hz, 1H), 5.81 (d, J=7.5 Hz, 1H), 5.56 (d, J=5.4 Hz, 1H), 4.98 (s, 2H), 4.37 (s, 1H), 4.12-3.99 (m, 2H), 2.47-2.40 (m, 1H), 2.36-2.26 (m, 1H).

### Example 16

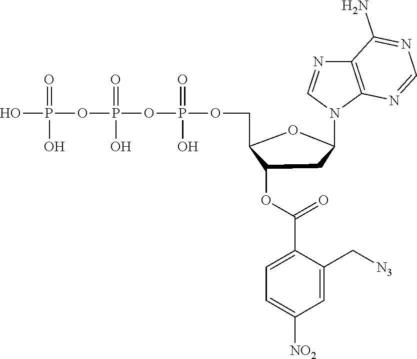 [SMILES: [N-]=[N+]=NCc1cc([N+](=O)[O-])ccc1C(=O)O[C@@H]1C[C@H](n2cnc3c(N)ncnc32)O[C@@H]1COP(=O)(O)OP(=O)(O)OP(=O)(O)O]

The product was synthesized similarly to Example 4 Steps 1-3, wherein 2-(1-azidoethyl)benzoic acid was replaced by 2-(azidomethyl)-4-nitrobenzoic acid (available from Beijing Okeanos Tech. Co., Ltd., cat. No.: OK-H-21004), yield for the last step was 40%, MW=695.

¹H NMR (400 MHz, DMSO-d₆) δ 8.58 (s, 1H), 8.42 (d, J=1.9 Hz, 1H), 8.34 (dd, J=8.5, 2.2 Hz, 1H), 8.29 (d, J=8.6 Hz, 1H), 8.15 (s, 1H), 7.29 (s, 2H), 6.51 (dd, J=9.0, 6.1 Hz, 1H), 5.77 (d, J=5.3 Hz, 1H), 5.02 (s, 2H), 4.47 (s, 1H), 4.14-4.00 (m, 2H), 3.17-3.08 (m, 1H), 2.75-2.70 (m, 1H).

### Example 17

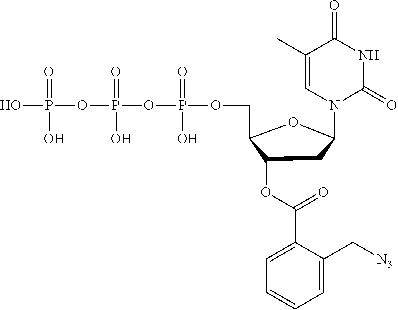 [SMILES: Cc1cn([C@H]2C[C@@H](OC(=O)c3ccccc3CN=[N+]=[N-])[C@@H](COP(=O)(O)OP(=O)(O)OP(=O)(O)O)O2)c(=O)[nH]c1=O]

The product was synthesized similarly to Example 1 Steps 1-3, wherein 2-(1-azidoethyl)benzoic acid was replaced by 2-(azidomethyl)benzoic acid (available from Beijing Okeanos Tech. Co., Ltd., cat. No.: OK-H-20003), yield for the last step was 45%, MW=641.

¹H NMR (400 MHz, DMSO-d₆) δ 11.30 (s, 1H), 7.99 (dd, J=9.6, 8.7 Hz, 2H), 7.68 (td, J=7.6, 1.1 Hz, 1H), 7.61-7.50 (m, 2H), 6.35 (dd, J=9.3, 5.7 Hz, 1H), 5.60 (d, J=5.3 Hz, 1H), 4.80 (s, 2H), 4.31 (s, 1H), 4.18-4.00 (m, 2H), 2.57-2.50 (m, 1H), 2.39 (dd, J=13.9, 5.7 Hz, 1H), 1.84 (s, 3H).

### Example 18

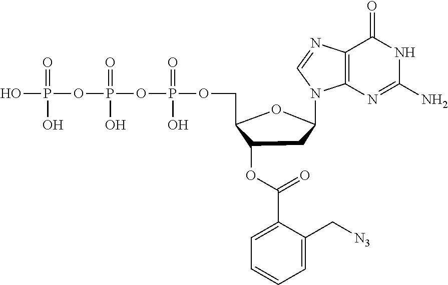 [SMILES: [N-]=[N+]=NCc1ccccc1C(=O)O[C@@H]1C[C@H](n2cnc3c(=O)[nH]c(N)nc32)O[C@@H]1COP(=O)(O)OP(=O)(O)OP(=O)(O)O]

The product was synthesized similarly to Example 2 Steps 1-3, wherein 2-(1-azidoethyl)benzoic acid was replaced by 2-(azidomethyl)benzoic acid (available from Beijing Okeanos Tech. Co., Ltd., cat. No.: OK-H-20003), yield for the last step was 40%, MW=666.

¹H NMR (400 MHz, DMSO-d₆) δ 10.70 (s, 1H), 8.03 (d, J=9.8 Hz, 2H), 7.69 (td, J=7.6, 1.2 Hz, 1H), 7.60-7.51 (m, 2H), 6.67 (s, 2H), 6.23 (dd, J=9.4, 5.8 Hz, 1H), 5.68 (d, J=4.9 Hz, 1H), 4.82 (s, 2H), 4.40-4.30 (m, 1H), 4.21-4.16 (m, 1H), 4.05-3.96 (m, 1H), 3.22-3.10 (m, 1H), 2.60-2.51 (m, 1H).

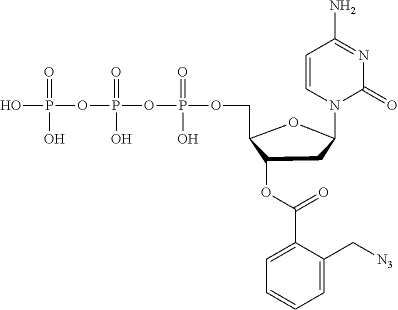 [SMILES: [N-]=[N+]=NCc1ccccc1C(=O)O[C@@H]1C[C@H](n2ccc(N)nc2=O)O[C@@H]1COP(=O)(O)OP(=O)(O)OP(=O)(O)O]

### Example 19

The product was synthesized similarly to Example 3 Steps 1-4, wherein 2-(1-azidoethyl)benzoic acid was replaced by 2-(azidomethyl)benzoic acid (available from Beijing Okeanos Tech. Co., Ltd., cat. No.: OK-H-20003), yield for the last two steps was 30%, MW=626.

¹H NMR (400 MHz, DMSO-d₆) δ 8.00 (dd, J=7.8, 1.1 Hz, 1H), 7.95 (d, J=7.5 Hz, 1H), 7.68 (td, J=7.6, 1.4 Hz, 1H), 7.61-7.50 (m, 2H), 7.29 (s, 1H), 7.07 (s, 1H), 6.37 (dd, J=9.2, 5.5 Hz, 1H), 5.82 (d, J=7.5 Hz, 1H), 5.51 (d, J=5.4 Hz, 1H), 4.81 (s, 2H), 4.32 (s, 1H), 4.13-3.97 (m, 2H), 2.41 (dd, J=13.8, 5.6 Hz, 1H), 2.30 (ddd, J=14.4, 7.5, 4.6 Hz, 1H).

### Example 20

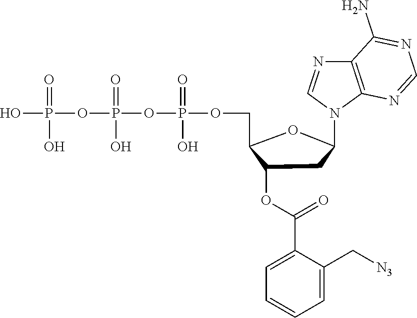 [SMILES: [N-]=[N+]=NCc1ccccc1C(=O)O[C@@H]1C[C@H](n2cnc3c(N)ncnc32)O[C@@H]1COP(=O)(O)OP(=O)(O)OP(=O)(O)O]

The product was synthesized similarly to Example 4 Steps 1-3, wherein 2-(1-azidoethyl)benzoic acid was replaced by 2-(azidomethyl)benzoic acid (available from Beijing Okeanos Tech. Co., Ltd., cat. No.: OK-H-20003), yield for the last step was 40%, MW=650.

¹H NMR (400 MHz, DMSO-d₆) δ 8.60 (s, 1H), 8.15 (s, 1H), 8.05 (d, J=6.5 Hz, 1H), 7.69 (t, J=8.2 Hz, 1H), 7.60-7.54 (m, 2H), 7.27 (s, 2H), 6.49 (dd, J=8.8, 5.8 Hz, 1H), 5.71 (d, J=5.6 Hz, 1H), 4.83 (s, 2H), 4.42-4.34 (m, 1H), 4.02-3.80 (m, 2H), 2.72-2.63 (m, 1H), 2.35-2.30 (m, 1H).

### Example 21

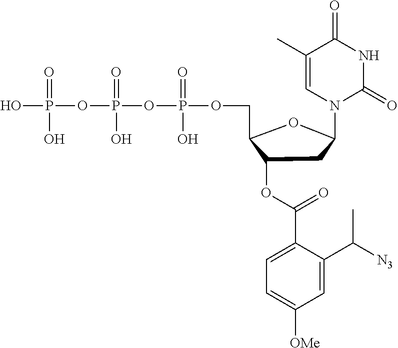 [SMILES: COc1ccc(C(=O)O[C@@H]2C[C@H](n3cc(C)c(=O)[nH]c3=O)O[C@@H]2COP(=O)(O)OP(=O)(O)OP(=O)(O)O)c(C(C)N=[N+]=[N-])c1]

The product was synthesized similarly to Example 1 Steps 1-3, wherein 2-(1-azidoethyl)benzoic acid was replaced by 2-(1-azidoethyl)-4-methoxy benzoic acid (available from Beijing Okeanos Tech. Co., Ltd., cat. No.: OK-H-21005), yield for the last step was 45%, MW=685.

¹H NMR (400 MHz, DMSO-d₆) δ 11.30 (s, 1H), 8.00-7.90 (m, 2H), 7.11 (d, J=2.6 Hz, 1H), 7.04 (dd, J=8.8, 2.6 Hz, 1H), 6.39-6.29 (m, 1H), 5.74-5.68 (m, 1H), 5.55 (d, J=5.1 Hz, 1H), 4.30 (s, 1H), 4.16-3.98 (m, 2H), 3.86 (s, 3H), 2.48-2.30 (m, 2H), 1.84 (s, 3H), 1.48 (dd, J=6.7, 1.4 Hz, 3H).

### Example 22

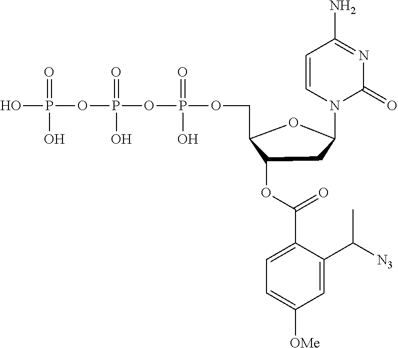 [SMILES: COc1ccc(C(=O)O[C@@H]2C[C@H](n3ccc(N)nc3=O)O[C@@H]2COP(=O)(O)OP(=O)(O)OP(=O)(O)O)c(C(C)N=[N+]=[N-])c1]

The product was synthesized similarly to Example 3 Steps 1-3, wherein 2-(1-azidoethyl)benzoic acid was replaced by 2-(1-azidoethyl)-4-methoxy benzoic acid (available from Beijing Okeanos Tech. Co., Ltd., cat. No.: OK-H-21005), yield for the last two steps was 30%, MW=670.

¹H NMR (400 MHz, DMSO-d₆) δ 8.00-7.89 (m, 2H), 7.32 (s, 1H), 7.11 (d, J=2.5 Hz, 2H), 7.05 (dd, J=8.8, 2.6 Hz, 1H), 6.41-6.31 (m, 1H), 5.82 (d, J=7.5 Hz, 1H), 5.74-5.68 (m, 1H), 5.49 (d, J=4.5 Hz, 1H), 4.30 (s, 1H), 4.10-4.00 (m, 2H), 3.86 (s, 3H), 2.42-2.36 (m, 1H), 2.33-2.23 (m, 1H), 1.48 (dd, J=6.6, 1.5 Hz, 3H).

### Example 23

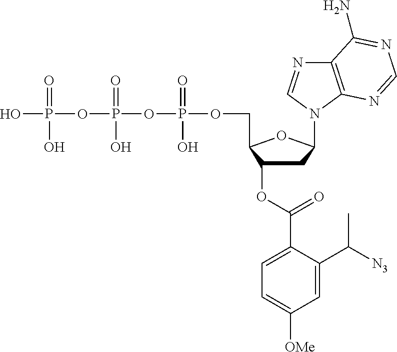

The product was synthesized similarly to Example 4 Steps 1-3, wherein 2-(1-azidoethyl)benzoic acid was replaced by 2-(1-azidoethyl)-4-methoxy benzoic acid (available from Beijing Okeanos Tech. Co., Ltd., cat. No.: OK-H-21005), the yield for the last step was 40%, MW=694.

¹H NMR (400 MHz, DMSO-d₆) δ 8.61 (d, J=3.4 Hz, 1H), 8.16 (d, J=1.4 Hz, 1H), 8.02 (dd, J=8.7, 1.7 Hz, 1H), 7.30 (s, 2H), 7.12 (d, J=2.5 Hz, 1H), 7.07 (dd, J=8.8, 2.6 Hz, 1H), 6.51-6.46 (m, 1H), 5.78-5.72 (m, 1H), 5.68 (d, J=5.2 Hz, 1H), 4.43-4.36 (m, 1H), 4.14-4.00 (m, 2H), 3.87 (s, 3H), 3.18-3.08 (m, 1H), 2.71-2.62 (m, 1H), 1.50 (dd, J=6.6, 2.0 Hz, 3H).

### Example 24

(1) Steps 1-2

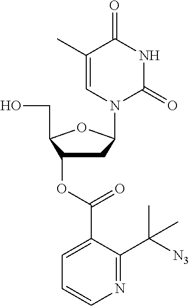

The product was synthesized similarly to Example 1 steps 1-2, wherein 2-(1-azidoethyl)benzoic acid was replaced by 2-(2-azidopropan-2-yl) nicotinic acid (available from Beijing Okeanos Tech. Co., Ltd., cat. No.: OK-H-20004), yield for the last step was 90%, MW=430.

¹H NMR (400 MHz, CDCl₃) δ 9.12 (s, 1H), 8.31 (dd, J=4.8, 1.7 Hz, 1H), 7.39 (dd, J=7.7, 1.6 Hz, 1H), 6.96 (dd, J=7.3, 5.2 Hz, 1H), 5.97 (t, J=7.2 Hz, 1H), 5.33 (dd, J=5.1, 2.7 Hz, 1H), 4.02 (d, J=2.3 Hz, 1H), 3.70 (d, J=2.4 Hz, 2H), 2.72 (s, 1H), 2.25 (dd, J=10.7, 5.1 Hz, 2H), 1.60 (s, 3H), 1.45 (s, 6H).

(2) Step 3

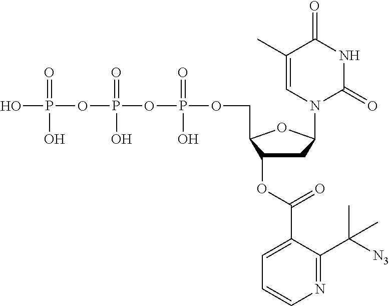

The product was synthesized similarly to Example 1 step 3, wherein 2-(1-azidoethyl)benzoic acid was replaced by 2-(2-azidopropan-2-yl) nicotinic acid (available from Beijing Okeanos Tech. Co., Ltd., cat. No.: OK-H-20004), the yield of this step was 35%, MW=670.

¹H NMR (400 MHz, DMSO-d₆) δ 11.30 (s, 1H), 8.67 (dd, J=4.7, 1.4 Hz, 1H), 8.02 (dd, J=7.7, 1.4 Hz, 1H), 7.96 (s, 1H), 7.47 (dd, J=7.7, 4.8 Hz, 1H), 6.29 (dd, J=9.2, 5.7 Hz, 1H), 5.61 (d, J=5.3 Hz, 1H), 4.31 (s, 1H), 4.19-4.03 (m, 2H), 2.56-2.52 (m, 1H), 2.36 (dd, J=13.9, 5.6 Hz, 1H), 1.84 (s, 3H), 1.70 (d, J=3.8 Hz, 6H).

### Example 25

(1) Steps 1-2

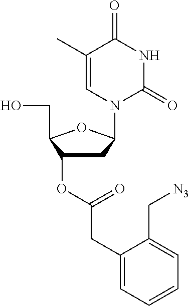

The product was synthesized similarly to Example 1 steps 1-2, wherein 2-(1-azidoethyl)benzoic acid was replaced by 2-(2-(azidomethyl)phenyl)acetic acid (available from Beijing Okeanos Tech. Co., Ltd., cat. No.: OK-H-20005), yield for the last step was 90%, MW=415.

¹H NMR (500 MHz, CDCl₃) δ 8.08 (s, 1H), 7.46 (d, J=1.1 Hz, 1H), 7.39-7.32 (m, 3H), 7.30 (d, J=6.8 Hz, 1H), 6.20 (dd, J=8.5, 5.9 Hz, 1H), 5.37-5.35 (m, 1H), 4.40 (s, 2H), 4.08 (q, J=2.4 Hz, 1H), 3.90 (qd, J=11.8, 2.6 Hz, 2H), 3.76 (s, 2H), 2.48-2.33 (m, 2H), 1.92 (d, J=1.0 Hz, 3H).

(2) Step 3

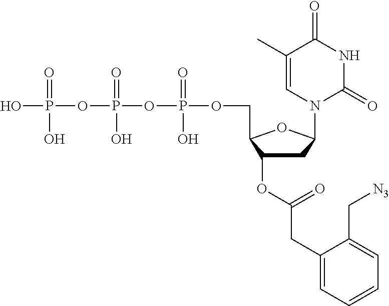

The product was synthesized similarly to Example 1 step 3, wherein 2-(1-azidoethyl)benzoic acid was replaced by 2-(2-(azidomethyl)phenyl)acetic acid (available from Beijing Okeanos Tech. Co., Ltd., cat. No.: OK-H-20005), the yield of this step was 40%, MW=655.

¹H NMR (400 MHz, DMSO-d₆) δ 11.33 (s, 1H), 7.91 (s, 1H), 7.43-7.28 (m, 4H), 6.26 (dd, J=9.4, 5.6 Hz, 1H), 5.34 (d, J=5.4 Hz, 1H), 4.50 (s, 2H), 4.09 (s, 1H), 4.05-3.95 (m, 2H), 3.84 (s, 2H), 2.44-2.31 (m, 1H), 2.18 (dd, J=13.9, 5.5 Hz, 1H), 1.82 (s, 3H).

### Example 26

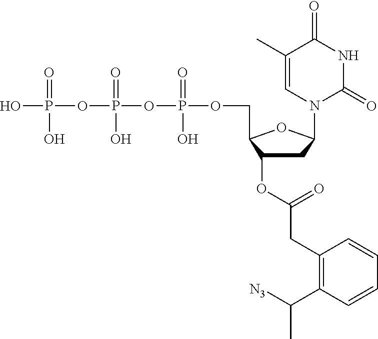

The product was synthesized similarly to Example 1 Steps 1-3, wherein 2-(1-azidoethyl)benzoic acid was replaced by 2-(2-(1-azidoethyl)phenyl)acetic acid (available from Beijing Okeanos Tech. Co., Ltd., cat. No.: OK-H-20006), yield for the last step was 40%, MW=669.

¹H NMR (400 MHz, DMSO-d₆) δ 11.29 (s, 1H), 7.87 (s, 1H), 7.44 (d, J=7.6 Hz, 1H), 7.35 (dt, J=7.9, 4.2 Hz, 1H), 7.30 (d, J=4.0 Hz, 2H), 6.26 (dd, J=9.5, 5.6 Hz, 1H), 5.33 (d, J=5.4 Hz, 1H), 5.05 (q, J=6.7 Hz, 1H), 4.10 (s, 1H), 4.02-3.94 (m, 2H), 3.90 (d, J=2 Hz, 2H), 2.42-2.33 (m, 1H), 2.21-2.12 (m, 1H), 1.81 (s, 3H), 1.45 (dd, J=6.7, 1.3 Hz, 3H).

### Example 27

(1) Steps 1-2

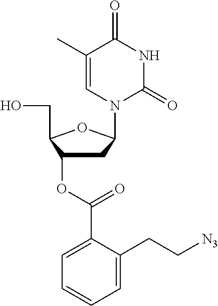

The product was synthesized similarly to Example 1 steps 1-2, wherein 2-(1-azidoethyl)benzoic acid was replaced by 2-(2-azidoethyl)benzoic acid (available from Beijing Okeanos Tech. Co., Ltd., cat. No.: OK-H-21006), yield for the last step was 90%, MW=415.

¹H NMR (500 MHz, CDCl₃) δ 8.40 (s, 1H), 7.98 (d, J=7.8 Hz, 1H), 7.57-7.49 (m, 2H), 7.39-7.30 (m, 2H), 6.36-6.29 (m, 1H), 5.64-5.56 (m, 1H), 4.26 (d, J=2.4 Hz, 1H), 4.02 (d, J=2.5 Hz, 2H), 3.54 (t, J=7.0 Hz, 2H), 3.27 (t, J=7.1 Hz, 2H), 2.60-2.50 (m, 2H), 1.95 (s, 3H).

(2) Step 3

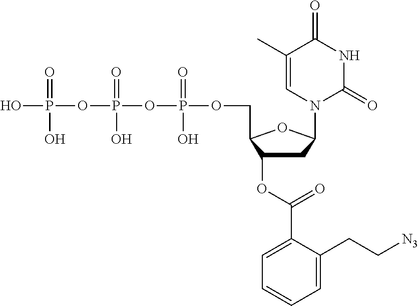

The product was synthesized similarly to Example 1 step 3, wherein 2-(1-azidoethyl)benzoic acid was replaced by 2-(2-azidoethyl)benzoic acid (available from Beijing Okeanos Tech. Co., Ltd., cat. No.: OK-H-21006), the yield of this step was 40%, MW=655.

¹H NMR (400 MHz, DMSO-d₆) δ 11.28 (s, 1H), 7.98-7.85 (m, 2H), 7.57 (t, J=7.4 Hz, 1H), 7.42 (t, J=7.7 Hz, 2H), 6.34 (dd, J=9.3, 5.7 Hz, 1H), 5.57 (d, J=5.3 Hz, 1H), 4.30 (s, 1H), 4.08-4.02 (m, 2H), 3.57 (t, J=6.9 Hz, 2H), 3.21 (t, J=6.9 Hz, 2H), 2.55-2.52 (m, 1H), 2.43-2.27 (m, 1H), 1.84 (s, 3H).

### Example 28

(1) Steps 1-2

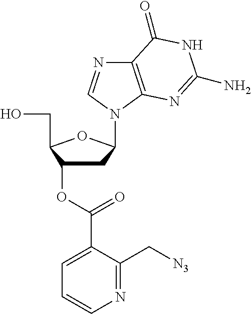

The product was synthesized similarly to Example 2 steps 1-2, wherein 2-(1-azidoethyl)benzoic acid was replaced by 2-(azidomethyl) nicotinic acid (available from Beijing Okeanos Tech. Co., Ltd., cat. No.: OK-H-21007), yield for the last step was 90%, MW=427.

¹H NMR (400 MHz, CDCl₃) δ 8.52 (s, 2H), δ=8.20 (d, 1H), δ=7.90 (d, 1H), δ=7.58 (s, 1H), δ=7.35 (dd, 1H), δ=6.26 (t, 1H), δ=5.59 (dq, 1H), δ=5.11 (s, 2H), δ=4.61 (dt, 1H), δ=3.90 (m, 2H), δ=2.38 (m, 2H).

(2) Step 3

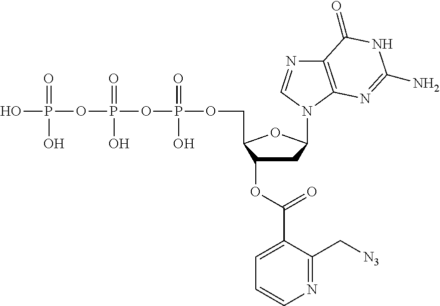

The product was synthesized similarly to Example 2 step 3, wherein 2-(1-azidoethyl)benzoic acid was replaced by 2-(azidomethyl) nicotinic acid (available from Beijing Okeanos Tech. Co., Ltd., cat. No.: OK-H-21007), the yield of this step was 30%, MW=667.

¹H NMR (400 MHz, DMSO-d₆) δ 10.76 (s, 1H), 8.83 (dd, J=4.8, 1.5 Hz, 1H), 8.40 (dd, J=7.9, 1.7 Hz, 1H), 8.00 (d, J=6.6 Hz, 1H), 7.59 (dd, J=7.9, 4.8 Hz, 1H), 6.73 (s, 2H), 6.23 (dd, J=9.3, 5.8 Hz, 1H), 5.72 (d, J=4.8 Hz, 1H), 4.85 (s, 2H), 4.36 (d, J=4.9 Hz, 1H), 4.27-4.16 (m, 1H), 4.01-3.93 (m, 1H), 3.26-3.18 (d, J=14.3 Hz, 1H), 2.56 (dd, J=13.7, 5.3 Hz, 1H).

### Example 29

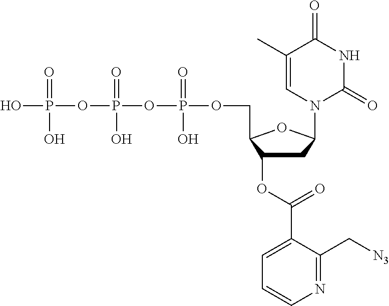

The product was synthesized similarly to Example 1 Steps 1-3, wherein 2-(1-azidoethyl)benzoic acid was replaced by 2-(azidomethyl) nicotinic acid (available from Beijing Okeanos Tech. Co., Ltd., cat. No.: OK-H-21007), yield for the last step was 40%, MW=642.

¹H NMR (400 MHz, DMSO-d₆) δ 11.29 (s, 1H), 8.81 (dd, J=4.8, 1.7 Hz, 1H), 8.39 (dd, J=7.9, 1.7 Hz, 1H), 7.89 (d, J=4.2 Hz, 1H), 7.58 (dd, J=7.9, 4.8 Hz, 1H), 6.35 (dd, J=9.0, 6.0 Hz, 1H), 5.59 (d, J=4.8 Hz, 1H), 4.83 (s, 2H), 4.35 (s, 1H), 4.14-3.97 (m, 2H), 2.46-2.36 (m, 2H), 1.83 (d, J=2.7 Hz, 3H).

### Example 30

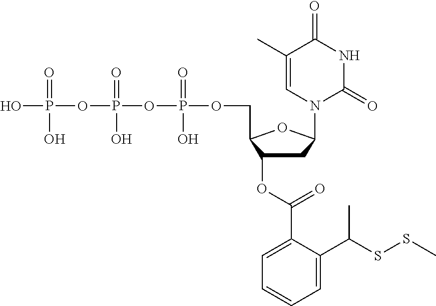

The product was synthesized similarly to Example 5 Steps 1-3, wherein 2-(1-(ethyldisulfaneyl)ethyl)benzoic acid was replaced by 2-(1-(methyldisulfaneyl)ethyl)benzoic acid (available from Beijing Okeanos Tech. Co., Ltd., cat. No.: OK-H-20007), yield for the last step was 30%, MW=692.

¹H NMR (400 MHz, D₂O) δ 7.90-7.82 (m, 2H), 7.69-7.58 (m, 2H), 7.46-7.39 (m, 1H), 6.47-6.35 (m, 1H), 5.73 (s, 1H), 5.10-5.01 (m, 1H), 4.57 (dd, J=13.6, 3.0 Hz, 1H), 4.41-4.24 (m, 2H), 2.65-2.52 (m, 2H), 1.98 (d, J=15.2 Hz, 3H), 1.94 (s, 3H), 1.68 (d, J=7.2 Hz, 3H).

### Example 31

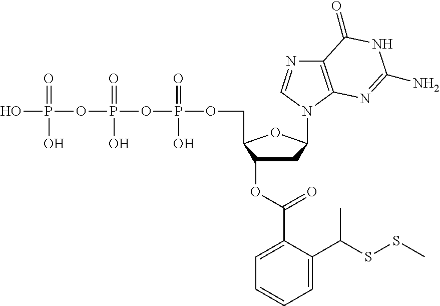

The product was synthesized similarly to Example 6 Steps 1-3, wherein 2-(1-(ethyldisulfaneyl)ethyl)benzoic acid was replaced by 2-(1-(methyldisulfaneyl)ethyl)benzoic acid (available from Beijing Okeanos Tech. Co., Ltd., cat. No.: OK-H-20007), yield for the last step was 30%, MW=717.

¹H NMR (400 MHz, D₂O) δ 8.14 (t, J=3.1 Hz, 1H), 7.82 (dd, J=8.1, 3.1 Hz, 1H), 7.55 (q, J=3.2 Hz, 2H), 7.38-7.34 (m, 1H), 6.31-6.22 (m, 1H), 5.76 (d, J=4.1 Hz, 1H), 5.10-5.00 (m, 1H), 4.65-4.55 (m, 1H), 4.36-4.21 (m, 2H), 3.02-2.94 (m, 1H), 2.71-2.61 (m, 1H), 1.94 (dd, J=22.2, 3.2 Hz, 3H), 1.62 (dd, J=7.2, 3.3 Hz, 3H).

### Example 32

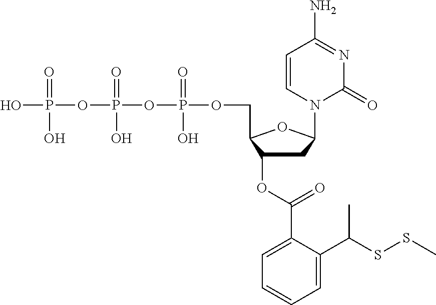

The product was synthesized similarly to Example 7 Steps 1-4, wherein 2-(1-(ethyldisulfaneyl)ethyl)benzoic acid was replaced by 2-(1-(methyldisulfaneyl)ethyl)benzoic acid (available from Beijing Okeanos Tech. Co., Ltd., cat. No.: OK-H-20007), yield for the last two steps was 28%, MW=677.

¹H NMR (400 MHz, D₂O) δ 8.08-8.00 (m, 1H), 7.91-7.84 (m, 1H), 7.72-7.59 (m, 2H), 7.49-7.40 (m, 1H), 6.48-6.40 (m, 1H), 6.19 (dd, J=7.7, 2.3 Hz, 1H), 5.73-5.64 (m, 1H), 5.13-5.02 (m, 1H), 4.60 (d, J=12.6 Hz, 1H), 4.36-4.27 (m, 2H), 2.71-2.64 (m, 1H), 2.55-2.44 (m, 1H), 2.00 (dd, J=9.5, 2.2 Hz, 3H), 1.69 (dd, J=7.2, 2.1 Hz, 3H).

### Example 33

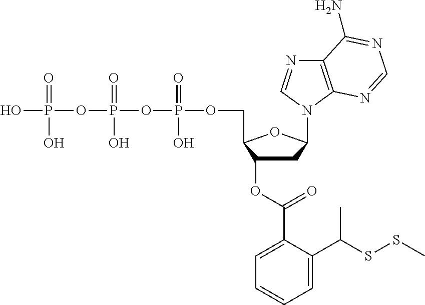

The product was synthesized similarly to Example 8 Steps 1-3, wherein 2-(1-(ethyldisulfaneyl)ethyl)benzoic acid was replaced by 2-(1-(methyldisulfaneyl)ethyl)benzoic acid (available from Beijing Okeanos Tech. Co., Ltd., cat. No.: OK-H-20007), yield for the last step was 35%, MW=701.

¹H NMR (400 MHz, D₂O) δ 8.53 (dd, J=5.2, 1.8 Hz, 1H), 8.18-8.10 (m, 1H), 7.83 (d, J=7.8 Hz, 1H), 7.55-7.51 (m, 2H), 7.38-7.34 (m, 1H), 6.49-6.40 (m, 1H), 5.81-5.73 (m, 1H), 5.07-5.00 (m, 1H), 4.67-4.57 (m, 1H), 4.39-4.18 (m, 2H), 3.06-2.95 (m, 1H), 2.75 (ddd, J=21.2, 14.2, 5.6 Hz, 1H), 1.93 (dd, J=25.1, 1.9 Hz, 3H), 1.60 (dd, J=6.9, 4.0 Hz, 3H).

### Example 34

(1) Step 1—

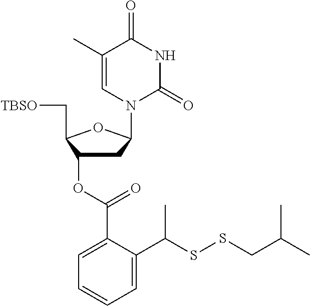

The product was synthesized similarly to Example 5 step 1, wherein 2-(1-(ethyldisulfaneyl)ethyl)benzoic acid was replaced by 2-(1-(isobutyldisulfaneyl)ethyl)benzoic acid (available from Beijing Okeanos Tech. Co., Ltd., cat. No.: OK-H-21008), the yield of this step was 79%, MW=608.

¹H NMR (400 MHz, CDCl₃) δ 8.66-8.39 (m, 1H), 7.89-7.80 (m, 1H), 7.63-7.47 (m, 3H), 7.33 (td, J=8.8, 4.5 Hz, 1H), 6.47-6.40 (m, 1H), 5.52-5.40 (m, 1H), 5.30-4.92 (m, 1H), 4.36-4.21 (m, 1H), 4.07-3.92 (m, 2H), 2.60-2.48 (m, 1H), 2.31-2.16 (m, 1H), 1.94 (s, 3H), 1.72-1.66 (m, 2H), 1.63-1.51 (m, 1H), 1.00 (d, J=6.8 Hz, 2H), 0.98-0.93 (m, 9H), 0.87-0.81 (m, 4H), 0.19-0.13 (m, 6H).

(2) Steps 2-3

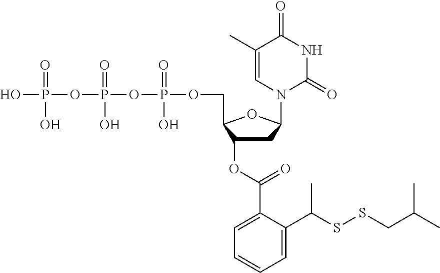

The product was synthesized similarly to Example 5 Steps 2-3, wherein 2-(1-(ethyldisulfaneyl)ethyl)benzoic acid was replaced by 2-(1-(isobutyldisulfaneyl)ethyl)benzoic acid (available from Beijing Okeanos Tech. Co., Ltd., cat. No.: OK-H-21008), yield for the last step was 32%, MW=734.

¹H NMR (400 MHz, DMSO-d₆) δ 11.31 (s, 1H), 7.96 (dd, J=8.2, 0.9 Hz, 1H), 7.85 (d, J=8.3 Hz, 1H), 7.62 (d, J=3.9 Hz, 2H), 7.46-7.41 (m, 1H), 6.33 (td, J=9.7, 5.6 Hz, 1H), 5.63-5.55 (m, 1H), 5.18-5.08 (m, 1H), 4.30 (d, J=23.4 Hz, 1H), 4.18-4.02 (m, 2H), 2.57-2.51 (m, 1H), 2.43-2.29 (m, 1H), 2.12-1.93 (m, 2H), 1.84 (s, 3H), 1.70-1.56 (m, 4H), 0.85-0.71 (m, 6H).

### Example 35

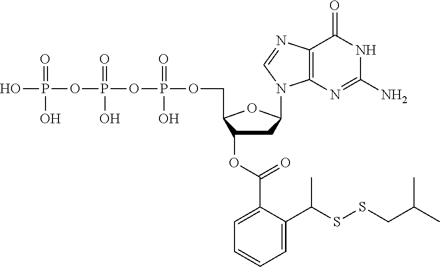

The product was synthesized similarly to Example 6 Steps 1-3, wherein 2-(1-(ethyldisulfaneyl)ethyl)benzoic acid was replaced by 2-(1-(isobutyldisulfaneyl)ethyl)benzoic acid (available from Beijing Okeanos Tech. Co., Ltd., cat. No.: OK-H-21008), the yield for the last step was 40%, MW=759.

¹H NMR (400 MHz, DMSO-d₆) δ 10.73 (s, 1H), 8.03 (d, J=6.9 Hz, 1H), 7.89 (d, J=7.6 Hz, 1H), 7.63 (d, J=2.8 Hz, 2H), 7.49-7.38 (m, 1H), 6.66 (s, 2H), 6.27-6.15 (m, 1H), 5.69 (dd, J=8.2, 4.0 Hz, 1H), 5.16 (q, J=6.9 Hz, 1H), 4.39-4.26 (m, 1H), 4.19 (dd, J=10.6, 5.1 Hz, 1H), 4.01 (dd, J=11.4, 5.9 Hz, 1H), 2.57-2.52 (m, 1H), 2.48-2.41 (m, 1H), 2.24-2.12 (m, 2H), 1.70 (dd, J=13.2, 6.6 Hz, 1H), 1.65 (d, J=6.8 Hz, 3H), 0.85-0.74 (m, 6H).

### Example 36

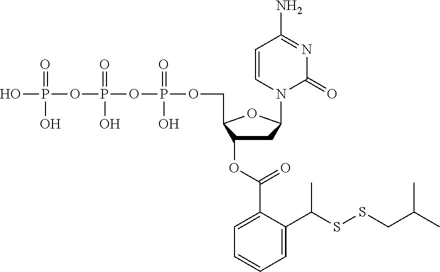

The product was synthesized similarly to Example 7 Steps 1-4, wherein 2-(1-(ethyldisulfaneyl)ethyl)benzoic acid was replaced by 2-(1-(isobutyldisulfaneyl)ethyl)benzoic acid (available from Beijing Okeanos Tech. Co., Ltd., cat. No.: OK-H-21008 (available from Beijing Okeanos Tech. Co., Ltd., cat. No.: OK-H-21008), the yield for the last two steps was 30%, MW=719.

¹H NMR (400 MHz, DMSO-d₆) δ 7.98 (dd, J=9.4, 7.6 Hz, 1H), 7.85 (d, J=7.8 Hz, 1H), 7.62 (d, J=4.1 Hz, 2H), 7.46-7.42 (m, 1H), 7.33 (s, 1H), 7.12 (s, 1H), 6.40-6.30 (m, 1H), 5.82 (dd, J=7.5, 1.0 Hz, 1H), 5.53 (d, J=4.4 Hz, 1H), 5.17-5.11 (m, 1H), 4.30 (d, J=21.7 Hz, 1H), 4.16-4.01 (m, 1H), 2.46-2.27 (m, 2H), 2.15-1.98 (m, 2H), 1.71-1.58 (m, 4H), 0.80 (d, J=2.5 Hz, 3H), 0.78 (d, J=2.6 Hz, 3H).

### Example 37

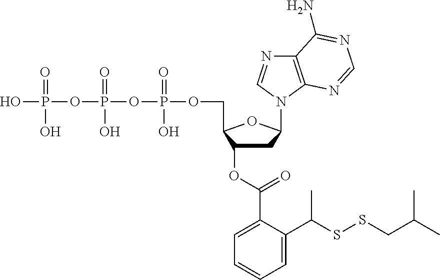

The product was synthesized similarly to Example 8 Steps 1-3, wherein 2-(1-(ethyldisulfaneyl)ethyl)benzoic acid was replaced by 2-(1-(isobutyldisulfaneyl)ethyl)benzoic acid (available from Beijing Okeanos Tech. Co., Ltd., cat. No.: OK-H-21008 (available from Beijing Okeanos Tech. Co., Ltd., cat. No.: OK-H-21008), the yield for the last step was 40%, MW=743.

¹H NMR (400 MHz, DMSO-d₆) δ 8.67 (d, J=8.3 Hz, 1H), 8.14 (d, J=5.9 Hz, 1H), 7.94-7.87 (m, 1H), 7.64 (d, J=3.9 Hz, 2H), 7.46 (dt, J=8.1, 4.2 Hz, 1H), 7.32 (s, 2H), 6.51-6.44 (m, 1H), 5.75 (s, 1H), 5.18 (q, J=7.0 Hz, 1H), 4.45-4.33 (m, 1H), 4.16-4.04 (m, 2H), 3.25-3.12 (m, 1H), 2.71-2.57 (m, 1H), 2.19-2.01 (m, 2H), 1.73-1.59 (m, 4H), 0.81 (d, J=6.7 Hz, 3H), 0.78 (d, J=6.7 Hz, 3H).

### Example 38

(1) Step 1

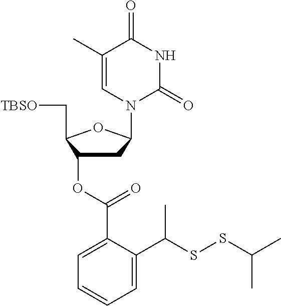

The product was synthesized similarly to Example 5 step 1, wherein 2-(1-(ethyldisulfaneyl)ethyl)benzoic acid was replaced by 2-(1-(isopropyldisulfaneyl)ethyl)benzoic acid (available from Beijing Okeanos Tech. Co., Ltd., cat. No.: OK-H-21009), the yield of this step was 80%, MW=594.

¹H NMR (500 MHz, CDCl₃) δ 8.51 (s, 1H), 7.85 (ddd, J=12.8, 7.8, 1.2 Hz, 1H), 7.64-7.57 (m, 2H), 7.57-7.50 (m, 1H), 7.37-7.29 (m, 1H), 6.45 (ddd, J=20.4, 9.2, 5.3 Hz, 1H), 5.50 (t, J=5.3 Hz, 1H), 5.28-5.15 (m, 1H), 4.32 (dd, J=36.3, 1.3 Hz, 1H), 4.07-3.96 (m, 2H), 2.59 (td, J=13.5, 5.3 Hz, 1H), 2.47-2.16 (m, 2H), 1.95 (s, 3H), 1.70 (dd, J=7.0, 1.0 Hz, 3H), 1.17-1.06 (m, 6H), 0.97 (s, 9H), 0.18 (s, 6H).

(2) Step 2

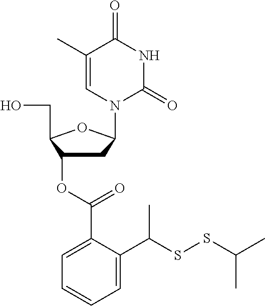

The product was synthesized similarly to Example 5 Step 2, wherein 2-(1-(ethyldisulfaneyl)ethyl)benzoic acid was replaced by 2-(1-(isopropyldisulfaneyl)ethyl)benzoic acid (available from Beijing Okeanos Tech. Co., Ltd., cat. No.: OK-H-21009), the yield of this step was 90%, MW=594.

¹H NMR (500 MHz, CDCl₃) δ 8.84 (s, 1H), 7.84 (ddd, J=7.4, 5.8, 1.3 Hz, 1H), 7.65-7.49 (m, 3H), 7.33 (td, J=7.7, 1.2 Hz, 1H), 6.37-6.27 (m, 1H), 5.61 (tt, J=4.7, 2.5 Hz, 1H), 5.22 (dq, J=13.9, 7.0 Hz, 1H), 4.30 (dq, J=19.8, 2.4 Hz, 1H), 4.03 (dd, J=5.7, 2.7 Hz, 2H), 2.60-2.31 (m, 3H), 1.95 (d, J=1.1 Hz, 3H), 1.70 (d, J=7.0 Hz, 3H), 1.15-1.07 (m, 6H).

(3) Step 3

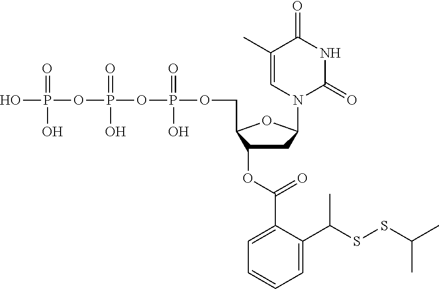

The product was synthesized similarly to Example 5 step 3, wherein 2-(1-(ethyldisulfaneyl)ethyl)benzoic acid was replaced by 2-(1-(isopropyldisulfaneyl)ethyl)benzoic acid (available from Beijing Okeanos Tech. Co., Ltd., cat. No.: OK-H-21009), the yield of this step was 40%, MW=720.

¹H NMR (400 MHz, DMSO-d₆) δ 11.29 (s, 1H), 7.98 (dd, J=6.6, 0.9 Hz, 1H), 7.85 (dd, J=7.7, 2.4 Hz, 1H), 7.61 (t, J=6.4 Hz, 2H), 7.45-7.40 (m, 1H), 6.36-6.31 (m, 1H), 5.60 (d, J=5.3 Hz, 1H), 5.11 (q, J=6.9 Hz, 1H), 4.30 (d, J=20.0 Hz, 1H), 4.12-4.04 (m, 2H), 2.58-2.51 (m, 1H), 2.46-2.29 (m, 2H), 1.84 (s, 3H), 1.64 (d, J=7.0 Hz, 3H), 1.06 (d, J=5.7 Hz, 6H).

### Example 39

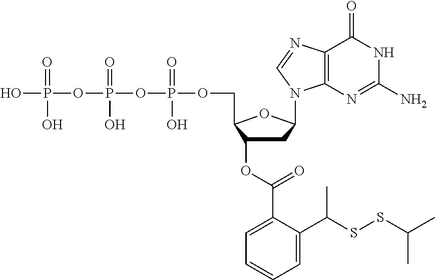

The product was synthesized similarly to Example 6 Steps 1-3, wherein 2-(1-(ethyldisulfaneyl)ethyl)benzoic acid was replaced by 2-(1-(isopropyldisulfaneyl)ethyl)benzoic acid (available from Beijing Okeanos Tech. Co., Ltd., cat. No.: OK-H-21009), yield for the last step was 40%, MW=745.

¹H NMR (400 MHz, DMSO-d₆) δ 10.57 (s, 1H), 7.98 (d, J=7.9 Hz, 1H), 7.88 (d, J=7.5 Hz, 1H), 7.63 (d, J=3.3 Hz, 2H), 7.47-7.40 (m, 1H), 6.63 (d, J=25.9 Hz, 2H), 6.27-6.16 (m, 1H), 5.68 (t, J=5.3 Hz, 1H), 5.32 (t, J=4.7 Hz, 1H), 5.18-5.10 (m, 1H), 4.37-4.28 (m, 1H), 4.02-3.96 (m, 1H), 2.68-2.66 (m, 1H), 2.61-2.56 (m, 1H), 2.33 (dt, J=3.5, 1.7 Hz, 1H), 1.65 (d, J=6.9 Hz, 3H), 1.13 (d, J=7.9 Hz, 6H).

### Example 40

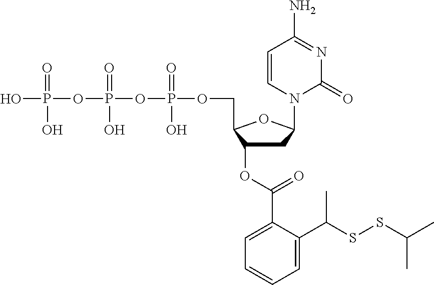

The product was synthesized similarly to Example 7 Steps 1-4, wherein 2-(1-(ethyldisulfaneyl)ethyl)benzoic acid was replaced by 2-(1-(isopropyldisulfaneyl)ethyl)benzoic acid (available from Beijing Okeanos Tech. Co., Ltd., cat. No.: OK-H-21009), yield for the last two steps was 30%, MW=705.

¹H NMR (400 MHz, DMSO-d₆) δ 8.00-7.93 (m, 1H), 7.84 (dd, J=7.6, 3.3 Hz, 1H), 7.62 (d, J=3.7 Hz, 2H), 7.47-7.38 (m, 1H), 7.28 (s, 1H), 7.07 (s, 1H), 6.38-6.33 (m, 1H), 5.81 (d, J=7.3 Hz, 1H), 5.52 (d, J=5.2 Hz, 1H), 5.11 (q, J=7.0 Hz, 1H), 4.30 (d, J=19.7 Hz, 1H), 4.14-3.97 (m, 2H), 2.47-2.38 (m, 2H), 2.34-2.31 (m, 1H), 1.64 (d, J=7.0 Hz, 3H), 1.10 (d, J=2.1 Hz, 6H).

### Example 41

(1) Step 1

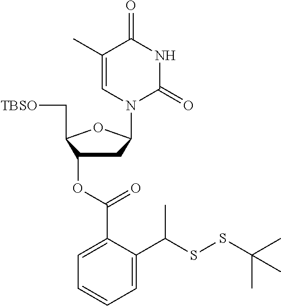

The product was synthesized similarly to Example 5 step 1, wherein 2-(1-(ethyldisulfaneyl)ethyl)benzoic acid was replaced by 2-(1-(tert-butyldisulfaneyl)ethyl)benzoic acid (available from Beijing Okeanos Tech. Co., Ltd., cat. No.: OK-H-21010), the yield of this step was 80%, MW=608.

¹H NMR (400 MHz, CDCl₃) δ 8.73 (d, J=5.8 Hz, 1H), 7.84 (ddd, J=7.8, 3.5, 1.5 Hz, 1H), 7.70-7.45 (m, 3H), 7.32 (td, J=7.6, 1.4 Hz, 1H), 6.46 (td, J=9.5, 5.3 Hz, 1H), 5.57-5.35 (m, 1H), 5.16 (p, J=7.1 Hz, 1H), 4.28 (dd, J=17.9, 1.7 Hz, 1H), 4.13-3.90 (m, 2H), 2.58 (ddd, J=14.7, 9.8, 5.3 Hz, 1H), 2.23 (dtd, J=16.2, 6.2, 2.7 Hz, 1H), 1.98-1.92 (m, 3H), 1.69 (d, J=6.7 Hz, 3H), 1.22 (d, J=11.2 Hz, 8H), 0.96 (s, 9H), 0.17 (s, 6H).

(2) Step 2

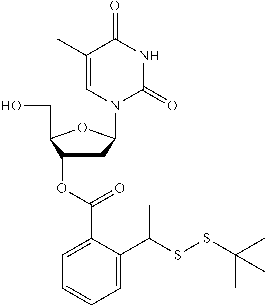

The product was synthesized similarly to Example 5 Step 2, wherein 2-(1-(ethyldisulfaneyl)ethyl)benzoic acid was replaced by 2-(1-(tert-butyldisulfaneyl)ethyl)benzoic acid (available from Beijing Okeanos Tech. Co., Ltd., cat. No.: OK-H-21010), the yield of this step was 90%, MW=494.

¹H NMR (400 MHz, CDCl₃) δ 8.90 (s, 1H), 7.83 (dt, J=7.9, 1.6 Hz, 1H), 7.62 (d, J=7.9 Hz, 1H), 7.55 (dtd, J=7.8, 4.0, 1.4 Hz, 2H), 7.32 (td, J=7.5, 1.4 Hz, 1H), 6.31 (ddd, J=8.4, 6.0, 2.8 Hz, 1H), 5.59 (dt, J=5.4, 2.5 Hz, 1H), 5.14 (dq, J=18.4, 7.0 Hz, 1H), 4.27 (dt, J=8.4, 2.5 Hz, 1H), 4.01 (d, J=2.6 Hz, 2H), 2.65-2.48 (m, 2H), 2.35 (s, 2H), 1.94 (d, J=1.2 Hz, 3H), 1.68 (dd, J=6.9, 1.4 Hz, 3H), 1.22 (d, J=2.9 Hz, 9H).

(3) Step 3

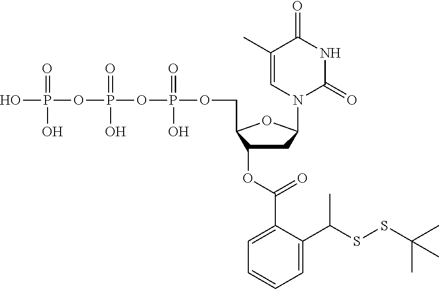

The product was synthesized similarly to Example 5 step 3, wherein 2-(1-(ethyldisulfaneyl)ethyl)benzoic acid was replaced by 2-(1-(tert-butyldisulfaneyl)ethyl)benzoic acid (available from Beijing Okeanos Tech. Co., Ltd., cat. No.: OK-H-21010), the yield of this step was 40%, MW=734.

¹H NMR (400 MHz, DMSO-d₆) δ 11.30 (s, 1H), 7.91 (d, J=7.2 Hz, 1H), 7.84 (d, J=8.0 Hz, 1H), 7.62 (d, J=4.6 Hz, 2H), 7.44-7.39 (m, 1H), 6.37-6.29 (m, 1H), 5.56 (t, J=5.0 Hz, 1H), 5.07-4.98 (m, 1H), 4.29 (d, J=28.0 Hz, 1H), 4.14-3.99 (m, 2H), 2.39 (dd, J=13.9, 5.9 Hz, 1H), 2.31 (dd, J=14.2, 5.7 Hz, 1H), 1.83 (s, 3H), 1.63 (dd, J=7.0, 1.1 Hz, 3H), 1.19 (d, J=2.5 Hz, 9H).

### Example 42

(1) Step 1

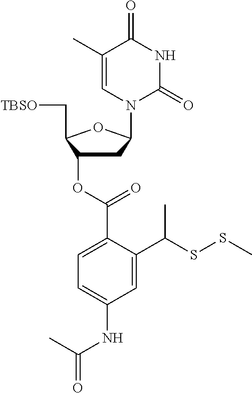

The product was synthesized similarly to Example 5 step 1, wherein 2-(1-(ethyldisulfaneyl)ethyl)benzoic acid was replaced by 4-acetamido-2-(1-(methyldisulfaneyl)ethyl)benzoic acid (available from Beijing Okeanos Tech. Co., Ltd., cat. No.: OK-H-21002), the yield of this step was 80%, MW=623.

¹H NMR (400 MHz, CDCl₃) δ 8.29 (s, 1H), 7.88 (d, J=8.6 Hz, 1H), 7.68 (s, 1H), 7.64-7.58 (m, 2H), 7.46 (s, 1H), 6.42 (dd, J=9.3, 5.1 Hz, 1H), 5.46 (d, J=5.9 Hz, 1H), 5.42-5.36 (m, 1H), 4.29 (d, J=1.8 Hz, 1H), 4.04-3.95 (m, 2H), 2.57 (dd, J=13.8, 5.3 Hz, 1H), 2.29-2.23 (m, 1H), 2.22 (s, 3H), 2.06 (s, 3H), 1.94 (d, J=1.2 Hz, 3H), 1.68 (d, J=7.0 Hz, 3H), 0.95 (s, 9H), 0.17 (s, 6H).

(2) Step 2

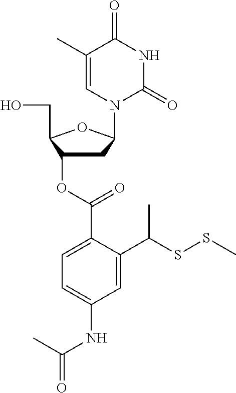

The product was synthesized similarly to Example 5 Step 2, wherein 2-(1-(ethyldisulfaneyl)ethyl)benzoic acid was replaced by 4-acetamido-2-(1-(methyldisulfaneyl)ethyl)benzoic acid (available from Beijing Okeanos Tech. Co., Ltd., cat. No.: OK-H-21002), the yield of this step was 75%, MW=509.

¹H NMR (400 MHz, CDCl₃) δ 8.08 (s, 1H), 7.89 (dd, J=8.5, 5.6 Hz, 1H), 7.66 (d, J=9.5 Hz, 2H), 7.54 (d, J=7.2 Hz, 1H), 7.45 (s, 1H), 6.29 (t, J=7.1 Hz, 1H), 5.59-5.56 (m, 1H), 5.41-5.35 (m, 1H), 4.29-4.25 (m, 1H), 4.01 (d, J=2.2 Hz, 2H), 3.01-2.93 (m, 1H), 2.58-2.55 (m, 1H), 2.23 (s, 3H), 2.09 (d, J=5.6 Hz, 3H), 1.95 (d, J=1.2 Hz, 3H), 1.68 (s, 3H).

(3) Step 3

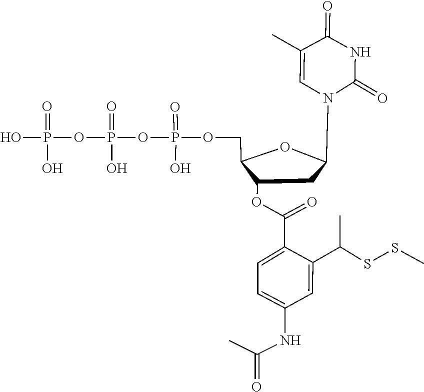 [SMILES: CSSC(C)c1cc(NC(C)=O)ccc1C(=O)O[C@@H]1C[C@H](n2cc(C)c(=O)[nH]c2=O)O[C@@H]1COP(=O)(O)OP(=O)(O)OP(=O)(O)O]

The product was synthesized similarly to Example 5 step 3, wherein 2-(1-(ethyldisulfaneyl)ethyl)benzoic acid was replaced by 4-acetamido-2-(1-(methyldisulfaneyl)ethyl)benzoic acid (available from Beijing Okeanos Tech. Co., Ltd., cat. No.: OK-H-21002), the yield of this step was 35%, MW=749.

¹H NMR (400 MHz, DMSO-d₆) δ 11.30 (s, 1H), 10.52 (d, J=3.1 Hz, 1H), 7.97 (d, J=4.1 Hz, 1H), 7.92-7.82 (m, 2H), 7.69 (d, J=8.8 Hz, 1H), 6.37-6.26 (m, 1H), 5.55 (d, J=5.3 Hz, 1H), 5.34-5.25 (m, 1H), 4.27 (d, J=16.7 Hz, 1H), 4.12-4.05 (m, 2H), 2.49-2.46 (m, 1H), 2.35 (td, J=13.9, 5.7 Hz, 1H), 2.11 (d, J=12.7 Hz, 3H), 2.08 (s, 3H), 1.84 (s, 3H), 1.61 (d, J=6.9 Hz, 3H).

### Example 43

(1) Step 1

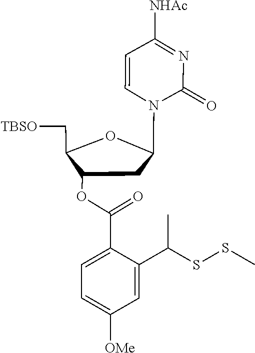 [SMILES: COc1ccc(C(=O)O[C@@H]2C[C@H](n3ccc(NC(C)=O)nc3=O)O[C@@H]2CO[Si](C)(C)C(C)(C)C)c(C(C)SSC)c1]

The product was synthesized similarly to Example 7 step 1, wherein 2-(1-(ethyldisulfaneyl)ethyl)benzoic acid was replaced by 4-methoxy-2-(1-(methyldisulfaneyl)ethyl)benzoic acid (available from Beijing Okeanos Tech. Co., Ltd., cat. No.: OK-H-21001), the yield of this step was 80%, MW=623.

¹H NMR (500 MHz, DMSO-d₆) δ 10.92 (s, 1H), 8.24 (d, J=7.5 Hz, 1H), 7.92 (dd, J=8.7, 4.0 Hz, 1H), 7.24 (d, J=7.5 Hz, 1H), 7.10 (d, J=2.6 Hz, 1H), 7.00 (ddd, J=8.8, 2.6, 1.4 Hz, 1H), 6.19 (q, J=6.3 Hz, 1H), 5.57 (d, J=8.0 Hz, 3H), 5.46-5.37 (m, 1H), 5.37-5.27 (m, 1H), 4.37 (dd, J=21.7, 2.9 Hz, 1H), 3.95 (dd, J=11.3, 3.2 Hz, 1H), 3.85 (s, 3H), 2.78-2.62 (m, 1H), 2.38-2.26 (m, 1H), 2.14-2.09 (m, 6H), 0.86 (s, 9H), 0.09 (s, 6H).

(2) Step 2

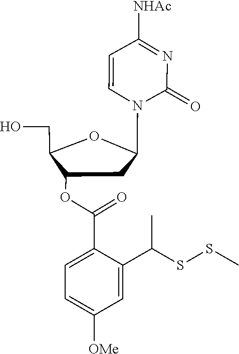 [SMILES: COc1ccc(C(=O)O[C@@H]2C[C@H](n3ccc(NC(C)=O)nc3=O)O[C@@H]2CO)c(C(C)SSC)c1]

The product was synthesized similarly to Example 7 Step 2, wherein 2-(1-(ethyldisulfaneyl)ethyl)benzoic acid was replaced by 4-methoxy-2-(1-(methyldisulfaneyl)ethyl)benzoic acid (available from Beijing Okeanos Tech. Co., Ltd., cat. No.: OK-H-21001), the yield of this step was 85%, MW=509.

¹H NMR (500 MHz, DMSO-d₆) δ 10.91 (s, 1H), 8.35 (d, J=7.5 Hz, 1H), 7.92 (dd, J=8.8, 4.1 Hz, 1H), 7.24 (d, J=7.5 Hz, 1H), 7.10 (d, J=2.7 Hz, 1H), 7.00 (ddd, J=8.8, 2.7, 1.3 Hz, 1H), 6.22 (dt, J=7.9, 6.0 Hz, 1H), 5.43 (dt, J=6.6, 2.1 Hz, 1H), 5.39-5.30 (m, 1H), 5.26 (t, J=5.3 Hz, 1H), 4.29 (dd, J=18.4, 2.7 Hz, 1H), 3.85 (s, 3H), 3.72 (dd, J=5.5, 3.2 Hz, 2H), 2.68-2.55 (m, 1H), 2.34 (dt, J=14.1, 7.0 Hz, 1H), 2.16-2.08 (m, 6H), 1.65 (d, J=7.0 Hz, 3H).

(3) Steps 3-4

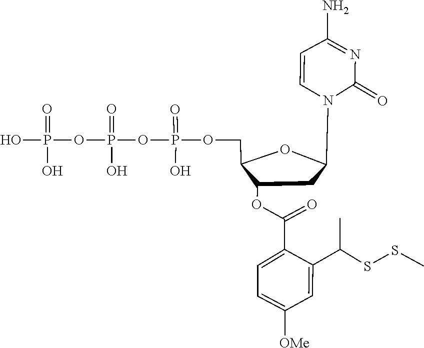 [SMILES: COc1ccc(C(=O)O[C@@H]2C[C@H](n3ccc(N)nc3=O)O[C@@H]2COP(=O)(O)OP(=O)(O)OP(=O)(O)O)c(C(C)SSC)c1]

The product was synthesized similarly to Example 7 steps 3-4, wherein 2-(1-(ethyldisulfaneyl)ethyl)benzoic acid was replaced by 4-methoxy-2-(1-(methyldisulfaneyl)ethyl)benzoic acid (available from Beijing Okeanos Tech. Co., Ltd., cat. No.: OK-H-21001), total yield of these two steps was 30%, MW=707.

¹H NMR (400 MHz, DMSO) δ 7.96-7.90 (m, 2H), 7.29 (s, 1H), 7.09 (d, J=2.3 Hz, 2H), 7.00 (dd, J=8.8, 2.5 Hz, 1H), 6.34 (dd, J=15.3, 6.6 Hz, 1H), 5.81 (d, J=7.5 Hz, 1H), 5.47 (d, J=4.3 Hz, 1H), 5.33 (q, J=7.0 Hz, 1H), 4.28 (d, J=17.7 Hz, 1H), 4.13-3.97 (m, 2H), 3.85 (s, 3H), 2.43-2.22 (m, 2H), 2.13 (d, J=10.6 Hz, 3H), 1.64 (d, J=7.0 Hz, 3H).

### Example 44

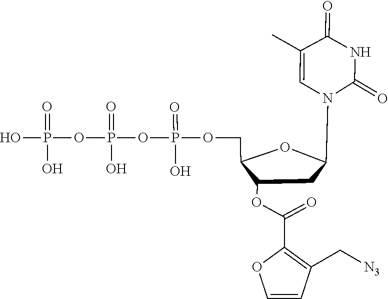 [SMILES: Cc1cn([C@H]2C[C@@H](OC(=O)c3occc3CN=[N+]=[N-])[C@@H](COP(=O)(O)OP(=O)(O)OP(=O)(O)O)O2)c(=O)[nH]c1=O]

The product was synthesized similarly to Example 5 Steps 1-3, wherein 2-(1-(ethyldisulfaneyl)ethyl)benzoic acid was replaced by 3-(azidomethyl)furan-2-carboxylic acid (available from Beijing Okeanos Tech. Co., Ltd., cat. No.: OK-H-21011), yield for the last step was 45%, MW=631.

¹H NMR (400 MHz, DMSO-d₆) δ 11.31 (s, 1H), 8.01 (d, J=1.6 Hz, 1H), 7.91 (d, J=22.7 Hz, 1H), 6.83 (d, J=1.6 Hz, 1H), 6.33 (dd, J=9.4, 5.7 Hz, 1H), 5.63-5.56 (m, 1H), 4.65 (s, 2H), 4.25 (s, 1H), 4.10-3.99 (m, 2H), 2.48-2.46 (m, 1H), 2.37-2.32 (m, 1H), 1.83 (s, 3H).

### Example 45

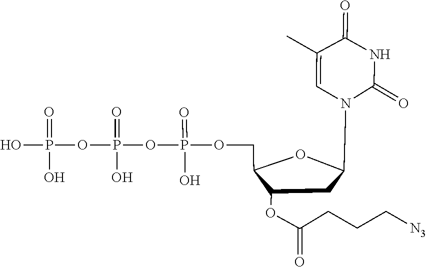 [SMILES: Cc1cn([C@H]2C[C@@H](OC(=O)CCCN=[N+]=[N-])[C@@H](COP(=O)(O)OP(=O)(O)OP(=O)(O)O)O2)c(=O)[nH]c1=O]

The product was synthesized similarly to Example 5 Steps 1-3, wherein 2-(1-(ethyldisulfaneyl)ethyl)benzoic acid was replaced by 4-azidobutanoic acid (available from Beijing Okeanos Tech. Co., Ltd., cat. No.: OK-H-20008), yield for the last step was 20%, MW=593.

¹H NMR (400 MHz, DMSO-d₆) δ 11.28 (s, 1H), 7.90 (d, J=0.9 Hz, 1H), 6.24 (dd, J=9.5, 5.6 Hz, 1H), 5.33 (d, J=5.5 Hz, 1H), 4.09 (s, 1H), 4.04-3.95 (m, 2H), 3.38 (t, J=6.8 Hz, 2H), 2.44 (t, J=7.3 Hz, 2H), 2.41-2.33 (m, 1H), 2.18 (dd, J=13.8, 5.6 Hz, 1H), 1.82 (s, 3H), 1.81-1.75 (m, 2H).

### II. Testing Examples

The inventor found that the nucleotide analogues of the present application led to both excellent blocking effect and excellent polymerization effect. Sequencing performed with these nucleotide analogues resulted in excellent effects, with both high mapping rate and low error rate.

Specifically, the inventor evaluated and tested the nucleotide analogues prepared in the above preparation examples on a high-throughput sequencer.

1. Evaluation of Blocking Effect

Nucleotide substrate: fluorescent labeled standard hot dNTP (four kinds) and standard cold dNTP (four kinds), with structures showed below, all available from MGISEQ-2000RS high-throughput sequencing kit (FCL SE50, MGI Tech Co., Ltd., cat. No. 1000012551). For each test, only one of the nucleotide analogues cold dNTP of the invention (dTTP, dATP, dCTP, and dGTP) was used. For ease of presentation, only testing results for dTTP were provided in the following table 1 for each kind of 3′-OH modification.

Sequencing was conducted according to the operation protocol of MGISEQ2000 sequencer, using the above nucleotide substrates and MGISEQ-2000RS high-throughput sequencing kit (FCL SE50).

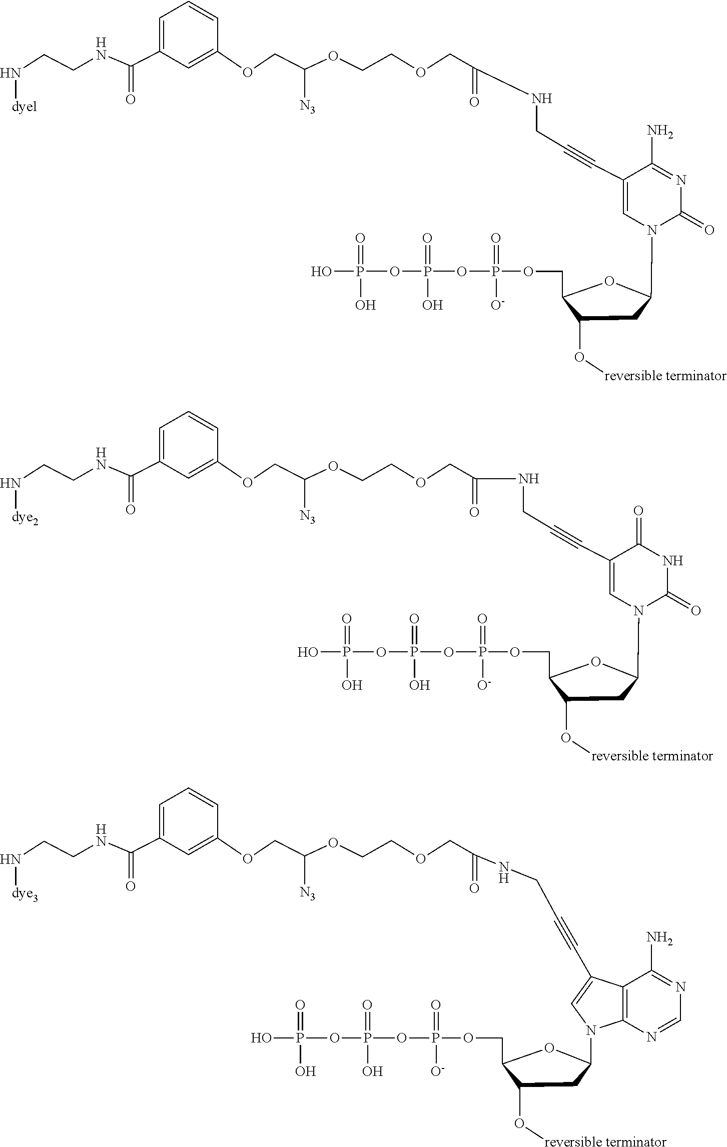

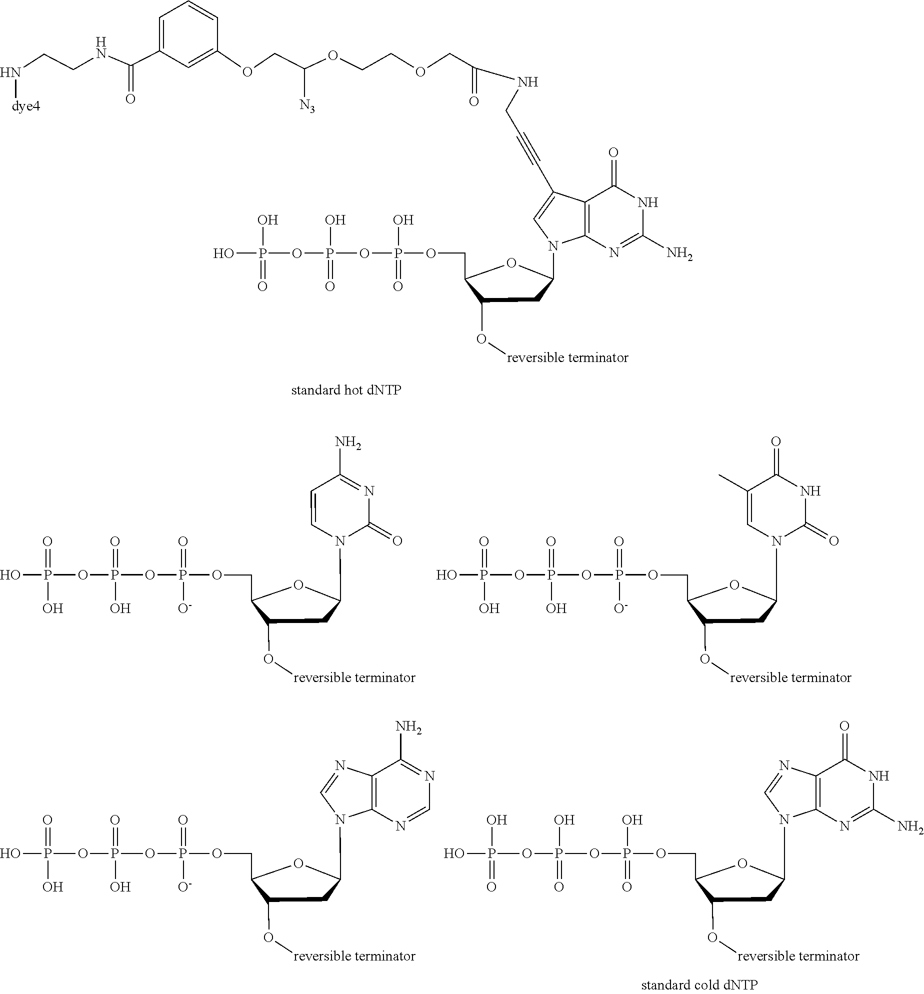

(1) Preparing DNA nanospheres by using E. coli sequencing library;

(2) Loading DNA nanospheres onto the MGISEQ2000 sequencing chip;

(3) Amounting the loaded sequencing chip onto the MGISEQ2000 sequencer and set the sequencing process;

(4) Performing Test Round 1: incorporating standard hot dNTP, taking photos for recording signal value, and then cleaving the blocking group with thpp reagent, 65° C. 1 min.

(5) Performing Test Round 2: incorporating standard cold dNTP, then incorporating standard hot dNTP, taking photos for recording signal value, and then cleaving blocking group with thpp reagent, 65° C. 1 min.

(6) Performing Test Round 3: incorporating standard hot dNTP, taking photos for recording signal value, and then cleaving blocking group with thpp reagent, 65° C. 1 min.

(7) Performing Test Round 4: incorporating nucleotide analogues cold dNTP of the invention (only one kind of cold dNTP incorporated for each test), then incorporating standard hot dNTP, taking photos for recording signal value, and then cleaving blocking group with thpp reagent, 65° C. 1 min.

(8) Performing Test Round 5: incorporating standard hot dNTP, taking photos for recording signal value.

(9) Evaluating Incorporation efficiency and Cleavage efficiency. The results were showed in Table 1.

Equation for Calculating Incorporation Efficiency:

$${E}_{I} = \frac{{c}_{3} - {c}_{4}}{{c}_{1} - {c}_{2}}$$

wherein:
EI (Incorporation efficiency) is the ratio of Incorporation efficiency values for testing nucleotide and comparative nucleotide;
C1 is signal value of Test Round 1;
C2 is signal value of Test Round 2;
C3 is signal value of Test Round 3;
C4 is signal value of Test Round 4;

Equation for Calculating Cleavage Efficiency:

$${E}_{c} = 1 - \frac{{Σ}_{C G T} ( \frac{{c}_{3} - {c}_{5}}{{c}_{3}} )}{3 E I} * 4$$

wherein:
Ec (Cleavage efficiency) is the ratio of Cleavage efficiency values for testing nucleotide and comparative nucleotide;
EI is the ratio of Incorporation efficiency values for testing nucleotide and comparative nucleotide;
C3 is signal value of Test Round 3;
C5 is signal value of Test Round 5;
CGT is the signals of base C, base G and base T in Test Round 3.

TABLE 1
Incorporation efficiency and Cleavage efficiency of nucleotide analogues
(only those of dTTP illustrated)

| | |
|---|---|
| 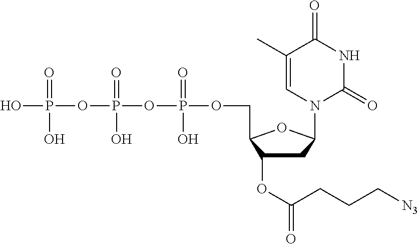 | Incorporation efficiency (E_{I}) 95% Cleavage efficiency (E_{C}) 36% |
| 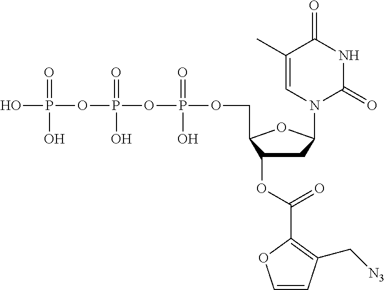 | Incorporation efficiency (E_{I}) 101% Cleavage efficiency (E_{C}) 5% |
| 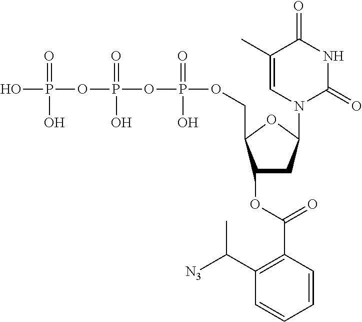 [SMILES: Cc1cn([C@H]2C[C@@H](OC(=O)c3ccccc3C(C)N=[N+]=[N-])[C@@H](COP(=O)(O)OP(=O)(O)OP(=O)(O)O)O2)c(=O)[nH]c1=O] | Incorporation efficiency (E_{I}) 101% Cleavage efficiency (E_{C}) 105% |
| 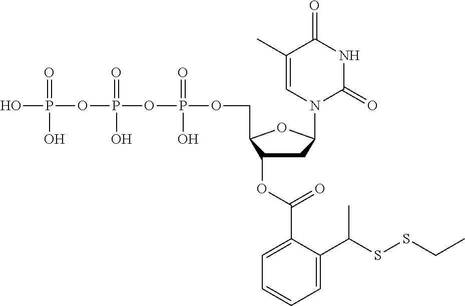 | Incorporation efficiency (E_{I}) 100% Cleavage efficiency (E_{C}) 110% |
| 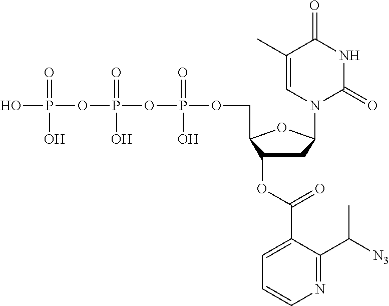 | Incorporation efficiency (E_{I}) 100% Cleavage efficiency (E_{C}) 115% |
| 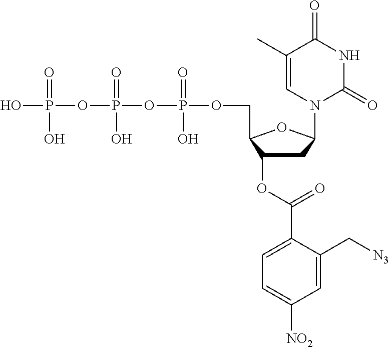 | Incorporation efficiency (E_{I}) 101% Cleavage efficiency (E_{C}) 112% |
| 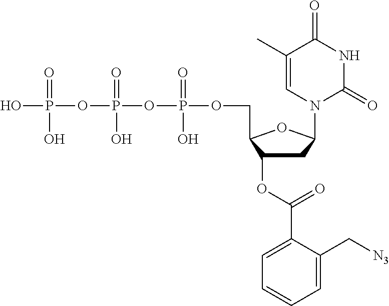 | Incorporation efficiency (E_{I}) 98% Cleavage efficiency (E_{C}) 91% |
| 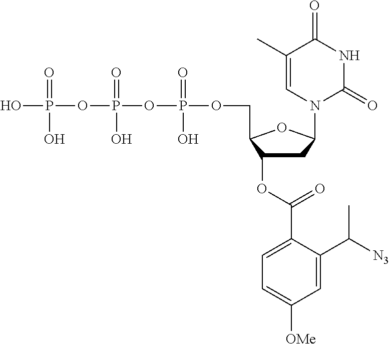 | Incorporation efficiency (E_{I}) 101% Cleavage efficiency (E_{C}) 98% |
| 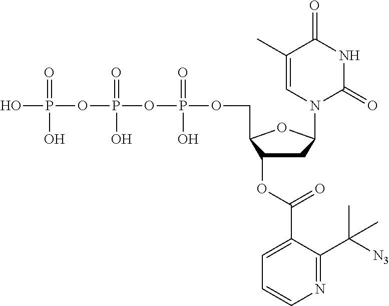 | Incorporation efficiency (E_{I}) 95% Cleavage efficiency (E_{C}) 110% |
| 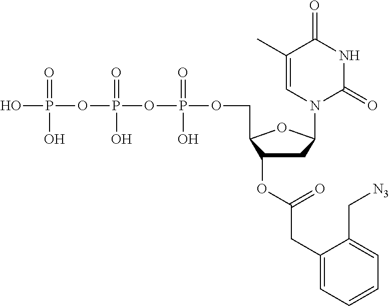 | Incorporation efficiency (E_{I}) 95% Cleavage efficiency (E_{C}) 89% |
| 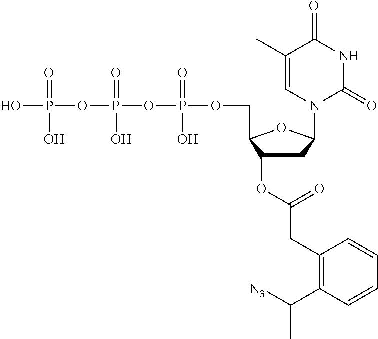 | Incorporation efficiency (E_{I}) 98% Cleavage efficiency (E_{C}) 91% |
| 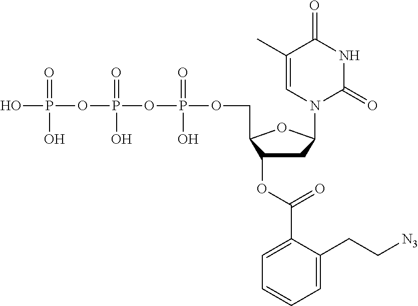 | Incorporation efficiency (E_{I}) 100% Cleavage efficiency (E_{C}) 100% |
| 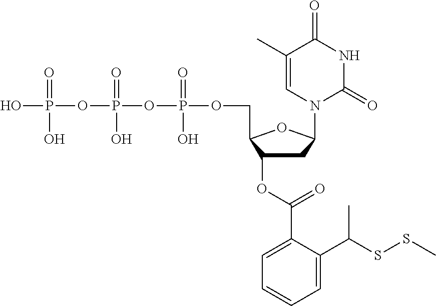 | Incorporation efficiency (E_{I}) 100% Cleavage efficiency (E_{C}) 111% |
| 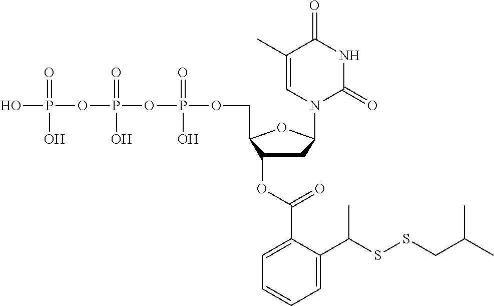 | Incorporation efficiency (E_{I}) 90% Cleavage efficiency (E_{C}) 98% |
| 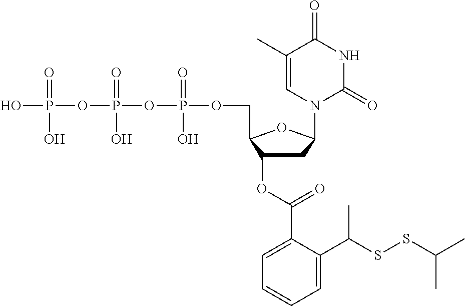 | Incorporation efficiency (E_{I}) 95% Cleavage efficiency (E_{C}) 103% |
| 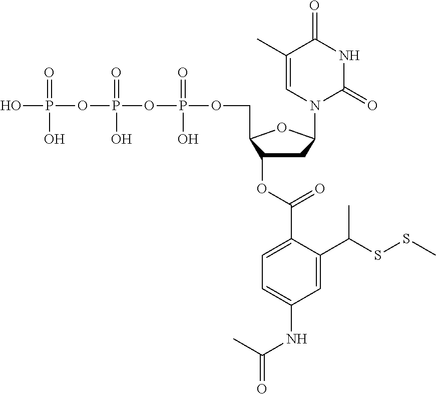 | Incorporation efficiency (E_{I}) 100% Cleavage efficiency (E_{C}) 98% |
| 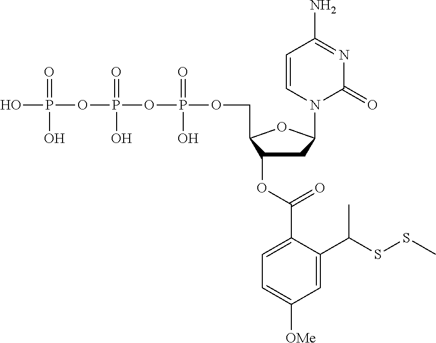 | Incorporation efficiency (E_{I}) 104% Cleavage efficiency (E_{C}) 101% |

2. Evaluation of Sequencing Effect

### Sequencing Example 1

Nucleotide substrates: fluorescent labeled standard hot dNTP (four kinds, all available from MGISEQ-2000RS high-throughput sequencing kit (FCL SE50), MGI Tech Co., Ltd., cat. No. 1000012551); and nucleotide analogues cold dNTP of the invention (four kinds, named as AEB) with structures showed below.

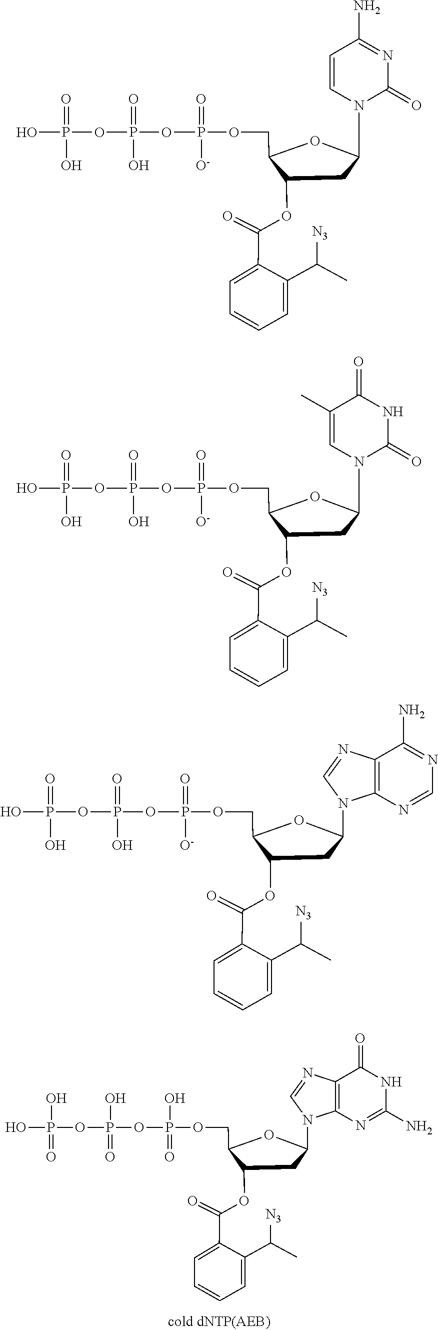 [SMILES: CC(N=[N+]=[N-])c1ccccc1C(=O)O[C@@H]1C[C@H](n2ccc(N)nc2=O)O[C@@H]1COP(=O)([O-])OP(=O)(O)OP(=O)(O)O ; Cc1cn([C@H]2C[C@@H](OC(=O)c3ccccc3C(C)N=[N+]=[N-])[C@@H](COP(=O)([O-])OP(=O)(O)OP(=O)(O)O)O2)c(=O)[nH]c1=O ; CC(N=[N+]=[N-])c1ccccc1C(=O)O[C@@H]1C[C@H](n2cnc3c(N)ncnc32)O[C@@H]1COP(=O)([O-])OP(=O)(O)OP(=O)(O)O ; CC(N=[N+]=[N-])c1ccccc1C(=O)O[C@@H]1C[C@H](n2cnc3c(=O)[nH]c(N)nc32)O[C@@H]1COP(=O)(O)OP(=O)(O)OP(=O)(O)O]

Sequencing was conducted according to the operation protocol of MGISEQ2000 sequencer, using the above nucleotide substrates and MGISEQ-2000RS high-throughput sequencing kit (FCL SE50).

(1) Preparing DNA nanospheres by using E. coli sequencing library;

(2) Loading DNA nanospheres onto the MGISEQ2000 sequencing chip;

(3) Amounting the loaded sequencing chip onto the MGISEQ2000 sequencer and set the sequencing process, hot dNTP incorporation: 60° C. 2 min; cold dNTP incorporation: 60° C. 2 min; signal acquisition; cleavage of blocking group: 65° C. 2 min;

(4) Conducting Basecall analysis on offline data and output results of sequencing index including mapping rate, error rate, Q30, etc. The results were showed in Table 2.

TABLE 2
Results of Basecall analysis

| Nucleotide analogues cold dNTP Mix | AEB |
|---|---|
| Reference | Ecoli.fa |
| Cycle Number | 50 |
| Q30(%) | 84.61 |
| Lag (1%) | 0.18 |
| Lag (2%) | 0.23 |
| Runon (1%) | 0.3 |
| Runon (2%) | 0.37 |
| ESR (%) | 78.17 |
| Mapping Rate, % | 98.8 |
| AvgError Rate, % | 0.84 |
| AvgError Rate!N, % | 0.81 |

### Sequencing Example 2

Nucleotide substrates: fluorescent labeled standard hot dNTP (four kinds, all available from MGISEQ-2000RS high-throughput sequencing kit (FCL SE50), MGI Tech Co., Ltd., cat. No. 1000012551); nucleotide analogues cold dNTP of the invention (four kinds, named as SSEB) with structures showed below.

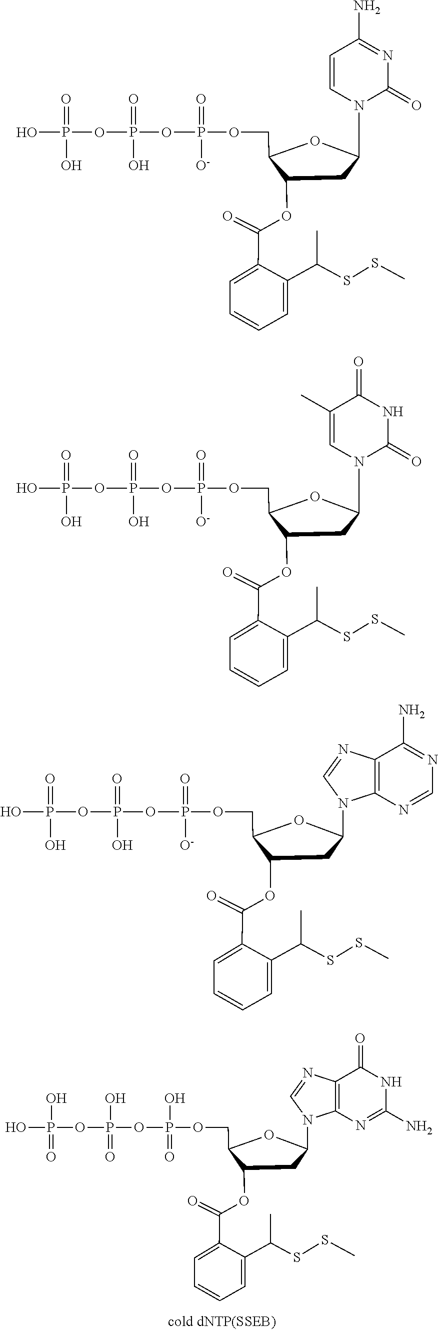 [SMILES: CSSC(C)c1ccccc1C(=O)O[C@@H]1C[C@H](n2ccc(N)nc2=O)O[C@@H]1COP(=O)([O-])OP(=O)(O)OP(=O)(O)O ; CSSC(C)c1ccccc1C(=O)O[C@@H]1C[C@H](n2cc(C)c(=O)[nH]c2=O)O[C@@H]1COP(=O)([O-])OP(=O)(O)OP(=O)(O)O ; CSSC(C)c1ccccc1C(=O)O[C@@H]1C[C@H](n2cnc3c(N)ncnc32)O[C@@H]1COP(=O)([O-])OP(=O)(O)OP(=O)(O)O ; CSSC(C)c1ccccc1C(=O)O[C@@H]1C[C@H](n2cnc3c(=O)[nH]c(N)nc32)O[C@@H]1COP(=O)(O)OP(=O)(O)OP(=O)(O)O]

Sequencing procedure was same as that above in Sequencing Example 1. Results of Basecall analysis were showed in Table 3.

TABLE 3
Results of Basecall analysis

| Nucleotide analogues cold dNTP Mix | SSEB |
|---|---|
| Reference | Ecoli.fa |
| Cycle Number | 50 |
| Q30(%) | 91.89 |
| Lag(%) | 0.11 |
| Runon(%) | 0.23 |
| ESR(%) | 90.33 |
| Mapping Rate, % | 98.59 |
| AvgError Rate, % | 0.13 |

### Sequencing Example 3

Nucleotide substrates: fluorescent labeled standard hot dNTP (four kinds, all available from MGISEQ-2000RS high-throughput sequencing kit (FCL SE50), MGI Tech Co., Ltd., cat. No. 1000012551); nucleotide analogues cold dNTP of the invention (four kinds, named as AZBN) with structures showed below.

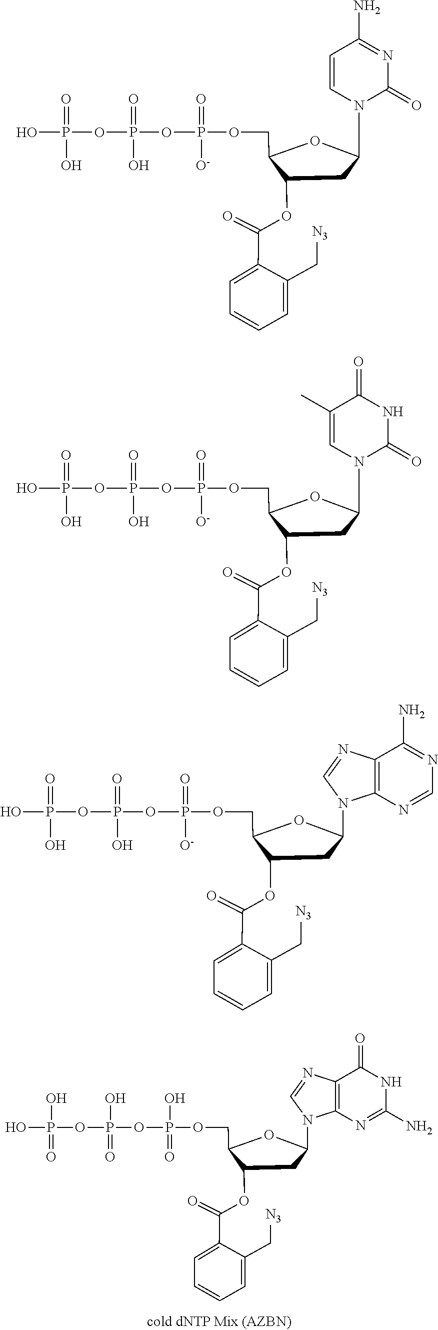 [SMILES: [N-]=[N+]=NCc1ccccc1C(=O)O[C@@H]1C[C@H](n2ccc(N)nc2=O)O[C@@H]1COP(=O)([O-])OP(=O)(O)OP(=O)(O)O ; Cc1cn([C@H]2C[C@@H](OC(=O)c3ccccc3CN=[N+]=[N-])[C@@H](COP(=O)([O-])OP(=O)(O)OP(=O)(O)O)O2)c(=O)[nH]c1=O ; [N-]=[N+]=NCc1ccccc1C(=O)O[C@@H]1C[C@H](n2cnc3c(N)ncnc32)O[C@@H]1COP(=O)([O-])OP(=O)(O)OP(=O)(O)O ; [N-]=[N+]=NCc1ccccc1C(=O)O[C@@H]1C[C@H](n2cnc3c(=O)[nH]c(N)nc32)O[C@@H]1COP(=O)(O)OP(=O)(O)OP(=O)(O)O]

Sequencing procedure was same as that above in Sequencing Example 1. Results of Basecall analysis were showed in Table 4.

TABLE 4
Results of Basecall analysis

| Nucleotide analogues cold dNTP Mix | AZBN |
|---|---|
| Reference | Ecoli.fa |
| Cycle Number | 50 |
| Q30(%) | 61.39 |
| Lag(%) | 0.03 |
| Runon(%) | 0.12 |
| ESR(%) | 62.17 |
| Mapping Rate, % | 5.75 |
| AvgError Rate, % | 4.94 |
| AvgError Rate!N, % | 4.89 |

### Sequencing Example 4: Sequencing with Hot T-SSEB

1. Synthesis of Nucleotide Analogues dTTP Carrying Fluorescent Group (Named as Hot T-SSEB)

(1) Step 1

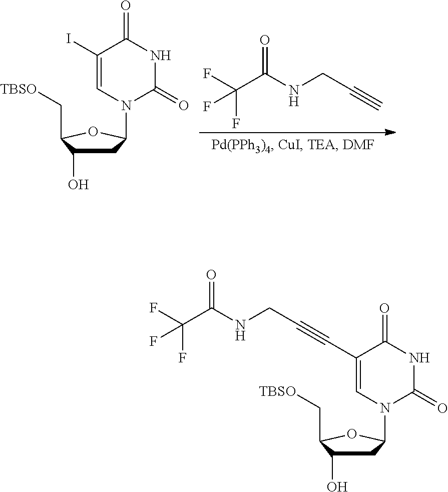

Iodinated nucleoside substrate T (Okeanos Tech, cat. No. OK-N-16001, Ig) was dissolved in DMF, followed by adding Pd(PPh3)₄ (10 mol %), CuI (15 mol %), triethylamine (3 eq) and substrate propargylamine (Okeanos Tech, cat. No. OK20A410, 1.5 eq). The mixture was allowed to react at 60° C. for 12 h. The reaction was quenched with water, extracted with DCM, concentrated, and purified by column chromatography to provide the product 1.1 g, as a white solid.

MS[ES(−)], m/z 491.1. ¹H NMR (400 MHz, DMSO-d₆) δ 11.67 (s, 1H), 10.00 (t, J=5.5 Hz, 1H), 7.94 (s, 1H), 6.12 (dd, J=7.6, 5.9 Hz, 1H), 5.28 (d, J=4.1 Hz, 1H), 4.26-4.12 (m, 3H), 3.88 (q, J=2.8 Hz, 1H), 3.81 (dd, J=11.5, 2.6 Hz, 1H), 3.73 (dd, J=11.5, 3.1 Hz, 1H), 2.17 (ddd, J=13.2, 6.0, 2.8 Hz, 1H), 2.05 (ddd, J=13.3, 7.7, 5.8 Hz, 1H), 0.87 (s, 9H), 0.08 (d, J=1.8 Hz, 6H).

(2) Step 2

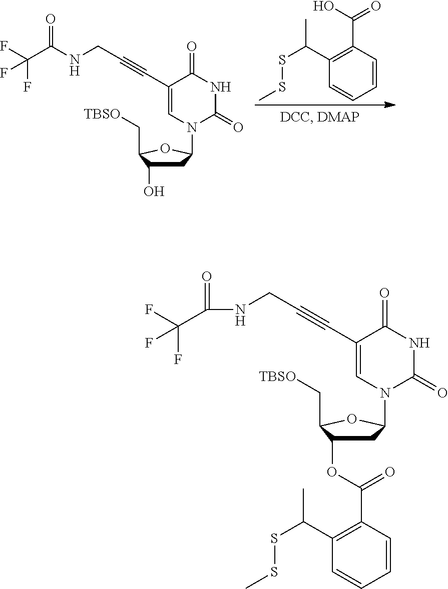

The nucleoside from Step 1 (300 mg) was dissolved in 10 mL of DMF, followed by adding DCC (1.2 eq) and DMAP (10% mol). The mixture was stirred for 30 minutes, added with disulfaneyl carboxylic acid substrate (OKeanos Tech, cat. No. OK20A420)(1.5 eq). The mixture was stirred for 12 hours and subjected directly to column chromatography to provide the product 359 mg, as a white solid.

MS[ES(−)], m/z 700.3. ¹H NMR (400 MHz, DMSO-d₆) δ 11.74 (s, 1H), 10.03 (t, J=5.5 Hz, 1H), 7.98 (s, 1H), 7.90-7.82 (m, 1H), 7.68-7.58 (m, 2H), 7.47-7.40 (m, 1H), 6.23-6.18 (m, 1H), 5.45-5.42 (m, 1H), 5.20-5.12 (m, 1H), 4.38-4.16 (m, 3H), 3.98-3.87 (m, 2H), 2.60-2.53 (m, 1H), 2.40-2.31 (m, 1H), 2.06 (d, J=0.6 Hz, 3H), 1.65 (dd, J=7.0, 1.0 Hz, 3H), 0.90 (s, 9H), 0.13 (d, J=1.2 Hz, 6H).

(3) Step 3

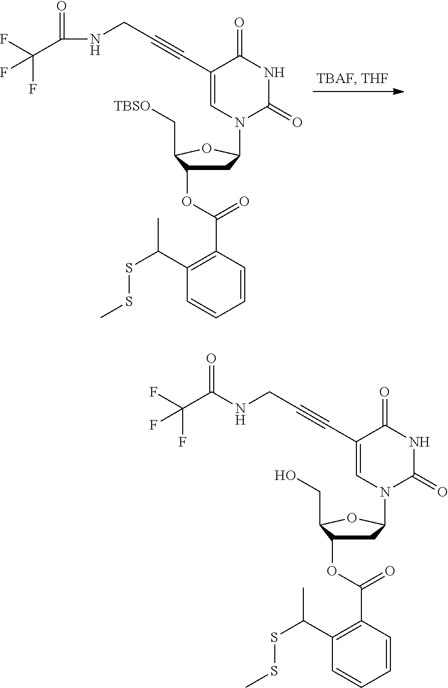

The nucleoside from Step 2 (300 mg) was dissolved in 10 mL of THF, followed by adding TBAF (2 eq, 1M in THF) at 0° C. The mixture was stirred at 0° C. for 30 minutes, and allowed to warm back to room temperature with stirring for 4 hours. The mixture was subjected directly to column chromatography to provide the product 200 mg, as a white solid.

MS[ES(−)], m/z 587.2. ¹H NMR (400 MHz, DMSO-d₆) δ 11.68 (d, J=3.2 Hz, 1H), 10.06 (t, J=5.6 Hz, 1H), 8.23 (d, J=3.2 Hz, 1H), 7.88-7.78 (m, 1H), 7.66-7.56 (m, 2H), 7.43-7.39 (m, 1H), 6.25-6.13 (m, 1H), 5.48-5.45 (m, 1H), 5.31 (t, J=5.2 Hz, 1H), 5.18-5.12 (m, 1H), 4.28-4.16 (m, 3H), 3.76-3.67 (m, 2H), 2.50-2.40 (m, 2H), 2.04 (d, J=9.2 Hz, 3H), 1.68-1.59 (m, 3H).

(4) Step 4

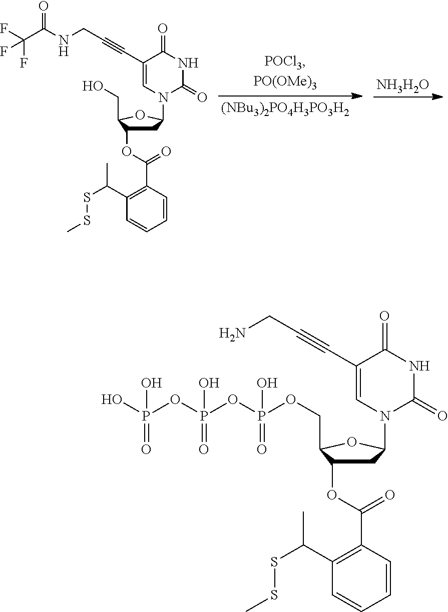

The nucleoside from Step 3 (200 mg) was dissolved in 5 mL of trimethyl phosphate. The mixture was added with phosphorus oxychloride (1.5 eq) at 0° C. with stirring at 0° C. for 120 minutes. The reaction mixture was added to a DMF solution (5 mL) of tributyl ammonium pyrophosphate (2 eq) with continuously stirring at 0° C. for 3 h. The reaction was quenched with 0.1 M TEAB buffer and separated by preparative HPLC reversed phase column (C18, mobile phase: 0.1 M TEAB-acetonitrile). After concentration, the residue was added to 3 mL of concentrated aqueous ammonia and allowed to react for 2 h, followed by preparative HPLC reversed phase column chromatography (C18, mobile phase: 0.1 M TEAB-acetonitrile) to provide the product 120 mg as a white solid.

MS[ES(−)], m/z 745.5. ¹H NMR (400 MHz, D₂O) δ 8.51 (s, 1H), 7.90 (dd, J=7.9, 1.4 Hz, 1H), 7.73 (d, J=7.8 Hz, 1H), 7.67 (td, J=7.6, 1.4 Hz, 1H), 7.48 (t, J=7.5 Hz, 1H), 6.45 (td, J=9.2, 5.7 Hz, 1H), 5.75 (t, J=4.7 Hz, 1H), 5.15-5.07 (m, 1H), 4.72-4.64 (m, 1H), 4.39 (d, J=3.3 Hz, 2H), 4.07 (s, 2H), 2.82-2.76 (m, 1H), 2.64-2.54 (m, 1H), 2.02 (d, J=6.4 Hz, 3H), 1.73 (dd, J=7.1, 2.2 Hz, 3H); 31P NMR (162 MHz, D2O) δ −9.79 (dd, J=20.3, 9.6 Hz, 1P), −11.68 (d, J=19.2 Hz, 1P), −22.82 (td, J=19.2, 6.1 Hz, 1P).

(5) Step 5

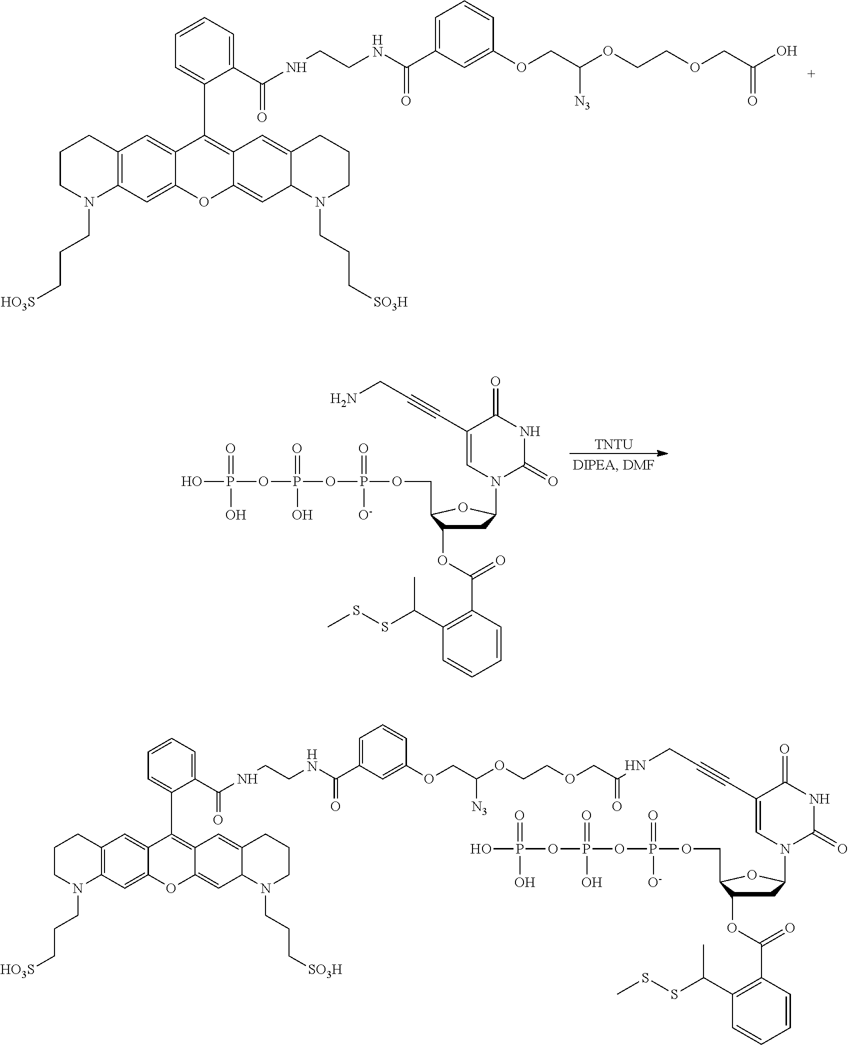

To a dried 20 mL round bottom flask equipped with a magnetic stirrer was added dye-linker solid (Okeanos Tech, OK-F-20211)(9.3 mg, 1 eq.), dissolved with appropriate amount of DMF (3 ml), followed by adding in sequence TNTU solid (7 mg, 0.02 mmol, 2 eq.) and DIPEA (2.6 mg, 0.02 mmol, 2 eq.), wherein DIPEA was diluted with 0.5 ml of DMF in a 1.5 ml PE tube and added into the mixture dropwise with stirring for about 1 hour. The mixture was sampled and dissolved in acetonitrile, and monitored with HPLC and MS for AF532-V4 raw material depletion. To the mixture was added solid hot T-mSSEB substrate (15 mg, 0.02 mmol, 1 eq.) at room temperature with stirring for 1 hour, and sampled and dissolved in acetonitrile, and monitored with HPLC and MS for reaction progress. The reaction was allowed to proceed overnight until a complete consumption of nhs ester, quenched with 0.1 M TEAB buffer, and separated with preparative HPLC reverse phase column chromatography (C18, mobile phase: 0.1 M TEAB-acetonitrile). The desired fractions were concentrated, and added into 3 mL of concentrated aqueous ammonia to react for 2 h, followed by purification with preparative HPLC reverse phase column chromatography (C18, mobile phase: 0.1 M TEAB-acetonitrile) to provide a solid 10 mg.

MS[ES(−)], m/z 1815.0. ¹H NMR (400 MHz, DMSO-d₆) δ 9.06 (d, J=6.7 Hz, 1H), 8.80 (d, J=47.0 Hz, 1H), 8.28 (t, J=7.2 Hz, 1H), 8.18-8.00 (m, 1H), 7.86 (d, J=7.7 Hz, 1H), 7.75-7.66 (m, 2H), 7.66-7.58 (m, 3H), 7.58-7.40 (m, 4H), 7.39-7.33 (m, 1H), 7.17-7.07 (m, 1H), 6.98 (s, 2H), 6.79 (s, 2H), 6.31-6.17 (m, 1H), 5.58 (d, J=3.5 Hz, 1H), 5.21-5.14 (m, 2H), 4.34 (d, J=19.3 Hz, 1H), 4.26 (dd, J=10.5, 3.9 Hz, 1H), 4.18 (dd, J=10.6, 4.5 Hz, 2H), 4.09 (d, J=3.5 Hz, 2H), 3.99 (s, 2H), 3.95-3.82 (m, 2H), 3.68 (s, 6H), 3.52 (s, 4H), 3.33-3.24 (m, 2H), 3.22-3.15 (m, 2H), 3.14-3.06 (m, 2H), 2.65 (t, J=7.6 Hz, 4H), 2.57 (t, J=7.1 Hz, 5H), 2.46-2.37 (m, 1H), 2.05 (d, J=16.8 Hz, 3H), 2.01-1.93 (m, 4H), 1.86-1.81 (m, 3H), 1.77-1.72 (m, 1H), 1.65 (d, J=6.9 Hz, 3H).

2. Sequencing Performed with Nucleotide Analogues Containing the Hot T-SSEB Synthesized Above

Nucleotide substrates: fluorescent labeled standard hot dNTP (available from MGISEQ-2000RS high-throughput sequencing kit (FCL SE50), MGI Tech Co., Ltd., cat. no. 1000012551), wherein hot dTTP was replaced by hot T-SSEB synthesized above, with structures showed below; and nucleotide analogues cold dNTP of the invention (four kinds, named as SSEB) with structures showed below.

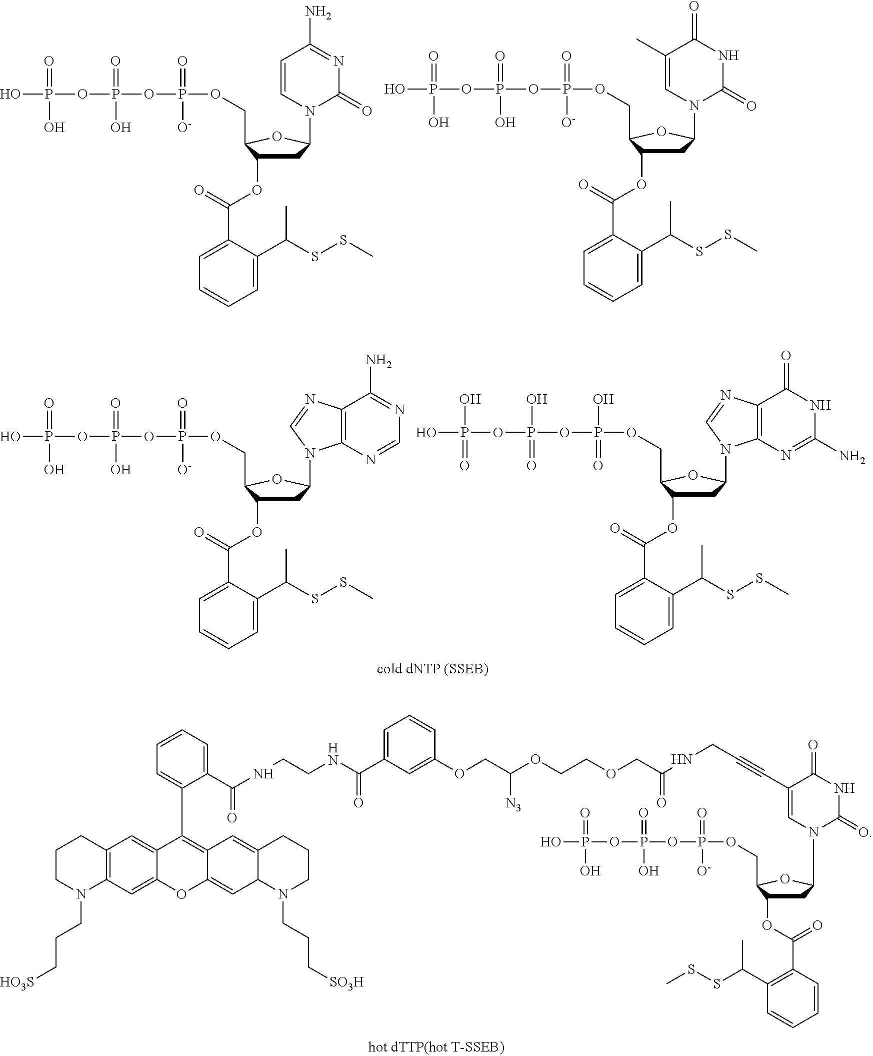

Sequencing procedure was same as that above in Sequencing Example 1. Results of Basecall analysis were showed in Table 5.

TABLE 5
Results of Basecall analysis

| | hot T-SSEB + cold dNTP-SSEB, |
|---|---|
| Nucleotide analogues | standard hot dNTP for others |
| Reference | Ecoli.fa |
| Cycle Number | 50 |
| Q30(%) | 87.24 |
| Lag(%) | 0.19 |
| Runon(%) | 0.24 |
| ESR(%) | 74.7 |
| Mapping Rate, % | 98.44 |
| AvgError Rate, % | 0.27 |

### Sequencing Example 5: Sequencing with SS-Hot G

1. Synthesis of Nucleotide Analogues dGTP Carrying Fluorescent Group (Named as SS-Hot G)

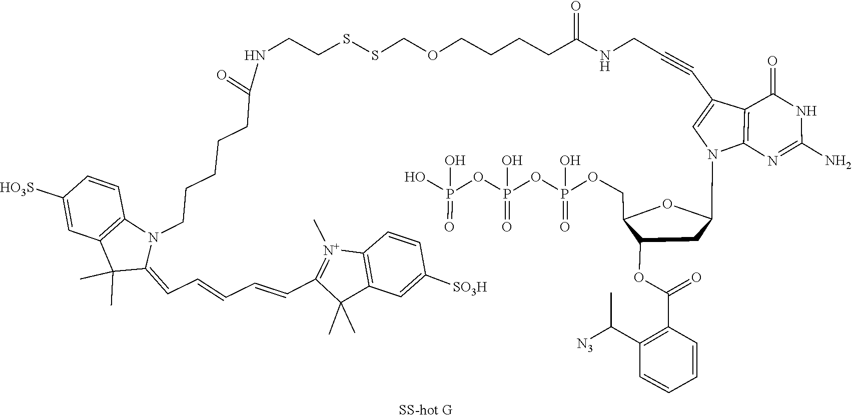 [SMILES: CC(N=[N+]=[N-])c1ccccc1C(=O)O[C@@H]1C[C@H](n2cc(C#CCNC(=O)CCCCOCSSCCNC(=O)CCCCCN3\C(=C/C=C/C=C/C4=[N+](C)c5ccc(S(=O)(=O)O)cc5C4(C)C)C(C)(C)c4cc(S(=O)(=O)O)ccc43)c3c(=O)[nH]c(N)nc32)O[C@@H]1COP(=O)(O)OP(=O)(O)OP(=O)(O)O]

The product was synthesized similarly to that in Sequencing Example 4, wherein an iodinated G-nucleoside substrate was used in place of the iodinated T-nucleoside substrate, and azdio carboxylic acid substrate was used in place of the disulfaneyl carboxylic acid substrate, both available from OKeanos Tech Co., Ltd. New dye-linker was required and available from MyChem LLC Co., Ltd. (cat. no. 110920Cy5). 10 mg of final product was obtained.

MS[ES(−)], m/z 1575. ¹H NMR (400 MHz, DMSO-d₆) δ 10.54 (s, 1H), 8.49 (s, 1H), 8.34 (t, J=13.1 Hz, 2H), 8.07 (t, J=5.5 Hz, 1H), 7.92 (dd, J=7.2, 4.6 Hz, 2H), 7.80 (d, J=1.7 Hz, 2H), 7.70 (d, J=7.4 Hz, 1H), 7.67-7.62 (m, 4H), 7.49 (t, J=7.4 Hz, 2H), 7.30 (d, J=8.3 Hz, 3H), 6.58 (t, J=12.3 Hz, 1H), 6.41 (s, 2H), 6.36-6.27 (m, 3H), 6.26-6.12 (m, 2H), 5.60 (dd, J=13.1, 6.4 Hz, 3H), 4.86 (s, 2H), 4.48 (dd, J=29.1, 16.7 Hz, 2H), 4.35-4.27 (m, 2H), 4.07 (d, J=4.8 Hz, 4H), 3.59 (s, 2H), 3.50 (d, J=5.7 Hz, 2H), 3.34-3.29 (m, 2H), 2.78 (t, J=6.8 Hz, 2H), 2.13 (t, J=6.7 Hz, 2H), 2.07 (t, J=7.2 Hz, 2H), 1.68 (s, 12H), 1.53-1.48 (m, 8H), 1.35 (dt, J=15.8, 8.1 Hz, 4H).

2. Sequencing Performed with Nucleotide Analogues Containing the SS-Hot G Synthesized Above

Nucleotide substrates: fluorescent labeled standard hot dNTP (available from MGISEQ-2000RS high-throughput sequencing kit (FCL SE50), MGI Tech Co., Ltd., cat. no. 1000012551), wherein hot dGTP was replaced by SS-hot G synthesized above; nucleotide analogues cold dNTP of the invention (four kinds, named as SS-cold) with structures showed below.

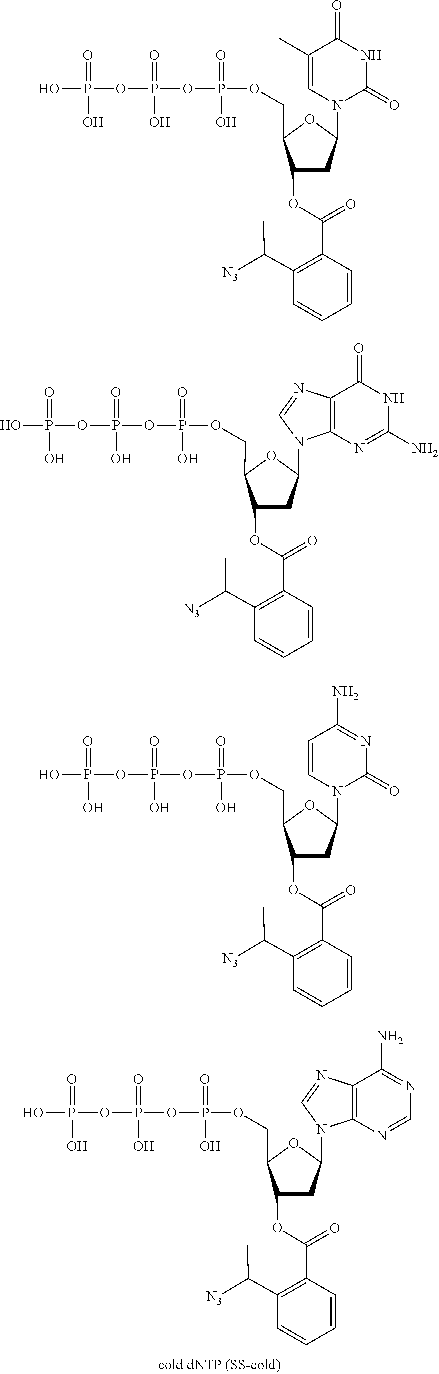

Sequencing procedure was same as that above in Sequencing Example 1. Results of Basecall analysis were showed in Table 6.

TABLE 6
Results of Basecall analysis

| | SS-hot G + SS-cold dNTP, |
|---|---|
| nucleotide analogues | standard hot dNTP for others |
| Reference | Ecoli.fa |
| Cycle Number | 50 |
| Q30(%) | 53.08 |
| Lag(%) | 0.24 |
| Runon(%) | 0.32 |
| ESR(%) | 66.96 |
| Mapping Rate, % | 0.13 |
| AvgError Rate, % | 5.14 |

1. A compound of formula (A) or a salt thereof, 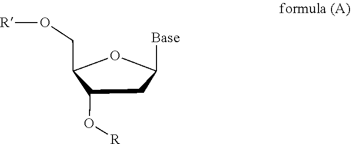
wherein:
R is a reversible blocking group, and R is selected from 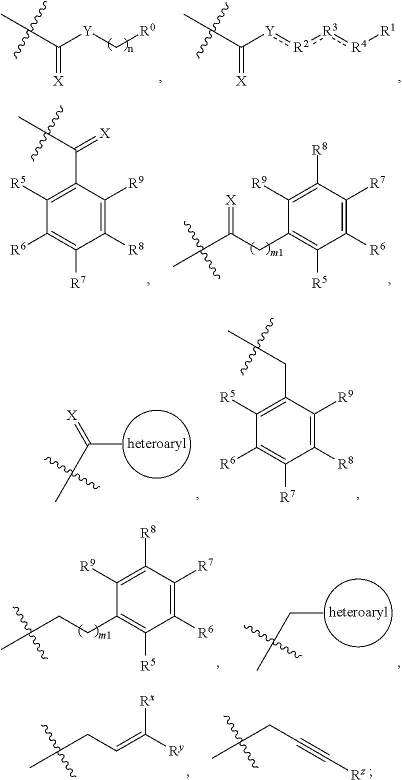
preferably, R is selected from 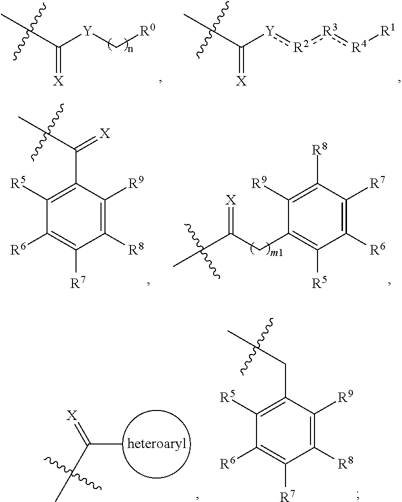
more preferably, R is selected from 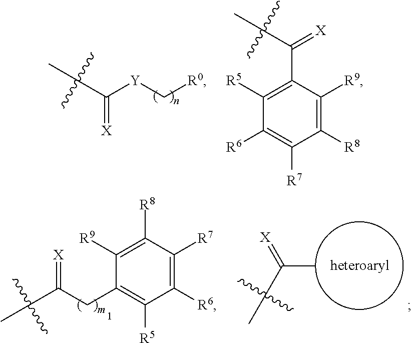
the heteroaryl is selected from the followings: 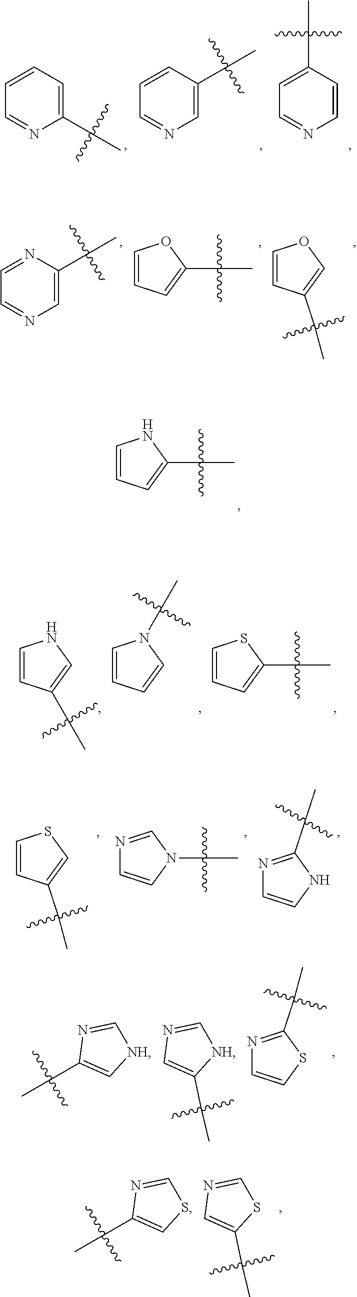
each heteroaryl is independently optionally substituted by one or more (e.g., 2, 3, 4, 5 or 6, preferably 2) R″s;
preferably, the heteroaryl is selected from the followings: 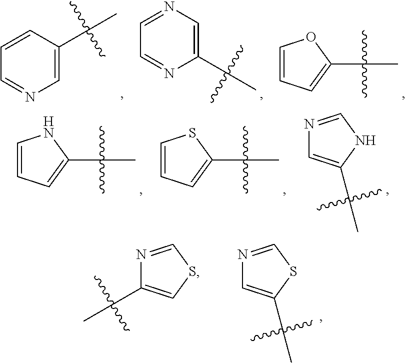
each heteroaryl is independently optionally substituted by one or more (e.g., 2, 3, 4, 5 or 6, preferably 2) R″s;
more preferably, the heteroaryl is selected from the followings: 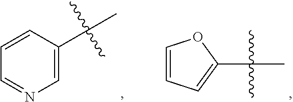 [SMILES: *c1cccnc1 ; *c1ccco1]
each heteroaryl is independently optionally substituted by one or more (e.g., 2, 3, 4, 5 or 6, preferably 2) R″s;
each X is independently selected from O, NH, S;
preferably, X is O;
each Y is independently selected from a direct bond, O, NH, S, CH, CH₂, C(CH₃)₂;
preferably, each Y is independently selected from a direct bond or CH₂;
R⁰ is selected from —N₃, —SS—C₁-C₆ alkyl (e.g., —SS-methyl, —SS-ethyl, —SS-isopropyl, —SS-tertbutyl), —ONH₂, —OCOR^{m}, —OCONHR^{m}, wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl);
preferably, R⁰ is —N₃ or —SS—C₁-C₆ alkyl (e.g., —SS-methyl, —SS-ethyl, —SS-isopropyl, —SS-tertbutyl);
more preferably, R⁰ is —N₃;
R¹ is selected from —N₃, —SS—C₁-C₆ alkyl (e.g., —SS-methyl, —SS-ethyl, —SS-isopropyl, —SS-tertbutyl), —ONH₂, —OCOR^{m}, —OCONHR^{m}, wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl);
preferably, R¹ is —N₃ or —SS—C₁-C₆ alkyl (e.g., —SS-methyl, —SS-ethyl, —SS-isopropyl, —SS-tertbutyl);
more preferably, R¹ is —N₃;
R², R³, and R⁴ are each independently selected from O, NH, S, CH, CH₂, C(CH₃)₂;
preferably, R², R³, and R⁴ are each independently selected from O, NH, S, CH, CH₂, C(CH₃)₂, and meet the following conditions: when R² is selected from O or S, R³ and R⁴ are CH₂; when R³ is selected from O or S, R² and R⁴ are CH₂; when R⁴ is selected from O or S, R² and R³ are CH₂; when R² is C(CH₃)₂, R³ and R⁴ are CH₂; when R³ is C(CH₃)₂, R² and R⁴ are CH₂; when R⁴ is C(CH₃)₂, R² and R³ are CH₂; when Y is CH, R² is CH, and R³ and R⁴ are CH₂;
more preferably, R², R³, and R⁴ are each independently selected from CH, CH₂;
most preferably, R² and R³ are CH, and R⁴ is CH₂; [symbol]
represents a single bond [symbol] or a double bond [symbol] ;
each R″ is independently selected from H, —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy), 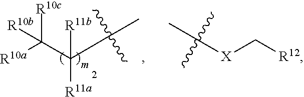
and is not H at the same time;
preferably, when each heteroaryl is independently optionally substituted by one R″,
each R″ is independently selected from 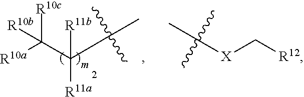
preferably, R″ is 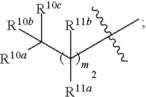
or preferably, when each heteroaryl is independently optionally substituted by more (e.g., 2, 3, 4, 5 or 6, preferably 2) R″s, one of the R″ is selected from 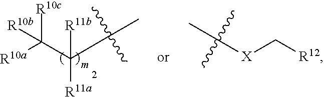
and the remaining R″(s) is(are) each independently selected from —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy);
or more preferably, when each heteroaryl is independently optionally substituted by more (e.g., 2, 3, 4, 5 or 6, preferably 2) R″s, one of the R″ is 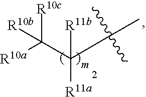
and the remaining R″(s) is(are) each independently selected from nitro, aliphatic alkyl (e.g., C₁-C₆alkyl), F, I, Br, Cl;
or most preferably, when each heteroaryl is independently optionally substituted by more (e.g., 2, 3, 4, 5 or 6, preferably 2) R″s, one of the R″ is 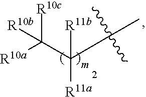
and the remaining R″(s) is(are) each independently selected from nitro, C₁-C₆ alkyl, preferably, the remaining R″(s) is(are) C₁-C₆ alkyl;
R⁵, R⁶, R⁷, R⁸, R⁹, R^{x}, R^{y}, and R^{z} are each independently selected from H, —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy), C₁-C₆ alkyl-C(═O)—NH₂—, 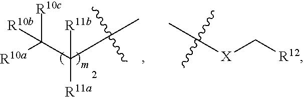
and R¹, R⁶, R⁷, R⁸, and R⁹ are not H at the same time;
preferably, any one of R⁵, R⁶, R⁷, R⁸, and R⁹ is selected from 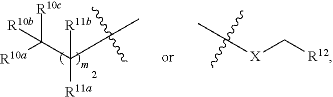
and the remaining four thereof are each independently selected from H, —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy), C₁-C₆ alkyl-C(═O)—NH₂—;
more preferably, any one of R⁵, R⁶, R⁷, R⁸, and R⁹ is 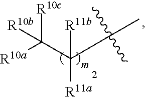
and the remaining four thereof are each independently selected from H, nitro, C₁-C₆ alkyl, C₁-C₆ alkoxy, C₁-C₆ alkyl-C(═O)—NH₂—;
further preferably, of R⁵, R⁶, R⁷, R⁸, and R⁹, R⁵ or R⁹ is 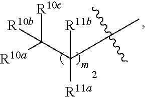
and the remaining four thereof are each independently selected from H, nitro, methoxy, acetamido;
most preferably, of R⁵, R⁶, R⁷, R⁸, and R⁹, R⁵ or R⁹ is 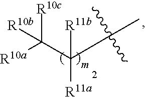
R⁷ is selected from H, nitro, methoxy, acetamido, and the remaining three thereof are H;
preferably, any one of R^{x} and R^{y} is selected from 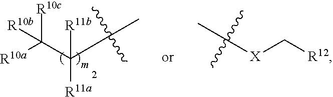
and the other is selected from H, —N₃, nitro, amino, sulfo, carboxyl, aliphatic alkyl (e.g., C₁-C₆alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy);
more preferably, any one of R^{x} and R^{y} is selected from 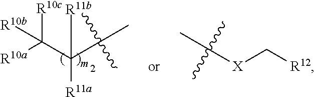
and the other is H;
most preferably, any one of R^{x} and R^{Y} is 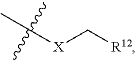
and the other is H;
preferably, R^{z} is selected from 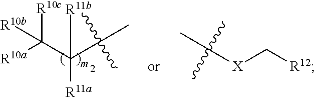
more preferably, R^{z} is 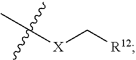
R^{10a}, R^{10b}, R^{10c}, R^{11a}, R^{11b}, and R¹² are each independently selected from H, —N₃, —SS—C₁-C₆ alkyl (e.g., —SS-methyl, —SS-ethyl, —SS-isopropyl, —SS-tertbutyl or —SS-isobutyl), —ONH₂, —OCOR^{m}, —OCONHR^{m}, aliphatic alkyl (e.g., C₁-C₆ alkyl, particularly such as methyl, ethyl, isopropyl, tertbutyl), aromatic alkyl (e.g., phenyl C₁-C₆ alkyl), cycloalkyl (e.g., C₃-C₆ cycloalkyl), wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl), and R^{10a}, R^{10b}, and R^{10c} are not H at the same time;
preferably, any one of R^{10a}, R^{10b}, and R^{10c} is selected from —N₃, —SS—C₁-C₆ alkyl (e.g., —SS-methyl, —SS-ethyl, —SS-isopropyl, —SS-tertbutyl or —SS-isobutyl), —ONH₂, —OCOR^{m}, —OCONHR^{m}, and the other two are each independently selected from H, aliphatic alkyl (e.g., C₁-C₆ alkyl, particularly such as methyl, ethyl, isopropyl, tertbutyl), aromatic alkyl (e.g., phenyl C₁-C₆ alkyl), cycloalkyl (e.g., C₃-C₆ cycloalkyl), wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl);
more preferably, any one of R^{10a}, R^{10b}, and R^{10c} is —N₃ or —SS—C₁-C₆ alkyl (e.g., —SS-methyl, —SS-ethyl, —SS-isopropyl, —SS-tertbutyl or —SS-isobutyl), and the other two are each independently selected from H, aliphatic alkyl (e.g., C₁-C₆ alkyl, particularly such as methyl, ethyl, isopropyl, tertbutyl);
most preferably, any one of R^{10a}, R^{10b}, and R^{10c} is —N₃, —SS-methyl, —SS-ethyl, —SS-isopropyl, —SS-tertbutyl or —SS-isobutyl, and the other two are each independently selected from H or methyl;
preferably, R^{11a} and R^{11b} are each independently selected from H or aliphatic alkyl (e.g., C₁-C₆ alkyl, particularly such as methyl, ethyl, isopropyl, tertbutyl);
more preferably, R^{11a} and R^{11b} are H;
preferably, R¹² is selected from —N₃, —SS—C₁-C₆ alkyl (e.g., —SS-methyl, —SS-ethyl, —SS-isopropyl, —SS-tertbutyl), —ONH₂, —OCOR^{m}, —OCONHR^{m}, wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl);
more preferably, R¹² is —N₃ or —SS—C₁-C₆ alkyl (e.g., —SS-methyl, —SS-ethyl, —SS-isopropyl, —SS-tertbutyl);
most preferably, R¹² is —N₃;
n is selected from 1, 2, 3, 4, or 5; preferably, n is selected from 1, 2, or 3; more preferably, n is 2;
each m₁ is independently selected from 1, 2, 3, 4, 5, and 6; preferably, m₁ is 1;
each m₂ is independently selected from 0, 1, 2, 3, 4, 5, and 6; preferably, each m₂ is independently selected from 0 or 1;
most preferably, 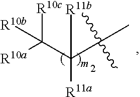
as a whole, is selected from 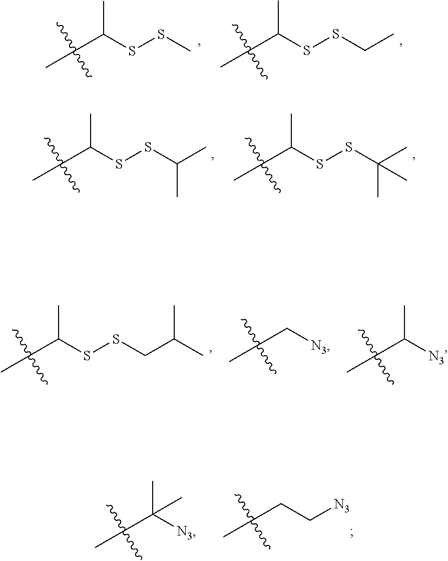
R′ is selected from H, monophosphate group 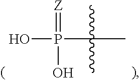
diphosphate group 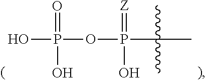
triphosphate group 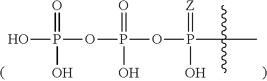
or tetraphosphate group 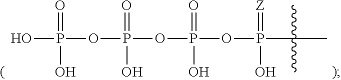
preferably, R′ is a triphosphate group 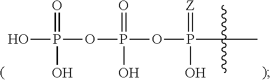
each Z is independently selected from O, S, BH; preferably, Z is O;
Base is selected from a base, a deaza base or a tautomer thereof, for example, Base is selected from adenine, 7-deazaadenine, thymine, uracil, cytosine, guanine, 7-deazaguanine or a tautomer thereof;
preferably, Base is selected from

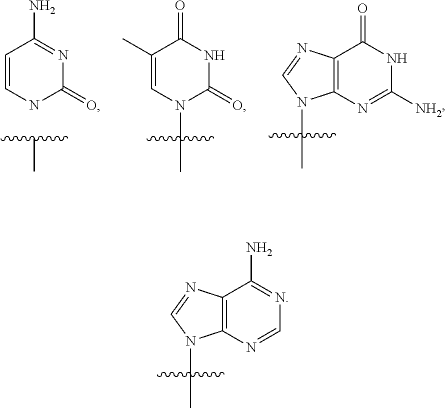

2. The compound or a salt thereof according to claim 1, wherein the compound has a structure represented by the formula (I), 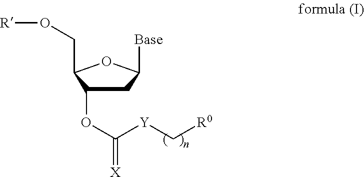
wherein:
X is selected from O, NH, S;
preferably, X is O;
Y is selected from a direct bond, O, NH, S, CH, CH₂, C(CH₃)₂;
preferably, Y is CH₂;
R⁰ is selected from —N₃, —SS—C₁-C₆ alkyl (e.g., —SS-methyl, —SS-ethyl, —SS-isopropyl, —SS-tertbutyl), —ONH₂, —OCOR^{m}, —OCONHR^{m}, wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl);
preferably, R⁰ is —N₃ or —SS—C₁-C₆ alkyl (e.g., —SS-methyl, —SS-ethyl, —SS-isopropyl, —SS-tertbutyl);
more preferably, R⁰ is —N₃;
n is selected from 1, 2, 3, 4, and 5;
preferably, n is selected from 1, 2, and 3;
more preferably, n is 2;
R′ is selected from H, monophosphate group 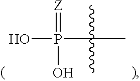
diphosphate group 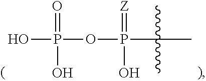
triphosphate group 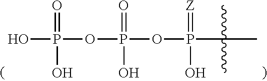
or tetraphosphate group 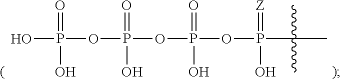
preferably, R is a triphosphate group 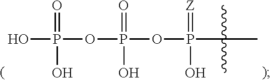
each Z is independently selected from O, S, BH;
preferably, Z is O;
Base is selected from a base, a deaza base or a tautomer thereof, for example, Base is selected from adenine, 7-deazaadenine, thymine, uracil, cytosine, guanine, 7-deazaguanine or a tautomer thereof;
preferably, Base is selected from

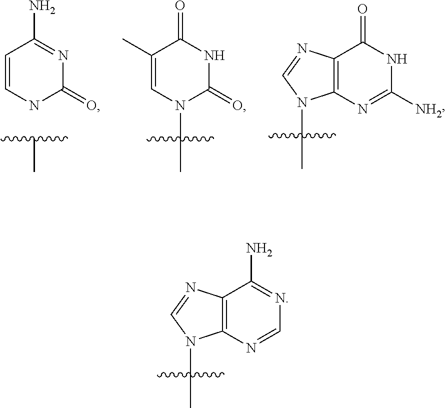

3. The compound or a salt thereof according to claim 1, wherein the compound has a structure represented by the formula (III) 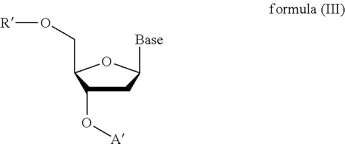
wherein:
A′ is selected from 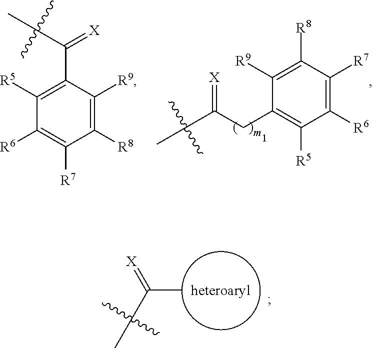
the heteroaryl is selected from the followings: 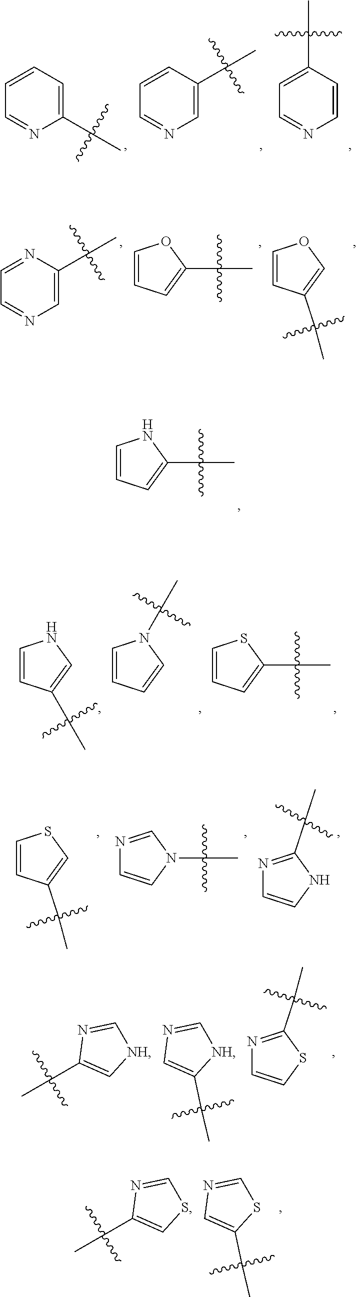
each heteroaryl is independently optionally substituted by one or more (e.g., 2, 3, 4, 5 or 6, preferably 2) R″s;
preferably, the heteroaryl is selected from the followings: 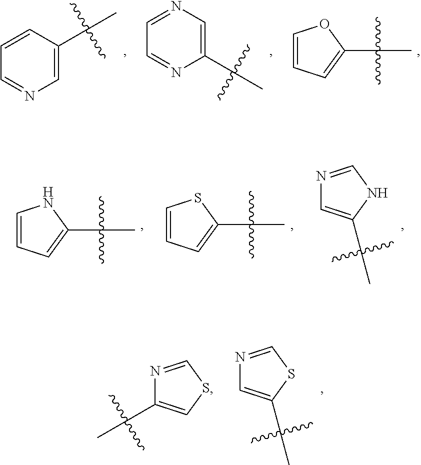
each heteroaryl is independently optionally substituted by one or more (e.g., 2, 3, 4, 5 or 6, preferably 2) R″s;
more preferably, the heteroaryl is selected from the followings: 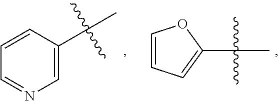 [SMILES: *c1cccnc1 ; *c1ccco1]
each heteroaryl is independently optionally substituted by one or more (e.g., 2, 3, 4, 5 or 6, preferably 2) R″s;
or more preferably, the heteroaryl is selected from the followings: 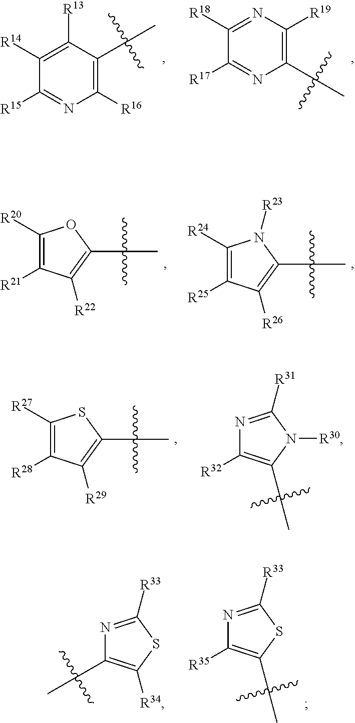
most preferably, the heteroaryl is selected from the followings: 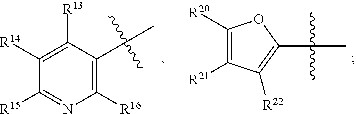
each R″ is independently selected from H, —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy), 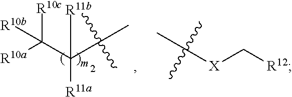
preferably, when each heteroaryl is independently optionally substituted by one R″, each R″ is independently selected from 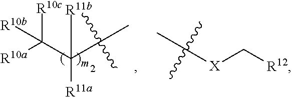
preferably, R″ is 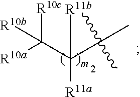
or preferably, when each heteroaryl is independently optionally substituted by more (e.g., 2, 3, 4, 5 or 6, preferably 2) R″s, one of the R″ is selected from 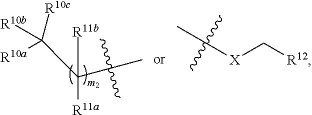
and the remaining R″(s) is(are) each independently selected from —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy);
or more preferably, when each heteroaryl is independently optionally substituted by more (e.g., 2, 3, 4, 5 or 6, preferably 2) R″s, one of the R″ is 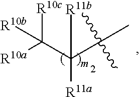
and the remaining R″(s) is(are) each independently selected from nitro, aliphatic alkyl (e.g., C₁-C₆alkyl), F, I, Br, Cl;
or most preferably, when each heteroaryl is independently optionally substituted by more (e.g., 2, 3, 4, 5 or 6, preferably 2) R″s, one of the R″ is 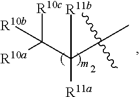
and the remaining R″(s) is(are) each independently selected from nitro, C₁-C₆ alkyl, preferably, the remaining R″(s) is(are) C₁-C₆ alkyl, more preferably, the remaining R″(s) is(are) methyl;
of R¹³, R¹⁴, R¹⁵, and R¹⁶, R¹⁶ is 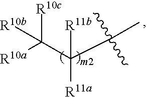
R¹³, R¹⁴, and R¹⁵ are each independently selected from H, nitro, C₁-C₆ alkyl (e.g., methyl);
preferably, of R¹³, R¹⁴, R¹⁵, and R¹⁶, R¹⁶ is 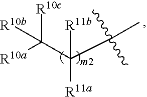
R¹³, R¹⁴, and R¹⁵ are H;
of R¹⁷, R¹⁸, and R¹⁹, R¹⁹ is 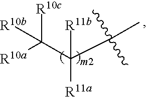
R¹⁷ and R¹⁸ are each independently selected from H, nitro, C₁-C₆ alkyl (e.g., methyl);
preferably, of R¹⁷, R¹⁸, and R¹⁹, R¹⁹ is 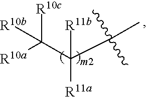
R¹⁷ and R¹⁸ are H;
of R²⁰, R²¹, and R²², R²² is 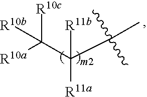
R²⁰ and R²¹ are each independently selected from H, nitro, C₁-C₆ alkyl (e.g., methyl);
preferably, of R²⁰, R²¹, and R²², R²² is 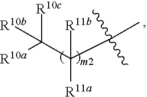
R²⁰ and R²¹ are H;
of R²³, R²⁴, R²⁵, and R²⁶, R²⁶ is 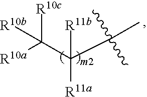
R²³, R²⁴, and R²⁵ are each independently selected from H, nitro, C₁-C₆ alkyl (e.g., methyl);
preferably, of R²³, R²⁴, R²⁵, and R²⁶, R²⁶ is 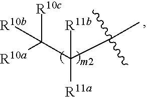
R²³, R²⁴, and R²⁵ are H;
of R²⁷, R²⁸, and R²⁹, R²⁹ is 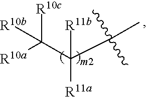
R²⁷ and R²⁸ are each independently selected from H, nitro, C₁-C₆ alkyl (e.g., methyl);
preferably, of R²⁷, R²⁸, and R²⁹, R²⁹ is 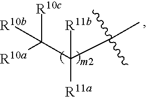
R²⁷ and R²⁸ are H;
of R³⁰, R³¹, and R³², R³² is 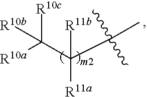
R³⁰ and R³¹ are each independently selected from H, nitro, C₁-C₆ alkyl (e.g., methyl);
preferably, of R³⁰, R³¹, and R³², R³² is 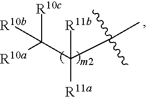
R³⁰ and R³¹ are H;
of R³³, R³⁴, and R³⁵, R³⁴ or R³⁵ is 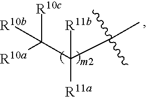
R³³ is selected from H, nitro, C₁-C₆alkyl (e.g., methyl);
preferably, of R³³, R³⁴, and R³⁵, R³⁴ or R³⁵ is 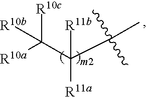
and R³³ is H or methyl;
X is selected from O, NH, S;
preferably, X is O;
R⁵, R⁶, R⁷, R⁸, and R⁹ are each independently selected from H, —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy), C₁-C₆ alkyl-C(═O)—NH₂—, 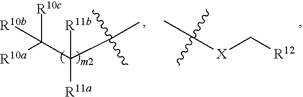
and R⁵, R⁶, R⁷, R⁸, and R⁹ are not H at the same time;
preferably, any one of R⁵, R⁶, R⁷, R⁸, and R⁹ is selected from 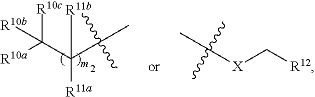
and the remaining four thereof are each independently selected from H, —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy), C₁-C₆ alkyl-C(═O)—NH₂—;
more preferably, any one of R⁵, R⁶, R⁷, R⁸, and R⁹ is 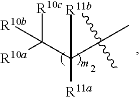
and the remaining four thereof are each independently selected from H, nitro, C₁-C₆ alkyl, C₁-C₆ alkoxy, C₁-C₆ alkyl-C(═O)—NH₂—;
further preferably, of R⁵, R⁶, R⁷, R⁸, and R⁹, R⁵ or R⁹ is 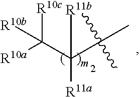
and the remaining four thereof are each independently selected from H, nitro, methoxy, acetamido;
most preferably, of R⁵, R⁶, R⁷, R⁸, and R⁹, R⁵ or R⁹ is 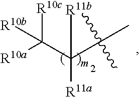
R⁷ is selected from H, nitro, methoxy, acetamido, and the remaining three thereof are H;
R^{10a}, R^{10b}, R^{10c}, R^{11a}, R^{11b}, and R¹² are each independently selected from H, —N₃, —SS—C₁-C₆ alkyl (e.g., —SS-methyl, —SS-ethyl, —SS-isopropyl, —SS-tertbutyl or —SS-isobutyl), —ONH₂, —OCOR^{m}, —OCONHR^{m}, aliphatic alkyl (e.g., C₁-C₆ alkyl, particularly such as methyl, ethyl, isopropyl, tertbutyl), aromatic alkyl (e.g., phenyl C₁-C₆ alkyl), cycloalkyl (e.g., C₃-C₆ cycloalkyl), wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl), and R^{10a}, R^{10b}, and R^{10c} are not H at the same time;
preferably, any one of R^{10a}, R^{10b}, and R^{10c} is selected from —N₃, —SS—C₁-C₆ alkyl (e.g., —SS-methyl, —SS-ethyl, —SS-isopropyl, —SS-tertbutyl or —SS-isobutyl), —ONH₂, —OCOR^{m}, —OCONHR^{m}, and the other two are each independently selected from H, aliphatic alkyl (e.g., C₁-C₆ alkyl, particularly such as methyl, ethyl, isopropyl, tertbutyl), aromatic alkyl (e.g., phenyl C₁-C₆ alkyl), cycloalkyl (e.g., C₃-C₆ cycloalkyl), wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl);
more preferably, any one of R^{10a}, R^{10b}, and R^{10c} is —N₃ or —SS—C₁-C₆ alkyl (e.g., —SS-methyl, —SS-ethyl, —SS-isopropyl, —SS-tertbutyl, —SS-isobutyl), and the other two are each independently selected from H, C₁-C₆ alkyl (e.g., methyl, ethyl, isopropyl, tertbutyl);
most preferably, any one of R^{10a}, R^{10b}, and R^{10c} is —N₃, —SS-methyl, —SS-ethyl, —SS-isopropyl, —SS-tertbutyl, —SS-isobutyl, and the other two are each independently selected from H, methyl;
preferably, R^{11a} and R^{11b} are each independently selected from H, aliphatic alkyl (e.g., C₁-C₆ alkyl, particularly such as methyl, ethyl, isopropyl, tertbutyl);
more preferably, R^{11a} and R^{11b} are H;
preferably, R¹² is selected from —N₃, —SS—C₁-C₆ alkyl (e.g., —SS-methyl, —SS-ethyl, —SS-isopropyl, —SS-tertbutyl), —ONH₂, —OCOR^{m}, —OCONHR^{m}, wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl);
more preferably, R¹² is —N₃ or —SS—C₁-C₆ alkyl (e.g., —SS-methyl, —SS-ethyl, —SS-isopropyl, —SS-tertbutyl);
most preferably, R¹² is —N₃;
m₁ is selected from 1, 2, 3, 4, 5, and 6; preferably, m₁ is 1;
m₂ is selected from 0, 1, 2, 3, 4, 5, and 6; preferably, m₂ is selected from 0 or 1;
most preferably, 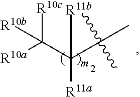
as a whole, is selected from 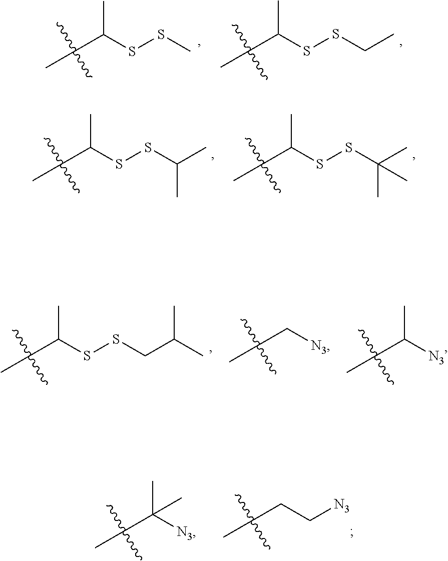
R′ is selected from H, monophosphate group 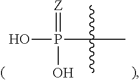
diphosphate group 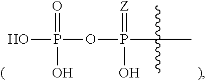
triphosphate group 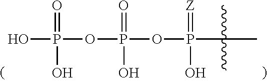
or tetraphosphate group 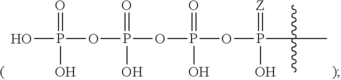
preferably, R′ is a triphosphate group 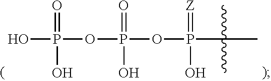
each Z is independently selected from O, S, BH;
preferably, Z is O;
Base is selected from a base, a deaza base or a tautomer thereof, for example, Base is selected from adenine, 7-deazaadenine, thymine, uracil, cytosine, guanine, 7-deazaguanine or a tautomer thereof;
preferably, Base is selected from

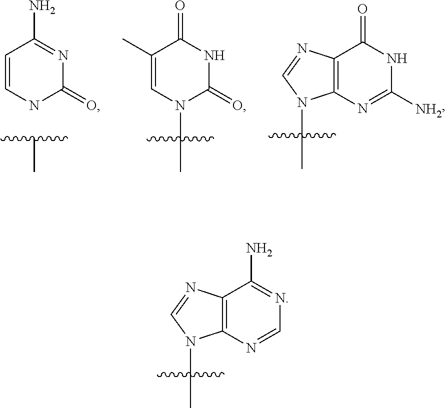

4. The compound or a salt thereof according to claim 1, wherein the compound is selected from the followings:

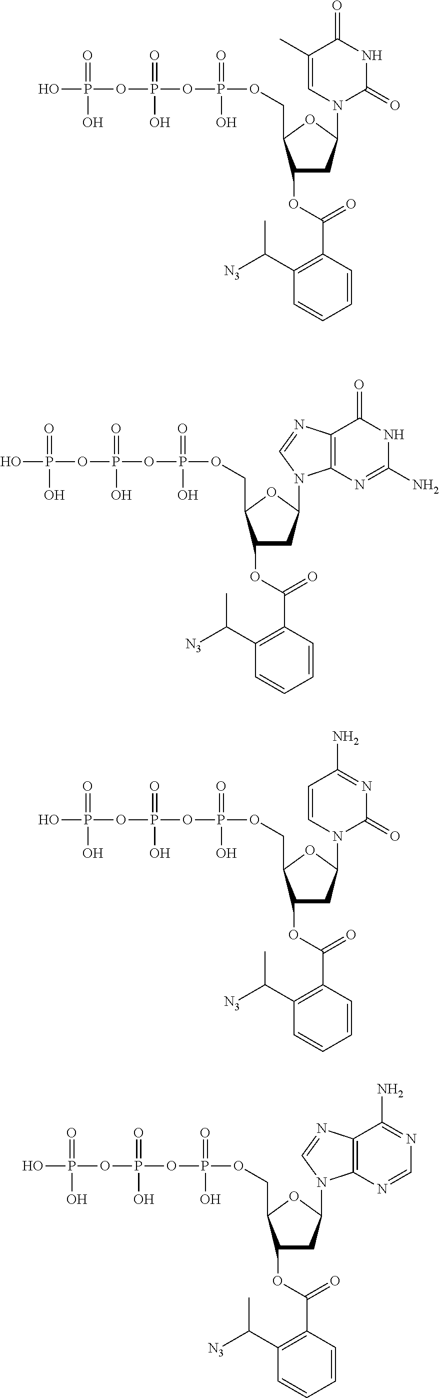 [SMILES: Cc1cn([C@H]2C[C@@H](OC(=O)c3ccccc3C(C)N=[N+]=[N-])[C@@H](COP(=O)(O)OP(=O)(O)OP(=O)(O)O)O2)c(=O)[nH]c1=O ; CC(N=[N+]=[N-])c1ccccc1C(=O)O[C@@H]1C[C@H](n2cnc3c(=O)[nH]c(N)nc32)O[C@@H]1COP(=O)(O)OP(=O)(O)OP(=O)(O)O ; CC(N=[N+]=[N-])c1ccccc1C(=O)O[C@@H]1C[C@H](n2ccc(N)nc2=O)O[C@@H]1COP(=O)(O)OP(=O)(O)OP(=O)(O)O ; CC(N=[N+]=[N-])c1ccccc1C(=O)O[C@@H]1C[C@H](n2cnc3c(N)ncnc32)O[C@@H]1COP(=O)(O)OP(=O)(O)OP(=O)(O)O]

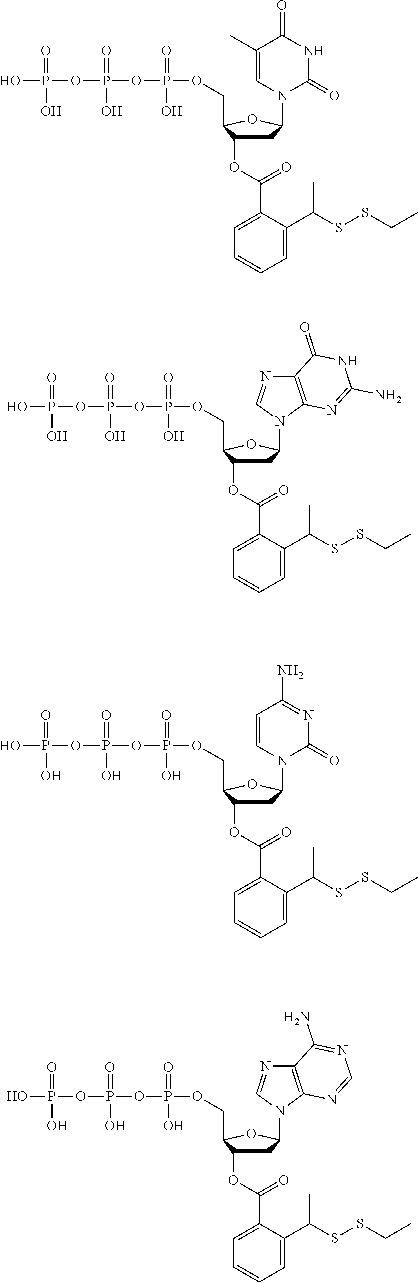

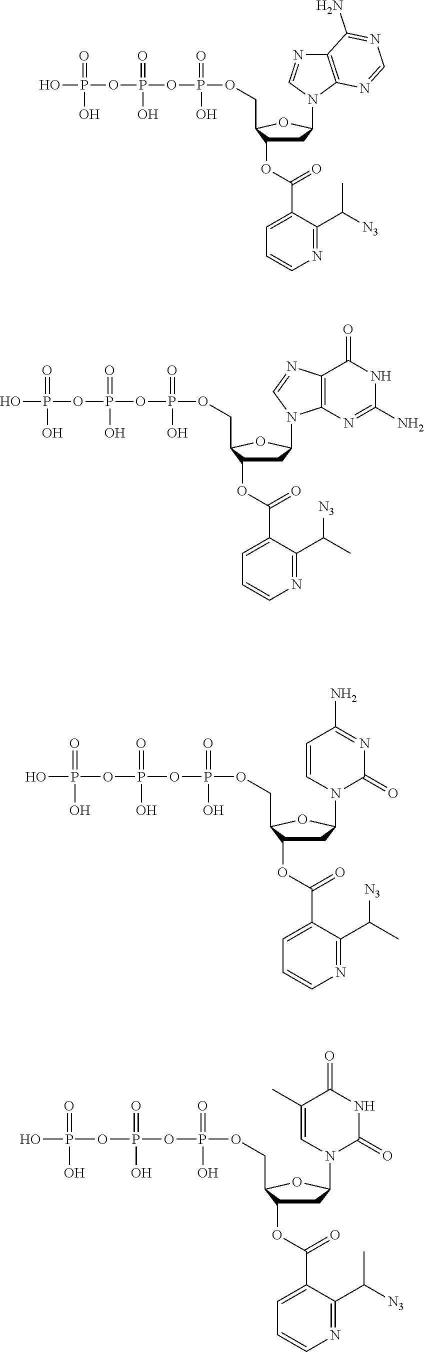

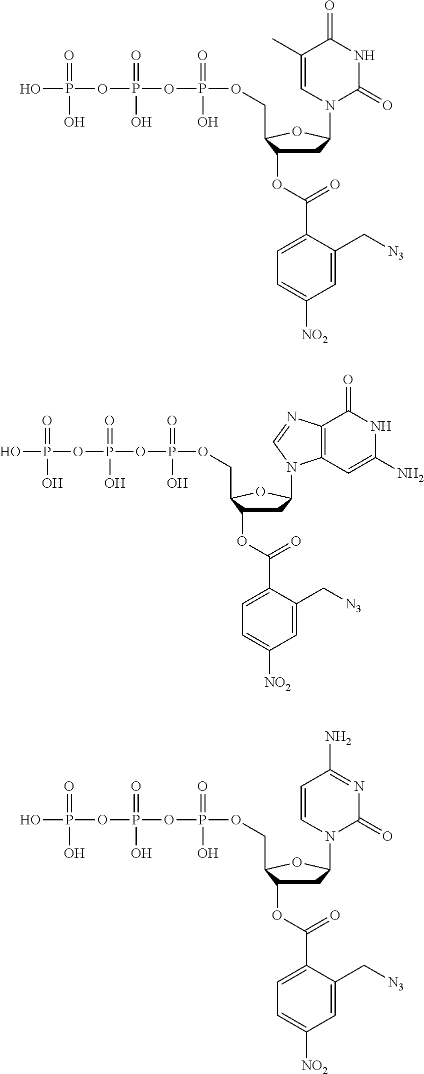

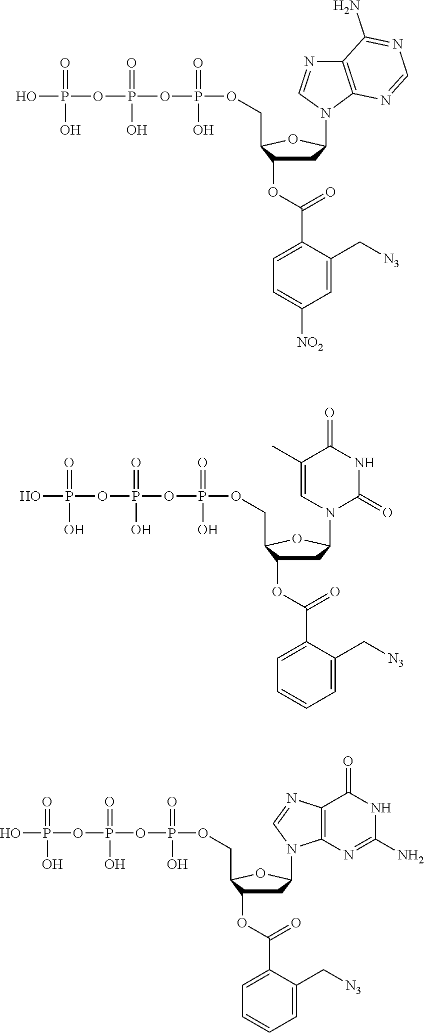

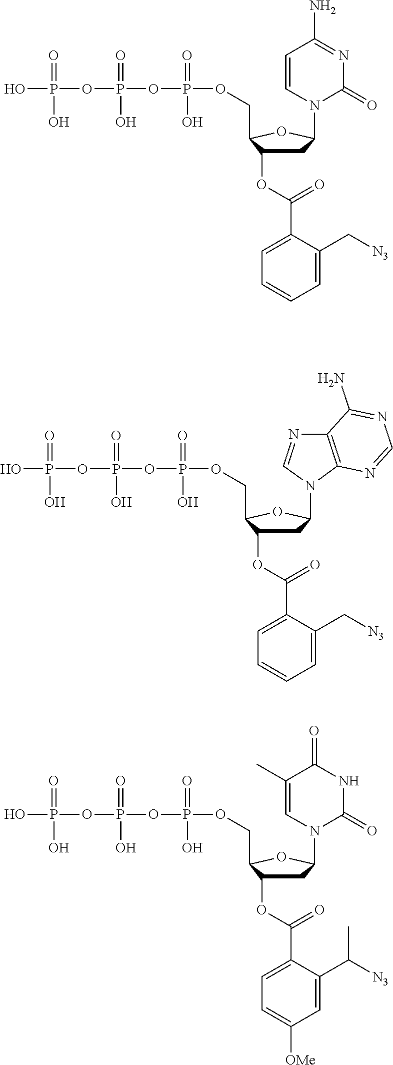

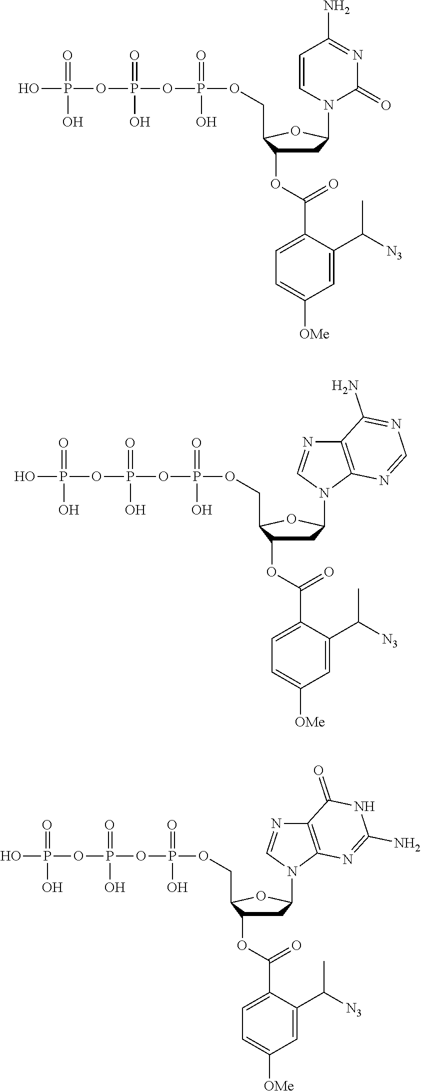

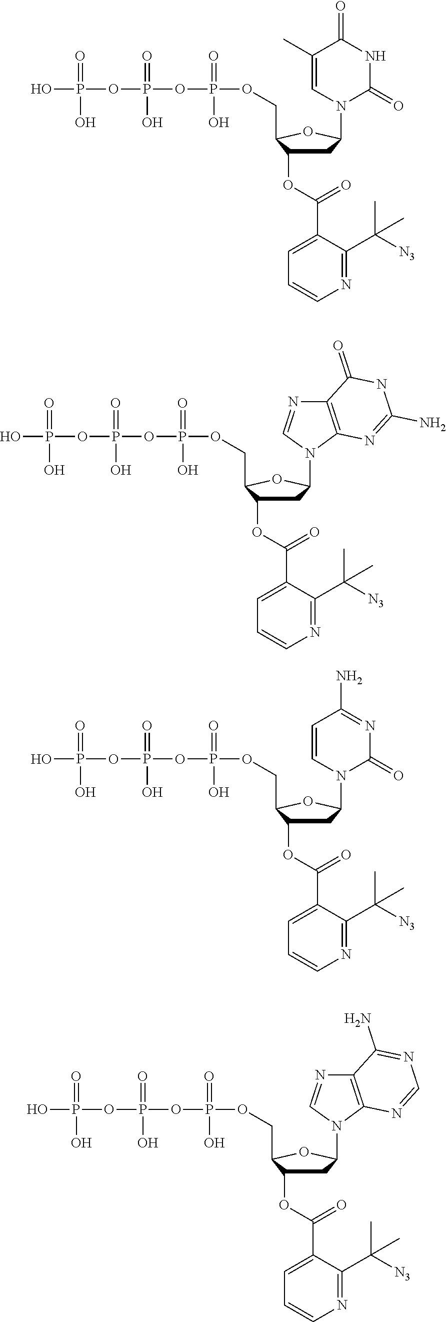

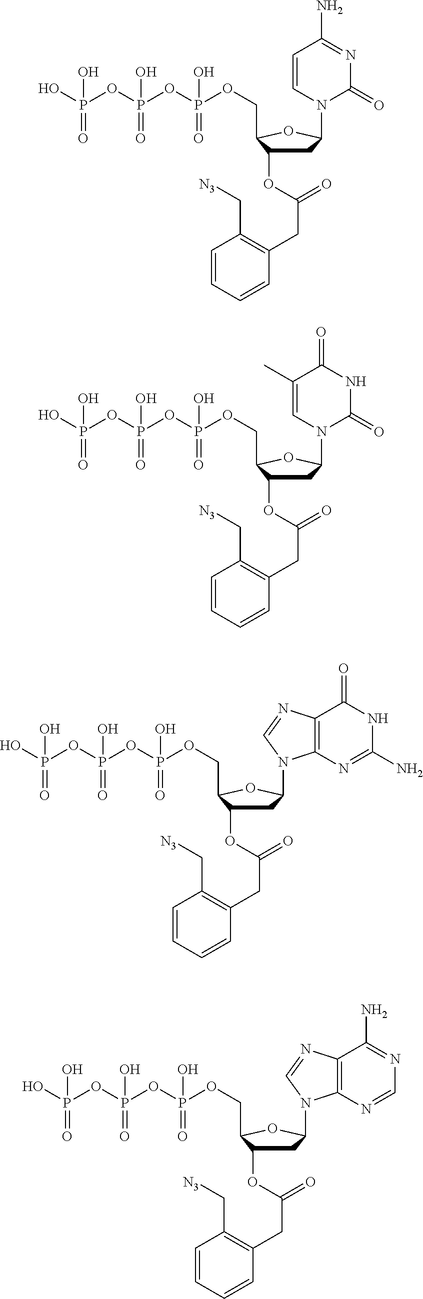

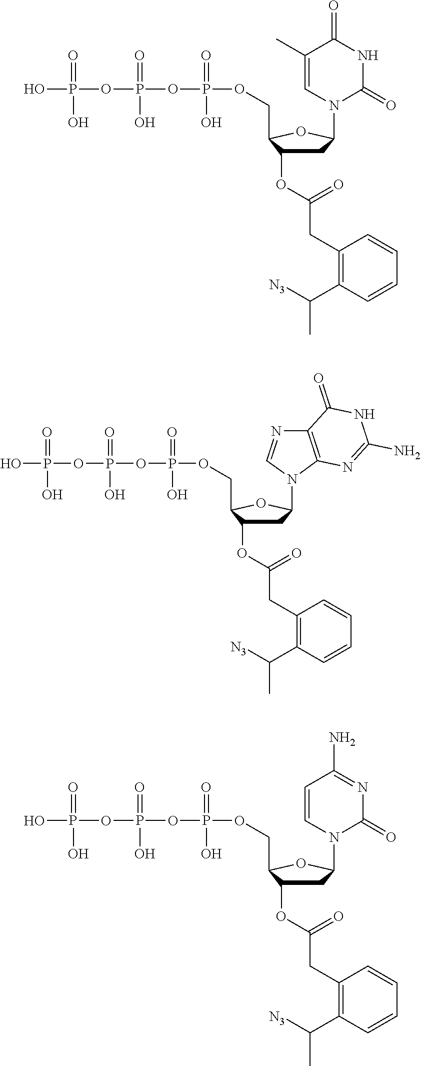

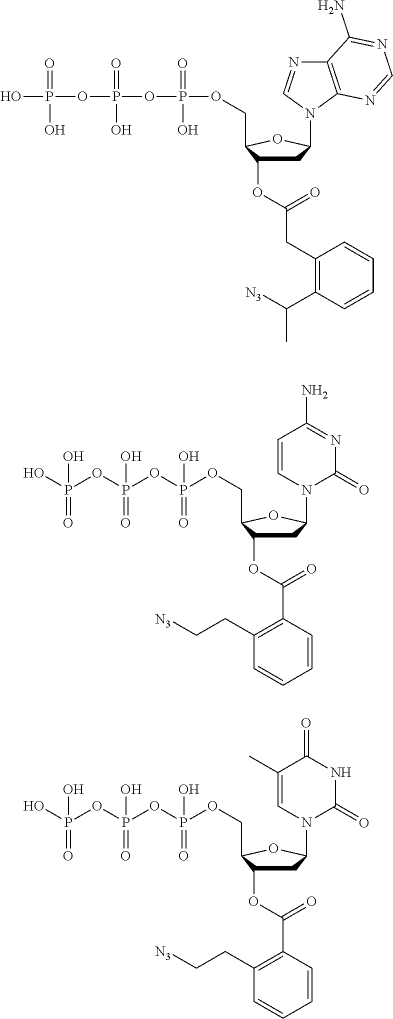

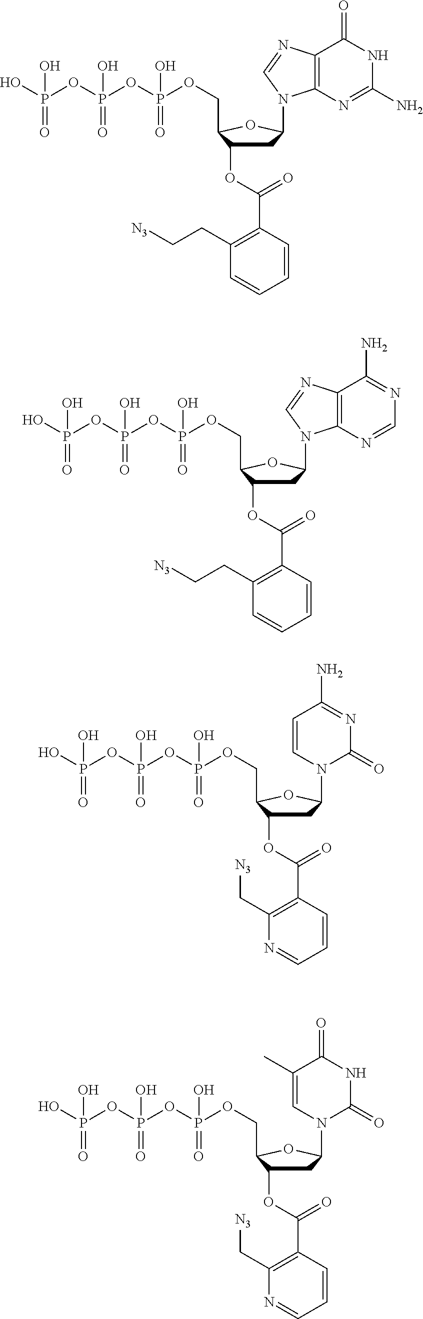

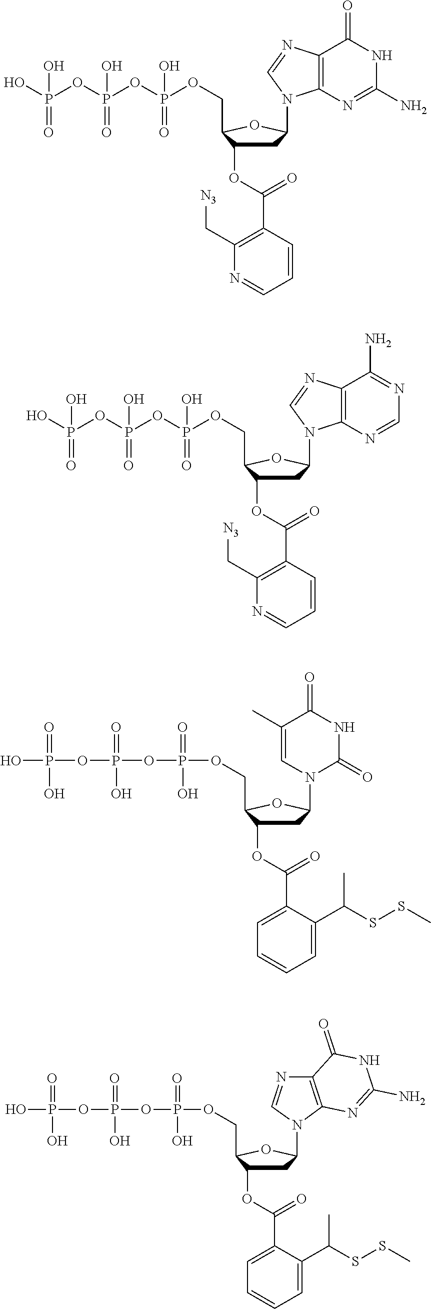

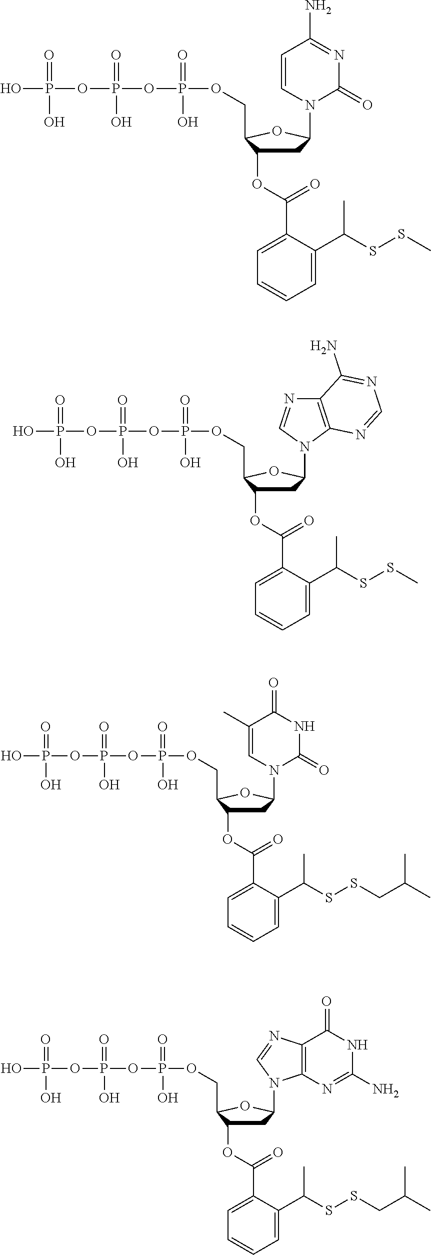

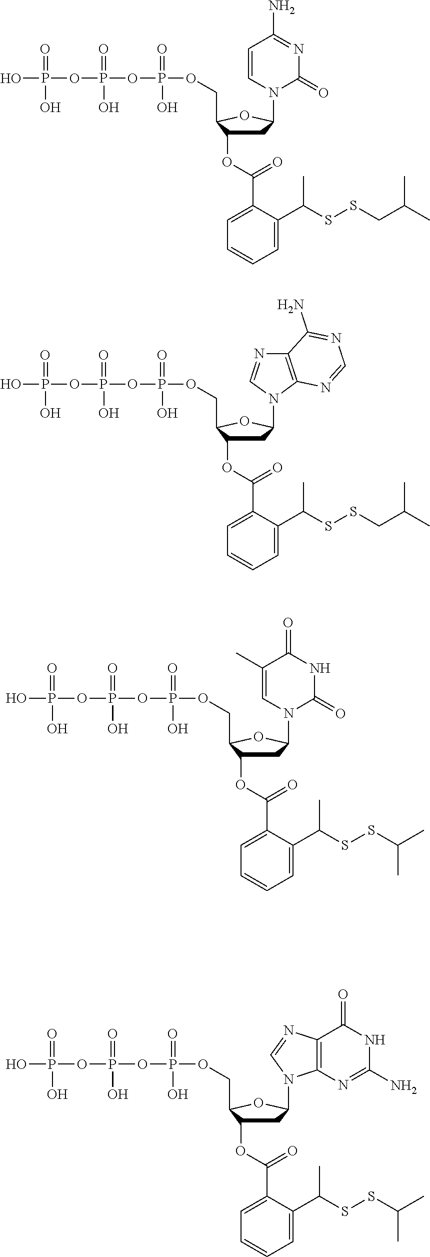

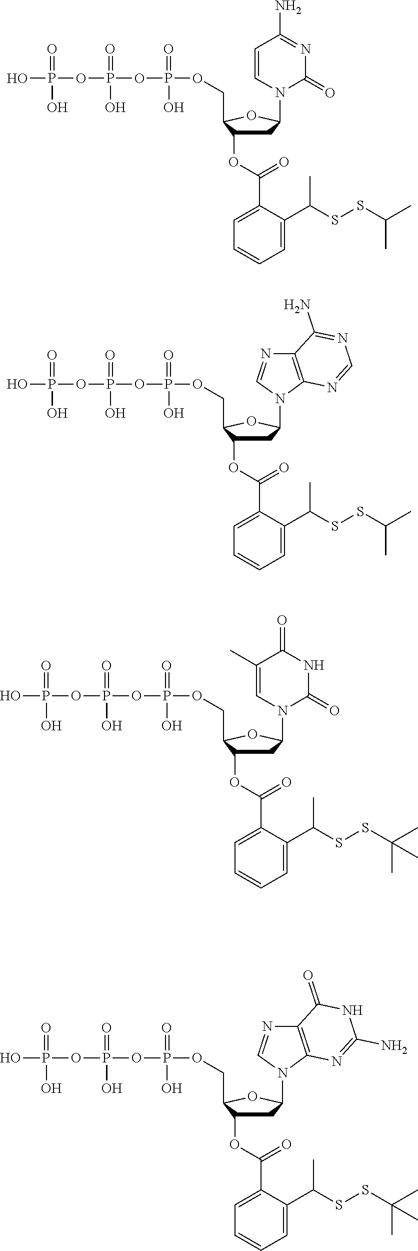

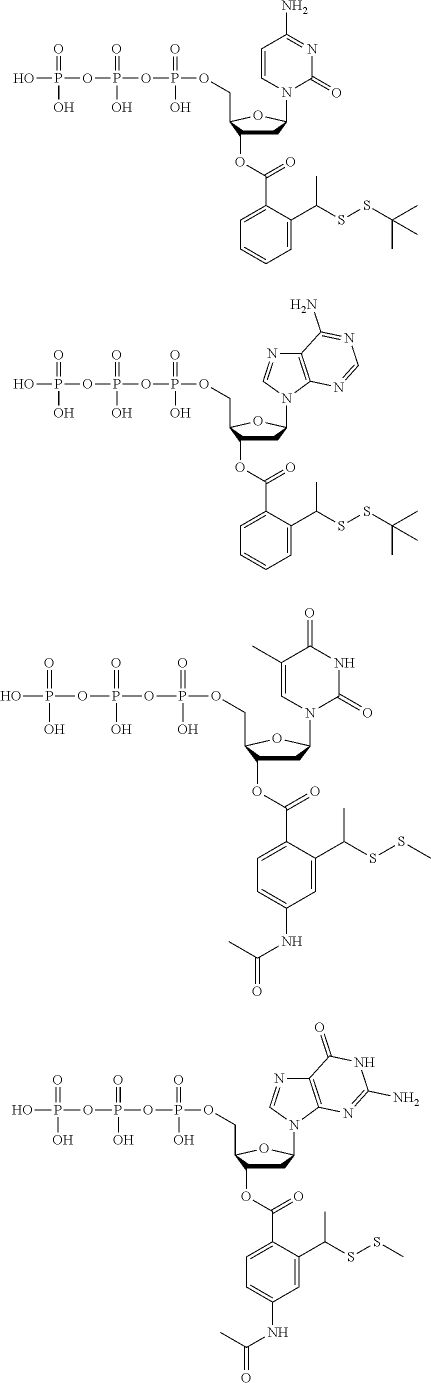

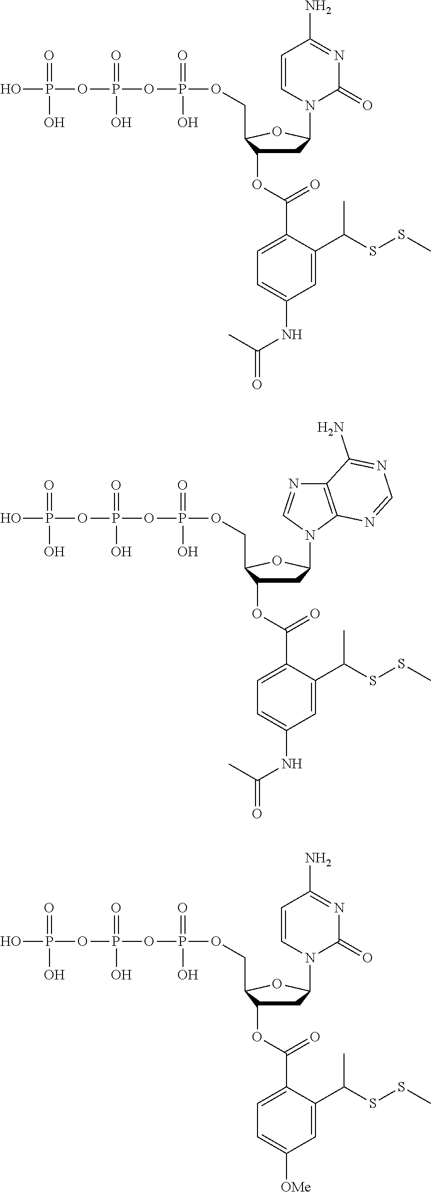

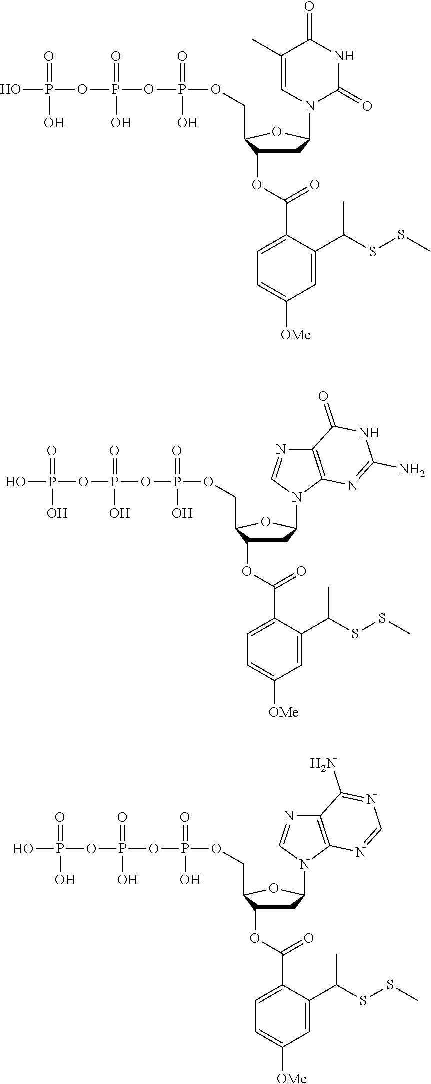

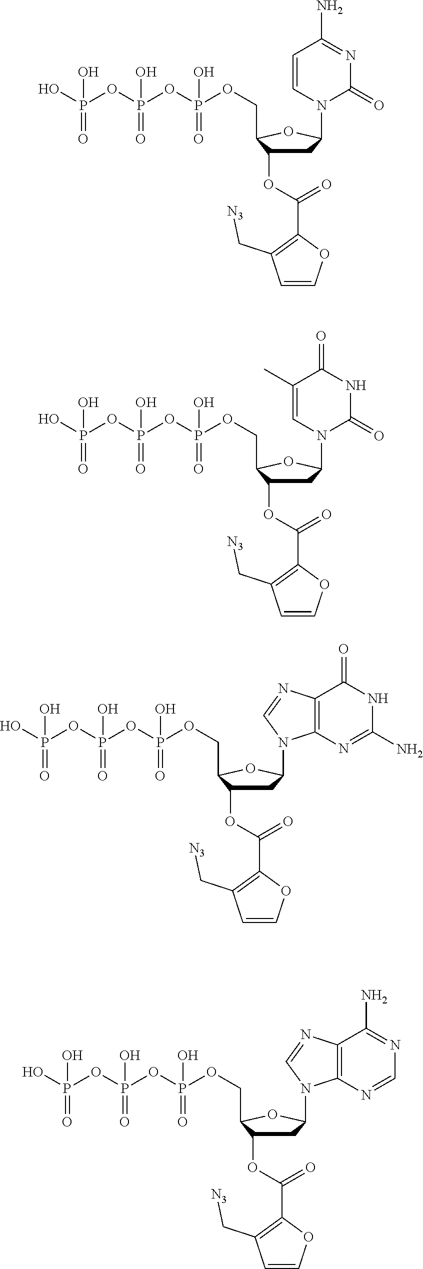

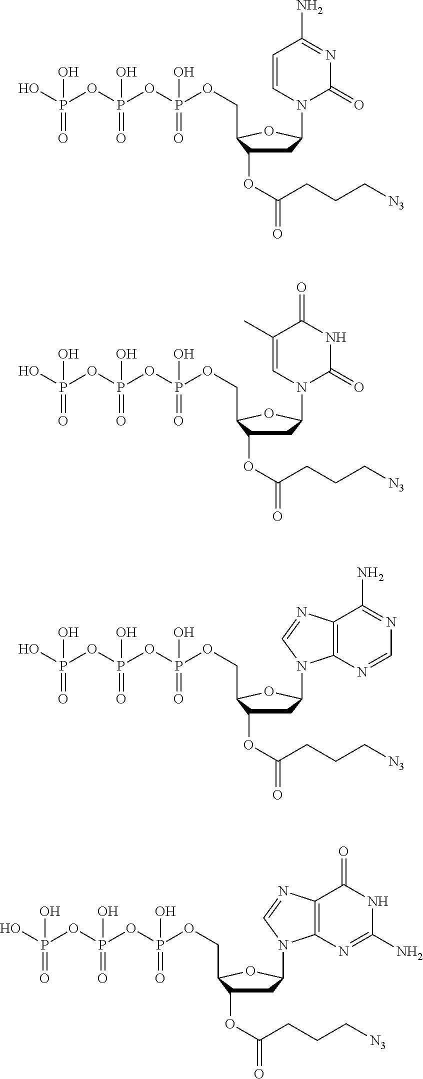

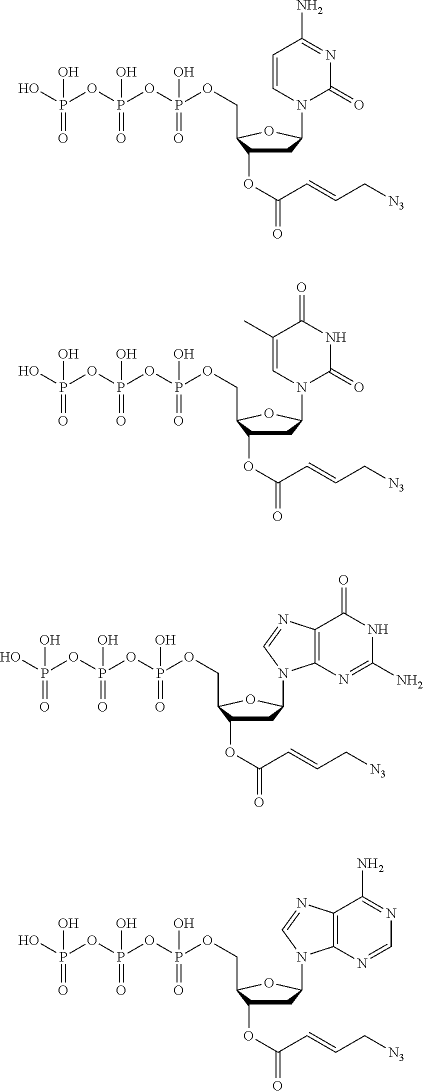

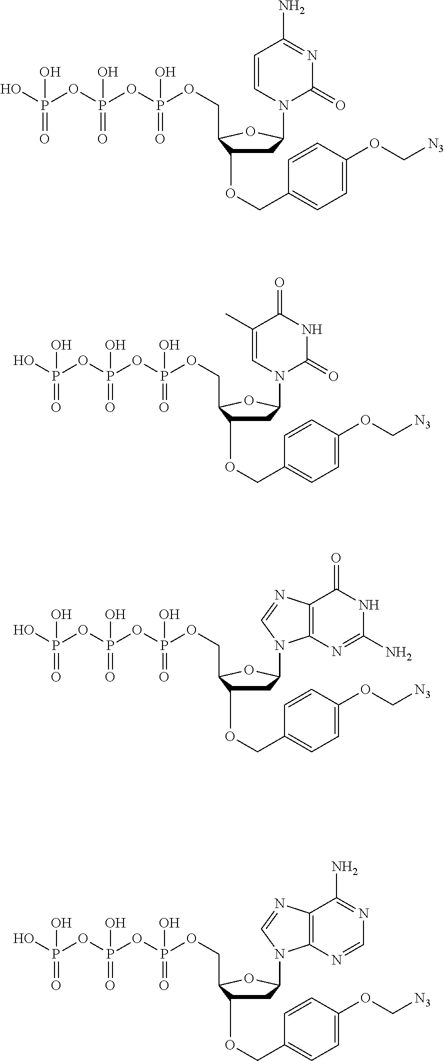

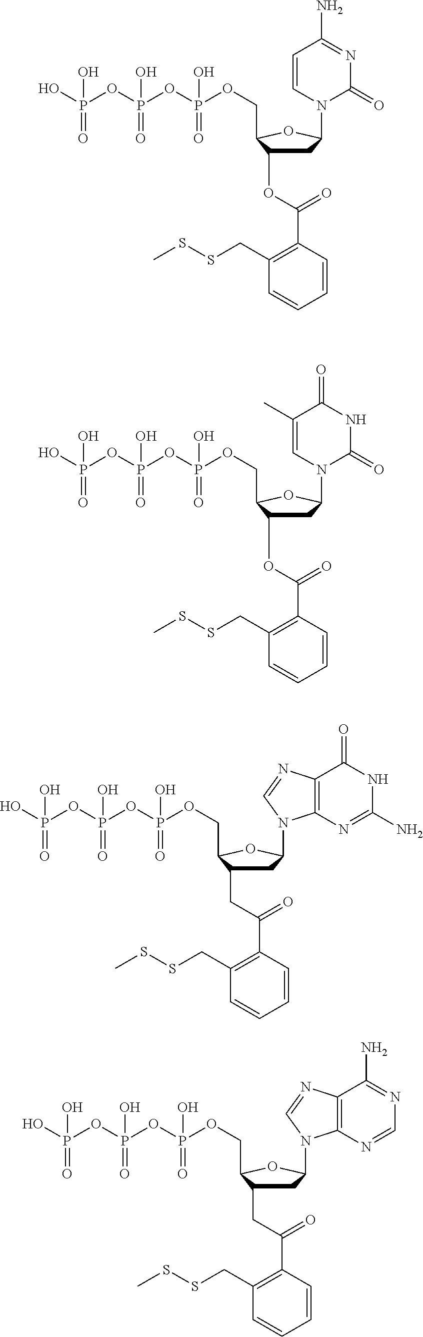

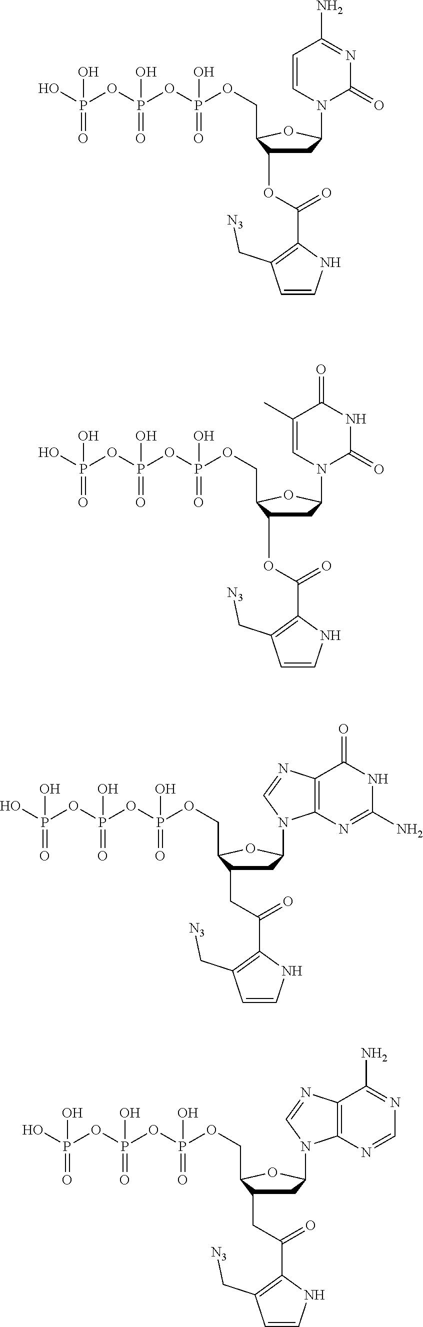

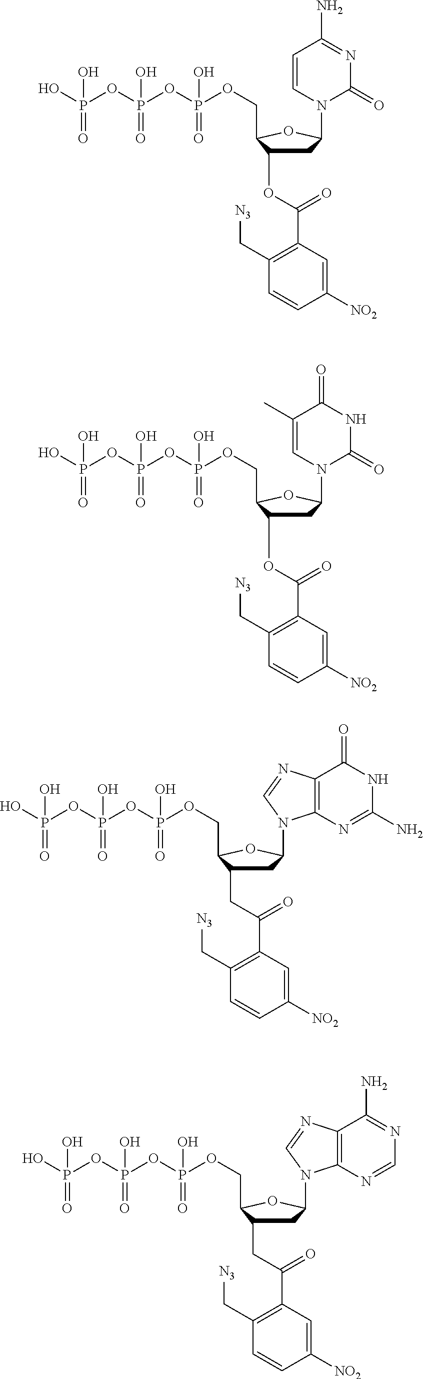

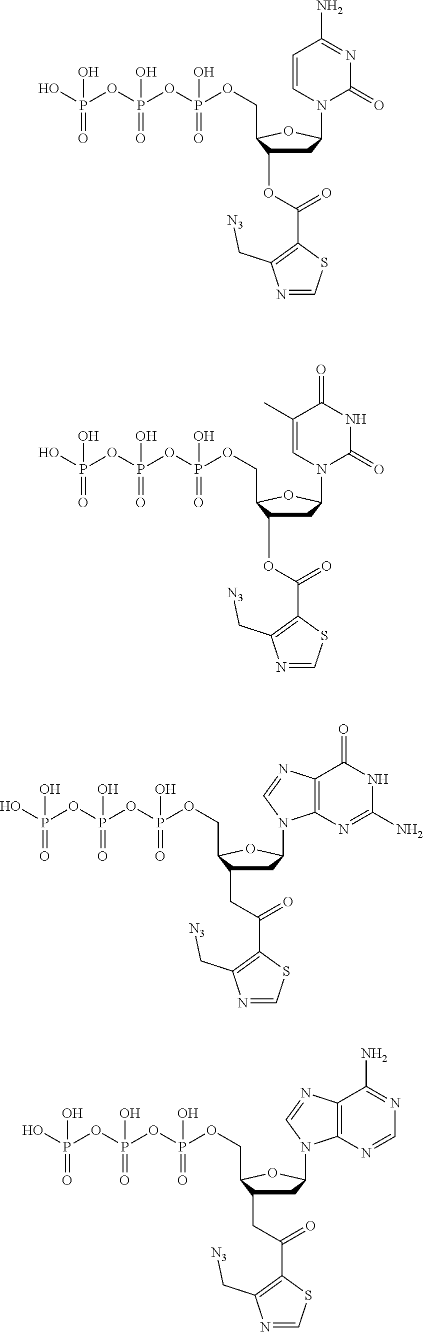

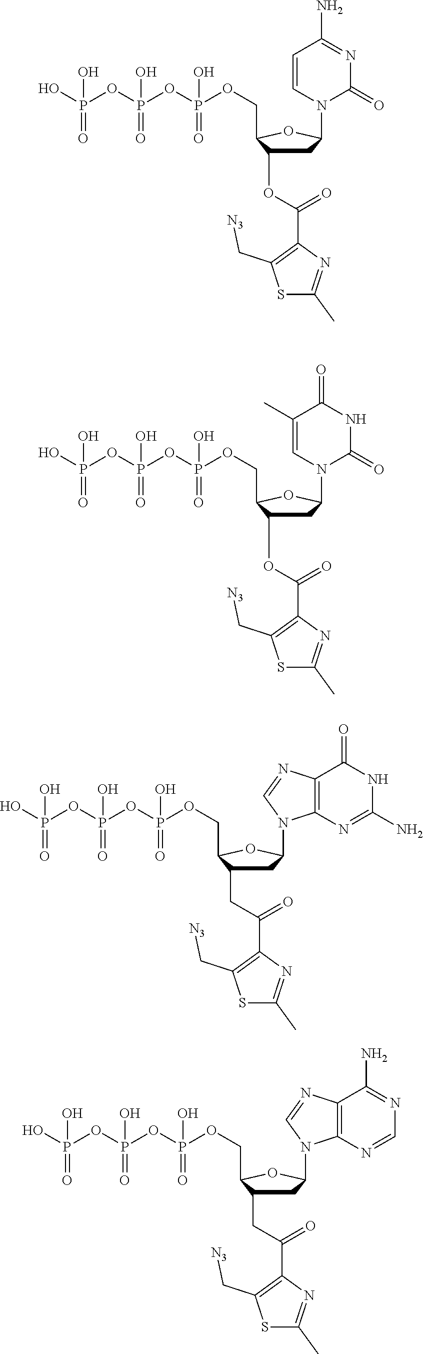

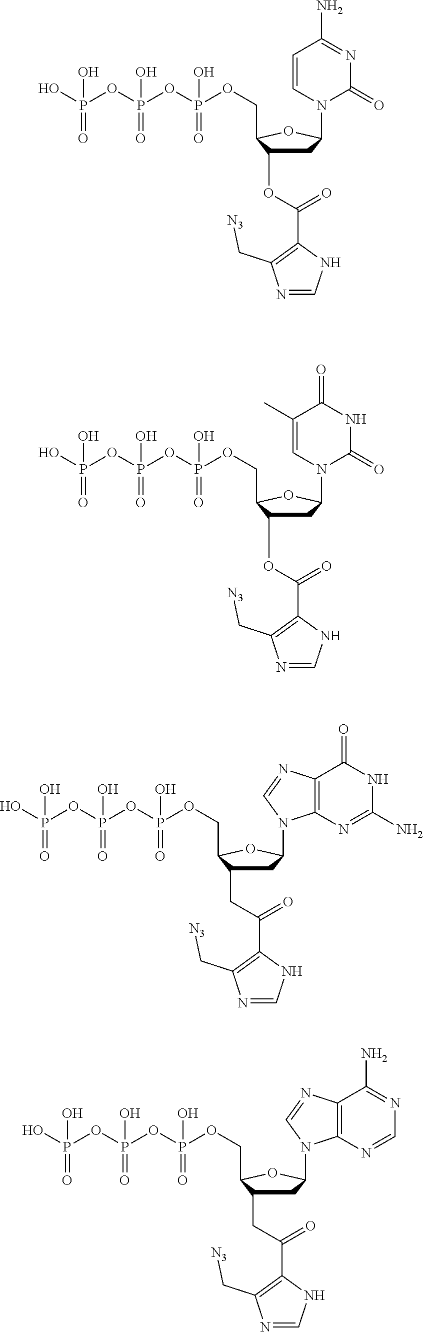

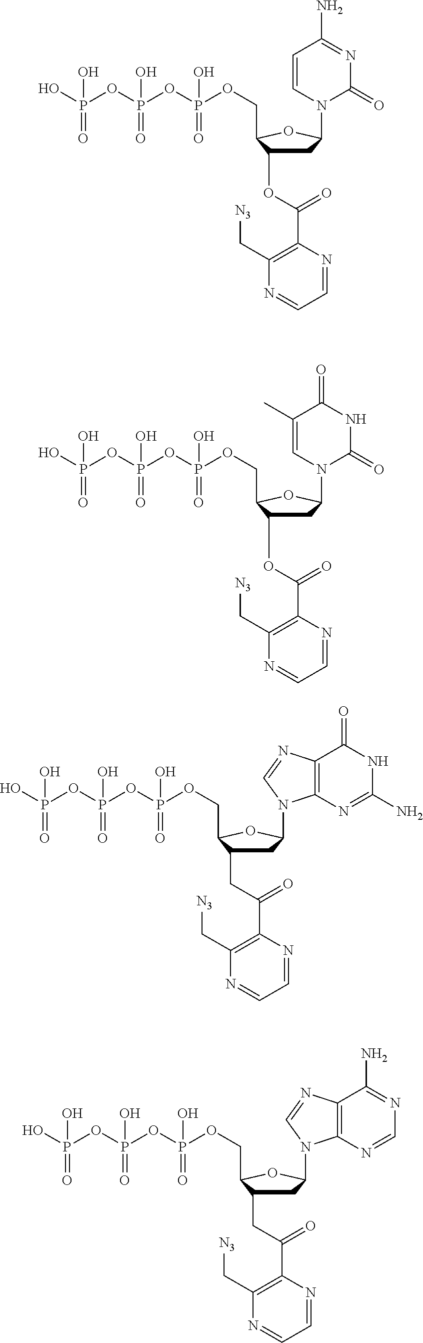

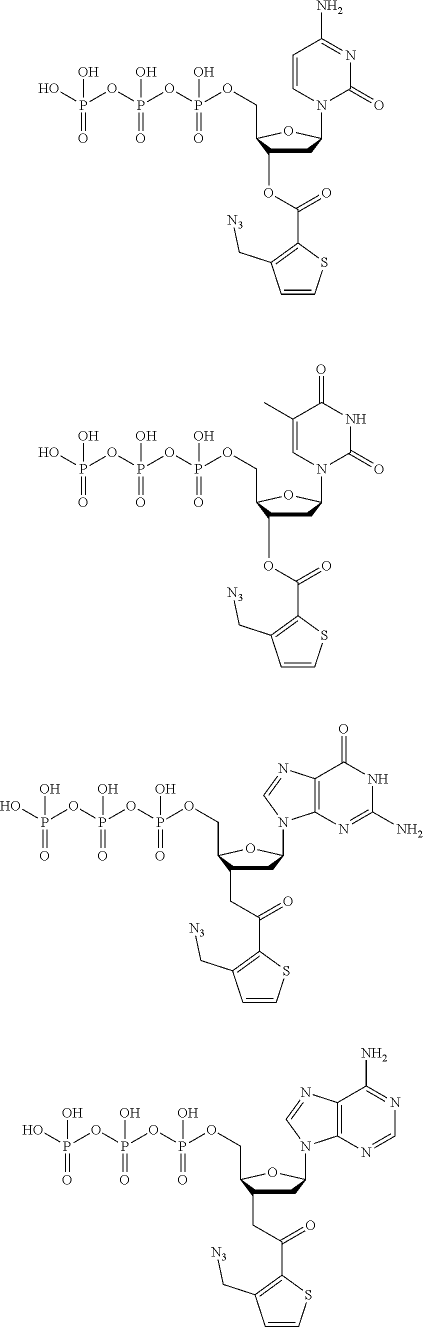

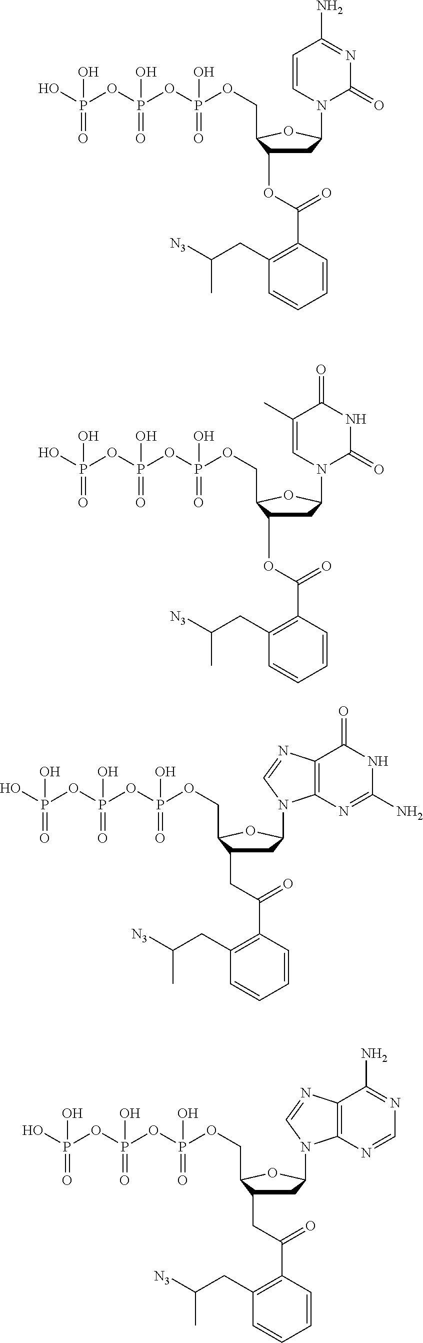

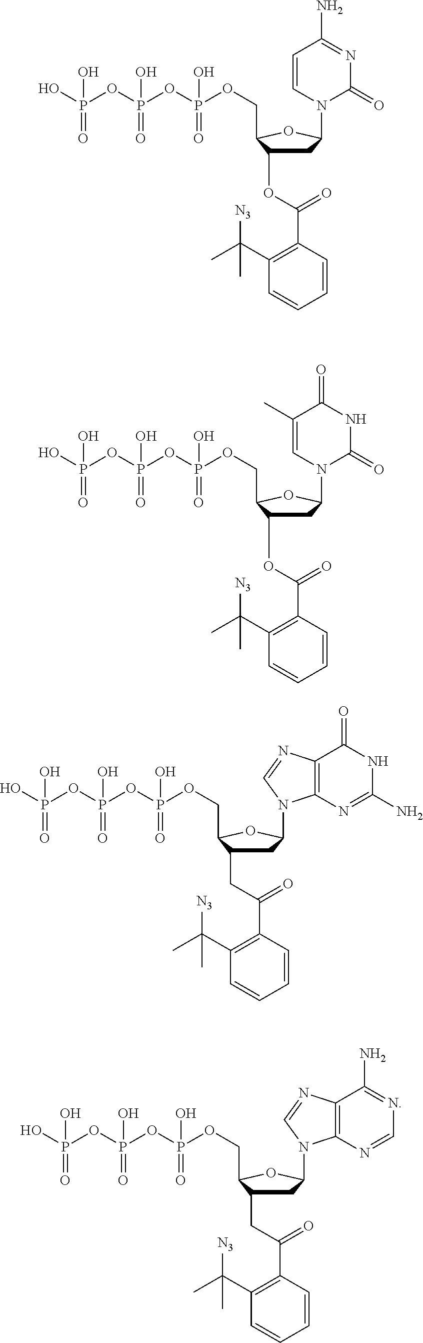

5. The compound or a salt thereof according to claim 1, which carries an additional detectable label (e.g., a fluorescent label);
preferably, the additional detectable label carried by the compound or a salt thereof is introduced by an affinity reagent (e.g., antibody, aptamer, Affimer, Knottin), wherein the affinity reagent carries the detectable label, and the affinity reagent can specifically recognize and bind to an epitope of the compound or a salt thereof;
preferably, the additional detectable label (e.g., a fluorescent label) is linked to the compound or a salt thereof optionally via a linker;
preferably, the additional detectable label (e.g., a fluorescent label) is linked to the Base of the compound or a salt thereof optionally via a linker;
preferably, the linker is a cleavable linker or a non-cleavable linker;
preferably, the cleavable linker is selected from the group consisting of a linker capable of being cleaved by electrophilic reaction, a linker capable of being cleaved by nucleophilic reaction, a linker capable of being cleaved by photolysis, a linker capable of being cleaved under reductive conditions, a linker capable of being cleaved under oxidative conditions, a safety-catch linker, a linker capable of being cleaved by elimination mechanisms, or any combination thereof;
preferably, the detectable label is selected from the followings: 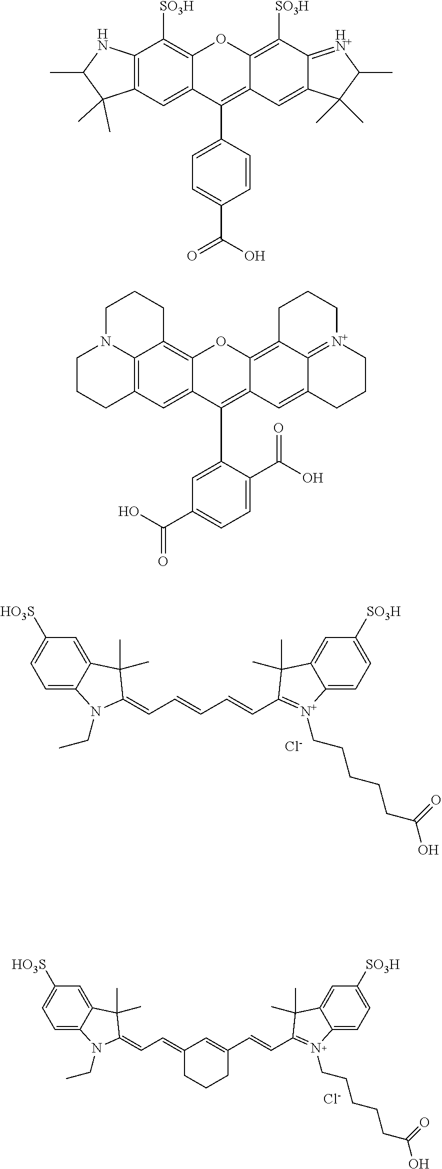 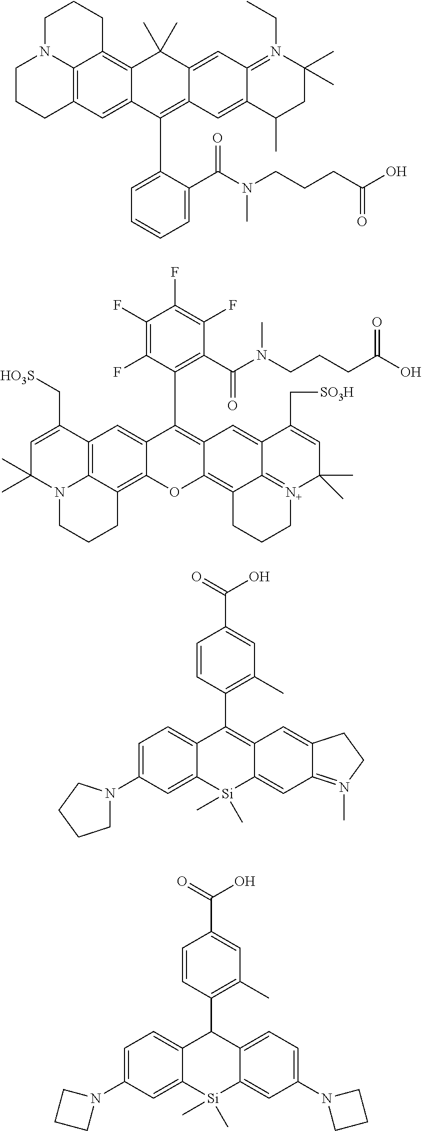 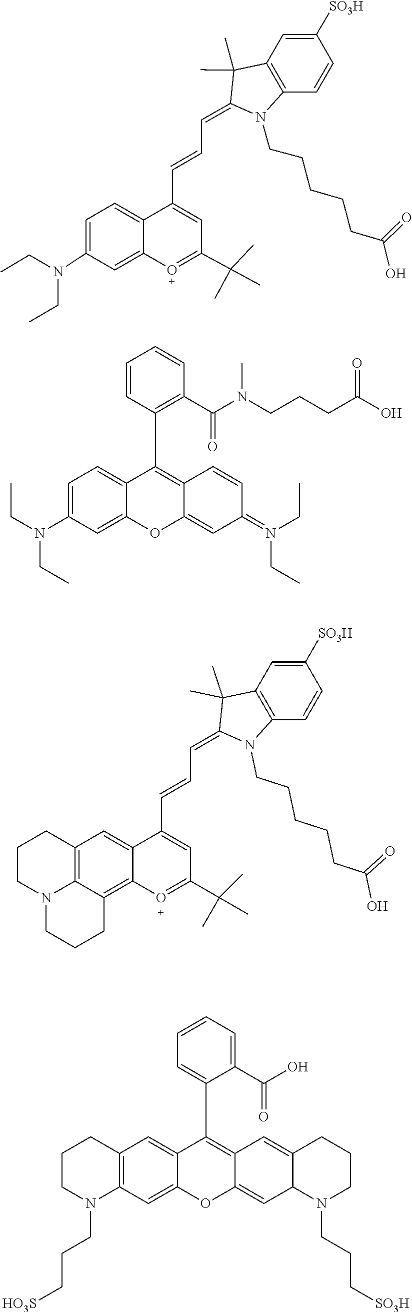 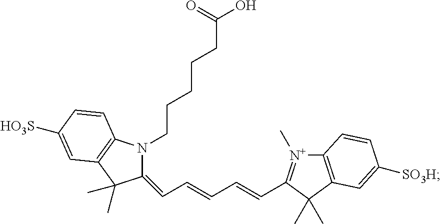 [SMILES: C[N+]1=C(/C=C/C=C/C=C2\N(CCCCCC(=O)O)c3ccc(S(=O)(=O)O)cc3C2(C)C)C(C)(C)c2cc(S(=O)(=O)O)ccc21]
preferably, if Base is different, the detectable label (e.g., a fluorescent label) varies.

6. A method for terminating a nucleic acid synthesis, which comprises incorporating the compound or a salt thereof according to claim 1, into a nucleic acid molecule to be terminated;
preferably, the incorporation of the compound or a salt thereof is achieved by a terminal transferase, a terminal polymerase or a reverse transcriptase;
preferably, the method comprises incorporating the compound or a salt thereof into the nucleic acid molecule to be terminated by using a polymerase;
preferably, the method comprises performing a nucleotide polymerization reaction using a polymerase under conditions where the polymerase is allowed to carry out the nucleotide polymerization reaction, thereby incorporating the compound or a salt thereof into the 3′ end of the nucleic acid molecule to be terminated.

7. A method for preparing a growing polynucleotide complementary to a target single-stranded polynucleotide in a sequencing reaction, which comprises incorporating the compound or a salt thereof according to claim 1, into the growing complementary polynucleotide, wherein the incorporation of the compound or a salt thereof prevents any subsequent nucleotides from being introduced into the growing complementary polynucleotide;
preferably, the incorporation of the compound or a salt thereof is achieved by a terminal transferase, a terminal polymerase or a reverse transcriptase;
preferably, the method comprises incorporating the compound or a salt thereof into the growing complementary polynucleotide by using a polymerase;
preferably, the method comprises performing a nucleotide polymerization reaction using a polymerase under conditions where the polymerase is allowed to carry out the nucleotide polymerization reaction, thereby incorporating the compound or a salt thereof into the 3′ end of the growing complementary polynucleotide.

8. A method for determining the sequence of a target single-stranded polynucleotide, which comprises:
1) monitoring the incorporation of nucleotides complementary to the target single-stranded polynucleotide in a growing nucleic acid strand, wherein at least one complementary nucleotide incorporated is the compound or a salt thereof according to claim 1, and the compound or a salt thereof carries an additional detectable label (e.g., a fluorescent label), and,
2) detecting the detectable label to determine the incorporated nucleotide;
preferably, the additional detectable label (e.g., a fluorescent label) is linked to the compound or a salt thereof optionally via a linker;
preferably, the linker is a cleavable linker or a non-cleavable linker;
preferably, the additional detectable label carried by the compound or a salt thereof is introduced by an affinity reagent (e.g., antibody, aptamer, Affimer, Knottin), wherein the affinity reagent carries the detectable label, and the affinity reagent can specifically recognize and bind to an epitope of the compound or a salt thereof;
preferably, the cleavable linker is selected from the group consisting of a linker capable of being cleaved by electrophilic reaction, a linker capable of being cleaved by nucleophilic reaction, a linker capable of being cleaved by photolysis, a linker capable of being cleaved under reductive conditions, a linker capable of being cleaved under oxidative conditions, a safety-catch linker, a linker capable of being cleaved by elimination mechanisms, or any combination thereof;
preferably, the additional detectable label (e.g., a fluorescent label) is linked to the Base of the compound or a salt thereof optionally via a linker;
preferably, the detectable label is selected from the followings:
preferably, the detectable label is selected from the following: 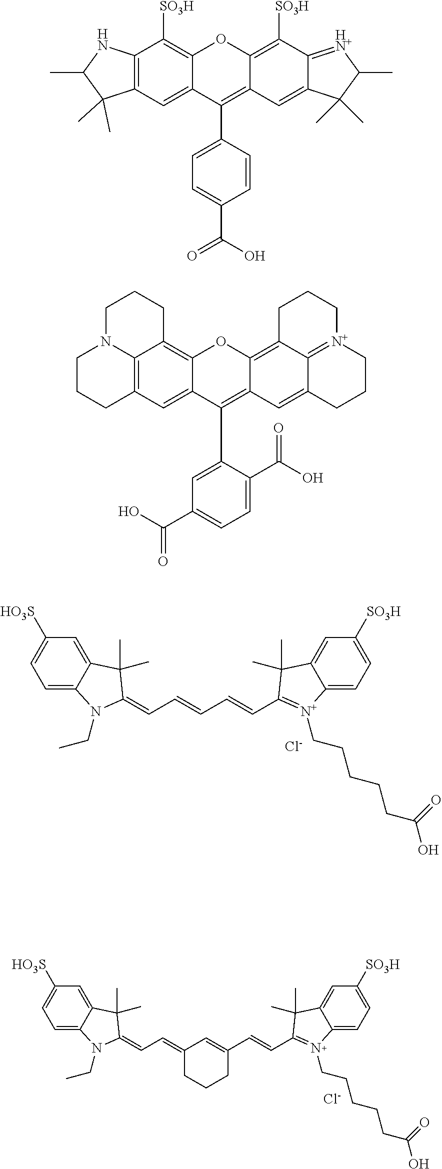 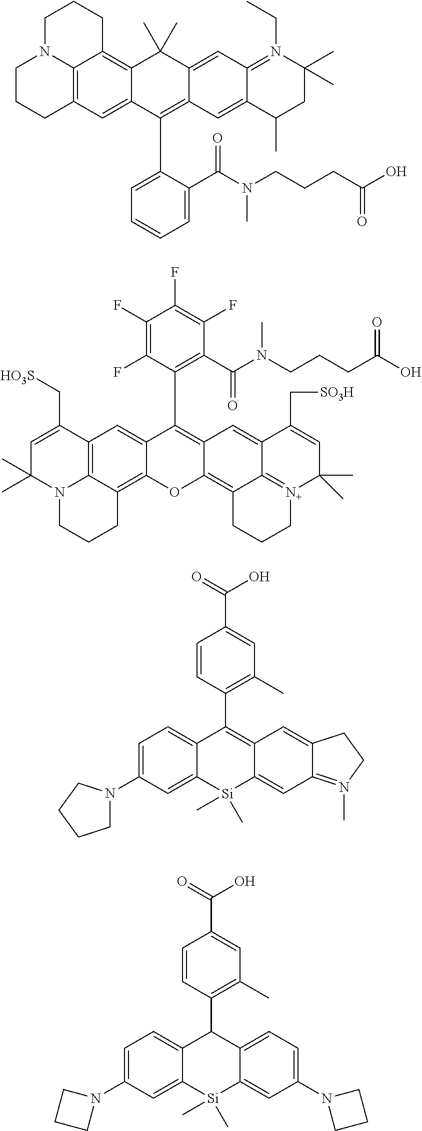 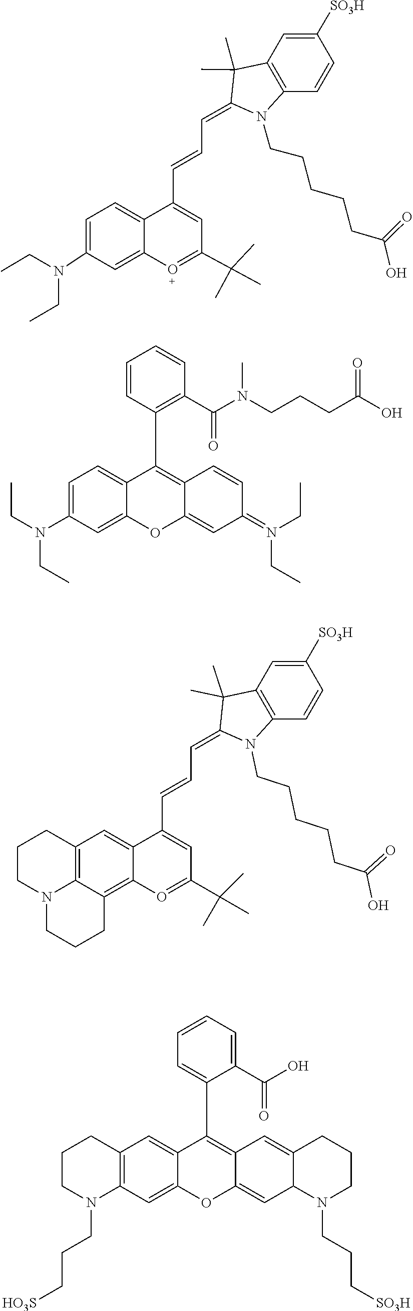 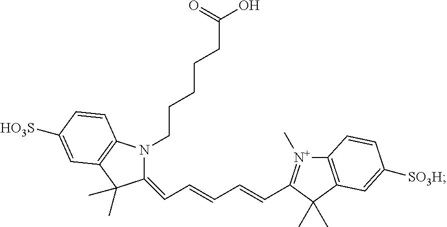 [SMILES: C[N+]1=C(/C=C/C=C/C=C2\N(CCCCCC(=O)O)c3ccc(S(=O)(=O)O)cc3C2(C)C)C(C)(C)c2cc(S(=O)(=O)O)ccc21]
preferably, if Base is different, the detectable label (e.g., a fluorescent label) carried by the compound or a salt thereof varies;
preferably, the reversible blocking group (R) in the compound or a salt thereof and the detectable label are removed prior to introducing a next complementary nucleotide;
preferably, the reversible blocking group and the detectable label are removed simultaneously; or
preferably, the reversible blocking group and the detectable label are removed sequentially; for example, after the detectable label is removed, the reversible blocking group is removed, or, after the reversible blocking group is removed, the detectable label is removed.

9. The method according to claim 8, which comprises the following steps of:
(a) providing a plurality of different nucleotides, wherein the plurality of different nucleotides are the compound of formula (A) or a salt thereof, wherein each nucleotide carries an additional detectable label that can be distinguished from an additional detectable label carried by another nucleotide during detection; 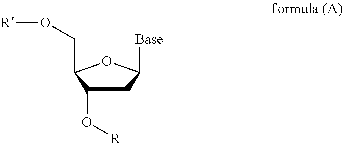
wherein:
R is a reversible blocking group, and R is selected from 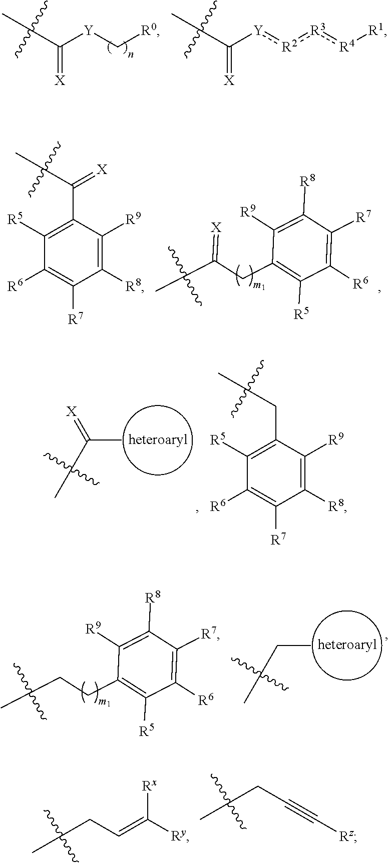
preferably, R is selected from 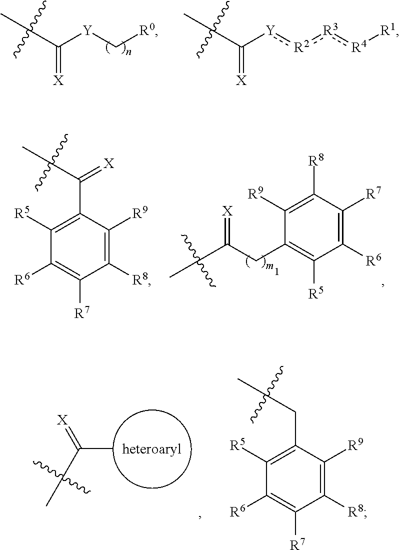
more preferably, R is selected from 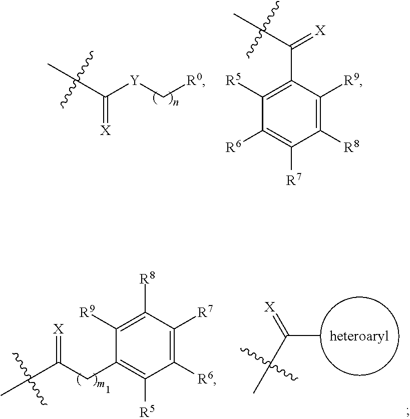
the heteroaryl is selected from the followings: 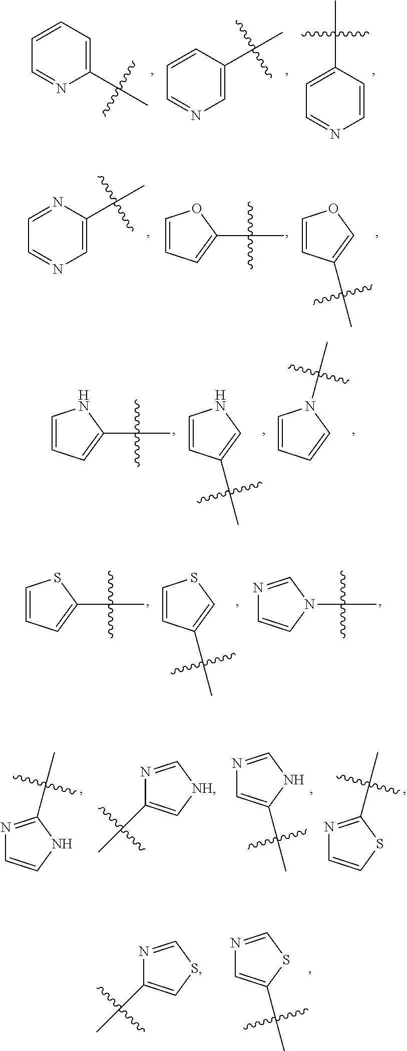
each heteroaryl is independently optionally substituted by one or more (e.g., 2, 3, 4, 5 or 6, preferably 2) R″s;
preferably, the heteroaryl is selected from the followings: 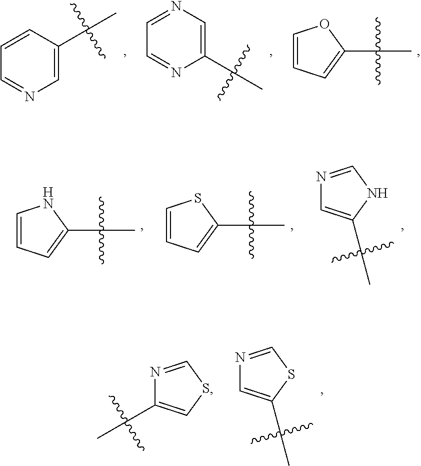
each heteroaryl is independently optionally substituted by one or more (e.g., 2, 3, 4, 5 or 6, preferably 2) R″s;
more preferably, the heteroaryl is selected from the followings: 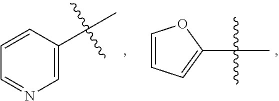 [SMILES: *c1cccnc1 ; *c1ccco1]
each heteroaryl is independently optionally substituted by one or more (e.g., 2, 3, 4, 5 or 6, preferably 2) R″s;
each X is independently selected from O, NH, S;
preferably, X is O;
each Y is independently selected from a direct bond, O, NH, S, CH, CH₂, C(CH₃)₂;
preferably, each Y is independently selected from a direct bond or CH₂;
R⁰ is selected from —N₃, —SS—C₁-C₆ alkyl (e.g., —SS-methyl, —SS-ethyl, —SS-isopropyl, —SS-tertbutyl), —ONH₂, —OCOR^{m}, —OCONHR^{m}, wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl);
preferably, R⁰ is —N₃ or —SS—C₁-C₆ alkyl (e.g., —SS-methyl, —SS-ethyl, —SS-isopropyl, —SS-tertbutyl);
more preferably, R⁰ is —N₃;
R¹ is selected from —N₃, —SS—C₁-C₆ alkyl (e.g., —SS-methyl, —SS-ethyl, —SS-isopropyl, —SS-tertbutyl), —ONH₂, —OCOR^{m}, —OCONHR^{m}, wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl);
preferably, R¹ is —N₃ or —SS—C₁-C₆ alkyl (e.g., —SS-methyl, —SS-ethyl, —SS-isopropyl, —SS-tertbutyl);
more preferably, R¹ is —N₃;
R², R³, and R⁴ are each independently selected from O, NH, S, CH, CH₂, C(CH₃)₂;
preferably, R², R³, and R⁴ are each independently selected from O, NH, S, CH, CH₂, C(CH₃)₂, and meet the following conditions: when R² is selected from O or S, R³ and R⁴ are CH₂; when R³ is selected from O or S, R² and R⁴ are CH₂; when R⁴ is selected from O or S, R² and R³ are CH₂; when R² is C(CH₃)₂, R³ and R⁴ are CH₂; when R³ is C(CH₃)₂, R² and R⁴ are CH₂; when R⁴ is C(CH₃)₂, R² and R³ are CH₂; when Y is CH, R² is CH, and R³ and R⁴ are CH₂;
more preferably, R², R³, and R⁴ are each independently selected from CH, CH₂;
most preferably, R² and R³ are CH, and R⁴ is CH₂; 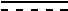
represents a single bond 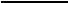
or a double bond 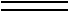
each R″ is independently selected from H, —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy), 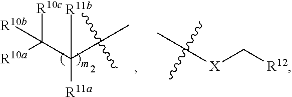
and is not H at the same time;
preferably, when each heteroaryl is independently optionally substituted by one R″, each R″ is independently selected from 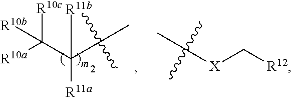
preferably, R″ is 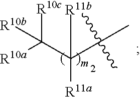
or preferably, when each heteroaryl is independently optionally substituted by more (e.g., 2, 3, 4, 5 or 6, preferably 2) R″s, one of the R″ is selected from 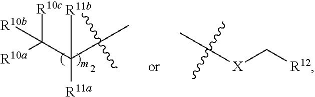
and the remaining R″(s) is(are) each independently selected from —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy);
or more preferably, when each heteroaryl is independently optionally substituted by more (e.g., 2, 3, 4, 5 or 6, preferably 2) R″s, one of the R″ is 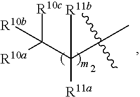
and the remaining R″(s) is(are) each independently selected from nitro, aliphatic alkyl (e.g., C₁-C₆ alkyl), F, I, Br, Cl;
or most preferably, when each heteroaryl is independently optionally substituted by more (e.g., 2, 3, 4, 5 or 6, preferably 2) R″s, one of the R″ is 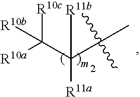
and the remaining R″(s) is(are) each independently selected from nitro, C₁-C₆ alkyl, preferably, the remaining R″(s) is(are) C₁-C₆ alkyl;
R⁵, R⁶, R⁷, R⁸, R⁹, R^{x}, R^{y}, and R^{z} are each independently selected from H, —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy), C₁-C₆ alkyl-C(═O)—NH₂—, 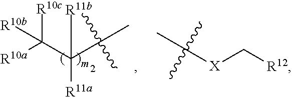
and R⁵, R⁶, R⁷, R⁸, and R⁹ are not H at the same time;
preferably, any one of R⁵, R⁶, R⁷, R⁸, and R⁹ is selected from 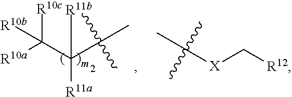
or
and the remaining four thereof are each independently selected from H, —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy), C₁-C₆ alkyl-C(═O)—NH₂—;
more preferably, any one of R⁵, R⁶, R⁷, R⁸, and R⁹ is 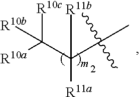
and the remaining four thereof are each independently selected from H, nitro, C₁-C₆ alkyl, C₁-C₆ alkoxy, C₁-C₆ alkyl-C(═O)—NH₂—;
further preferably, of R⁵, R⁶, R⁷, R⁸, and R⁹, R⁵ or R⁹ is 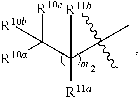
and the remaining four thereof are each independently selected from H, nitro, methoxy, acetamido;
most preferably, of R⁵, R⁶, R⁷, R⁸, and R⁹, R⁵ or R⁹ is 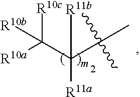
R⁷ is selected from H, nitro, methoxy, acetamido, and the remaining three thereof are H;
preferably, any one of R^{x} and R^{y} is selected from 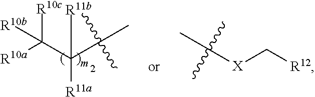
and the other is selected from H, —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy);
more preferably, any one of R^{x} and R^{y} is selected from 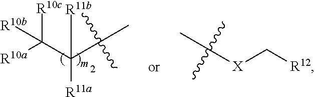
and the other is H;
most preferably, any one of R^{x} and R^{y} is 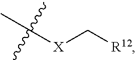
and the other is H;
preferably, R^{z} is selected from 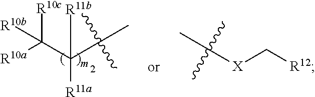
more preferably, R^{z} is 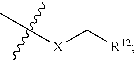
R^{10a}, R^{10b}, R^{10c}, R^{11a}, R^{11b}, and R¹² are each independently selected from H, —N₃, —SS—C₁-C₆ alkyl (e.g., —SS-methyl, —SS-ethyl, —SS-isopropyl, —SS-tertbutyl or —SS-isobutyl), —ONH₂, —OCOR^{m}, —OCONHR^{m}, aliphatic alkyl (e.g., C₁-C₆ alkyl, particularly such as methyl, ethyl, isopropyl, tertbutyl), aromatic alkyl (e.g., phenyl C₁-C₆ alkyl), cycloalkyl (e.g., C₃-C₆ cycloalkyl), wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl), and R^{10a}, R^{10b}, and R^{10c} are not H at the same time;
preferably, any one of R^{10a}, R^{10b}, and R^{10c} is selected from —N₃, —SS—C₁-C₆ alkyl (e.g., —SS-methyl, —SS-ethyl, —SS-isopropyl, —SS-tertbutyl or —SS-isobutyl), —ONH₂, —OCOR^{m}, —OCONHR^{m}, and the other two are each independently selected from H, aliphatic alkyl (e.g., C₁-C₆ alkyl, particularly such as methyl, ethyl, isopropyl, tertbutyl), aromatic alkyl (e.g., phenyl C₁-C₆ alkyl), cycloalkyl (e.g., C₃-C₆ cycloalkyl), wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl);
more preferably, any one of R^{10a}, R^{10b}, and R^{10c} is —N₃ or —SS—C₁-C₆ alkyl (e.g., —SS-methyl, —SS-ethyl, —SS-isopropyl, —SS-tertbutyl or —SS-isobutyl), and the other two are each independently selected from H, aliphatic alkyl (e.g., C₁-C₆ alkyl, particularly such as methyl, ethyl, isopropyl, tertbutyl);
most preferably, any one of R^{10a}, R^{10b}, and R^{10c} is —N₃, —SS-methyl, —SS-ethyl, —SS-isopropyl, —SS-tertbutyl or —SS-isobutyl, and the other two are each independently selected from H or methyl;
preferably, R^{11a} and R^{11b} are each independently selected from H or aliphatic alkyl (e.g., C₁-C₆ alkyl, particularly such as methyl, ethyl, isopropyl, tertbutyl);
more preferably, R^{11a} and R^{11b} are H;
preferably, R¹² is selected from —N₃, —SS—C₁-C₆ alkyl (e.g., —SS-methyl, —SS-ethyl, —SS-isopropyl, —SS-tertbutyl), —ONH₂, —OCOR^{m}, —OCONHR^{m}, wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl);
more preferably, R¹² is —N₃ or —SS—C₁-C₆ alkyl (e.g., —SS-methyl, —SS-ethyl, —SS-isopropyl, —SS-tertbutyl);
most preferably, R¹² is —N₃;
n is selected from 1, 2, 3, 4, or 5; preferably, n is selected from 1, 2, or 3; more preferably, n is 2;
each m₁ is independently selected from 1, 2, 3, 4, 5, and 6; preferably, m₁ is 1;
each m₂ is independently selected from 0, 1, 2, 3, 4, 5, and 6; preferably, each m₂ is independently selected from 0 or 1;
most preferably, 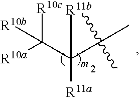
as a whole, is selected from 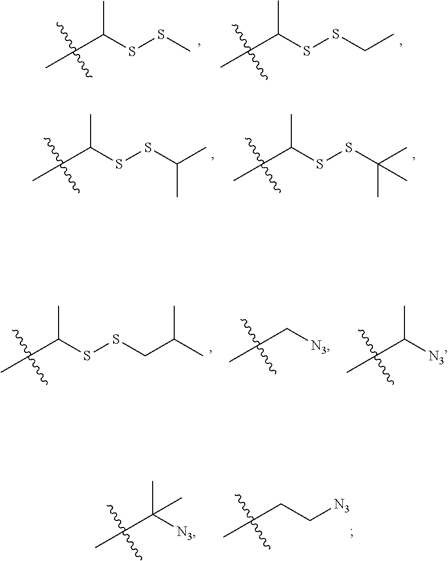
R′ is selected from H, monophosphate group 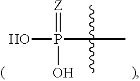
diphosphate group 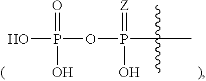
triphosphate group 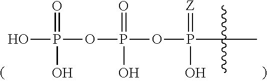
or tetraphosphate group 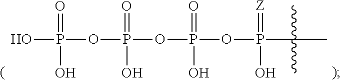
preferably, R′ is a triphosphate group 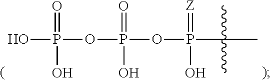
each Z is independently selected from O, S, BH; preferably, Z is O;
Base is selected from a base, a deaza base or a tautomer thereof, for example, Base is selected from adenine, 7-deazaadenine, thymine, uracil, cytosine, guanine, 7-deazaguanine or a tautomer thereof;
preferably, Base is selected from 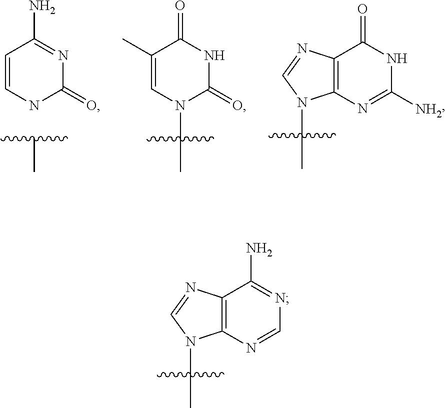
(b) incorporating the plurality of different nucleotides into a sequence complementary to a target single-stranded polynucleotide;
(c) detecting the additional detectable labels carried by the nucleotides in step (b) to determine the types of nucleotides incorporated;
(d) removing the reversible blocking groups and the detectable labels carried by the nucleotides in step (b); and
(e) optionally repeating steps (b)-(d) one or more times;
whereby the sequence of the target single-stranded polynucleotide is determined.

10. The method according to claim 8, which comprises the following steps of:
(1) providing a first nucleotide, a second nucleotide, a third nucleotide and a fourth nucleotide, wherein at least one of the four nucleotides is the compound of formula (A) or a salt thereof, the Bases contained in the four nucleotides are different from each other, and the four nucleotides carry an additional detectable label (e.g., a fluorescent label), preferably, the additional detectable label carried by the four nucleotides is introduced by an affinity reagent (e.g., antibody, aptamer, Affimer, Knottin), wherein the affinity reagent carries the detectable label, and the affinity reagent can specifically recognize and bind to an epitope of each nucleotide, or preferably, the four nucleotides are linked to the additional detectable label optionally via a linker, or more preferably, the Base of the four nucleotides is linked to the additional detectable label optionally via a linker, most preferably, the additional detectable labels carried by the four nucleotides are different from each other: 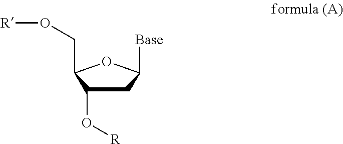
wherein:
R is a reversible blocking group, and R is selected from 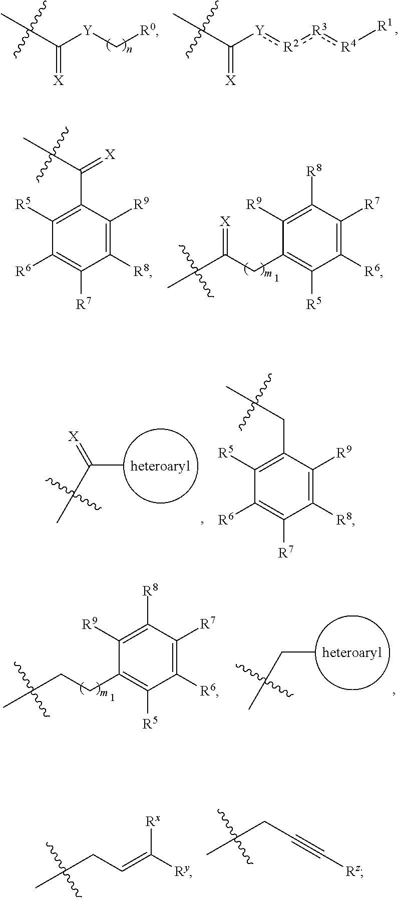
preferably, R is selected from 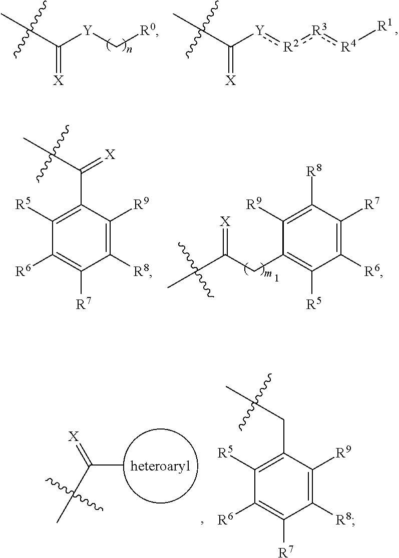
more preferably, R is selected from 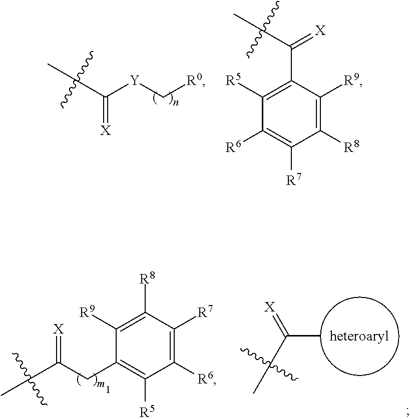
the heteroaryl is selected from the followings: 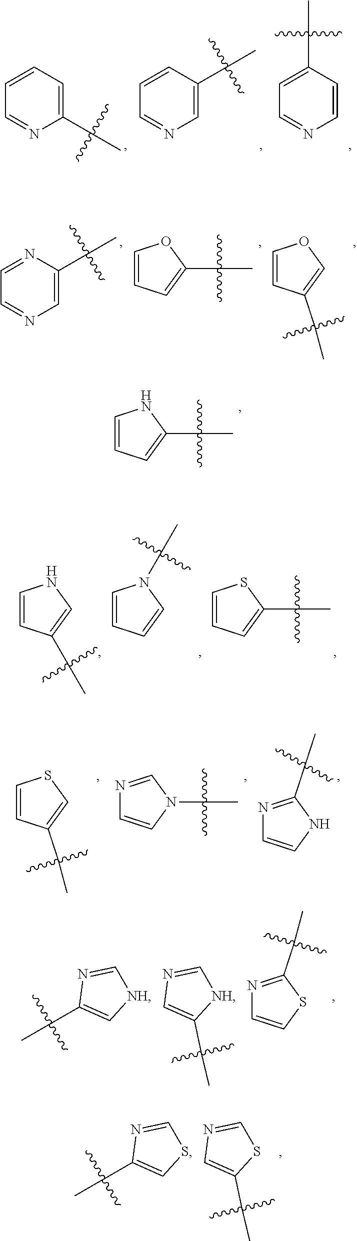
each heteroaryl is independently optionally substituted by one or more (e.g., 2, 3, 4, 5 or 6, preferably 2) R″s;
preferably, the heteroaryl is selected from the followings: 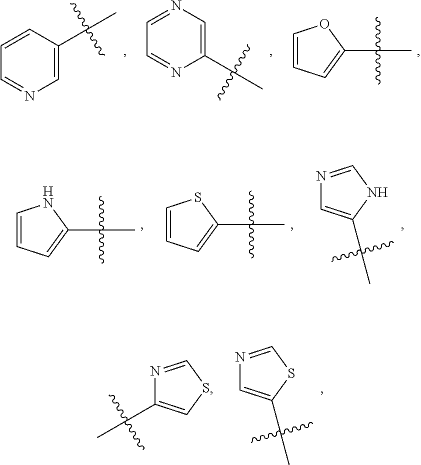
each heteroaryl is independently optionally substituted by one or more (e.g., 2, 3, 4, 5 or 6, preferably 2) R″s;
more preferably, the heteroaryl is selected from the followings: 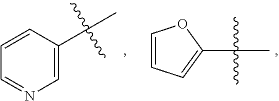 [SMILES: *c1cccnc1 ; *c1ccco1]
each heteroaryl is independently optionally substituted by one or more (e.g., 2, 3, 4, 5 or 6, preferably 2) R″s;
each X is independently selected from O, NH, S;
preferably, X is O;
each Y is independently selected from a direct bond, O, NH, S, CH, CH₂, C(CH₃)₂;
preferably, each Y is independently selected from a direct bond or CH₂;
R⁰ is selected from —N₃, —SS—C₁-C₆ alkyl (e.g., —SS-methyl, —SS-ethyl, —SS-isopropyl, —SS-tertbutyl), —ONH₂, —OCOR^{m}, —OCONHR^{m}, wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl);
preferably, R⁰ is —N₃ or —SS—C₁-C₆ alkyl (e.g., —SS-methyl, —SS-ethyl, —SS-isopropyl, —SS-tertbutyl);
more preferably, R⁰ is —N₃;
R¹ is selected from —N₃, —SS—C₁-C₆ alkyl (e.g., —SS-methyl, —SS-ethyl, —SS-isopropyl, —SS-tertbutyl), —ONH₂, —OCOR^{m}, —OCONHR^{m}, wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl);
preferably, R¹ is —N₃ or —SS—C₁-C₆ alkyl (e.g., —SS-methyl, —SS-ethyl, —SS-isopropyl, —SS-tertbutyl);
more preferably, R¹ is —N₃;
R², R³, and R⁴ are each independently selected from O, NH, S, CH, CH₂, C(CH₃)₂;
preferably, R², R³, and R⁴ are each independently selected from O, NH, S, CH, CH₂, C(CH₃)₂, and meet the following conditions: when R² is selected from O or S, R³ and R⁴ are CH₂; when R³ is selected from O or S, R² and R⁴ are CH₂; when R⁴ is selected from O or S, R² and R³ are CH₂; when R² is C(CH₃)₂, R³ and R⁴ are CH₂; when R³ is C(CH₃)₂, R² and R⁴ are CH₂; when R⁴ is C(CH₃)₂, R² and R³ are CH₂; when Y is CH, R² is CH, and R³ and R⁴ are CH₂;
more preferably, R², R³, and R⁴ are each independently selected from CH, CH₂;
most preferably, R² and R³ are CH, and R⁴ is CH₂; [symbol]
represents a single bond [symbol] or a double bond [symbol] ;
each R″ is independently selected from H, —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy), 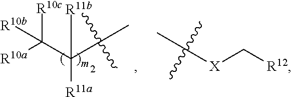
and is not H at the same time;
preferably, when each heteroaryl is independently optionally substituted by one R″, each R″ is independently selected from 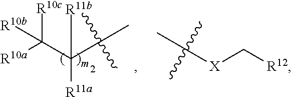
preferably, R″ is 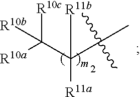
or preferably, when each heteroaryl is independently optionally substituted by more (e.g., 2, 3, 4, 5 or 6, preferably 2) R″s, one of the R″ is selected from 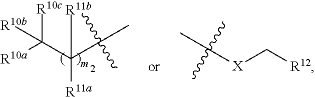
and the remaining R″(s) is(are) each independently selected from —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy);
or more preferably, when each heteroaryl is independently optionally substituted by more (e.g., 2, 3, 4, 5 or 6, preferably 2) R″s, one of the R″ is 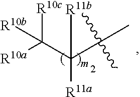
and the remaining R″(s) is(are) each independently selected from nitro, aliphatic alkyl (e.g., C₁-C₆ alkyl), F, I, Br, Cl;
or most preferably, when each heteroaryl is independently optionally substituted by more (e.g., 2, 3, 4, 5 or 6, preferably 2) R″s, one of the R″ is 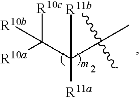
and the remaining R″(s) is(are) each independently selected from nitro, C₁-C₆ alkyl, preferably, the remaining R″(s) is(are) C₁-C₆ alkyl;
R⁵, R⁶, R⁷, R⁸, R⁹, R¹⁰, R^{y}, and R^{z} are each independently selected from H, —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy), C₁-C₆ alkyl-C(═O)—NH₂—, 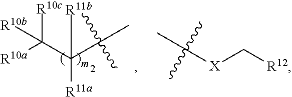
and R⁵, R⁶, R⁷, R⁸, and R⁹ are not H at the same time;
preferably, any one of R⁵, R⁶, R⁷, R⁸, and R⁹ is selected from 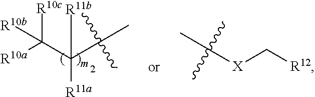
and the remaining four thereof are each independently selected from H, —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy), C₁-C₆ alkyl-C(═O)—NH₂—;
more preferably, any one of R⁵, R⁶, R⁷, R⁸, and R⁹ is 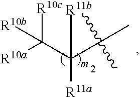
and the remaining four thereof are each independently selected from H, nitro, C₁-C₆ alkyl, C₁-C₆ alkoxy, C₁-C₆ alkyl-C(═O)—NH₂—;
further preferably, of R⁵, R⁶, R⁷, R⁸, and R⁹, R⁵ or R⁹ is 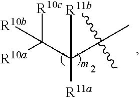
and the remaining four thereof are each independently selected from H, nitro, methoxy, acetamido;
most preferably, of R⁵, R⁶, R⁷, R⁸, and R⁹, R⁵ or R⁹ is 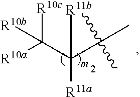
R⁷ is selected from H, nitro, methoxy, acetamido, and the remaining three thereof are H;
preferably, any one of R^{x} and R^{y} is selected from 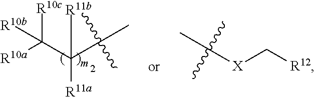
and the other is selected from H, —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy);
more preferably, any one of R^{x} and R^{y} is selected from 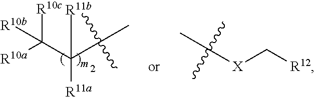
and the other is H;
most preferably, any one of R^{x} and R^{y} is 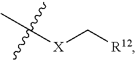
and the other is H;
preferably, R^{z} is selected from 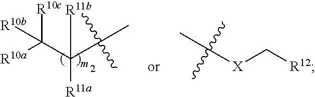
more preferably, R^{z} is 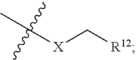
R^{10a}, R^{10b}, R^{10c}, R^{11a}, R^{11b}, and R¹² are each independently selected from H, —N₃, —SS—C₁-C₆ alkyl (e.g., —SS-methyl, —SS-ethyl, —SS-isopropyl, —SS-tertbutyl or —SS-isobutyl), —ONH₂, —OCOR^{m}, —OCONHR^{m}, aliphatic alkyl (e.g., C₁-C₆ alkyl, particularly such as methyl, ethyl, isopropyl, tertbutyl), aromatic alkyl (e.g., phenyl C₁-C₆ alkyl), cycloalkyl (e.g., C₃-C₆ cycloalkyl), wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl), and R^{10a}, R^{10b}, and R^{10c} are not H at the same time;
preferably, any one of R^{10a}, R^{10b}, and R^{10c} is selected from —N₃, —SS—C₁-C₆ alkyl (e.g., —SS-methyl, —SS-ethyl, —SS-isopropyl, —SS-tertbutyl or —SS-isobutyl), —ONH₂, —OCOR^{m}, —OCONHR^{m}, and the other two are each independently selected from H, aliphatic alkyl (e.g., C₁-C₆ alkyl, particularly such as methyl, ethyl, isopropyl, tertbutyl), aromatic alkyl (e.g., phenyl C₁-C₆ alkyl), cycloalkyl (e.g., C₃-C₆ cycloalkyl), wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl);
more preferably, any one of R^{10a}, R^{10b}, and R^{10c} is —N₃ or —SS—C₁-C₆ alkyl (e.g., —SS-methyl, —SS-ethyl, —SS-isopropyl, —SS-tertbutyl or —SS-isobutyl), and the other two are each independently selected from H, aliphatic alkyl (e.g., C₁-C₆ alkyl, particularly such as methyl, ethyl, isopropyl, tertbutyl);
most preferably, any one of R^{10a}, R^{10b}, and R^{10c} is —N₃, —SS-methyl, —SS-ethyl, —SS-isopropyl, —SS-tertbutyl or —SS-isobutyl, and the other two are each independently selected from H or methyl;
preferably, R^{11a} and R^{11b} are each independently selected from H or aliphatic alkyl (e.g., C₁-C₆ alkyl, particularly such as methyl, ethyl, isopropyl, tertbutyl);
more preferably, R^{11a} and R^{11b} are H;
preferably, R¹² is selected from —N₃, —SS—C₁-C₆ alkyl (e.g., —SS-methyl, —SS-ethyl, —SS-isopropyl, —SS-tertbutyl), —ONH₂, —OCOR^{m}, —OCONHR^{m}, wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl);
more preferably, R¹² is —N₃ or —SS—C₁-C₆ alkyl (e.g., —SS-methyl, —SS-ethyl, —SS-isopropyl, —SS-tertbutyl);
most preferably, R¹² is —N₃;
n is selected from 1, 2, 3, 4, or 5; preferably, n is selected from 1, 2, or 3; more preferably, n is 2;
each m₁ is independently selected from 1, 2, 3, 4, 5, and 6; preferably, m₁ is 1;
each m₂ is independently selected from 0, 1, 2, 3, 4, 5, and 6; preferably, each m₂ is independently selected from 0 or 1;
most preferably, 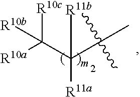
as a whole, is selected from 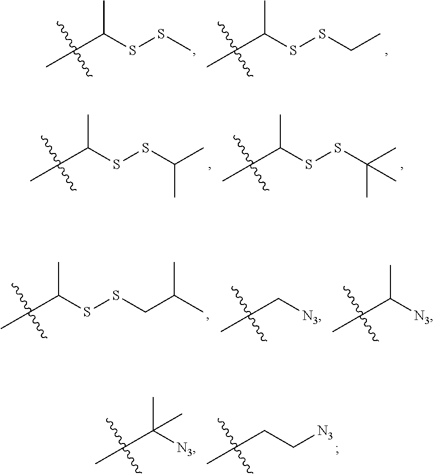
R′ is selected from H, monophosphate group 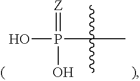
diphosphate group 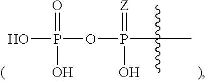
triphosphate group 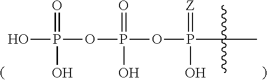
or tetraphosphate group 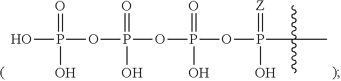
preferably, R′ is a triphosphate group 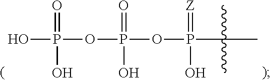
each Z is independently selected from O, S, BH; preferably, Z is O;
Base is selected from a base, a deaza base or a tautomer thereof, for example, Base is selected from adenine, 7-deazaadenine, thymine, uracil, cytosine, guanine, 7-deazaguanine or a tautomer thereof;
preferably, Base is selected from 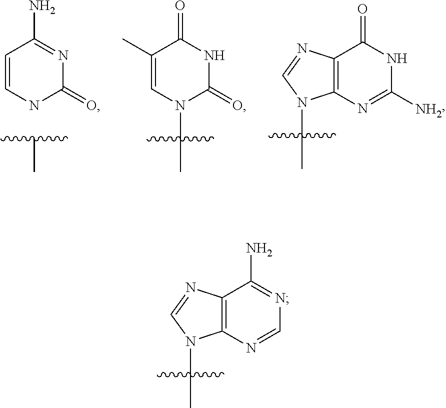
(2) contacting the four nucleotides with a target single-stranded polynucleotide; removing the nucleotides that are not incorporated into the growing nucleic acid strand; detecting the detectable labels carried by the nucleotides that are incorporated into the growing nucleic acid strand; removing the reversible blocking groups and the detectable labels carried by the nucleotides that are incorporated into the growing nucleic acid strand;
optionally, it also includes (3): repeat the step (2) one or more times.

11. The method according to claim 8, which comprises the following steps of:
(a) providing a mixture comprising a duplex, at least one compound of formula (A) or a salt thereof, polymerase and an excision reagent; wherein the duplex comprises a growing nucleic acid strand and a nucleic acid strand to be sequenced; wherein the compound or a salt thereof carries an additional detectable label (e.g., a fluorescent label), preferably, the additional detectable label carried by the compound or a salt thereof is introduced by an affinity reagent (e.g., antibody, aptamer, Affimer, Knottin), wherein the affinity reagent carries the detectable label, and the affinity reagent can specifically recognize and bind to an epitope of the compound or a salt thereof, or preferably, the compound or a salt thereof is linked to the additional detectable label optionally via a linker, or more preferably, the Base of the compound or a salt thereof is linked to the additional detectable label optionally via a linker: 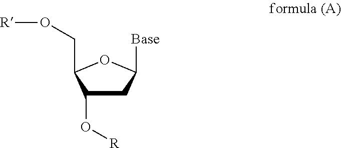
wherein:
R is a reversible blocking group, and R is selected from 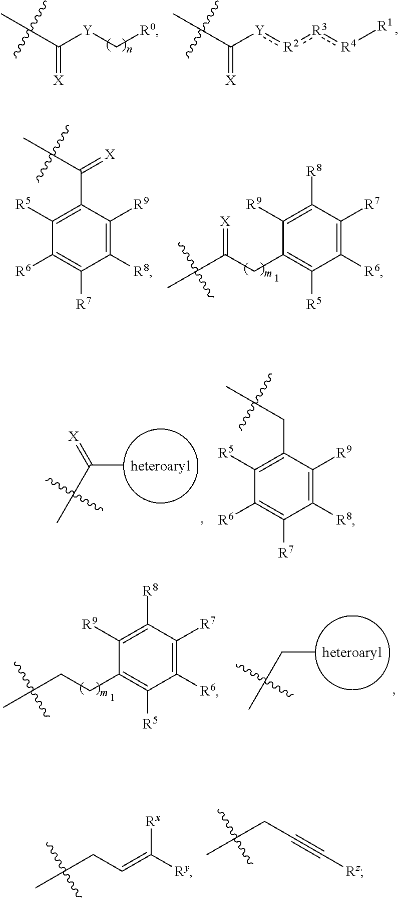
preferably, R is selected from 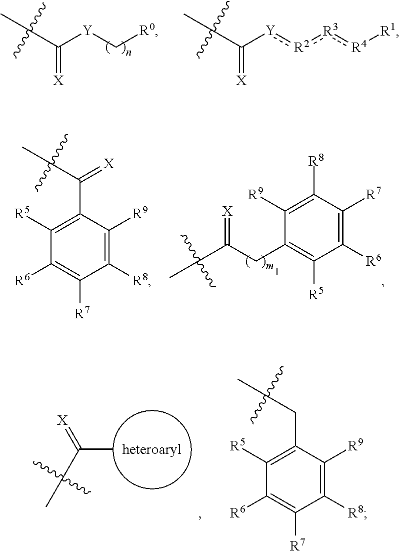
more preferably, R is selected from 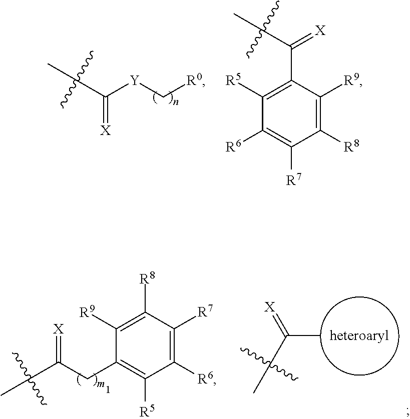
the heteroaryl is selected from the followings: 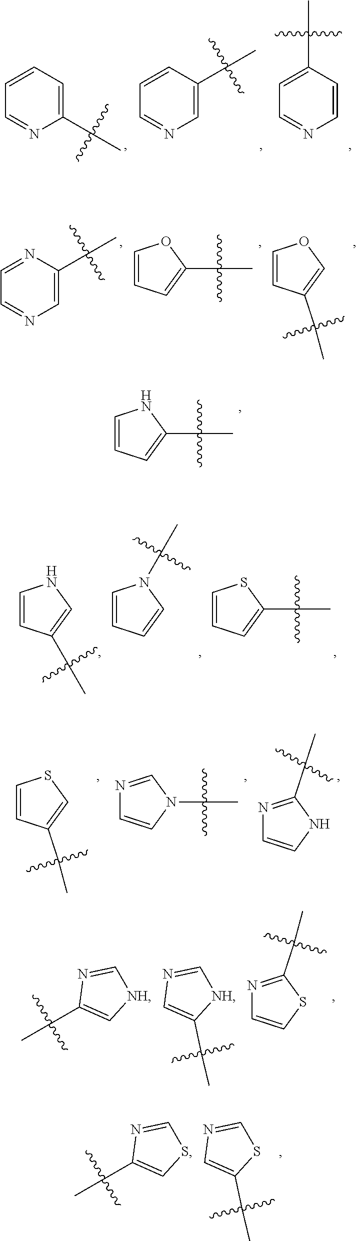
each heteroaryl is independently optionally substituted by one or more (e.g., 2, 3, 4, 5 or 6, preferably 2) R″s;
preferably, the heteroaryl is selected from the followings: 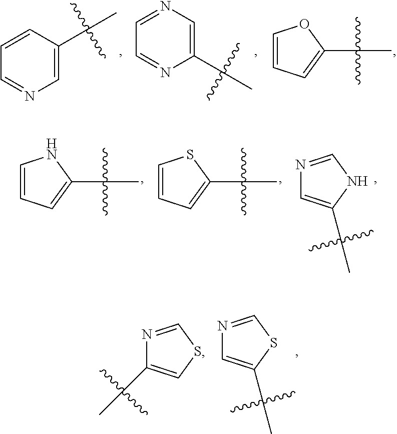
each heteroaryl is independently optionally substituted by one or more (e.g., 2, 3, 4, 5 or 6, preferably 2) R″s;
more preferably, the heteroaryl is selected from the followings: 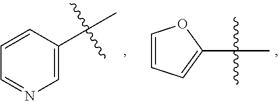 [SMILES: *c1cccnc1 ; *c1ccco1]
each heteroaryl is independently optionally substituted by one or more (e.g., 2, 3, 4, 5 or 6, preferably 2) R″s;
each X is independently selected from O, NH, S;
preferably, X is O;
each Y is independently selected from a direct bond, O, NH, S, CH, CH₂, C(CH₃)₂;
preferably, each Y is independently selected from a direct bond or CH₂;
R⁰ is selected from —N₃, —SS—C₁-C₆ alkyl (e.g., —SS-methyl, —SS-ethyl, —SS-isopropyl, —SS-tertbutyl), —ONH₂, —OCOR^{m}, —OCONHR^{m}, wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl);
preferably, R⁰ is —N₃ or —SS—C₁-C₆ alkyl (e.g., —SS-methyl, —SS-ethyl, —SS-isopropyl, —SS-tertbutyl);
more preferably, R⁰ is —N₃;
R¹ is selected from —N₃, —SS—C₁-C₆ alkyl (e.g., —SS-methyl, —SS-ethyl, —SS-isopropyl, —SS-tertbutyl), —ONH₂, —OCOR^{m}, —OCONHR^{m}, wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl);
preferably, R¹ is —N₃ or —SS—C₁-C₆ alkyl (e.g., —SS-methyl, —SS-ethyl, —SS-isopropyl, —SS-tertbutyl);
more preferably, R¹ is —N₃;
R², R³, and R⁴ are each independently selected from O, NH, S, CH, CH₂, C(CH₃)₂;
preferably, R², R³, and R⁴ are each independently selected from O, NH, S, CH, CH₂, C(CH₃)₂, and meet the following conditions: when R² is selected from O or S, R³ and R⁴ are CH₂; when R³ is selected from O or S, R² and R⁴ are CH₂; when R⁴ is selected from O or S, R² and R³ are CH₂; when R² is C(CH₃)₂, R³ and R⁴ are CH₂; when R³ is C(CH₃)₂, R² and R⁴ are CH₂; when R⁴ is C(CH₃)₂, R² and R³ are CH₂; when Y is CH, R² is CH, and R³ and R⁴ are CH₂;
more preferably, R², R³, and R⁴ are each independently selected from CH, CH₂;
most preferably, R² and R³ are CH, and R⁴ is CH₂; [symbol]
represents a single bond [symbol] or a double bond [symbol] ;
each R″ is independently selected from H, —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy), 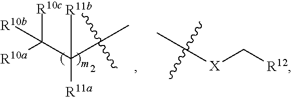
and is not H at the same time;
preferably, when each heteroaryl is independently optionally substituted by one R″, each R″ is independently selected from 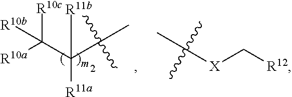
preferably, R″ is 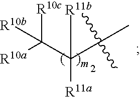
or preferably, when each heteroaryl is independently optionally substituted by more (e.g., 2, 3, 4, 5 or 6, preferably 2) R″s, one of the R″ is selected from 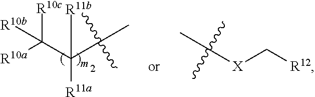
and the remaining R″(s) is(are) each independently selected from —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy);
or more preferably, when each heteroaryl is independently optionally substituted by more (e.g., 2, 3, 4, 5 or 6, preferably 2) R″s, one of the R″ is 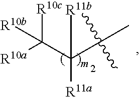
and the remaining R″(s) is(are) each independently selected from nitro, aliphatic alkyl (e.g., C₁-C₆ alkyl), F, I, Br, Cl;
or most preferably, when each heteroaryl is independently optionally substituted by more (e.g., 2, 3, 4, 5 or 6, preferably 2) R″s, one of the R″ is 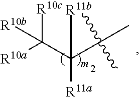
and the remaining R″(s) is(are) each independently selected from nitro, C₁-C₆ alkyl, preferably, the remaining R″(s) is(are) C₁-C₆ alkyl;
R⁵, R⁶, R⁷, R⁸, R⁹, RN, R^{y}, and R^{z} are each independently selected from H, —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy), C₁-C₆ alkyl-C(═O)—NH₂—, 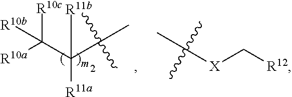
and R⁵, R⁶, R⁷, R⁸, and R⁹ are not H at the same time;
preferably, any one of R⁵, R⁶, R⁷, R⁸, and R⁹ is selected from 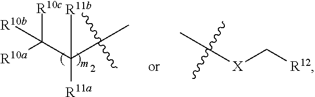
and the remaining four thereof are each independently selected from H, —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy), C₁-C₆ alkyl-C(═O)—NH₂—;
more preferably, any one of R⁵, R⁶, R⁷, R⁸, and R⁹ is 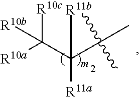
and the remaining four thereof are each independently selected from H, nitro, C₁-C₆ alkyl, C₁-C₆ alkoxy, C₁-C₆ alkyl-C(═O)—NH₂—;
further preferably, of R⁵, R⁶, R⁷, R⁸, and R⁹, R⁵ or R⁹ is 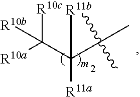
and the remaining four thereof are each independently selected from H, nitro, methoxy, acetamido;
most preferably, of R⁵, R⁶, R⁷, R⁸, and R⁹, R⁵ or R⁹ is 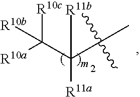
R⁷ is selected from H, nitro, methoxy, acetamido, and the remaining three thereof are H;
preferably, any one of R^{x} and R^{y} is selected from 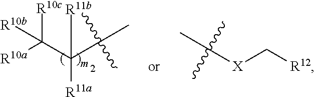
and the other is selected from H, —N₃, nitro, amino, sulfo, carboxyl, aliphatic alkyl (e.g., C₁-C₆ alkyl), cycloalkyl (e.g., C₃-C₆ cycloalkyl), aromatic alkyl (e.g., phenyl C₁-C₆ alkyl), F, I, Br, Cl, alkoxy (e.g., C₁-C₆ alkoxy);
more preferably, any one of R^{x} and R^{y} is selected from 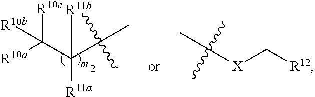
and the other is H;
most preferably, any one of R^{x} and R^{y} is 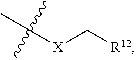
and the other is H;
preferably, R^{z} is selected from 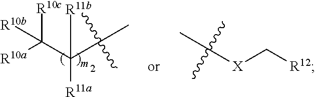
more preferably, R^{z} is 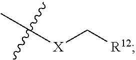
R^{10a}, R^{10b}, R^{10c}, R^{11a}, R^{11b}, and R¹² are each independently selected from H, —N₃, —SS—C₁-C₆ alkyl (e.g., —SS-methyl, —SS-ethyl, —SS-isopropyl, —SS-tertbutyl or —SS-isobutyl), —ONH₂, —OCOR^{m}, —OCONHR^{m}, aliphatic alkyl (e.g., C₁-C₆ alkyl, particularly such as methyl, ethyl, isopropyl, tertbutyl), aromatic alkyl (e.g., phenyl C₁-C₆ alkyl), cycloalkyl (e.g., C₃-C₆ cycloalkyl), wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl), and R^{10a}, R^{10b}, and R^{10c} are not H at the same time;
preferably, any one of R^{10a}, R^{10b}, and R^{10c} is selected from —N₃, —SS—C₁-C₆ alkyl (e.g., —SS-methyl, —SS-ethyl, —SS-isopropyl, —SS-tertbutyl or —SS-isobutyl), —ONH₂, —OCOR^{m}, —OCONHR^{m}, and the other two are each independently selected from H, aliphatic alkyl (e.g., C₁-C₆ alkyl, particularly such as methyl, ethyl, isopropyl, tertbutyl), aromatic alkyl (e.g., phenyl C₁-C₆ alkyl), cycloalkyl (e.g., C₃-C₆ cycloalkyl), wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl);
more preferably, any one of R^{10a}, R^{10b}, and R^{10c} is —N₃ or —SS—C₁-C₆ alkyl (e.g., —SS-methyl, —SS-ethyl, —SS-isopropyl, —SS-tertbutyl or —SS-isobutyl), and the other two are each independently selected from H, aliphatic alkyl (e.g., C₁-C₆ alkyl, particularly such as methyl, ethyl, isopropyl, tertbutyl);
most preferably, any one of R^{10a}, R^{10b}, and R^{10c} is —N₃, —SS-methyl, —SS-ethyl, —SS-isopropyl, —SS-tertbutyl or —SS-isobutyl, and the other two are each independently selected from H or methyl;
preferably, R^{11a} and R^{11b} are each independently selected from H or aliphatic alkyl (e.g., C₁-C₆ alkyl, particularly such as methyl, ethyl, isopropyl, tertbutyl);
more preferably, R^{11a} and R^{11b} are H;
preferably, R¹² is selected from —N₃, —SS—C₁-C₆ alkyl (e.g., —SS-methyl, —SS-ethyl, —SS-isopropyl, —SS-tertbutyl), —ONH₂, —OCOR^{m}, —OCONHR^{m}, wherein each R^{m} is independently selected from aliphatic alkyl (e.g., C₁-C₆ alkyl), cycloalkyl (e.g., C₃-C₆ cycloalkyl) or aromatic alkyl (e.g., phenyl C₁-C₆ alkyl);
more preferably, R¹² is —N₃ or —SS—C₁-C₆ alkyl (e.g., —SS-methyl, —SS-ethyl, —SS-isopropyl, —SS-tertbutyl);
most preferably, R¹² is —N₃;
n is selected from 1, 2, 3, 4, or 5; preferably, n is selected from 1, 2, or 3; more preferably, n is 2;
each m₁ is independently selected from 1, 2, 3, 4, 5, and 6; preferably, m₁ is 1;
each m₂ is independently selected from 0, 1, 2, 3, 4, 5, and 6; preferably, each m₂ is independently selected from 0 or 1;
most preferably, 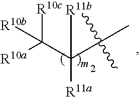
as a whole, is selected from 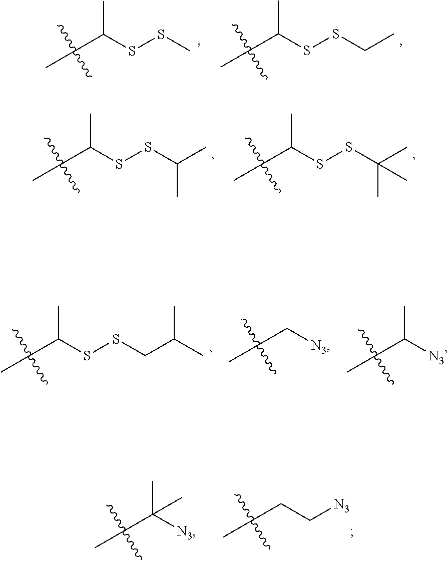
R′ is selected from H, monophosphate group 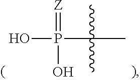
diphosphate group 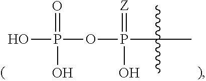
triphosphate group 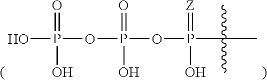
or tetraphosphate group 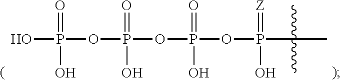
preferably, R′ is a triphosphate group 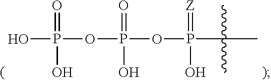
each Z is independently selected from O, S, BH; preferably, Z is O;
Base is selected from a base, a deaza base or a tautomer thereof, for example, Base is selected from adenine, 7-deazaadenine, thymine, uracil, cytosine, guanine, 7-deazaguanine or a tautomer thereof;
preferably, Base is selected from 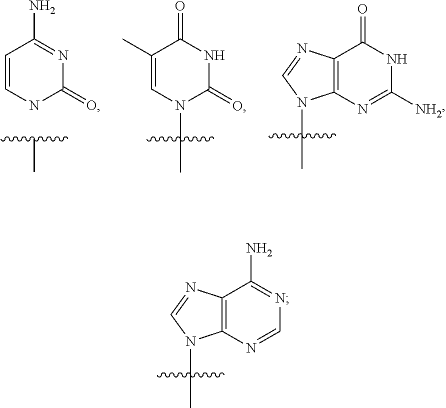
(b) carrying out a reaction comprising the following steps (i), (ii) and (iii), optionally, repeating the steps for one or more times:
step (i): incorporating the compound or a salt thereof into the growing nucleic acid strand using a polymerase to form a nucleic acid intermediate containing the reversible blocking group and the detectable label;
step (ii): detecting the detectable label contained in the nucleic acid intermediate;
step (iii): removing the reversible blocking group and/or the detectable label contained in the nucleic acid intermediate by using the excision reagent;
preferably, the removal of the reversible blocking group and the removal of the detectable label are performed simultaneously, or, the removal of the reversible blocking group and the removal of the detectable label are performed sequentially (for example, the reversible blocking group is removed first, or the detectable label is removed first);
preferably, the excision reagent used for the removal of the reversible blocking group is the same as that used for the removal of the detectable label;
preferably, the excision reagent used for the removal of the reversible blocking group is different from that used for the removal of the detectable label.

12. The method according to claim 11, wherein the duplex is linked to a support;
preferably, the growing nucleic acid strand is a primer;
preferably, the primer is annealed to the nucleic acid strand to be sequenced to form the duplex;
preferably, the duplex, the compound or a salt thereof, and the polymerase together form a reaction system containing a solution phase and a solid phase;
preferably, the Bases contained in the compounds or salts thereof are different from each other;
preferably, the additional detectable label carried by the compound or a salt thereof are different from each other;
preferably, the compound or a salt thereof is incorporated into the growing nucleic acid strand using a polymerase under conditions where the polymerase is allowed to carry out a nucleotide polymerization reaction, thereby forming a nucleic acid intermediate containing reversible blocking group and the detectable label;
preferably, the polymerase is selected from KOD polymerase or its mutants thereof (e.g., KOD POL151, KOD POL157, KOD POL171, KOD POL174, KOD POL376, KOD POL391);
preferably, before any step of detecting the detectable label contained in the nucleic acid intermediate, the solution phase of the reaction system in the previous step is removed, and the duplex linked to the support is retained;
preferably, the excision reagent is in contact with the duplex or the growing nucleic acid strand in the reaction system containing a solution phase and a solid phase;
preferably, the excision reagent can remove the reversible blocking group and the additional detectable label carried by the compound that is incorporated into the growing nucleic acid strand, without affecting the phosphodiester bond on the backbone of the duplex;
preferably, after any step of removing the reversible blocking group and/or additional detectable label contained in the nucleic acid intermediate, the solution phase of the reaction system in this step is removed;
preferably, a washing operation is performed after any step comprising a removal operation;
preferably, after step (ii), the method further comprises: determining the type of compound incorporated into the growing nucleic acid strand in step (i) according to the signal detected in step (ii), and determining the type of nucleotide at a corresponding position in the nucleic acid strand to be sequenced based on the principle of base complementary pairing.

13. A kit comprising at least one compound or a salt thereof according to claim 1;
preferably, the kit comprises a first compound, a second compound, a third compound and a fourth compound, the first, second, third and fourth compounds are each independently the compound or a salt thereof according to claim 1;
preferably, in the first compound, the Base is selected from adenine, 7-deazaadenine or a tautomer thereof (e.g., 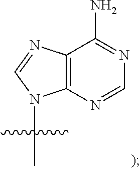 [SMILES: *n1cnc2c(N)ncnc21]
in the second compound, the Base is selected from thymine, uracil or a tautomer thereof (e.g., 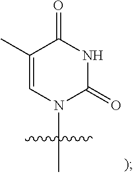 [SMILES: *n1cc(C)c(=O)[nH]c1=O]
in the third compound, the Base is selected from cytosine or a tautomer thereof (e.g., 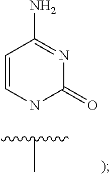
in the fourth compound, the Base is selected from guanine, 7-deazaguanine or a tautomer thereof (e.g., 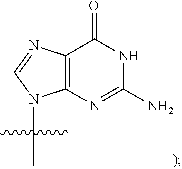 [SMILES: *n1cnc2c(=O)[nH]c(N)nc21]
preferably, the first, second, third and fourth compounds carry additional detectable labels;
preferably, the additional detectable labels carried by the first, second, third and fourth compounds are introduced by an affinity reagent (e.g., antibody, aptamer, Affimer, Knottin), wherein the affinity reagent carries the detectable label, and the affinity reagent can specifically recognize and bind to the epitope of the first, second, third or fourth compound;
preferably, the first, second, third and fourth compounds are linked to the additional detectable label optionally via a linker;
preferably, the Base of the first, second, third or fourth compound is linked to the additional detectable label optionally via a linker;
preferably, the Bases contained in the first, second, third and fourth compounds are different from each other;
preferably, the additional detectable labels carried by the first, second, third and fourth compounds are different from each other;
preferably, the linker is a cleavable linker or a non-cleavable linker;
preferably, the cleavable linker is selected from the group consisting of a linker capable of being cleaved by electrophilic reaction, a linker capable of being cleaved by nucleophilic reaction, a linker capable of being cleaved by photolysis, a linker capable of being cleaved under reductive conditions, a linker capable of being cleaved under oxidative conditions, a safety-catch linker, a linker capable of being cleaved by elimination mechanisms, or any combination thereof;
preferably, the additional detectable label (e.g., a fluorescent label) is linked to the Base of the compound or a salt thereof optionally via a linker;
preferably, the detectable label is selected from the followings:

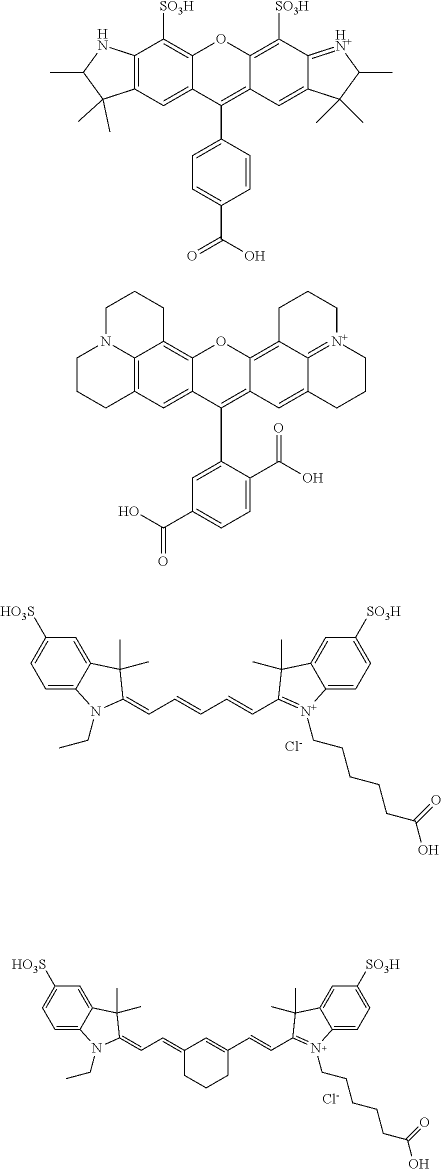

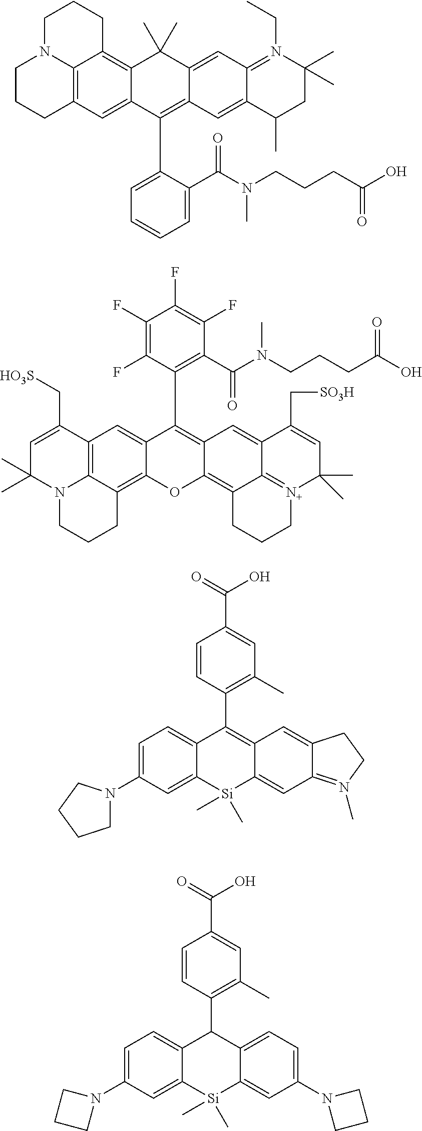

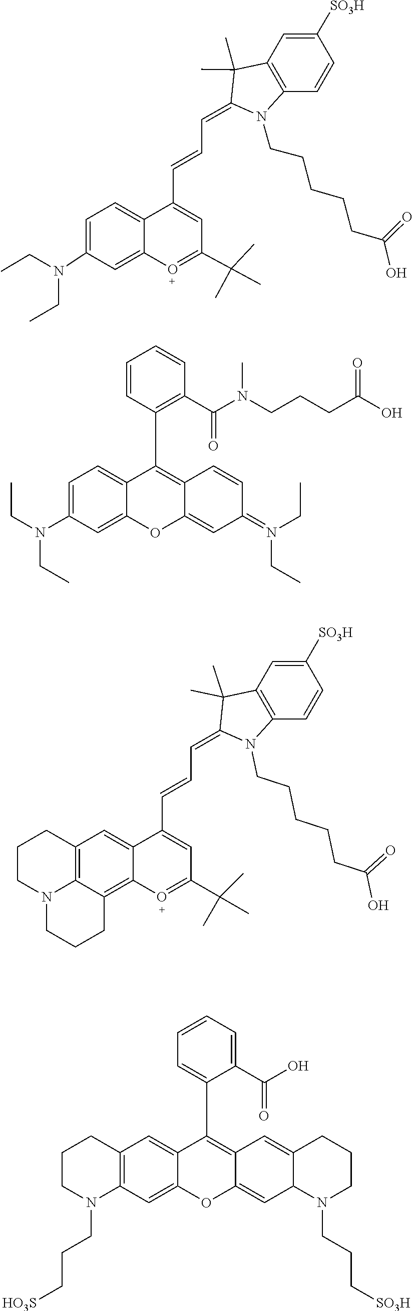

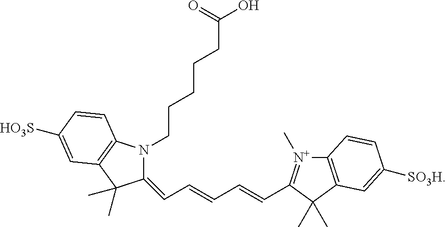 [SMILES: C[N+]1=C(/C=C/C=C/C=C2\N(CCCCCC(=O)O)c3ccc(S(=O)(=O)O)cc3C2(C)C)C(C)(C)c2cc(S(=O)(=O)O)ccc21]

14. The kit according to claim 13, wherein the kit further comprises an reagent for pretreating the nucleic acid molecule; a support for linking nucleic acid molecule to be sequenced; a reagent for linking the nucleic acid molecule to be sequenced to the support (for example, covalently or non-covalently linking); a primer for initiating a nucleotide polymerization reaction; a polymerase for carrying out the nucleotide polymerization reaction; one or more buffer solutions; one or more washing solutions; or any combination thereof.

15. Use of the compound or a salt thereof according to claim 1 for determining the sequence of a target single-stranded polynucleotide.

16. The compound or a salt thereof according to claim 4, which carries an additional detectable label (e.g., a fluorescent label);
preferably, the additional detectable label carried by the compound or a salt thereof is introduced by an affinity reagent (e.g., antibody, aptamer, Affimer, Knottin), wherein the affinity reagent carries the detectable label, and the affinity reagent can specifically recognize and bind to an epitope of the compound or a salt thereof;
preferably, the additional detectable label (e.g., a fluorescent label) is linked to the compound or a salt thereof optionally via a linker;
preferably, the additional detectable label (e.g., a fluorescent label) is linked to the Base of the compound or a salt thereof optionally via a linker;
preferably, the linker is a cleavable linker or a non-cleavable linker;
preferably, the cleavable linker is selected from the group consisting of a linker capable of being cleaved by electrophilic reaction, a linker capable of being cleaved by nucleophilic reaction, a linker capable of being cleaved by photolysis, a linker capable of being cleaved under reductive conditions, a linker capable of being cleaved under oxidative conditions, a safety-catch linker, a linker capable of being cleaved by elimination mechanisms, or any combination thereof;
preferably, the detectable label is selected from the followings: 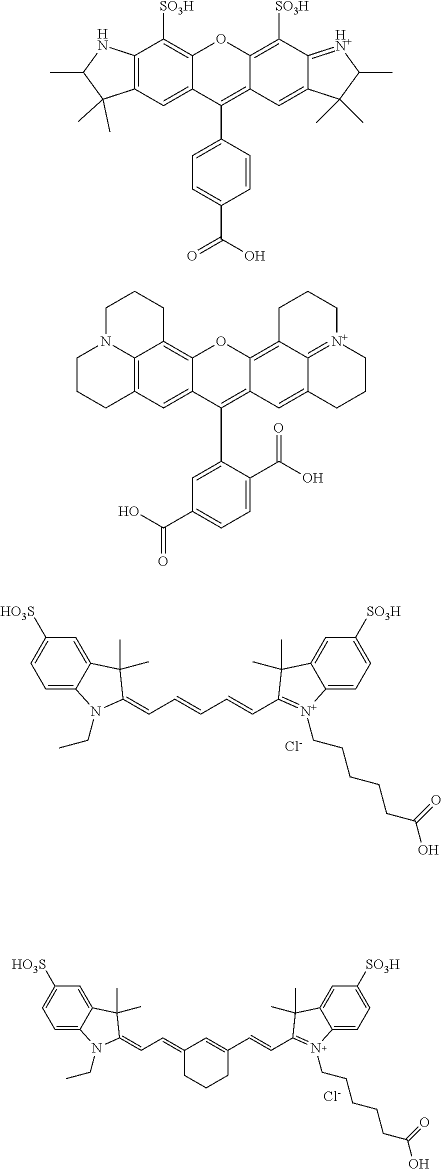 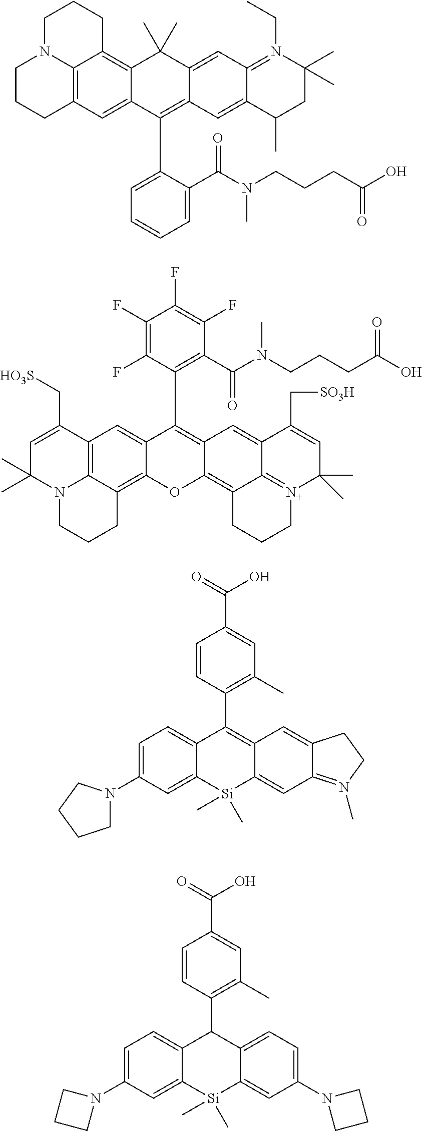 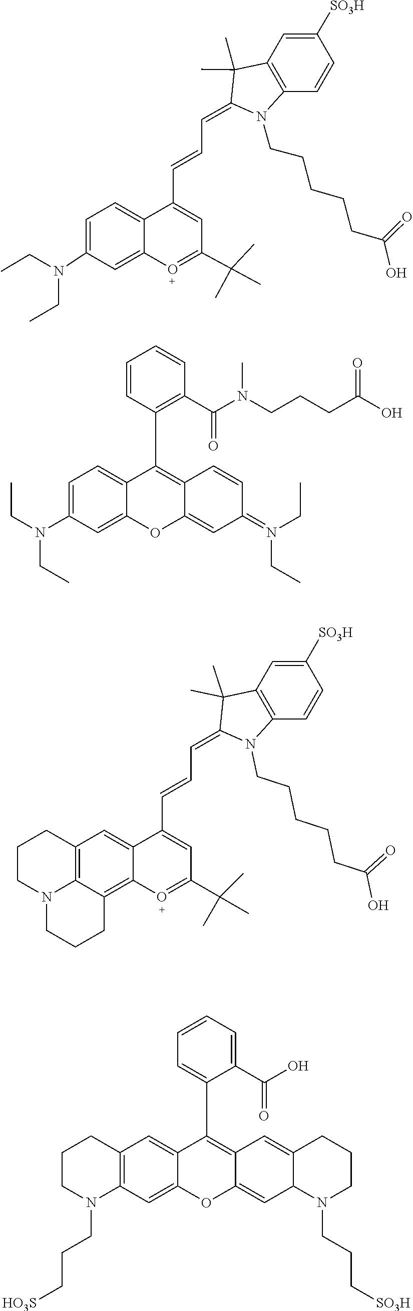 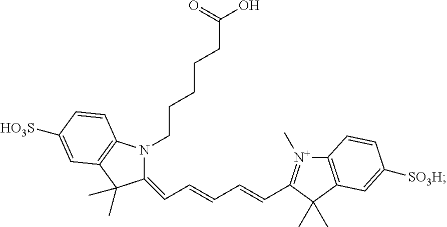 [SMILES: C[N+]1=C(/C=C/C=C/C=C2\N(CCCCCC(=O)O)c3ccc(S(=O)(=O)O)cc3C2(C)C)C(C)(C)c2cc(S(=O)(=O)O)ccc21]
preferably, if Base is different, the detectable label (e.g., a fluorescent label) varies.

17. A kit comprising at least one compound or a salt thereof according to claim 4;
preferably, the kit comprises a first compound, a second compound, a third compound and a fourth compound, the first, second, third and fourth compounds are each independently the compound or a salt thereof according to claim 4;
preferably, in the first compound, the Base is selected from adenine, 7-deazaadenine or a tautomer thereof (e.g., 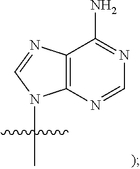 [SMILES: *n1cnc2c(N)ncnc21]
in the second compound, the Base is selected from thymine, uracil or a tautomer thereof (e.g., 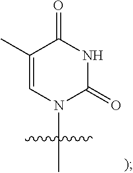 [SMILES: *n1cc(C)c(=O)[nH]c1=O]
in the third compound, the Base is selected from cytosine or a tautomer thereof (e.g., 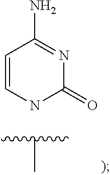
in the fourth compound, the Base is selected from guanine, 7-deazaguanine or a tautomer thereof (e.g., 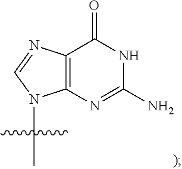 [SMILES: *n1cnc2c(=O)[nH]c(N)nc21]
preferably, the first, second, third and fourth compounds carry additional detectable labels;
preferably, the additional detectable labels carried by the first, second, third and fourth compounds are introduced by an affinity reagent (e.g., antibody, aptamer, Affimer, Knottin), wherein the affinity reagent carries the detectable label, and the affinity reagent can specifically recognize and bind to the epitope of the first, second, third or fourth compound;
preferably, the first, second, third and fourth compounds are linked to the additional detectable label optionally via a linker;
preferably, the Base of the first, second, third or fourth compound is linked to the additional detectable label optionally via a linker;
preferably, the Bases contained in the first, second, third and fourth compounds are different from each other;
preferably, the additional detectable labels carried by the first, second, third and fourth compounds are different from each other;
preferably, the linker is a cleavable linker or a non-cleavable linker;
preferably, the cleavable linker is selected from the group consisting of a linker capable of being cleaved by electrophilic reaction, a linker capable of being cleaved by nucleophilic reaction, a linker capable of being cleaved by photolysis, a linker capable of being cleaved under reductive conditions, a linker capable of being cleaved under oxidative conditions, a safety-catch linker, a linker capable of being cleaved by elimination mechanisms, or any combination thereof;
preferably, the additional detectable label (e.g., a fluorescent label) is linked to the Base of the compound or a salt thereof optionally via a linker;
preferably, the detectable label is selected from the followings:

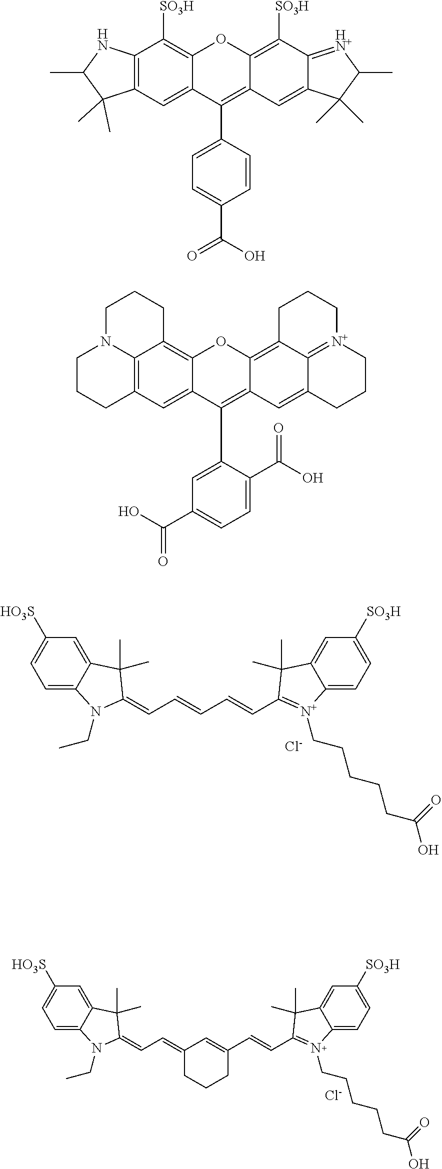

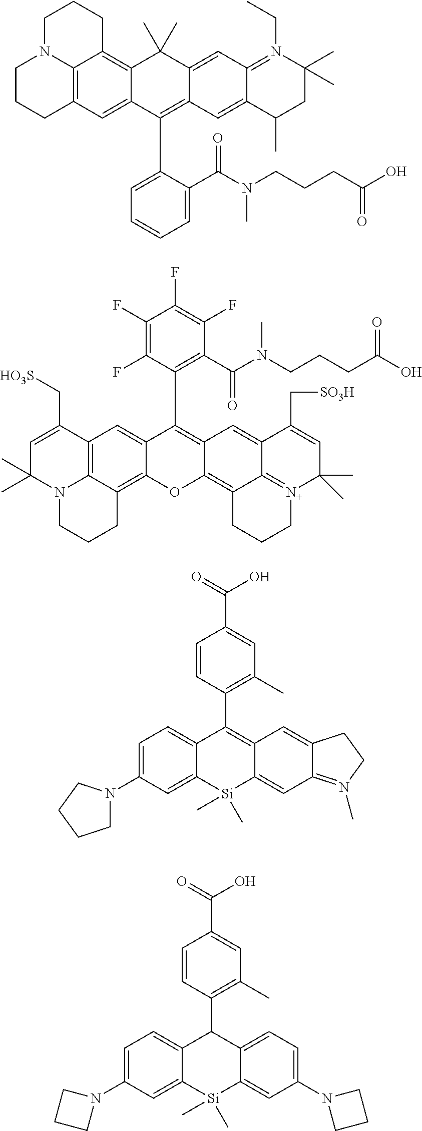

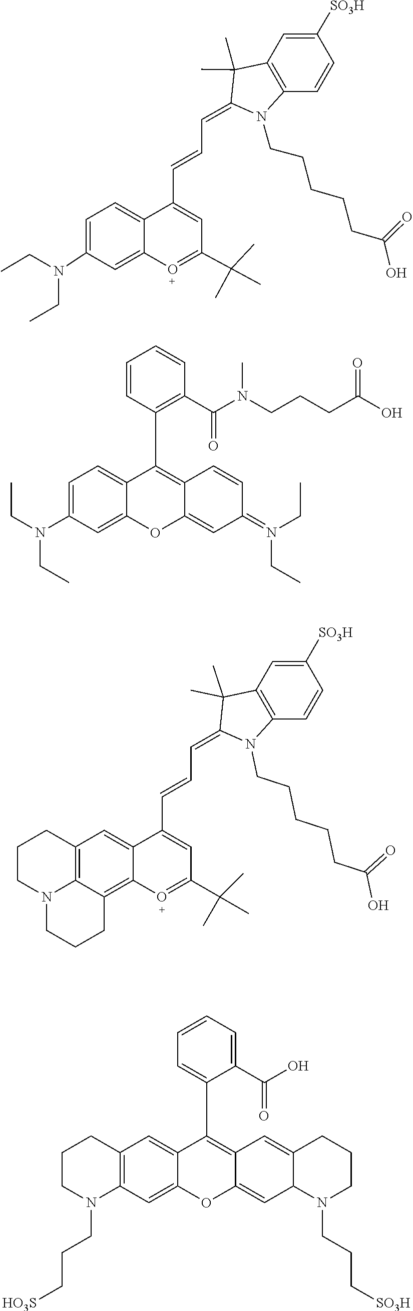

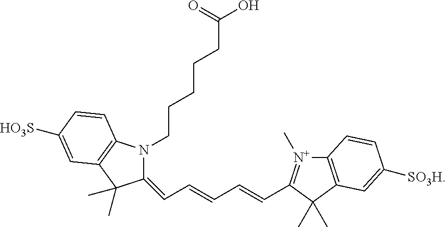 [SMILES: C[N+]1=C(/C=C/C=C/C=C2\N(CCCCCC(=O)O)c3ccc(S(=O)(=O)O)cc3C2(C)C)C(C)(C)c2cc(S(=O)(=O)O)ccc21]

18. The kit according to claim 17, wherein the kit further comprises an reagent for pretreating the nucleic acid molecule; a support for linking nucleic acid molecule to be sequenced; a reagent for linking the nucleic acid molecule to be sequenced to the support (for example, covalently or non-covalently linking); a primer for initiating a nucleotide polymerization reaction; a polymerase for carrying out the nucleotide polymerization reaction; one or more buffer solutions; one or more washing solutions; or any combination thereof.

19. Use of the compound or a salt thereof according to claim 4 for determining the sequence of a target single-stranded polynucleotide.
